SEQUENCE LISTING (72 sequences)

SEQ ID NO: 1 - DNA, 4664 bp, Homo sapiens
  misc_feature 1..4664: note = NM_004448.3 (2014-05-26)
  source 1..4664: mol_type = unassigned DNA
gcttgctcccaatcacaggagaaggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaaaaaaaaaaaaaaaaaa

SEQ ID NO: 2 - protein, 1255 aa, Homo sapiens [sequence omitted]

SEQ ID NO: 3 - DNA, 3780 bp, Canis lupus
  misc_feature 1..3780: note = NM_001003217.1 (2014-02-23)
  source 1..3780: mol_type = unassigned DNA; note = Canine
atggagctggcggcctggtgccgctgggggctccttctcgccctcctgccctccggagccgcgggcacccaagtgtgcaccggcacagacatgaagctccggctcccggccagtcccgagacccacctggatatgctccgccacctgtaccagggctgtcaagtggtacaggggaacctggagctcacttacctgcctgccaatgccagcctgtccttcctgcaggatatccaggaggtgcagggctatgtgctcattgctcacagccaagtgaggcagatcccactgcagaggctacgaattgtgcgaggcacccagctctttgaggacaactacgccctggccgtgctggacaatggagacccgctggagggtggcatccctgcaccaggggcggcccaaggagggctgcgggagctgcagcttcgaagcctcacagagatcctgaagggaggggtcttgattcagcggagcccgcagctctgccaccaggacacgattttatggaaggacgtcttccataagaacaaccagctggccctcacgctgatagacaccaaccgcttttcggcctgcccgccctgttctccagcttgtaaagacgcccactgctggggggccagctccggggactgtcagagcttgacgcggactgtctgtgccgggggctgtgcccgctgcaagggcccacaacccaccgactgctgccacgagcagtgtgctgctggctgcacgggccccaagcactctgactgcctggcctgccttcacttcaaccacagtggcatctgtgagctgcactgcccagccctggtcacctacaacacggacaccttcgaatccatgcccaaccctgagggccgatataccttcggggccagctgtgtgacctcctgtccctacaactacctgtctacggatgtgggatcctgcaccctggtctgtcccctgaacaaccaagaggtgacggctgaggatgggacacagcggtgcgagaaatgcagcaagccctgtgcccgagtgtgctacggtctgggcatggagcacctgcgagaggtgagagcggtcaccagtgcgaacatccaggagtttgccggctgcaagaagatctttggaagcctggcatttttgccagagagctttgatggggacccagcctccaacactgcccccctacagcctgagcagctcagagtgtttgaggctctggaggagatcacaggttacctgtacatctcagcgtggccagacagcctgcctaacctcagtgtcttccagaacctgcgagtaatccggggacgagttctgcatgatggtgcctactcgctgaccctgcaagggctgggcatcagctggctggggctgcgctcgctgcgggaactgggcagtgggctggccctcatccaccgcaacgcccgcctttgcttcgtgcacacggtgccctgggaccagctcttccggaacccccaccaggccctgctccatagtgccaaccggccagaggaggagtgcgtgggcgagggcctggcctgctacccctgtgcccatgggcactgctggggtccagggcccacccagtgcgtcaactgcagccaattcctccggggccaggagtgcgtggaggaatgccgagtactgcaggggctgccccgagagtatgtgaaggacaggtactgtctaccgtgccactcagagtgtcagccccagaatggctcagtgacctgtttcggatcggaggctgaccagtgtgtggcctgcgcccactacaaggaccctcccttctgtgtggctcgctgccccagtggtgtgaaacctgacctgtccttcatgcccatctggaagttcgcagatgaggagggcacttgccagccgtgccccatcaactgcacccactcctgtgcggacctggacgagaagggctgtcccgccgagcagagagccagccctgtgacatccatcattgccgctgtggtgggcattctgctggctgtggtcgtggggctggtcctcggcatcctgatcaagcgaaggcggcagaagatccggaagtacactatgcggaggctgctgcaggaaaccgagctggtggagccgctgacgcctagtggagcgatgcccaaccaggctcagatgcggatcctgaaagagacagagctgaggaaggtgaaggtgcttggatccggagcttttggcacagtctacaagggcatctggatccctgatggggaaaatgtgaaaatcccagtggccatcaaagtgttgagggaaaacacatctcccaaagccaacaaagaaatcttggacgaagcatatgtgatggctggagtgggctccccgtatgtgtcccgcctcctgggcatctgcctgacatccacggtgcagctggtgacacagcttatgccctacggctgcctcttagaccatgtccgagaacaccgtgggcgcctgggctcccaggacttgctgaactggtgtgtgcagattgccaaggggatgagctacttggaggatgtccggctggtgcacagggacctggctgcccggaatgtgctggtcaagagtcccaaccatgtcaagattacagatttcgggctggctcggttgctggacatcgacgagacagagtaccatgcggatgggggcaaggtgcccatcaagtggatggcgctggagtccattcctccgcggcggttcacccaccagagtgatgtgtggagctatggtgtgactgtgtgggaactgatgacttttggggccaaaccttatgatgggatcccagcccgggagatccctgacctgctggagaagggggaacggctgccccagccccccatctgcaccattgatgtctacatgatcatggtcaagtgctggatgatagactctgaatgccgaccccggttccgggagttggtggccgaattctcacgtatggccagggacccccagcgctttgtggtcattcagaatgaagacttgggccccgccagccccttggacagcaccttctaccgttcactactggaagatgatgacatgggggacctggtggatgctgaggagtacctggtaccccagcagggtttcttctgcccagaacctaccccaggggctgggggcactgcccaccgacggcaccgcagctcatccaccaggaatggcggtggtgagctgactctaggactggagccctccgaggaggagccccccaagtctccactggcaccctcagagggcgctggctctgacgtgtttgatggtgacttgggaatgggggcagccaaggggctgcagagccttccctcacaggaccccagccctctccagcggtacagtgaggaccctacggtacccttgccccctgagactgatggtaaggttgcccccctgacctgcagcccccagcctgaatatgtgaaccagccagaagtttggccgcagcccccccttgccctagaaggccctttgcctccttcccgaccggctggtgccactctggaaaggcccaagactctgtcccccaagactctctcccctggcaagaatggggttgtcaaagacgtttttgcctttgggagtgctgtggagaatccggagtacctggcaccccggggcagagctgcccctcagccccaccctcctccagccttcagcccagcctttgacaacctgtattactgggaccaggatccatcagagcggggctctccacccagcacctttgaagggacccctacagcagagaacccggagtacctggggctggacgtgccagtgtga

SEQ ID NO: 4 - protein, 1259 aa, Canis lupus
  REGION 1..1259: note = NP_001003217.1 (2014-02-23)
  source 1..1259: note = Canine
MELAAWCRWGLLLALLPSGAAGTQVCTGTDMKLRLPASPETHLDMLRHLYQGCQVVQGNLELTYLPANASLSFLQDIQEVQGYVLIAHSQVRQIPLQRLRIVRGTQLFEDNYALAVLDNGDPLEGGIPAPGAAQGGLRELQLRSLTEILKGGVLIQRSPQLCHQDTILWKDVFHKNNQLALTLIDTNRFSACPPCSPACKDAHCWGASSGDCQSLTRTVCAGGCARCKGPQPTDCCHEQCAAGCTGPKHSDCLACLHFNHSGICELHCPALVTYNTDTFESMPNPEGRYTFGASCVTSCPYNYLSTDVGSCTLVCPLNNQEVTAEDGTQRCEKCSKPCARVCYGLGMEHLREVRAVTSANIQEFAGCKKIFGSLAFLPESFDGDPASNTAPLQPEQLRVFEALEEITGYLYISAWPDSLPNLSVFQNLRVIRGRVLHDGAYSLTLQGLGISWLGLRSLRELGSGLALIHRNARLCFVHTVPWDQLFRNPHQALLHSANRPEEECVGEGLACYPCAHGHCWGPGPTQCVNCSQFLRGQECVEECRVLQGLPREYVKDRYCLPCHSECQPQNGSVTCFGSEADQCVACAHYKDPPFCVARCPSGVKPDLSFMPIWKFADEEGTCQPCPINCTHSCADLDEKGCPAEQRASPVTSIIAAVVGILLAVVVGLVLGILIKRRRQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVREHRGRLGSQDLLNWCVQIAKGMSYLEDVRLVHRDLAARNVLVKSPNHVKITDFGLARLLDIDETEYHADGGKVPIKWMALESIPPRRFTHQSDVWSYGVTVWELMTFGAKPYDGIPAREIPDLLEKGERLPQPPICTIDVYMIMVKCWMIDSECRPRFRELVAEFSRMARDPQRFVVIQNEDLGPASPLDSTFYRSLLEDDDMGDLVDAEEYLVPQQGFFCPEPTPGAGGTAHRRHRSSSTRNGGGELTLGLEPSEEEPPKSPLAPSEGAGSDVFDGDLGMGAAKGLQSLPSQDPSPLQRYSEDPTVPLPPETDGKVAPLTCSPQPEYVNQPEVWPQPPLALEGPLPPSRPAGATLERPKTLSPKTLSPGKNGVVKDVFAFGSAVENPEYLAPRGRAAPQPHPPPAFSPAFDNLYYWDQDPSERGSPPSTFEGTPTAENPEYLGLDVPV

SEQ ID NO: 5 - DNA, 1311 bp, Homo sapiens
  misc_feature 1..1311: note = NM_000073.2 (2014-05-03)
  source 1..1311: mol_type = unassigned DNA
agtctagctgctgcacaggctggctggctggctggctgctaagggctgctccacgcttttgccggaggacagagactgacatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattgaactcaggactcagagtagtccaggtgttctcctcctattcagttcccagaatcaaagcaatgcattttggaaagctcctagcagagagactttcagccctaaatctagactcaaggttcccagagatgacaaatggagaagaaaggccatcagagcaaatttgggggtttctcaaataaaataaaaataaaaacaaatactgtgtttcagaagcgccacctattggggaaaattgtaaaagaaaaatgaaaagatcaaataaccccctggatttgaatataattttttgtgttgtaatttttatttcgtttttgtataggttataattcacatggctcaaatattcagtgaaagctctccctccaccgccatcccctgctacccagtgaccctgttgccctcttcagagacaaattagtttctcttttttttttttttttttttttttttgagacagtctggctctgtcacccaggctgaaatgcagtggcaccatctcggctcactgcaacctctgcctcctgggttcaagcgattctcctgcctcagcctcccgggcagctgggattacaggcacacactaccacacctggctaatttttgtatttttagtagagacagggttttgctctgttggccaagctggtctcgaactcctgacctcaagtgatccgcccgcctc

SEQ ID NO: 6 - protein, 182 aa, Homo sapiens
  REGION 1..182: note = NP_000064.1 (2014-05-03)
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 7 - DNA, 771 bp, Homo sapiens
  misc_feature 1..771: note = NM_000732.4 (2014-05-03)
  source 1..771: mol_type = unassigned DNA
agagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagaaaaaaaaaaaaaaaaaaaaaaaaaaaaaa

SEQ ID NO: 8 - protein, 171 aa, Homo sapiens
  REGION 1..171: note = NP_000723.1 (2014-05-03)
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 9 - DNA, 1534 bp, Homo sapiens
  misc_feature 1..1534: note = NM_000733.3 (2014-05-03)
  source 1..1534: mol_type = unassigned DNA
tattgtcagagtcctcttgtttggccttctaggaaggctgtgggacccagctttcttcaaccagtccaggtggaggcctctgccttgaacgtttccaagtgaggtaaaacccgcaggcccagaggcctctctacttcctgtgtggggttcagaaaccctcctcccctcccagcctcaggtgcctgcttcagaaaatgaagtagtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaccccccctccactgtaggccactggatggtcatttgcatctccgtaaatgtgctctgctcctcagctgagagagaaaaaaataaactgtatttggctgcaagaaaaaaaaaaaaaaaaaaaaaa

SEQ ID NO: 10 - protein, 207 aa, Homo sapiens
  REGION 1..207: note = NP_000724.1 (2014-05-03)
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 11 - DNA, 1436 bp, Canis lupus
  misc_feature 1..1436: note = NM_001003379.1 (2014-02-22)
  source 1..1436: mol_type = unassigned DNA; note = Canine
ataaacgttagttactatttttatcaggactcctgggacccctatctcactaatttccttaaagacagtataatacagcagctcacacagactttcggattcagaaaaacagttgtgtcgggccttgggtaaattatgtaaggcaagcctcagtttgctcagcggtaaaacgaggaaagtaataagccacccgcctccgccattttgggtagaataagggtgcatccagtgagagaaggatgcagtcgaggaacctctggagaattctgggactctgtctcttatcagttggtgcttgggggcaggacgaggatttcaaagcttctgatgacttgacaagtatatctccagagaaacggtttaaggtctccatctctggaaccgaggtagtggtgacatgccctgatgtttttggatatgataatataaaatgggaaaaaaatgataaccttgtggaaggtgctagtaacagagagctatctcagaaggagttttcagaagtggacgacagtggttattatgcctgctatgcagattccataaaggagaagagctatctctacctgagagcaagagtgtgtgcaaactgcatagaggtgaatctgatggcagtggtcacaatcattgtagctgacatctgccttactctggggttgctgctgatggtgtattactggagcaagactagaaaggccaatgccaagcctgtgatgagaggaacaggtgccggcagcaggcccaggggacaaaacaaggagaagccaccacctgttcccaatccagactacgagcccatccggaaaggccagcaggacctgtattctggcctgaatcagagaggcatctgacggctcctgaggacacggcctccccagggcccaggtcttggtgtctccaggtcctgctactcccagtaccctgggtaaatcttgaaccccagaagagaattattcctctgccttctggagaactaactcccagcctgcagccttatccccagcaccctccaacccgccttctctgctggcacttggtcctgcaatatcacctcctcatcatggccactcaccgcccccccaccagccagactgccctctggtcggggtatttatttctgttaccctgacgcccccaccatcaccaattccttcctacccttcagaggtatccttgctcccttccgtacccctctcccggacagaacctgcccccatcccttactatcccacctacctttccgtttttccagctctctctttggtgaccctctgtggggatggactaggtaactctggtagaggtcctgccccatccatgaccttggcccagagccaccctctgccagcaggcccctggatgatcatttgcattcttacaaatgtgctaggctccttgacagctagagagaaaataataaagtgtatttggttgaaaaa

SEQ ID NO: 12 - protein, 202 aa, Canis lupus
  REGION 1..202: note = NP_001003379.1 (2014-02-22)
  source 1..202: note = Canine
MQSRNLWRILGLCLLSVGAWGQDEDFKASDDLTSISPEKRFKVSISGTEVVVTCPDVFGYDNIKWEKNDNLVEGASNRELSQKEFSEVDDSGYYACYADSIKEKSYLYLRARVCANCIEVNLMAVVTIIVADICLTLGLLLMVYYWSKTRKANAKPVMRGTGAGSRPRGQNKEKPPPVPNPDYEPIRKGQQDLYSGLNQRGI

SEQ ID NO: 13 - DNA, 45 bp, synthetic construct
  misc_feature 1..45: note = Peptide linker (G4S)3
  source 1..45: mol_type = other DNA
ggcggcggaggatctggcggaggtggaagtgggggaggcggatct

SEQ ID NO: 14 - protein, 15 aa, synthetic construct
  REGION 1..15: note = Peptide linker (G4S)3
GGGGSGGGGSGGGGS

SEQ ID NO: 15 - protein, 119 aa, synthetic construct
  REGION 1..119: note = OKT3 VH
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSS

SEQ ID NO: 16 - protein, 107 aa, synthetic construct
  REGION 1..107: note = OKT3 VL
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 17 - protein, 119 aa, synthetic construct
  REGION 1..119: note = OKT3 VH C105S
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSS

SEQ ID NO: 18 - DNA, 723 bp, synthetic construct
  misc_feature 1..723: note = OKT3 scFv C105S
  source 1..723: mol_type = other DNA
caggtgcagctggtgcagagtggtggcggagtggtgcagcctggcagatccctgagactgtcttgcaaggccagcggctacaccttcacccggtacaccatgcactgggtgcgacaggcccctggcaagggcctggaatggatcggctacatcaacccctcccggggctacaccaactacaaccagaagttcaaggaccggttcaccatcagccgggacaactccaagaacaccgcctttctgcagatggactccctgcggcctgaggataccggcgtgtacttttgcgcccggtactacgacgaccactactctctggactactggggccagggaacccctgtgacagtgtctagcggagggggaggttcaggtggcggtggatcagggggcggaggctctgatatccagatgacccagtccccctccagcctgtctgcctctgtgggagacagagtgacaattacatgctccgccagctccagcgtgtcctacatgaattggtatcagcagacccctggcaaggctcccaagcggtggatctacgacacctccaagctggcctccggcgtgccctccagattttctggcagcggctccggcacagactatacctttacaatcagctccctgcagcccgaagatatcgccacctactactgccagcagtggtcctccaaccccttcacctttggccaggggacaaaactgcagatcaccaga

SEQ ID NO: 19 - protein, 241 aa, synthetic construct
  REGION 1..241: note = OKT3 scFv C105S
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 20 - DNA, 1350 bp, synthetic construct
  misc_feature 1..1350: note = exemplary heavy chain N297A
  source 1..1350: mol_type = other DNA
gaggtgcagctggtggaatctggcggaggactggtgcagcctggcggctctctgagactgtcttgtgccgcctccggcttcaacatcaaggacacctacatccactgggtgcgacaggcccctggcaagggactggaatgggtggccagaatctaccccaccaacggctacaccagatacgccgactctgtgaagggccggttcaccatctccgccgacacctccaagaacaccgcctacctgcagatgaactccctgcgggccgaggacaccgccgtgtactactgtagtagatggggaggcgacggcttctacgccatggactattggggccagggcaccctcgtgaccgtgtcctctgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcaaagtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaa

SEQ ID NO: 21 - DNA, 1350 bp, synthetic construct
  misc_feature 1..1350: note = exemplary heavy chain K322A
  source 1..1350: mol_type = other DNA
gaggtgcagctggtggaatctggcggaggactggtgcagcctggcggctctctgagactgtcttgtgccgcctccggcttcaacatcaaggacacctacatccactgggtgcgacaggcccctggcaagggactggaatgggtggccagaatctaccccaccaacggctacaccagatacgccgactctgtgaagggccggttcaccatctccgccgacacctccaagaacaccgcctacctgcagatgaactccctgcgggccgaggacaccgccgtgtactactgtagtagatggggaggcgacggcttctacgccatggactattggggccagggcaccctcgtgaccgtgtcctctgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaa

SEQ ID NO: 22 - DNA, 1350 bp, synthetic construct
  misc_feature 1..1350: note = exemplary heavy chain
  source 1..1350: mol_type = other DNA
gaggtgcagctggtggaatctggcggaggactggtgcagcctggcggctctctgagactgtcttgtgccgcctccggcttcaacatcaaggacacctacatccactgggtgcgacaggcccctggcaagggactggaatgggtggccagaatctaccccaccaacggctacaccagatacgccgactctgtgaagggccggttcaccatctccgccgacacctccaagaacaccgcctacctgcagatgaactccctgcgggccgaggacaccgccgtgtactactgtagtagatggggaggcgacggcttctacgccatggactattggggccagggcaccctcgtgaccgtgtcctctgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgacagtgctgcaccaggactggctgaacggcaaagagtacaagtgcaaagtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaa

SEQ ID NO: 23 - protein, 450 aa, synthetic construct
  REGION 1..450: note = exemplary heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 642 bp, synthetic construct
  misc_feature 1..642: note = HER2-BsAb light chain
  source 1..642: mol_type = other DNA
gacatccagatgacccagagcccttccagcctgtccgcctctgtgggcgacagagtgaccatcacctgtcgggcctcccaggacgtgaacaccgccgtggcttggtatcagcagaagcccggcaaggcccccaagctgctgatctactccgcctccttcctgtactccggcgtgccctccagattctccggcagcagatctggcaccgactttaccctgaccatctccagcctgcagcccgaggacttcgccacctactactgccagcagcactacaccaccccccccacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgc

SEQ ID NO: 25 - protein, 216 aa, synthetic construct
  REGION 1..216: note = HER2-BsAb light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTS

SEQ ID NO: 26 - DNA, 1350 bp, synthetic construct
  misc_feature 1..1350: note = HER2-BsAb heavy chain
  source 1..1350: mol_type = other DNA
gaggtgcagctggtggaatctggcggaggactggtgcagcctggcggctctctgagactgtcttgtgccgcctccggcttcaacatcaaggacacctacatccactgggtgcgacaggcccctggcaagggactggaatgggtggccagaatctaccccaccaacggctacaccagatacgccgactctgtgaagggccggttcaccatctccgccgacacctccaagaacaccgcctacctgcagatgaactccctgcgggccgaggacaccgccgtgtactactgtagtagatggggaggcgacggcttctacgccatggactattggggccagggcaccctcgtgaccgtgtcctctgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaa

SEQ ID NO: 27 - protein, 450 aa, synthetic construct
  REGION 1..450: note = HER2-BsAb heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1416 bp, synthetic construct
  misc_feature 1..1416: note = HER2-BsAb light chain fusion polypeptide
  source 1..1416: mol_type = other DNA
gacatccagatgacccagagcccttccagcctgtccgcctctgtgggcgacagagtgaccatcacctgtcgggcctcccaggacgtgaacaccgccgtggcttggtatcagcagaagcccggcaaggcccccaagctgctgatctactccgcctccttcctgtactccggcgtgccctccagattctccggcagcagatctggcaccgactttaccctgaccatctccagcctgcagcccgaggacttcgccacctactactgccagcagcactacaccaccccccccacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgcactagtggcggcggaggatctggcggaggtggaagtgggggaggcggatctcaggtgcagctggtgcagagtggtggcggagtggtgcagcctggcagatccctgagactgtcttgcaaggccagcggctacaccttcacccggtacaccatgcactgggtgcgacaggcccctggcaagggcctggaatggatcggctacatcaacccctcccggggctacaccaactacaaccagaagttcaaggaccggttcaccatcagccgggacaactccaagaacaccgcctttctgcagatggactccctgcggcctgaggataccggcgtgtacttttgcgcccggtactacgacgaccactactctctggactactggggccagggaacccctgtgacagtgtctagcggagggggaggttcaggtggcggtggatcagggggcggaggctctgatatccagatgacccagtccccctccagcctgtctgcctctgtgggagacagagtgacaattacatgctccgccagctccagcgtgtcctacatgaattggtatcagcagacccctggcaaggctcccaagcggtggatctacgacacctccaagctggcctccggcgtgccctccagattttctggcagcggctccggcacagactatacctttacaatcagctccctgcagcccgaagatatcgccacctactactgccagcagtggtcctccaaccccttcacctttggccaggggacaaaactgcagatcaccaga

SEQ ID NO: 29 - protein, 472 aa, synthetic construct
  REGION 1..472: note = HER2-BsAb light chain fusion polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 30 - protein, 462 aa, synthetic construct
  REGION 1..462: note = HER2-BsAb light chain fusion polypeptide (5aa scFv linker)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 31 - protein, 467 aa, synthetic construct
  REGION 1..467: note = HER2-BsAb light chain fusion polypeptide (10aa scFv linker)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 32 - protein, 477 aa, synthetic construct
  REGION 1..477: note = HER2-BsAb light chain fusion polypeptide (20aa scFv linker)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 33 - protein, 482 aa, synthetic construct
  REGION 1..482: note = HER2-BsAb light chain fusion polypeptide (25aa scFv linker)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 34 - protein, 487 aa, synthetic construct
  REGION 1..487: note = HER2-BsAb light chain fusion polypeptide (30aa scFv linker)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 35 - protein, 17 aa, synthetic construct
  REGION 1..17: note = T(G4S)3 Linker
TSGGGGSGGGGSGGGGS

SEQ ID NO: 36 - protein, 5 aa, synthetic construct
  REGION 1..5: note = G4S Linker
GGGGS

SEQ ID NO: 37 - protein, 10 aa, synthetic construct
  REGION 1..10: note = (G4S)2 Linker
GGGGSGGGGS

SEQ ID NO: 38 - protein, 15 aa, synthetic construct
  REGION 1..15: note = (G4S)3 Linker
GGGGSGGGGSGGGGS

SEQ ID NO: 39 - protein, 20 aa, synthetic construct
  REGION 1..20: note = (G4S)4 Linker
GGGGSGGGGSGGGGSGGGGS

SEQ ID NO: 40 - protein, 25 aa, synthetic construct
  REGION 1..25: note = (G4S)5 Linker
GGGGSGGGGSGGGGSGGGGSGGGGS

SEQ ID NO: 41 - protein, 30 aa, synthetic construct
  REGION 1..30: note = (G4S)6 Linker
GGGGSGGGGSGGGGSGGGGSGGGGSGGGGS

SEQ ID NO: 42 - protein, 462 aa, synthetic construct
  REGION 1..462: note = HER2-BsAb light chain fusion polypeptide (5aa scFv linker) + disulfide mut
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 43 - protein, 467 aa, synthetic construct
  REGION 1..467: note = HER2-BsAb light chain fusion polypeptide (10aa scFv linker) + disulfide mut
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 44 - protein, 472 aa, synthetic construct
  REGION 1..472: note = HER2-BsAb light chain fusion polypeptide (15aa scFv linker) + disulfide mut
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 45 - protein, 477 aa, synthetic construct
  REGION 1..477: note = HER2-BsAb light chain fusion polypeptide (20aa scFv linker) + disulfide mut
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 46 - protein, 483 aa, synthetic construct
  REGION 1..483: note = HER2-BsAb light chain fusion polypeptide (25aa scFv linker) + disulfide mut
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQCGTKLQITR

SEQ ID NO: 47 - protein, 487 aa, synthetic construct
  REGION 1..487: note = HER2-BsAb light chain fusion polypeptide (30aa scFv linker) + disulfide mut
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 48 - protein, 231 aa, synthetic construct
  REGION 1..231: note = huOKT3scFv(C105S) (5 aa scFv linker)
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 49 - protein, 236 aa, synthetic construct
  REGION 1..236: note = huOKT3scFv(C105S) (10 aa scFv linker)
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 50 - protein, 246 aa, synthetic construct
  REGION 1..246: note = huOKT3scFv(C105S) (20 aa scFv linker)
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 51 - protein, 251 aa, synthetic construct
  REGION 1..251: note = huOKT3scFv(C105S) (25 aa scFv linker)
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 52 - protein, 256 aa, synthetic construct
  REGION 1..256: note = huOKT3scFv(C105S) (30 aa scFv linker)
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 53 - protein, 231 aa, synthetic construct
  REGION 1..231: note = huOKT3scFv(C105S) + disulfide mut (5 aa scFv linker)
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 54 - protein, 236 aa, synthetic construct
  REGION 1..236: note = huOKT3scFv(C105S) + disulfide mut (10 aa scFv linker)
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 55 - protein, 241 aa, synthetic construct
  REGION 1..241: note = huOKT3scFv(C105S) + disulfide mut (15 aa scFv linker)
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 56 - protein, 246 aa, synthetic construct
  REGION 1..246: note = huOKT3scFv(C105S) + disulfide mut (20 aa scFv linker)
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 57 - protein, 252 aa, synthetic construct
  REGION 1..252: note = huOKT3scFv(C105S) + disulfide mut (25 aa scFv linker)
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQCGTKLQITR

SEQ ID NO: 58 - protein, 256 aa, synthetic construct
  REGION 1..256: note = huOKT3scFv(C105S) + disulfide mut (30 aa scFv linker)
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 59 - protein, 241 aa, synthetic construct
  REGION 1..241: note = huOKT3scFv + disulfide mut (15 aa scFv linker)
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 60 - protein, 472 aa, synthetic construct
  REGION 1..472: note = Her2-BsAb light chain fusion polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 61 - protein, 214 aa, synthetic construct
  REGION 1..214: note = Her2-bsab light chain sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 450 aa, synthetic construct
  REGION 1..450: note = her2 bsab heavy chain sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 450 aa, synthetic construct
  REGION 1..450: note = Trastuzumab heavy chain K322A
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 119 aa, synthetic construct
  REGION 1..119: note = huOKT3 VH C105S + VH44
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSS

SEQ ID NO: 65 - protein, 107 aa, synthetic construct
  REGION 1..107: note = huOKT3 VL + VL100
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 66 - protein, 241 aa, synthetic construct; identical to SEQ ID NO: 59
  REGION 1..241: note = huOKT3 scFv; 15 aa linker

SEQ ID NO: 67 - DNA, 1416 bp, synthetic construct
  misc_feature 1..1416: note = DNA encoding sequence of SEQ ID NO:60
  source 1..1416: mol_type = other DNA
gatattcagatgactcagtctccctcttccctgtccgcttcagtcggcgatcgggtcactattacttgtcgggcttcacaggatgtcaacacagccgtggcttggtaccagcagaagcccgggaaagcacctaagctgctgatctactctgccagtttcctgtattctggcgtcccaagtaggttttcaggctcccggagcggaactgacttcaccctgacaatttccagcctgcagcccgaggattttgctacctactattgccagcagcattatactacccccccaacattcggccagggcacaaaagtcgaaatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgcactagtggaggaggaggtagcggaggaggaggttctggcggagggggttcccaggtgcagctggtgcagagcggaggaggagtggtgcagccaggaaggagcctgcgactgtcttgcaaggctagtggctacaccttcacacgatatactatgcactgggtgaggcaggcacctggtaaaggcctggagtggatcggctacattaacccctctaggggatacaccaactataatcagaagttcaaagacaggttcaccatctcacgcgataactccaagaataccgccttcctgcagatggactccctgcggcccgaagatacaggcgtgtatttttgcgctagatactatgacgatcattactgtctggactattggggacaggggacccctgtgacagtgtccagcggtggaggagggtcaggtggaggagggagcggtggcggagggtctgacatccagatgacccagtccccatctagtctgagcgcctctgtgggcgatagagtgactattacctgcagtgcttcatccagcgtgagctacatgaactggtatcagcagacacccggaaaggcacctaaacgctggatctacgatactagcaagctggcctctggcgtgcccagtcgattcagtggttcaggctccggaaccgactataccttcaccatctctagtctgcagcctgaggatattgccacatactattgtcagcagtggtcatccaatccattcacttttgggcagggtaccaaactgcagattacaagg

SEQ ID NO: 68 - DNA, 1350 bp, synthetic construct
  misc_feature 1..1350: note = DNA encoding sequence of SEQ ID NO:62
  source 1..1350: mol_type = other DNA
gaagtgcagctggtcgagagcggaggaggtctggtgcagcccggaggttccctgagactgtcctgtgccgcatctgggtttaatatcaaggacacatacatccactgggtgagacaggcacccggcaaaggactggagtgggtcgccaggatctaccctaccaacgggtacacaagatatgctgactctgtgaagggccggttcaccatctccgccgatactagcaaaaacaccgcttacctgcagatgaattccctgagggcagaagataccgctgtctactactgttcaagatgggggggggatggtttttacgctatggattattggggccagggcaccctggtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 69 - DNA, 1410 bp, synthetic construct
  misc_feature 1..1410: note = HER2-C825 Light Chain
  source 1..1410: mol_type = other DNA
gatattcagatgactcagtctccctcttccctgtccgcttcagtcggcgatcgggtcactattacttgtcgggcttcacaggatgtcaacacagccgtggcttggtaccagcagaagcccgggaaagcacctaagctgctgatctactctgccagtttcctgtattctggcgtcccaagtaggttttcaggctcccggagcggaactgacttcaccctgacaatttccagcctgcagcccgaggattttgctacctactattgccagcagcattatactacccccccaacattcggccagggcacaaaagtcgaaatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgcggtggtggtggtagcggcggcggtggaagcgcatcccatgtgaaactgcaggaaagcggcccaggtctggtccagccatcccagtctctgagcctgacatgcactgtgagcggattctctctgacagactatggggtgcactgggtcagacagagtccaggaaaggggctggagtggctgggcgtcatctggtcaggcggagggactgcttataacaccgcactgatcagcagactgaatatctaccgcgacaactctaaaaatcaggtgttcctggagatgaacagtctgcaggccgaagataccgctatgtactattgcgccaggcggggcagctacccttataattactttgacgcttggggttgtggcaccacagtgacagtctccagcggtggaggagggagtggtggaggagggtcaggtggaggagggtcccaggcagtggtcattcaggagtctgccctgactaccccccctggagaaaccgtgacactgacttgcggatctagtacaggggcagtgactgcctccaactatgcaaattgggtccaggaaaagcctgatcactgtttcactggcctgatcggtggccataacaatcgaccacccggagtgccagctaggttttcaggttccctgatcggcgacaaagccgctctgaccattgctggcacccagacagaggatgaagcaatctacttttgtgccctgtggtattccgatcactgggtcattgggggggggacacgtctgactgtgctgggg

SEQ ID NO: 70 - DNA, 1350 bp, synthetic construct; identical to SEQ ID NO: 68
  misc_feature 1..1350: note = HER2-C825 Heavy Chain
  source 1..1350: mol_type = other DNA

SEQ ID NO: 71 - protein, 470 aa, synthetic construct
  REGION 1..470: note = HER2-C825 Light Chain sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSASHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLG

SEQ ID NO: 72 - protein, 450 aa, synthetic construct; identical to SEQ ID NO: 62
  REGION 1..450: note = HER2-C825 Heavy Chain sequence

(19) United States
(12) Patent Application Publication
(10) Pub. No.: US 2026/0062501 A1
(43) Pub. Date: Mar. 5, 2026

(54) BISPECIFIC HER2 AND CD3 BINDING MOLECULES

(71) Applicant: MEMORIAL SLOAN KETTERING CANCER CENTER, New York, NY (US)

(72) Inventors: Nai-Kong V. CHEUNG, New York, NY (US); Andres LOPEZ-ALBAITERO, New York, NY (US); Hong XU, New York, NY (US)

(73) Assignee: MEMORIAL SLOAN KETTERING CANCER CENTER, New York, NY (US)

(21) Appl. No.: 19/094,615

(22) Filed: Mar. 28, 2025

Related U.S. Application Data
(63) Continuation of application No. 18/790,952, filed on Jul. 31, 2024, now abandoned.
Application No. 18/790,952 is a continuation of application No. 18/511,880, filed on Nov. 16, 2023, now abandoned.
Application No. 18/511,880 is a continuation of application No. 16/481,446, now Pat. No. 11,820,832.
Application No. 16/481,446 is a continuation-in-part of application No. PCT/US2015/041989, filed on Jul. 24, 2015.
(60) Provisional application No. 62/029,342, filed on Jul. 25, 2014.

(51) Int. Cl. C07K 16/46; A61K 39/00; A61K 39/395; A61K 40/10; A61K 40/42; A61K 45/06; A61P 35/00; A61P 35/02; A61P 35/04; C07K 16/28; C07K 16/32

(52) U.S. Cl. CPC C07K 16/468; A61K 39/3955; A61K 39/39558; A61K 40/10; A61K 40/4204; A61K 40/4205; A61K 45/06; A61P 35/00; A61P 35/02; A61P 35/04; C07K 16/2809; C07K 16/32; A61K 2039/505; A61K 2239/31; A61K 2239/38; A61K 2239/49; A61K 2239/59; C07K 2317/31; C07K 2317/35; C07K 2317/40; C07K 2317/622; C07K 2317/732; C07K 2317/76; C07K 2317/92

(57) ABSTRACT

Provided herein are compositions, methods, and uses involving bispecific binding molecules that specifically bind to HER2, a receptor tyrosine kinase, and to CD3, a T cell receptor, and mediate T cell cytotoxicity for managing and treating disorders, such as cancer. Also provided herein are uses and methods for managing and treating HER2-related cancers.

### CROSS REFERENCE TO RELATED APPLICATIONS

This application is a continuation of U.S. application Ser. No. 18/790,952, filed Jul. 31, 2024, which is a continuation of U.S. application Ser. No. 18/511,880, filed Nov. 16, 2023, which is a continuation of U.S. application Ser. No. 16/481,446, filed Jul. 26, 2019, now U.S. Pat. No. 11,820,832, which is the U.S. National Stage Application of International Application No. PCT/US2017/015278, filed Jan. 27, 2017, which is a continuation-in-part of International Patent Application No. PCT/US2015/041989, filed Jul. 24, 2015, which claims the benefit of U.S. Provisional Application No. 62/029,342, filed Jul. 25, 2014, each of which is incorporated by reference herein in its entirety.

### SEQUENCE LISTING

The instant application contains a Sequence Listing which has been submitted electronically in XML file format and is hereby incorporated by reference in its entirety. Said XML copy, created on Oct. 27, 2025, is named 115872-3240_SL.xml and is 135,934 bytes in size.

### 1. FIELD

Provided herein are compositions, methods, and uses involving bispecific binding molecules that specifically bind to HER2, a receptor tyrosine kinase, and to CD3, a T cell receptor, and mediate T cell cytotoxicity for managing and treating disorders, such as cancer.

### 2. BACKGROUND

HER2 is a receptor tyrosine kinase of the epidermal growth factor receptor family. Amplification or overexpression of HER2 has been demonstrated in the development and progression of cancers. Herceptin® (trastuzumab) is an anti-HER2 monoclonal antibody approved for treating HER2-positive metastatic breast cancer and HER2-positive gastric cancer (Trastuzumab [Highlights of Prescribing Information]. South San Francisco, CA: Genentech, Inc.; 2014). Ertumaxomab is a tri-specific HER2-CD3 antibody with intact Fc-receptor binding (see, for example, Kiewe et al. 2006, Clin Cancer Res, 12 (10): 3085-3091). Ertumaxomab is a rat-mouse antibody; therefore, upon administration to humans, a human anti-mouse antibody response and a human anti-rat antibody response are expected. 2502A, the parental antibody of ertumaxomab, has low affinity for HER2 and low avidity (Diermeier-Daucher et al., MAbs, 2012, 4 (5): 614-622). There is a need for therapies capable of mediating T cell cytotoxicity in HER2-positive cancers.

### 3. SUMMARY

In a specific embodiment, provided herein is a method of treating a HER2-positive cancer in a subject in need thereof, comprising administering to the subject a therapeutically effective amount of a bispecific binding molecule comprising an aglycosylated monoclonal antibody that is an immunoglobulin that binds to HER2, said immunoglobulin comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, and wherein the first and second light chain fusion polypeptides are identical, and wherein the cancer expresses a low level of HER2, and preferably wherein the cancer is not a head and neck cancer.

In a specific embodiment, the cancer is deemed to express a low level of HER2 when the cancer expresses a lower level of HER2 than the level of HER2 expressed by cancers that are indicated for treatment with trastuzumab and are of the same tissue type as the HER2-positive cancer.

In a specific embodiment, the cancer is deemed to express a low level of HER2 when the cancer has been determined not to overexpress HER2 based on the following characterization of the cancer: (a) a first determination of a level of HER2 in a test specimen comprising cells of the cancer is reported as negative, or (b) a first determination of a level of HER2 in a test specimen comprising cells of the cancer is reported as equivocal, and a second determination of a level of HER2 in a test specimen comprising cells of the cancer is reported as equivocal or negative. In a specific embodiment, the determination of the level of HER2 in the test specimen is reported as negative when the level of HER2 in the test specimen is characterized as (i) (1) immunohistochemistry (IHC) 1+, wherein the level of HER2 in the test specimen is characterized as IHC 1+ when the test specimen exhibits an incomplete HER2 membrane staining that is faint/barely perceptible and within greater than 10% of the invasive tumor cells, wherein the staining is readily appreciated using a low-power objective; (2) IHC 0, wherein the level of HER2 in the test specimen is characterized as IHC 0 when the test specimen exhibits no HER2 staining observed, wherein the lack of staining is readily appreciated using a low-power objective, or a HER2 membrane staining that is incomplete and is faint/barely perceptible and within less than or equal to 10% of the invasive tumor cells, wherein the staining is readily appreciated using a low-power objective; or (ii) in situ hybridization (ISH) negative, wherein the level of HER2 in the test specimen is characterized as ISH negative when the test specimen exhibits (1) a single-probe average HER2 copy number of less than 4.0 signals per cell; or (2) a dual-probe HER2/CEP17 ratio of less than 2.0 with an average HER2 copy number of less than 4.0 signals per cell. In a specific embodiment, the determination of the level of HER2 in the test specimen is reported as equivocal when the level of HER2 in the test specimen is characterized as: (i) IHC 2+, wherein the level of HER2 in the test specimen is characterized as IHC 2+ when the test specimen exhibits (1) a circumferential HER2 membrane staining that is incomplete and/or weak/moderate and within greater than 10% of invasive tumor cells, wherein the staining is observed in a homogenous and contiguous population, and wherein the staining is readily appreciated using a low-power objective; or (2) a complete and circumferential HER2 membrane staining that is intense and within less than or equal to 10% of invasive tumor cells, wherein the staining is readily appreciated using a low-power objective; or (ii) ISH equivocal, wherein the level of HER2 in the test specimen is characterized as ISH equivocal when the test specimen exhibits (1) a single-probe ISH average HER2 copy number of greater than or equal to 4.0 and less than 6.0 signals/cell, wherein the copy number is determined by counting at least 20 cells within the area and is observed in a homogenous and contiguous population; or (2) a dual-probe HER2/CEP17 ratio of less than 2.0 with an average HER2 copy number of greater than or equal to 4.0 and less than 6.0 signals per cell, wherein the copy number is determined by counting at least 20 cells within the area and is observed in a homogenous and contiguous population.

In a specific embodiment, the cancer is deemed to express a low level of HER2 when a level of HER2 in a test specimen comprising cells of the cancer is characterized as IHC 2+ or less according to applicable American Society of Clinical Oncology/College of American Pathologists guideline recommendations for human epidermal growth factor receptor 2 testing in cancer. In a preferred embodiment, the cancer is deemed to express a low level of HER2 when a level of HER2 in a test specimen comprising cells of the cancer is characterized as IHC 2+ or less according to applicable American Society of Clinical Oncology/College of American Pathologists guideline recommendations for human epidermal growth factor receptor 2 testing in breast cancer. In a specific embodiment, a level of HER2 in a test specimen comprising cells of the cancer is characterized as IHC 2+. In a specific embodiment, the level of HER2 in the test specimen is characterized as IHC 2+ when the test specimen exhibits (1) a circumferential HER2 membrane staining that is incomplete and/or weak/moderate and within greater than 10% of invasive tumor cells, wherein the staining is observed in a homogenous and contiguous population, and wherein the staining is readily appreciated using a low-power objective; or (2) a complete and circumferential HER2 membrane staining that is intense and within less than or equal to 10% of invasive tumor cells, wherein the staining is readily appreciated using a low-power objective. In a specific embodiment, a level of HER2 in a test specimen comprising cells of the cancer is characterized as IHC 1+. In a specific embodiment, the level of HER2 in the test specimen is characterized as IHC 1+ when the test specimen exhibits an incomplete HER2 membrane staining that is faint/barely perceptible and within greater than 10% of the invasive tumor cells, wherein the staining is readily appreciated using a low-power objective. In a specific embodiment, a level of HER2 in a test specimen comprising cells of the cancer is characterized as IHC 0. In a specific embodiment, the level of HER2 in the test specimen is characterized as IHC 0 when the test specimen exhibits no HER2 staining observed, wherein the lack of staining is readily appreciated using a low-power objective, or a HER2 membrane staining that is incomplete and is faint/barely perceptible and within less than or equal to 10% of the invasive tumor cells, wherein the staining is readily appreciated using a low-power objective.

In a specific embodiment, the HER2-positive cancer that expresses a low level of HER2 is a programmed death-ligand 1 (PDL1)-positive cancer. In a specific embodiment, the HER2-positive cancer overexpresses PDL1 relative to expression of PDL 1 in analogous noncancerous cells of the same tissue type as the cancer. In a specific embodiment, the HER2-positive cancer is deemed to overexpress PDL1 when a test specimen comprising cells of the cancer expresses a detectable level of PDL1 above background. In a specific embodiment, the cancer is resistant to PDL1 blockade with an anti-PDL1 therapy. In a specific embodiment, the anti-PDL 1 therapy is an anti-PDL1 antibody. In a specific embodiment, the anti-PDL1 antibody is atezolizumab. In a specific embodiment, the cancer is resistant to programmed cell death protein 1 (PD1) blockade with an anti-PD1 therapy. In a specific embodiment, the anti-PD1 therapy is an anti-PD1 antibody. In a specific embodiment, the anti-PD1 antibody is pembrolizumab.

In a specific embodiment, the HER2-positive cancer that expresses a low level of HER2 is breast cancer, gastric cancer, an osteosarcoma, desmoplastic small round cell cancer, ovarian cancer, prostate cancer, pancreatic cancer, glioblastoma multiforme, gastric junction adenocarcinoma, gastroesophageal junction adenocarcinoma, cervical cancer, salivary gland cancer, soft tissue sarcoma, leukemia, melanoma, Ewing's sarcoma, rhabdomyosarcoma, or neuroblastoma. In a specific embodiment, the cancer is gastric cancer or breast cancer. In a specific embodiment of a method described herein, the HER2-positive cancer that expresses a low level of HER2 is a metastatic tumor. In a specific embodiment, the metastatic tumor is a peritoneal metastasis. In a specific embodiment, the cancer is resistant to treatment with trastuzumab, cetuximab, lapatinib, erlotinib, or any other small molecule or antibody that targets the HER family of receptors.

In a specific embodiment, also provided herein is a method of treating a HER2-positive cancer in a subject in need thereof, comprising administering to the subject a therapeutically effective amount of a bispecific binding molecule comprising an aglycosylated monoclonal antibody that is an immunoglobulin that binds to HER2, said immunoglobulin comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, and wherein the first and second light chain fusion polypeptides are identical, and wherein the cancer is not indicated for treatment with trastuzumab, and preferably wherein the cancer is not a head and neck cancer. In a specific embodiment, the cancer is determined not to be indicated for treatment with trastuzumab based on the following characterization of the cancer: (a) a first determination of a level of HER2 in a test specimen comprising cells of the cancer is reported as negative, or (b) a first determination of a level of HER2 in a test specimen comprising cells of the cancer is reported as equivocal, and a second determination of a level of HER2 in a test specimen comprising cells of the cancer is reported as equivocal or negative. In a specific embodiment, the determination of the level of HER2 in the test specimen is reported as negative when the level of HER2 in the test specimen is characterized as (i) (1) immunohistochemistry (IHC) 1+, wherein the level of HER2 in the test specimen is characterized as IHC 1+ when the test specimen exhibits an incomplete HER2 membrane staining that is faint/barely perceptible and within greater than 10% of the invasive tumor cells, wherein the staining is readily appreciated using a low-power objective; (2) IHC 0, wherein the level of HER2 in the test specimen is characterized as IHC 0 when the test specimen exhibits no HER2 staining observed, wherein the lack of staining is readily appreciated using a low-power objective, or a HER2 membrane staining that is incomplete and is faint/barely perceptible and within less than or equal to 10% of the invasive tumor cells, wherein the staining is readily appreciated using a low-power objective; or (ii) in situ hybridization (ISH) negative, wherein the level of HER2 in the test specimen is characterized as ISH negative when the test specimen exhibits (1) a single-probe average HER2 copy number of less than 4.0 signals per cell; or (2) a dual-probe HER2/CEP17 ratio of less than 2.0 with an average HER2 copy number of less than 4.0 signals per cell. In a specific embodiment, the determination of the level of HER2 in the test specimen is reported as equivocal when the level of HER2 in the test specimen is characterized as: (i) IHC 2+, wherein the level of HER2 in the test specimen is characterized as IHC 2+ when the test specimen exhibits (1) a circumferential HER2 membrane staining that is incomplete and/or weak/moderate and within greater than 10% of invasive tumor cells, wherein the staining is observed in a homogenous and contiguous population, and wherein the staining is readily appreciated using a low-power objective; or (2) a complete and circumferential HER2 membrane staining that is intense and within less than or equal to 10% of invasive tumor cells, wherein the staining is readily appreciated using a low-power objective; or (ii) ISH equivocal, wherein the level of HER2 in the test specimen is characterized as ISH equivocal when the test specimen exhibits (1) a single-probe ISH average HER2 copy number of greater than or equal to 4.0 and less than 6.0 signals/cell, wherein the copy number is determined by counting at least 20 cells within the area and is observed in a homogenous and contiguous population; or (2) a dual-probe HER2/CEP17 ratio of less than 2.0 with an average HER2 copy number of greater than or equal to 4.0 and less than 6.0 signals per cell, wherein the copy number is determined by counting at least 20 cells within the area and is observed in a homogenous and contiguous population. In a specific embodiment, the HER2-positive cancer is a programmed death-ligand 1 (PDL1)-positive cancer. In a specific embodiment, the HER2-positive cancer overexpresses PDL1 relative to expression of PDL 1 in analogous noncancerous cells of the same tissue type as the cancer. In a specific embodiment, the HER2-positive cancer is deemed to overexpress PDL1 when a test specimen comprising cells of the cancer expresses a detectable level of PDL1 above background. In a specific embodiment, the cancer is resistant to PDL1 blockade with an anti-PDL1 therapy. In a specific embodiment, the anti-PDL1 therapy is an anti-PDL1 antibody. In a specific embodiment, the anti-PDL1 antibody is atezolizumab. In a specific embodiment, the cancer is resistant to programmed cell death protein 1 (PD1) blockade with an anti-PD1 therapy. In a specific embodiment, the anti-PD1 therapy is an anti-PD1 antibody. In a specific embodiment, the anti-PD1 antibody is pembrolizumab. In a specific embodiment, the HER2-positive that is not indicated for treatment with trastuzumab is breast cancer, gastric cancer, an osteosarcoma, desmoplastic small round cell cancer, ovarian cancer, prostate cancer, pancreatic cancer, glioblastoma multiforme, gastric junction adenocarcinoma, gastroesophageal junction adenocarcinoma, cervical cancer, salivary gland cancer, soft tissue sarcoma, leukemia, melanoma, Ewing's sarcoma, rhabdomyosarcoma, or neuroblastoma. In a specific embodiment, the HER2-positive that is not indicated for treatment with trastuzumab is gastric cancer or breast cancer. In a specific embodiment, the HER2-positive that is not indicated for treatment with trastuzumab is a metastatic tumor. In a specific embodiment, the metastatic tumor is a peritoneal metastasis. In a specific embodiment, the cancer is resistant to treatment with trastuzumab, cetuximab, lapatinib, erlotinib, or any other small molecule or antibody that targets the HER family of receptors.

Also provided herein is a method of treating a HER2-positive, PDL 1-positive cancer in a subject in need thereof, comprising administering to the subject a therapeutically effective amount of a bispecific binding molecule comprising an aglycosylated monoclonal antibody that is an immunoglobulin that binds to HER2, said immunoglobulin comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, and wherein the first and second light chain fusion polypeptides are identical, wherein the cancer is resistant to PDL1 blockade with an anti-PDL1 therapy and/or is resistant to PD1 blockade with an anti-PD1 therapy. In a specific embodiment, the HER2-positive cancer overexpresses PDL1 relative to expression of PDL 1 in analogous noncancerous cells of the same tissue type as the cancer. In a specific embodiment, the HER2-positive cancer is deemed to overexpress PDL1 when a test specimen comprising cells of the cancer expresses a detectable level of PDL1 above background. In a specific embodiment, the anti-PDL1 therapy is an anti-PDL1 antibody. In a specific embodiment, the anti-PDL1 antibody is atezolizumab. In a specific embodiment, the anti-PD1 therapy is an anti-PD1 antibody. In a specific embodiment, the anti-PD1 antibody is pembrolizumab. In a specific embodiment, the HER2-positive cancer is breast cancer, gastric cancer, an osteosarcoma, desmoplastic small round cell cancer, squamous cell carcinoma of head and neck cancer, ovarian cancer, prostate cancer, pancreatic cancer, glioblastoma multiforme, gastric junction adenocarcinoma, gastroesophageal junction adenocarcinoma, cervical cancer, salivary gland cancer, soft tissue sarcoma, leukemia, melanoma, Ewing's sarcoma, rhabdomyosarcoma, or neuroblastoma. In a specific embodiment, the HER2-positive cancer is a metastatic tumor. In a specific embodiment, the metastatic tumor is a peritoneal metastasis. In a specific embodiment, the cancer is resistant to treatment with trastuzumab, cetuximab, lapatinib, erlotinib, or any other small molecule or antibody that targets the HER family of receptors.

In a specific embodiment of a method described herein, the sequence of each heavy chain is any of SEQ ID NOs: 23, 27, 62 or 63. In a specific embodiment, the sequence of each light chain is SEQ ID NO: 25. In a specific embodiment, the sequence of the peptide linker is any of SEQ ID NOs: 14 or 35-41. In a specific embodiment, the sequence of a V_{H} domain in the first scFv is any of SEQ ID NOs: 15, 17 or 64. In a specific embodiment, the sequence of an intra-scFv peptide linker between a V_{H} domain and a V_{L} domain in the first scFv is any of SEQ ID NOs: 14 or 35-41. In a specific embodiment, the sequence of a V_{L} domain in the first scFv is any of SEQ ID NOs: 16 or 65. In a specific embodiment, the sequence of the scFv is any of SEQ ID NOs: 19 or 48-59. In a specific embodiment, the sequence of the first light chain fusion polypeptide is any of SEQ ID NOs: 29, 34, 42-47, or 60. In a specific embodiment, the sequence of each heavy chain is SEQ ID NO: 27 and the sequence of each light chain is SEQ ID NO: 25. In a specific embodiment, the sequence of the scFv is SEQ ID NO: 19. In a specific embodiment, the peptide linker is 5-30, 5-25, 5-15, 10-30, 10-20, 10-15, 15-30, or 15-25 amino acids in length. In a specific embodiment, the sequence of the peptide linker is SEQ ID NO: 14. In a specific embodiment, the sequence of the first light chain fusion polypeptide is SEQ ID NO: 60. In a specific embodiment, the sequence of the heavy chain is SEQ ID NO: 62 and the sequence of each light chain fusion polypeptide is SEQ ID NO: 60. In a specific embodiment, the sequence of the first light chain fusion polypeptide is SEQ ID NO: 47. In a specific embodiment, the sequence of the heavy chain is SEQ ID NO: 27 and the sequence of each light chain fusion polypeptide is SEQ ID NO: 47. In a specific embodiment, the sequence of the first light chain fusion polypeptide is SEQ ID NO: 29. In a specific embodiment, the sequence of the heavy chain is SEQ ID NO: 27 and the sequence of each light chain fusion polypeptide is SEQ ID NO: 29. In a specific embodiment, the K_{D} of the bispecific binding molecule is between 70 nM and 1 μM for CD3. In a specific embodiment, the bispecific binding molecule does not bind an Fc receptor in its soluble or cell-bound form. In a specific embodiment, the heavy chain has been mutated to destroy an N-linked glycosylation site. In a specific embodiment, the heavy chain has an amino acid substitution to replace an asparagine that is an N-linked glycosylation site, with an amino acid that does not function as a glycosylation site. In a specific embodiment, the heavy chain has been mutated to destroy a C1q binding site. In a specific embodiment, the bispecific binding molecule does not activate complement. In a specific embodiment, the scFv is disulfide stabilized.

In a specific embodiment of a method described herein, the administering is intravenous. In a specific embodiment of a method described herein, the administering is intraperitoneal, intrathecal, intraventricular in the brain, or intraparenchymal in the brain. In a specific embodiment of a method described herein, the administering is performed in combination with multi-modality anthracycline-based therapy.

In a specific embodiment of a method described herein, the method further comprises administering to the subject doxorubicin, cyclophosphamide, paclitaxel, docetaxel, and/or carboplatin. In a specific embodiment of a method described herein, the method further comprises administering to the subject radiotherapy. In a specific embodiment of a method described herein, the method further comprises administering to the subject an agent that increases cellular HER2 expression.

In a specific embodiment of a method described herein, the bispecific binding molecule is not bound to a T cell during said administering step.

In a specific embodiment of a method described herein, the method further comprises administering T cells to the subject. In a specific embodiment, the T cells are bound to molecules identical to said bispecific binding molecule.

In a specific embodiment of a method described herein, the subject is a human. In a specific embodiment, the subject is a canine.

In a specific embodiment of a method described herein, the bispecific binding molecule is contained in a pharmaceutical composition, which pharmaceutical composition further comprises a pharmaceutically acceptable carrier.

Also provided herein is a method of treating a HER2-positive cancer in a subject in need thereof, comprising administering to the subject a therapeutically effective amount of a cell expressing a bispecific binding molecule of the invention, wherein the cancer expresses a low level of HER2, and preferably wherein the cancer is not a head and neck cancer. In a specific embodiment, the sequence of the heavy chain of the bispecific binding molecule is SEQ ID NO: 27. In a specific embodiment, the nucleotide sequence encoding the heavy chain of the bispecific binding molecule is SEQ ID NO: 26.

Also provided herein is a method of treating a HER2-positive cancer in a subject in need thereof, comprising administering to the subject (a) a therapeutically effective amount of an ex vivo cell comprising a vector comprising (i) a first polynucleotide comprising nucleotide sequences encoding a light chain fusion polypeptide comprising an immunoglobulin light chain fused to a scFv, via a peptide linker, operably linked to a first promoter, and (ii) a a second polynucleotide encoding an immunoglobulin heavy chain that binds to HER2 operably linked to a second promoter, wherein the light chain binds to HER2 and wherein the scFv binds to CD3, or (b) a therapeutically effective amount of an ex vivo cell comprising a mixture of polynucleotides comprising (i) a first polynucleotide comprising nucleotide sequences encoding a light chain fusion polypeptide comprising an immunoglobulin light chain fused to a scFv, via a peptide linker, operably linked to a first promoter, and (ii) a second polynucleotide encoding an immunoglobulin heavy chain that binds to HER2 operably linked to a second promoter; and wherein the cancer expresses a low level of HER2, and wherein the cancer is not a head and neck cancer. In a specific embodiment, the sequence of the heavy chain is SEQ ID NO: 27. In a specific embodiment, the nucleotide sequence encoding the heavy chain is SEQ ID NO: 26.

Also provided herein is a method of treating a HER2-positive cancer in a subject in need thereof, comprising administering to the subject a therapeutically effective amount of a cell expressing a bispecific binding molecule of the invention, wherein the cancer is not indicated for treatment with trastuzumab, and preferably wherein the cancer is not a head and neck cancer. In a specific embodiment, the sequence of the heavy chain of the bispecific binding molecule is SEQ ID NO: 27. In a specific embodiment, the nucleotide sequence encoding the heavy chain of the bispecific binding molecule is SEQ ID NO: 26.

Also provided herein is a method of treating a HER2-positive cancer in a subject in need thereof, comprising administering to the subject (a) a therapeutically effective amount of an ex vivo cell comprising a vector comprising (i) a first polynucleotide comprising nucleotide sequences encoding a light chain fusion polypeptide comprising an immunoglobulin light chain fused to a scFv, via a peptide linker, operably linked to a first promoter, and (ii) a a second polynucleotide encoding an immunoglobulin heavy chain that binds to HER2 operably linked to a second promoter, wherein the light chain binds to HER2 and wherein the scFv binds to CD3, or (b) a therapeutically effective amount of an ex vivo cell comprising a mixture of polynucleotides comprising (i) a first polynucleotide comprising nucleotide sequences encoding a light chain fusion polypeptide comprising an immunoglobulin light chain fused to a scFv, via a peptide linker, operably linked to a first promoter, and (ii) a second polynucleotide encoding an immunoglobulin heavy chain that binds to HER2 operably linked to a second promoter; and wherein the cancer is not indicated for treatment with trastuzumab, and wherein the cancer is not a head and neck cancer.

In certain embodiments, provided herein is a bispecific binding molecule comprising an aglycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, and wherein the first and second light chain fusion polypeptides are identical.

In certain embodiments of the bispecific binding molecule, the sequence of each heavy chain is any of SEQ ID NOs: 23 or 27. In certain embodiments of the bispecific binding molecule, the sequence of each light chain is SEQ ID NO: 25. In certain embodiments of the bispecific binding molecule, the sequence of the peptide linker is SEQ ID NO: 14. In certain embodiments of the bispecific binding molecule, the sequence of a V_{H} domain in the first scFv is any of SEQ ID NOs: 15 or 17. In certain embodiments of the bispecific binding molecule, the sequence of an intra-scFv peptide linker between a V_{H} domain and a V_{L} domain in the first scFv is of SEQ ID NO: 14. In certain embodiments of the bispecific binding molecule, the sequence of a V_{L} domain in the first scFv is of SEQ ID NO: 16. In certain embodiments of the bispecific binding molecule, the sequence of the scFv is SEQ ID NO: 19. In certain embodiments of the bispecific binding molecule, the sequence of the first light chain fusion polypeptide is SEQ ID NO: 29.

In certain embodiments of the bispecific binding molecule, the sequence of each heavy chain is any of SEQ ID NOs: 23, 27, 62 or 63. In certain embodiments of the bispecific binding molecule, the sequence of each light chain is SEQ ID NO: 25. In certain embodiments of the bispecific binding molecule, the sequence of the peptide linker is any of SEQ ID NOs: 14 or 35-41. In certain embodiments of the bispecific binding molecule, the sequence of a V_{H} domain in the first scFv is any of SEQ ID NOs: 15, 17 or 64. In certain embodiments of the bispecific binding molecule, the sequence of an intra-scFv peptide linker between a V_{H} domain and a V_{L} domain in the first scFv is any of SEQ ID NOs: 14 or 35-41. In certain embodiments of the bispecific binding molecule, the sequence of a V_{L} domain in the first scFv is any of SEQ ID NOs: 16 or 65. In certain embodiments of the bispecific binding molecule, the sequence of the scFv is any of SEQ ID NOs: 19 or 48-59. In certain embodiments of the bispecific binding molecule, the sequence of the first light chain fusion polypeptide is any of SEQ ID NOs: 29, 34, 42-47, or 60.

In certain embodiments of the bispecific binding molecule, the sequence of each heavy chain is SEQ ID NO: 27 and the sequence of each light chain is SEQ ID NO: 25. In certain embodiments of the bispecific binding molecule, the sequence of the scFv is SEQ ID NO: 19. In certain embodiments of the bispecific binding molecule, the sequence of the heavy chain is SEQ ID NO: 27, the sequence of each light chain is SEQ ID NO: 25 and the sequence of the scFv is SEQ ID NO: 19. In certain embodiments of the bispecific binding molecule, the peptide linker is 5-30, 5-25, 5-15, 10-30, 10-20, 10-15, 15-30, or 15-25 amino acids in length. In certain embodiments, the sequence of the peptide linker is SEQ ID NO: 14.

In certain embodiments, the sequence of the first light chain fusion polypeptide is SEQ ID NO: 60. In certain embodiments, the sequence of the heavy chain is SEQ ID NO: 62 and the sequence of each light chain fusion polypeptide is SEQ ID NO: 60.

In certain embodiments, the sequence of the first light chain fusion polypeptide is SEQ ID NO: 47. In certain embodiments, the sequence of the heavy chain is SEQ ID NO: 27 and the sequence of each light chain fusion polypeptide is SEQ ID NO: 47.

In certain embodiments, the sequence of the first light chain fusion polypeptide is SEQ ID NO: 29. In certain embodiments, the sequence of the heavy chain is SEQ ID NO: 27 and the sequence of each light chain fusion polypeptide is SEQ ID NO: 29.

In certain embodiments of the bispecific binding molecule, the K_{D} is between 70 nM and 1 μM for CD3.

In certain embodiments of the bispecific binding molecule, the scFv of the bispecific binding molecule comprises one or more mutations to stabilize disulfide binding. In certain embodiments of the bispecific binding molecule, the stabilization of disulfide binding prevents aggregation of the bispecific binding molecule. In certain embodiments of the bispecific binding molecule, the stabilization of disulfide binding reduces aggregation of the bispecific binding molecule as compared to aggregation of the bispecific binding molecule without the stabilization of disulfide binding. In certain embodiments of the bispecific binding molecule, the one or more mutations to stabilize disulfide binding comprise a V_{H} G44C mutation and a V_{L} Q100C mutation (e.g., as present in SEQ ID NOS: 54-59). In certain embodiments of the bispecific binding molecule, the one or more mutations to stabilize disulfide binding are the replacement of the amino acid residue at V_{H}44 (according to the Kabat numbering system) with a cysteine and the replacement of the amino acid residue at V_{L}100 (according to the Kabat numbering system) with a cysteine so as to introduce a disulfide bond between V_{H}44 and V_{L}100 (e.g., as present in SEQ ID NOS: 54-59).

In certain embodiments of the bispecific binding molecule, the bispecific binding molecule does not bind an Fc receptor in its soluble or cell-bound form. In certain embodiments of the bispecific binding molecule, the heavy chain has been mutated to destroy an N-linked glycosylation site. In certain embodiments of the bispecific binding molecule, the heavy chain has an amino acid substitution to replace an asparagine that is an N-linked glycosylation site, with an amino acid that does not function as a glycosylation site. In certain embodiments of the bispecific binding molecule, the heavy chain has been mutated to destroy a C1q binding site. In certain embodiments, the bispecific binding molecule does not activate complement.

In certain embodiments, provided herein is a bispecific binding molecule comprising an aglycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, wherein the first and second light chain fusion polypeptides are identical, and wherein (a) the sequence of each heavy chain is SEQ ID NO: 62; and (b) the sequence of each light chain fusion polypeptide is SEQ ID NO: 60.

In certain embodiments, provided herein is a bispecific binding molecule comprising an aglycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, wherein the first and second light chain fusion polypeptides are identical, and wherein (a) the sequence of each heavy chain is SEQ ID NO: 27; and (b) the sequence of each light chain fusion polypeptide is SEQ ID NO: 47.

In certain embodiments, provided herein is a bispecific binding molecule comprising an aglycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, wherein the first and second light chain fusion polypeptides are identical, and wherein (a) the sequence of each heavy chain is SEQ ID NO: 27; and (b) the sequence of each light chain fusion polypeptide is SEQ ID NO: 29.

In certain embodiments, provided herein is a polynucleotide comprising nucleotide sequences encoding a light chain fusion polypeptide comprising an immunoglobulin light chain fused to a scFv, via a peptide linker, wherein the light chain binds to HER2 and wherein the scFv binds to CD3. In certain embodiments of the polynucleotide, the sequence of the light chain is SEQ ID NO: 25. In certain embodiments of the polynucleotide, the nucleotide sequence encoding the light chain is SEQ ID NO: 24. In certain embodiments of the polynucleotide, the sequence of the scFv is SEQ ID NO: 19. In certain embodiments of the polynucleotide, the nucleotide sequence encoding the scFv is SEQ ID NO: 18. In certain embodiments of the polynucleotide, the sequence of the light chain is SEQ ID NO: 25 and the sequence of the scFv is SEQ ID NO: 19. In certain embodiments of the polynucleotide, the nucleotide sequence encoding the light chain is SEQ ID NO: 24 and the nucleotide sequence encoding the scFv is SEQ ID NO: 18. In certain embodiments of the polynucleotide, the peptide linker is 5-30, 5-25, 5-15, 10-30, 10-20, 10-15, 15-30, or 15-25 amino acids in length. In certain embodiments of the polynucleotide, the sequence of the peptide linker is SEQ ID NO: 14. In certain embodiments of the polynucleotide, the nucleotide sequence encoding the peptide linker is SEQ ID NO: 13.

In certain embodiments, provided herein is a vector comprising a polynucleotide encoding nucleotide sequences encoding a light chain fusion polypeptide comprising an immunoglobulin light chain fused to a scFv, via a peptide linker, wherein the light chain binds to HER2 and wherein the scFv binds to CD3, operably linked to a promoter. In certain embodiments, provided herein is an ex vivo cell comprising the polynucleotide provided herein operably linked to a promoter. In certain embodiments, provided herein is an ex vivo cell comprising the vector.

In certain embodiments, provided herein is a vector comprising (i) a first polynucleotide comprising nucleotide sequences encoding a light chain fusion polypeptide comprising an immunoglobulin light chain fused to a scFv, via a peptide linker, wherein the light chain binds to HER2 and wherein the scFv binds to CD3 operably linked to a first promoter, and (ii) a second polynucleotide encoding an immunoglobulin heavy chain that binds to HER2 operably linked to a second promoter. In certain embodiments, provided herein is an ex vivo cell comprising the vector.

In certain embodiments, provided herein is a method of producing a bispecific binding molecule comprising (a) culturing the cell comprising the vector comprising (i) a first polynucleotide comprising nucleotide sequences encoding a light chain fusion polypeptide comprising an immunoglobulin light chain fused to a scFv, via a peptide linker, wherein the light chain binds to HER2 and wherein the scFv binds to CD3 operably linked to a first promoter, and (ii) a second polynucleotide encoding an immunoglobulin heavy chain that binds to HER2 operably linked to a second promoter, to express the first and second polynucleotides such that a bispecific binding molecule comprising said light chain fusion polypeptide and said immunoglobulin heavy chain is expressed, and (b) recovering the bispecific binding molecule.

In certain embodiments, provided herein is a pharmaceutical composition comprising a therapeutically effective amount of (i) the first polynucleotide operably linked to the first promoter, and (ii) the second polynucleotide encoding an immunoglobulin heavy chain that binds to HER2 operably linked to the second promoter. In certain embodiments, provided herein is a pharmaceutical composition comprising a therapeutically effective amount of a vector comprising (i) the first polynucleotide operably linked to the first promoter, and (ii) the second polynucleotide encoding an immunoglobulin heavy chain that binds to HER2 operably linked to the second promoter. In certain embodiments, the vector is a viral vector.

In certain embodiments, provided herein is a mixture of polynucleotides comprising (i) a polynucleotide comprising nucleotide sequences encoding a light chain fusion polypeptide comprising an immunoglobulin light chain fused to a scFv, via a peptide linker, wherein the light chain binds to HER2 and wherein the scFv binds to CD3 operably linked to a first promoter, and (ii) a second polynucleotide encoding an immunoglobulin heavy chain that binds to HER2 operably linked to a second promoter. In certain embodiments of the mixture of polypeptides, the sequence of the heavy chain is SEQ ID NO: 27. In certain embodiments of the mixture of polypeptides, the nucleotide sequence encoding the heavy chain is SEQ ID NO: 26. In certain embodiments, provided herein is an ex vivo cell comprising the mixture of polynucleotides provided herein.

In certain embodiments, provided herein is a method of producing a bispecific binding molecule, comprising (i) culturing the cell comprising the mixture of polynucleotides to express the first and second polynucleotides such that a bispecific binding molecule comprising said light chain fusion polypeptide and said immunoglobulin heavy chain is produced, and (ii) recovering the bispecific binding molecule.

In certain embodiments, provided herein is a method of producing a bispecific binding molecule, comprising (i) expressing the mixture of polynucleotides such that a bispecific binding molecule comprising said first light chain fusion polypeptide and said immunoglobulin heavy chain is produced, and (ii) recovering the bispecific binding molecule.

In certain embodiments, provided herein is a method of making a therapeutic T cell comprising binding a bispecific binding molecule described herein to a T cell. In certain embodiments, the T cell is a human T cell. In certain embodiments, the binding is noncovalently.

In certain embodiments, provided herein is a pharmaceutical composition comprising a therapeutically effective amount of the bispecific binding molecule and a pharmaceutically acceptable carrier.

In certain embodiments, provided herein is a pharmaceutical composition comprising a therapeutically effective amount of the bispecific binding molecule, a pharmaceutically acceptable carrier, and T cells. In certain embodiments, the T cells are bound to the bispecific binding molecule. In certain embodiments, the binding of the T cells to the bispecific binding molecule is noncovalently. In certain embodiments, the T cells are administered to a subject for treatment of a HER2-positive cancer in the subject. In certain embodiments, the T cells are autologous to the subject to whom they are administered. In certain embodiments, the T cells are allogeneic to the subject to whom they are administered. In certain embodiments, the T cells are human T cells.

In certain embodiments, provided herein is a method of treating a HER2-positive cancer in a subject in need thereof comprising administering a pharmaceutical composition provided herein. In certain embodiments, provided herein is a method of treating a HER2-positive cancer in a subject in need thereof comprising administering a therapeutically effective amount of a bispecific binding molecule provided herein. In certain embodiments, the HER2-positive cancer is breast cancer, gastric cancer, an osteosarcoma, desmoplastic small round cell cancer, squamous cell carcinoma of head and neck cancer, ovarian cancer, prostate cancer, pancreatic cancer, glioblastoma multiforme, gastric junction adenocarcinoma, gastroesophageal junction adenocarcinoma, cervical cancer, salivary gland cancer, soft tissue sarcoma, leukemia, melanoma, Ewing's sarcoma, rhamdomyosarcoma, neuroblastoma, small cell lung cancer, or any other neoplastic tissue that expresses the HER2 receptor. In certain embodiments, the HER2-positive cancer is a primary tumor or a metastatic tumor, e.g., a brain or peritoneal metastases.

In certain embodiments of the method of treating, the administering is intravenous. In certain embodiments of the method of treating, the administering is intraperitoneal, intrathecal, intraventricular, or intraparenchymal. In certain embodiments of the method of treating, the method further comprises administering to the subject doxorubicin, cyclophosphamide, paclitaxel, docetaxel, and/or carboplatin. In certain embodiments of the method of treating, the method further comprises administering to the subject radiotherapy. In certain embodiments of the method of treating, the administering is performed in combination with multi-modality anthracycline-based therapy. In certain embodiments of the method of treating, the administering is performed in combination with cytoreductive chemotherapy. In a specific embodiment, the administering is performed after treating the subject with cytoreductive chemotherapy. In certain embodiments of the method of treating, the bispecific binding molecule is not bound to a T cell. In certain embodiments of the method of treating, the bispecific binding molecule is bound to a T cell. In certain embodiments of the method of treating, the binding of the bispecific binding molecule to the T cell is non-covalently. In certain embodiments of the method of treating, the administering is performed in combination with T cell infusion. In a specific embodiment, the administering is performed after treating the patient with T cell infusion. In certain embodiments, the T cell infusion is performed with T cells that are autologous to the patient to whom the T cells are administered. In certain embodiments, the T cell infusion is performed with T cells that are allogeneic to the patient to whom the T cells are administered. In certain embodiments, the T cells can be bound to molecules identical to a bispecific binding molecule as described herein. In certain embodiments, the binding of the T cells to the molecules identical to a bispecific binding molecule is noncovalently. In certain embodiments, the T cells are human T cells.

In certain embodiments of the method of treating, the method further comprises administering to the subject an agent that increases cellular HER2 expression. In certain embodiments of the method of treating, the HER2-positive cancer is resistant to treatment with trastuzumab, cetuximab, lapatinib, erlotinib, or any other small molecule or antibody that targets the HER family of receptors. In certain embodiments of the method of treating, the subject is a human. In certain embodiments of the method of treating, the subject is a canine.

### 4. BRIEF DESCRIPTION OF THE DRAWINGS

FIG. 1A, FIG. 1B, FIG. 1C, FIG. 1D, and FIG. 1E describe HER2-BsAb. FIG. 1A depicts a schematic of the HER2-BsAb. The arrow points to the N297A mutation introduced into the heavy chain to remove glycosylation. FIG. 1B depicts the purity of HER2-BsAb as demonstrated under reducing SDS-PAGE conditions. FIG. 1C depicts the purity of HER2-BsAb as demonstrated by SEC-HPLC. FIG. 1D demonstrates that the N297A mutation in the human IgG1-Fc inhibits binding to the CD16A Fc receptor. FIG. 1E demonstrates that the N297A mutation in the human IgG1-Fc inhibits binding to the CD32A Fc receptor.

FIG. 2A and FIG. 2B demonstrate that HER2-BsAb binds to a breast cancer cell line and to T cells. FIG. 2A depicts the staining of AU565 breast cancer cells with trastuzumab (left) or with HER2-BsAb (right). FIG. 2B depicts the staining of CD3+ T cells with huOKT3 (left) or with HER2-BsAb (right).

FIG. 3 demonstrates that HER2-BsAb displays potent cytotoxic T lymphocyte activity in a 4 hour ⁵¹Cr release assay. For a description of trastuzumab-mOKT3, see, Thakur et al., 2010, Curr Opin Mol Ther, 12:340.

FIG. 4 compares the HER2 expression against HER2-BsAb T cell cytotoxicity in a panel of cancer cell lines.

FIG. 5A and FIG. 5B demonstrate that HER2-BsAb-redirected T cell cytotoxicity is antigen specific. FIG. 5A demonstrates that HER2-BsAb mediates T cell cytotoxicity against the HER2-positive cell line, UM SCC 47, but not the HER2-negative cell line HTB-132. FIG. 5B demonstrates that huOKT3 and trastuzumab can block the ability of HER2-BsAb to mediate T cell cytotoxicity.

FIG. 6 demonstrates that HER2-BsAb detects low levels of HER2 by comparing the HER2-BsAb mediated T cell cytotoxicity to the HER2 threshold of detection by flow cytometry.

FIG. 7A, FIG. 7B, and FIG. 7C provide the specificity, affinity, and antiproliferative action of HER2-BsAb. FIG. 7A demonstrates that pre-incubation of the HER2-positive SKOV3 ovarian carcinoma cell line blocks binding of HER2-BsAb. FIG. 7B demonstrates that SKOV3 cells labeled with dilutions of trastuzumab or with HER2-BsAb display similar curves when mean fluorescence intensity (MFI) is plotted against antibody concentration. Fib. 7C demonstrates the antiproliferative action of HER2-BsAb compared against trastuzumab in the trastuzumab sensitive breast cancer cell line SKBR3.

FIG. 8 demonstrates that HER2-BsAb is effective against squamous cell carcinoma of the head and neck (SCCHN) cell lines. A panel of SCCHN cells were analyzed for HER2-BsAb-mediated cytotoxicity and EC50 and compared to the expression level of HER2 in each cell line as determined by flow cytometry and by qRT-PCR.

FIG. 9A, FIG. 9B, and FIG. 9C. HER2-BsAb mediates T cell cytotoxicity against SCCHN resistant to other HER targeted therapies. FIG. 9A demonstrates that the SCCHN cell line PCI-30 expresses EGFR and HER2. FIG. 9B demonstrates that PCI-30 cells are resistant to HER-targeted therapies lapatinib, erlotinib, neratinib, trastuzumab, and cetuximab. FIG. 9C demonstrates that PCI-30 cells are sensitive to T cells in the presence of HER2-BsAb. Data represents the average of three different cytotoxicity assays.

FIG. 10 demonstrates that HER2-BsAb is effective against osteosarcoma cell lines. A panel of osteosarcoma cell lines were analyzed for HER2-BsAb-mediated cytotoxicity and EC50 and compared to the expression level of HER2 in each cell line as determined by flow cytometry and by qRT-PCR

FIG. 11A, FIG. 11B, and FIG. 11C demonstrate that HER2-BsAb is effective against osteosarcoma cell lines resistant to other targeted therapies. FIG. 11A demonstrates that the osteosarcoma cell line U2OS expresses EGFR and HER2. FIG. 11B demonstrates that USOS cells are resistant to HER-targeted therapies lapatinib, erlotinib, neratinib, trastuzumab, and cetuximab. FIG. 11C demonstrates that USOS cells are sensitive to T cells in the presence of HER2-BsAb. Data represents the average of three different cytotoxicity assays.

FIG. 12A, FIG. 12B, FIG. 12C and FIG. 12D demonstrate that HER2-BsAb is effective against the HeLa cervical carcinoma cell line resistant to other targeted therapies. FIG. 12A demonstrates that HeLa cells express EGFR and HER2. FIG. 12B demonstrates that HeLa cells are resistant to HER-targeted therapies lapatinib, erlotinib, neratinib, trastuzumab, and cetuximab. FIG. 12C demonstrates that HeLa cells are sensitive to T cells in the presence of HER2-BsAb. Data represents the average of three different cytotoxicity assays. FIG. 12D demonstrates that pre-treatment with lapatinib enhances HeLa sensitivity to HER2-BsAb.

FIG. 13 demonstrates that HER2-BsAb reduces tumor growth in vivo. FIG. 13 demonstrates that HER2-BsAb protects against tumor progression in implanted MCF7 breast cancer cells mixed with PBMCs.

FIG. 14 demonstrates that HER2-BsAb protects against tumor progression in implanted HCC1954 breast cancer mixed with peripheral blood mononuclear cells (PBMC) in vivo.

FIG. 15 demonstrates that HER2-BsAb protects against a metastatic model of tumor progression induced by intravenous introduction of luciferase-tagged MCF7 cells in vivo.

FIG. 16A, FIG. 16B, FIG. 16C, and FIG. 16D demonstrate that HER2-BsAb blocks the metastatic tumor growth of luciferase-tagged MCF7 cells in vivo. FIG. 16A represents mice without treatment. FIG. 16B represents mice treated with PBMC and HER2-C825. FIG. 16C represents mice treated with HER2-BsAb. FIG. 16D represents mice treated with PBMC and HER2-BsAb.

FIG. 17A, FIG. 17B, and FIG. 17C describe HER2-BsAb. FIG. 17A depicts a schematic of the HER2-BsAb. The arrow points to the N297A mutation introduced into the heavy chain to remove glycosylation. FIG. 17B depicts the purity of HER2-BsAb as demonstrated under reducing SDS-PAGE conditions. FIG. 17C depicts the purity of HER2-BsAb as demonstrated by size exclusion chromatography high performance liquid chromatography (SEC-HPLC).

FIG. 18A, FIG. 18B, and FIG. 18C demonstrate that HER2-BsAb has the same specificity, similar affinity, and antiproliferative effects as trastuzumab.

FIG. 19A and FIG. 19B demonstrate that HER2-BsAb redirected T cell cytotoxicity is HER2-specific and dependent on CD3.

FIG. 20 depicts HER2 expression and half maximal effective concentration (EC50) in the presence of ATC and HER2-BsAb in 35 different cell lines from different tumor systems.

FIG. 21A, FIG. 21B, FIG. 21C, FIG. 21D, FIG. 21E, FIG. 21F, FIG. 21G, FIG. 21H, and FIG. 21I demonstrate that HER2-BsAb mediates cytotoxic responses against carcinoma cell lines resistant to other HER-targeted therapies.

FIG. 22 demonstrates that the EC50 of HER2-BsAb correlates with the HER2 level of expression determined by flow-cytometry. pM-picomolar; MFI=mean fluorescence intensity.

FIG. 23A, FIG. 23B, and FIG. 23C demonstrates that HER2-BsAb mediates T cell cytotoxicity against PD-L1-positive HCC1954 targets in a manner that is relatively insensitive to PD-1 blockade by pembrolizumab, even with PD-1 expression on effector T cells.

FIG. 24A and FIG. 24B demonstrates that HER2-BsAb mediates T cell cytotoxicity against PD-L1-positive HEK-293 targets in a manner that is relatively insensitive to PD-1 expression on effector T cells. The cytotoxicity is an average of 6 experiments.

FIG. 25A, FIG. 25B, FIG. 25C, and FIG. 25D demonstrate that HER2-BsAb is effective against HER2-positive xenografts.

FIG. 26A, FIG. 26B, FIG. 26C, FIG. 26D, and FIG. 26E demonstrate in vitro characterization of HER2-BsAb. FIG. 26A: HER2-BsAb has the same specificity as trastuzumab. Pre-Incubation of the HER2 (+) high SKOV3 cells with trastuzumab prevents HER2-BsAb binding. FIG. 26B: HER2-BsAb and trastuzumab have similar avidity for SKOV3 cells. Mean fluorescence intensity (“MFI”) was plotted against the antibody concentration. FIG. 26C: HER2-BsAb maintained same anti-proliferative effects as trastuzumab against the trastuzumab-sensitive SKBR3 cells. FIG. 26D: HER2-BsAb mediates T-cell cytotoxicity against the HER2 (+) MCF-7 cells but not the HER2 (−) HTB-132 cells. FIG. 26E: Blocking of HER2 or CD3 by trastuzumab or huOKT3, abrogates HER2-BsAb T-cell cytotoxicity. HER2 (+) SCCHN PCI-13 cells were used in the cytotoxicity assay. For this experiment 0.1 ug/mL of HER2-BsAb with 10 μg/mL of the blocking antibodies were used.

FIG. 27A and FIG. 27B demonstrate HER2-BsAb binding to T cells and redirecting T-cell killing. FIG. 27A: FACS histograms of HER2-BsAb binding to naïve T cells purified from fresh PBMC (left panel) or ATCs (right panel). Concentrations of BsAbs (μg/10⁶ cells) were recorded on the top of the left histogram, and Rituxan was used as negative control (mean fluorescence intensity set at 5). FIG. 27B: HER2-BsAb redirected T-cell killing of HER2 (+) AU565 breast cancer cells by 4-hour ⁵¹Cr release assay. BsAb was either mixed directly with T cells and AU565 together (mixing), or pre-incubated with T cells/target first (T cells pre-armed or AU565 pre-targeted), and unbound BsAb washed off before adding the other cells. ATC-to-target ratio was 10:1. Data points are shown as Mean±SEM.

FIG. 28A, FIG. 28B, FIG. 28C, and FIG. 28D demonstrate that HER2-BsAb mediates cytotoxic responses against carcinoma cell lines resistant to other HER targeted therapies. FIG. 28A, FIG. 28B, and FIG. 28C: Three representative cell lines were used for FACS assay (upper panel), proliferation assay (middle panel), and HER2-BsAb mediated CTL assay (lower panel): (FIG. 28A) SCCHN PCI-30, (FIG. 28B) breast carcinoma HCC-1954, and (FIG. 28C) osteosarcoma U2OS. FIG. 28D: HER2-BsAb EC50 inversely correlates with level of HER2 expression. Each of the cell lines used in a cytotoxicity assay (Table 9) was assayed at least twice. The EC50 was determined each time and averaged. These values (except those beyond assay limit 5 nM) were compared to HER2 expression (MFI).

FIG. 29A and FIG. 29B demonstrate that HER2-BsAb-mediated in vitro T-cell cytotoxicity was relatively insensitive to PD-L1 expression on the tumor targets or PD-1 expression on T cells. FIG. 29A: FACS analysis of PD-L1 expression in HCC1954 cells (left panel), of induced PD-1 expression in ATCs (middle panel), and HER2-BsAb-mediated cytotoxicity (right panel). FIG. 29B: FACS analysis of PD-L1 expression in HEK-293 cells (left panel), and HER2-BsAb mediated cytotoxicity using the ATCs as in FIG. 29A (middle panel). Mean±SEM (n=6).

FIG. 30A, FIG. 30B, FIG. 30C, FIG. 30D, and FIG. 30E demonstrate that HER2-BsAb is effective against HER2 (+) breast cancer cell line xenografts. Treatment schedules were marked on the figures, and doses of BsAbs and effector cells were detailed in the Results of Section 6.3.3. Data shown as mean±SEM (n=5). FIG. 30A: intravenous (“i.v.”) tumor plus i.v. effector cells model: Bioluminescence changes of MCF7 breast cancers during treatment. FIG. 30B and FIG. 30C: subcutaneous (“s.c.” tumor plus s.c. effector cells (mixing) model: % tumor growth of MCF7 (FIG. 30B), and tumor volume changes of HCC1954 (FIG. 30C). FIG. 30D: s.c. tumor plus i.v. effector cells model: tumor volume changes of HCC1954. FIG. 30E: HCC1954 s.c. tumor model as in (FIG. 30D), with treatments of one dose of PBMC (2×10⁷ cells i.v.) at day 14, and two doses of BsAbs (100 ug i.v.) at day 12 and 15. Representative images (200× magnifications) of IHC staining of tumor sections collected 5 days after i.v. PBMC were shown.

FIG. 31A and FIG. 31B demonstrate that HER2-BsAb is effective against HER2 (+) ovarian cancer cell line xenografts. Treatment schedules were marked on the figures, and doses of BsAbs and effector cells were detailed in the Results of Section 6.3.3. Data shown as mean±SEM (n=4). FIG. 31A: intraperitoneal (“i.p.”) tumor plus i.p./i.v. effector cells model: Bioluminescence changes of SKOV3-luc ovarian cancers during treatment. FIG. 31B: Representative bioluminescence images at the beginning (Day 13) and ending (Day 34) of the treatment were shown.

FIG. 32A, FIG. 32B, FIG. 32C, FIG. 32D, and FIG. 32E demonstrate that HER2-BsAb is effective against HER2 (+) PDXs. s.c. tumor plus i.v. effector cells model was used for PDXs. Treatment schedules were marked on the figures, and doses of BsAbs and effector cells were detailed in the Results of Section 6.3.3. Data shown as mean±SEM (n=5). FIG. 32A: Tumor volume changes of EK gastric cancer PDX. FIG. 32B: IHC images of CD3 staining from another experiment with similar setting as in FIG. 32A. Representative images (200× magnifications) of IHC staining of tumor sections collected 36 days after i.v. PBMC were shown. FIG. 32C: IHC images (200× magnifications) of HER2 staining of control treated tumor sections. FIG. 32D and FIG. 32E: Average tumor volume changes of M37 breast cancer PDX (FIG. 32D), and tumor growth of 5 individual mouse (black thin line) and averages (black thick line) in each group (FIG. 32E).

FIG. 33A and FIG. 33B demonstrate that HER2-BsAb binding to CD3 on T cells was functionally monovalent. FIG. 33A: Cytokine release from naïve T cells induced by 16.7 nM HER2-BsAb when compared to bivalent huOKT3 lgG and monovalent huOKT3 Fab, in the absence (left panel) or presence (right panel) of HER2 (+) NCI-N87 gastric tumor cells. Cytokine release level below detection level was assigned as 1 μg/ml. FIG. 33B: T cell proliferation stimulated by 67 nM of the related antibodies, in the absence of tumor targets. T cells only (Control) as the negative control. OD reading at 450 nm (AU) was shown. All data points are shown as Mean±SD.

FIG. 34 demonstrates that HER2-BsAb is effective against HER2 (+) breast cancer cell line xenografts that express PDL1 but are resistant to PD1 or PDL1 treatment. s.c. tumor plus i.v. effector cell model: tumor volume changes of HCC1954. Data shown as mean±SEM (n=5). Treatment schedules are marked on the figures. s.c. 5×10⁶ HCC1954 xenografts were treated with i.v. PBMC (7.5×10⁶, once per week for 2 weeks), and i.v. HER2-BsAb, anti-PD1 Pembrolizumab, or anti-PDL1 Atezolizumab (100 ug each, twice per week for 4 weeks). Tumors were completely eradicated with HER2-BsAb treatment, in contrast to no effect for treatment with PD1/PDL1 blockade (i.e., treatment with anti-PD1 Pembrolizumab or anti-PDL1 Atezolizumab).

### 5. DETAILED DESCRIPTION

Provided herein are bispecific binding molecules that bind to both HER2 and CD3. Also provided herein are isolated nucleic acids (polynucleotides), such as complementary DNA (cDNA), encoding such bispecific binding molecules or fragments thereof. Further provided are vectors (e.g., expression vectors) and cells (e.g., ex vivo cells) comprising nucleic acids (polynucleotides) or vectors (e.g., expression vectors) encoding such bispecific binding molecules or fragments thereof. Also provided herein are methods of making such bispecific binding molecules, cells, and vectors. Also provided herein are T cells bound to bispecific binding molecules provided herein. Also provided herein are methods of binding such bispecific binding molecules to T cells. In other embodiments, provided herein are methods and uses for treating HER2-positive cancers using the bispecific binding molecules, nucleic acids, vectors, and/or T cells described herein. Additionally, related compositions (e.g., pharmaceutical compositions), kits, and diagnostic methods are also provided herein.

In certain embodiments, provided herein are bispecific binding molecules that specifically bind to HER2 and to CD3, and invoke T cell cytotoxicity for treating cancer. Without being bound by any theory, it is believed that the bispecific binding molecules described herein not only bind tumors to T cells, they also cross-link CD3 on T cells and initiate the activation cascade, and, this way, T cell receptor (TCR)-based cytotoxicity is redirected to desired tumor targets, bypassing major histocompatibility complex (MHC) restrictions.

### 5.1 Bispecific Binding Molecules

Provided herein are bispecific binding molecules that bind to HER2 and CD3. A binding molecule, which can be used within the methods provided herein, is a bispecific binding molecule comprising an aglycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first fusion polypeptide, and wherein the second light chain is fused to a second scFv, via a peptide linker, to create a second fusion polypeptide, wherein the first and second scFv (i) are identical, and (ii) bind to CD3, and wherein the first and second fusion polypeptides are identical.

HER2 is a member of the epidermal growth factor receptor (EGFR) family of receptor tyrosine kinases. In a specific embodiment, HER2 is human HER2. GenBank™ accession number NM_004448.3 (SEQ ID NO: 1) provides an exemplary human HER2 nucleic acid sequence. GenBank™ accession number NP_004439.2 (SEQ ID NO: 2) provides an exemplary human HER2 amino acid sequence. In another specific embodiment, HER2 is canine HER2. GenBank™ accession number NM_001003217.1 (SEQ ID NO: 3) provides an exemplary canine HER2 nucleic acid sequence. GenBank™ accession number NP_001003217.1 (SEQ ID NO: 4) provides an exemplary canine HER2 amino acid sequence.

CD3 is a T cell co-receptor comprised of a gamma chain, a delta chain, and two epsilon chains. In a specific embodiment, CD3 is a human CD3. GenBank™ accession number NM_000073.2 (SEQ ID NO: 5) provides an exemplary human CD3 gamma nucleic acid sequence. GenBank™ accession number NP_000064.1 (SEQ ID NO: 6) provides an exemplary human CD3 gamma amino acid sequence. GenBank™ accession number NM_000732.4 (SEQ ID NO: 7) provides an exemplary human CD3 delta nucleic acid sequence. GenBank™ accession number NP_000723.1 (SEQ ID NO: 8) provides an exemplary human CD3 delta amino acid sequence. GenBank™ accession number NM_000733.3 (SEQ ID NO: 9) provides an exemplary human CD3 epsilon nucleic acid sequence. GenBank™ accession number NP_000724.1 (SEQ ID NO: 10) provides an exemplary human CD3 epsilon amino acid sequence. In another specific embodiment, CD3 is a canine CD3. GenBank™ accession number NM_001003379.1 (SEQ ID NO: 11) provides an exemplary canine CD3 epsilon nucleic acid sequence. GenBank™ accession number NP_001003379.1 (SEQ ID NO: 12) provides an exemplary canine CD3 epsilon amino acid sequence.

The immunoglobulin in the bispecific binding molecules of the invention can be, as non-limiting examples, a monoclonal antibody, a naked antibody, a chimeric antibody, a humanized antibody, or a human antibody. As used herein, the term “immunoglobulin” is used consistent with its well known meaning in the art, and comprises two heavy chains and two light chains. Methods for making antibodies are described in Section 5.3.

A chimeric antibody is a recombinant protein that contains the variable domains including the complementarity-determining regions (CDRs) of an antibody derived from one species, preferably a rodent antibody, while the constant domains of the antibody molecule is derived from those of a human antibody. For veterinary applications, the constant domains of the chimeric antibody may be derived from that of other species, such as, for example, horse, monkey, cow, pig, cat, or dog.

A humanized antibody is an antibody produced by recombinant DNA technology, in which some or all of the amino acids of a human immunoglobulin light or heavy chain that are not required for antigen binding (e.g., the constant regions and the framework regions of the variable domains) are used to substitute for the corresponding amino acids from the light or heavy chain of the cognate, nonhuman antibody. By way of example, a humanized version of a murine antibody to a given antigen has on both of its heavy and light chains (1) constant regions of a human antibody; (2) framework regions from the variable domains of a human antibody; and (3) CDRs from the murine antibody. When necessary, one or more residues in the human framework regions can be changed to residues at the corresponding positions in the murine antibody so as to preserve the binding affinity of the humanized antibody to the antigen. This change is sometimes called “back mutation.” Similarly, forward mutations may be made to revert back to murine sequence for a desired reason, e.g., stability or affinity to antigen. Without being bound by any theory, humanized antibodies generally are less likely to elicit an immune response in humans as compared to chimeric human antibodies because the former contain considerably fewer non-human components.

The term “epitope” is art-recognized and is generally understood by those of skill in the art to refer to the region of an antigen that interacts with an antibody. An epitope of a protein antigen can be linear or conformational, or can be formed by contiguous or noncontiguous amino acid sequences of the antigen.

A scFv is an art-recognized term. An scFv comprises a fusion protein of the variable regions of the heavy (V_{H}) and light (V_{L}) chains of an immunoglobulin, wherein the fusion protein retains the same antigen specificity as the whole immunoglobulin. The V_{H} is fused to the V_{L} via a peptide linker (such a peptide linker is sometimes referred to herein as an “intra-scFv peptide linker”).

In certain embodiments of the invention, the scFv has a peptide linker that is between 5-30, 5-25, 5-15, 10-30, 10-20, 10-15, 15-30, or 15-25 amino acid residues in length. In certain embodiments, the scFv peptide linker displays one or more characteristics suitable for a peptide linker known to one of ordinary skill in the art. In certain embodiments, the scFv peptide linker comprises amino acids that allow for scFv peptide linker solubility, such as, for example, serine and threonine. In certain embodiments, the scFv peptide linker comprises amino acids that allow for scFv peptide linker flexibility, such as, for example, glycine. In certain embodiments, the scFv peptide linker connects the N-terminus of the V_{H} to the C-terminus of the V_{L} . . . In certain embodiments, the scFv peptide linker can connect the C-terminus of the V_{H} to the N-terminus of the V_{L} In certain embodiments, the scFv peptide linker is a linker as described in Table 1, below (e.g., any one of SEQ ID NOs: 14, or 35-41). In a preferred embodiment, the peptide linker is SEQ ID NO: 14.

In certain embodiments of the bispecific binding molecules of the invention, the scFv that binds to CD3 comprises the V_{H} and the V_{L} of a CD3-specific antibody known in the art, such as, for example, huOKT3 (see, for example, Adair et al., 1994, Hum Antibodies Hybridomas 5:41-47), YTH12.5 (see, for example Routledge et al., 1991, Eur J Immunol, 21:2717-2725), HUM291 (see, for example, Norman et al., 2000, Clinical Transplantation, 70 (12): 1707-1712), teplizumab (see, for example, Herold et al., 2009, Clin Immunol, 132:166-173), huCLB-T¾ (see, for example, Labrijn et al., 2013, Proceedings of the National Academy of Sciences, 110 (13): 5145-5150), otelixizumab (see, for example, Keymeulen et al., 2010, Diabetologia, 53:614-623), blinatumomab (see, for example, Cheadle, 2006, Curr Opin Mol Ther, 8 (1): 62-68), MT110 (see, for example, Silke and Gires, 2011, MAbs, 3 (1): 31-37), catumaxomab (see, for example, Heiss and Murawa, 2010, Int J Cancer, 127 (9): 2209-2221), 28F11 (see, for example, Canadian Patent Application CA 2569509 A1), 27H5 (see, for example, Canadian Patent Application CA 2569509 A1), 23F10 (see, for example, Canadian Patent Application CA 2569509 A1), 15C3 (see, for example, Canadian Patent Application CA 2569509 A1), visilizumab (see, for example, Dean et al., 2012, Swiss Med Wkly, 142: w13711), and Hum291 (see, for example, Dean et al., 2012, Swiss Med Wkly, 142: w13711).

In certain embodiments, the scFv in a bispecific binding molecule of the invention binds to the same epitope as a CD3-specific antibody known in the art. In a specific embodiment, the scFv in a bispecific binding molecule of the invention binds to the same epitope as the CD3-specific antibody huOKT3. Binding to the same epitope can be determined by assays known to one skilled in the art, such as, for example, mutational analyses or crystallographic studies. In certain embodiments, the scFv competes for binding to CD3 with an antibody known in the art. In a specific embodiment, the scFv in a bispecific binding molecule of the invention competes for binding to CD3 with the CD3-specific antibody huOKT3. Competition for binding to CD3 can be determined by assays known to one skilled in the art, such as, for example, flow cytometry. See, for example, Section 6.1.2.4. In certain embodiments, the scFv comprises a V_{H} with at least 85%, 90%, 95%, 98%, or at least 99% similarity to the V_{H} of a CD3-specific antibody known in the art. In certain embodiments, the scFv comprises the V_{H} of a CD3-specific antibody known in the art, comprising between 1 and 5 conservative amino acid substitutions. In certain embodiments, the scFv comprises a V_{L} with at least 85%, 90%, 95%, 98%, or at least 99% similarity to the V_{L} of a CD3-specific antibody known in the art. In certain embodiments, the scFv comprises the V_{L} of a CD3-specific antibody known in the art, comprising between 1 and 5 conservative amino acid substitutions.

Conservative amino acid substitutions are amino acid substitutions that occur within a family of amino acids, wherein the amino acids are related in their side chains. Generally, genetically encoded amino acids are divided into families: (1) acidic, comprising aspartate and glutamate; (2) basic, comprising arginine, lysine, and histidine; (3) non-polar, comprising isoleucine, alanine, valine, proline, methionine, leucine, phenylalanine, tryptophan; and (4) uncharged polar, comprising cysteine, threonine, glutamine, glycine, asparagine, serine, and tyrosine. In addition, an aliphatic-hydroxy family comprises serine and threonine. In addition, an amide-containing family comprises asparagine and glutamine. In addition, an aliphatic family comprises alanine, valine, leucine and isoleucine. In addition, an aromatic family comprises phenylalanine, tryptophan, and tyrosine. Finally, a sulfur-containing side chain family comprises cysteine and methionine. As an example, one skilled in the art would reasonably expect an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Preferred conservative amino acid substitution groups include: lysine-arginine, alanine-valine, phenylalanine-tyrosine, glutamic acid-aspartic acid, valine-leucine-isoleucine, cysteine-methionine, and asparagine-glutamine.

In a preferred embodiment, the scFv is derived from the huOKT3 antibody, and thus contains the V_{H} and V_{L} of huOKT3 monoclonal antibody (SEQ ID NOS: 15 and 16, respectively). See, for example, Van Wauwe et al., 1991, nature, 349:293-299. In specific embodiments of the bispecific binding molecule, the scFv is derived from the huOKT3 monoclonal antibody and has no more than 5 amino acid mutations relative to native huOKT3 V_{H} and V_{L} sequences. In certain embodiments of the bispecific binding molecule, the scFv is derived from the huOKT3 monoclonal antibody and comprises one or more mutations, relative to native huOKT3 V_{H} and V_{L} sequences, to stabilize disulfide binding. In certain embodiments of the bispecific binding molecule, the stabilization of disulfide binding prevents aggregation of the bispecific binding molecule. In certain embodiments of the bispecific binding molecule, the stabilization of disulfide binding reduces aggregation of the bispecific binding molecule as compared to aggregation of the bispecific binding molecule without the stabilization of disulfide binding. In certain embodiments of the bispecific binding molecule, the one or more mutations to stabilize disulfide binding comprise a V_{H} G44C mutation and a V_{L} Q100C mutation (e.g., as present in SEQ ID NOS: 54-59). In certain embodiments of the bispecific binding molecule, the one or more mutations to stabilize disulfide binding are the replacement of the amino acid residue at V_{H}44 (according to the Kabat numbering system) with a cysteine and the replacement of the amino acid residue at V_{L}100 (according to the Kabat numbering system) with a cysteine so as to introduce a disulfide bond between V_{H}44 and V_{L}100 (e.g., as present in SEQ ID NOS: 54-59). In an especially preferred embodiment, the scFv comprises the V_{H} of huOKT3 comprising the amino acid substitution at numbered position 105, wherein the cysteine is substituted with a serine (SEQ ID NO: 17). In certain embodiments, the sequence of the V_{H} of the scFv is as described in Table 4, below (e.g., any one of SEQ ID NOs: 15, 17, or 64). In certain embodiments, the sequence of the V_{L} of the scFv is as described in Table 5, below (e.g., any one of SEQ ID NOs: 16 or 65). In certain embodiments, the sequence of the scFv is as described in Table 6, below (e.g., any one of SEQ ID NOs: 19 or 48-59). In a preferred embodiment, the sequence of the scFv is SEQ ID NO: 19. In a specific embodiment, the scFv comprises a variant of the V_{H} of huOKT3 that has no more than 5 amino acid mutations relative to the native sequence of huOKT3 V_{H}. In a specific embodiment, the scFv comprises a variant of the V_{L} of huOKT3 that has no more than 5 amino acid mutations relative to the native sequence of huOKT3 V_{L}.

The sequences of the variable regions of an anti-CD3 scFv may be modified by insertions, substitutions and deletions to the extent that the resulting scFv maintains the ability to bind to CD3, as determined by, for example, ELISA, flow cytometry, and BiaCore™. The ordinarily skilled artisan can ascertain the maintenance of this activity by performing the functional assays as described herein below, such as, for example, binding analyses and cytotoxicity analyses.

In certain embodiments, the peptide linker conjugating the immunoglobulin light chain and the scFv is between 5-30, 5-25, 5-15, 10-30, 10-20, 10-15, 15-30, or 15-25 amino acids in length. In certain embodiments, the peptide linker displays one or more characteristics suitable for a peptide linker known to one of ordinary skill in the art. In certain embodiments, the peptide linker comprises amino acids that allow for peptide linker solubility, such as, for example, serine and threonine. In certain embodiments, the peptide linker comprises amino acids that allow for peptide linker flexibility, such as, for example, glycine. In certain embodiments, the sequence of the peptide linker conjugating the immunoglobulin light chain and the scFv is as described in Table 1, below (e.g., any one of SEQ ID NOs: 14 or 35-41). In preferred embodiments, the peptide linker is SEQ ID NO: 14.

In certain embodiments of the bispecific binding molecules of the invention, the immunoglobulin that binds to HER2 comprises the heavy chain and/or the light chain of a HER2-specific antibody known in the art, such as, for example, trastuzumab (see, for example, Baselga et al. 1998, Cancer Res 58 (13): 2825-2831), M-111 (see, for example, Higgins et al., 2011, J Clin Oncol, 29 (Suppl): Abstract TPS119), pertuzumab (see, for example, Franklin et al., 2004, Cancer Cell, 5:317-328), ertumaxomab (see, for example, Kiewe and Thiel, 2008, Expert Opin Investig Drugs, 17 (10): 1553-1558), MDXH210 (see, for example, Schwaab et al., 2001, Journal of Immunotherapy, 24 (1): 79-87), 2B1 (see, for example, Borghaei et al., 2007, J Immunother, 30:455-467), and MM-302 (see, for example, Wickham and Futch, 2012, Cancer Research, 72 (24): Supplement 3). In certain embodiments of the bispecific binding molecules of the invention, the immunoglobulin that binds to HER2 comprises the heavy chain of trastuzumab. In certain embodiments of the bispecific binding molecules of the invention, the immunoglobulin that binds to HER2 comprises the sequence as set forth in SEQ ID NO: 23. In certain embodiments of the bispecific binding molecules of the invention, the immunoglobulin that binds to HER2 comprises a variant of the heavy chain of trastuzumab (see, e.g., Table 2, below). In a specific embodiment of the bispecific binding molecules of the invention, the immunoglobulin that binds to HER2 comprises a variant of the light chain of trastuzumab that has no more than 5 amino acid mutations relative to the native sequence of trastuzumab. In certain embodiments of the bispecific binding molecules of the invention, the immunoglobulin that binds to HER2 comprises the light chain of trastuzumab (SEQ ID NO: 25). In certain embodiments of the bispecific binding molecules of the invention, the immunoglobulin that binds to HER2 comprises a variant of the light chain of trastuzumab. In a specific embodiment of the bispecific binding molecules of the invention, the immunoglobulin that binds to HER2 comprises a variant of the light chain of trastuzumab that has no more than 5 amino acid mutations relative to the native sequence of trastuzumab.

In certain embodiments of the bispecific binding molecules of the invention, the immunoglobulin that binds to HER2 binds to the same epitope as a HER2-specific antibody known in the art. In a specific embodiment, the immunoglobulin in a bispecific binding molecule of the invention binds to the same epitope as trastuzumab. Binding to the same epitope can be determined by assays known to one skilled in the art, such as, for example, mutational analyses or crystallographic studies. In certain embodiments, the immunoglobulin that binds to HER2 competes for binding to HER2 with an antibody known in the art. In a specific embodiment, the immunoglobulin in a bispecific binding molecule of the invention competes for binding to HER2 with trastuzumab. Competition for binding to HER2 can be determined by assays known to one skilled in the art, such as, for example, flow cytometry. See, for example, Section 6.1.2.4. In certain embodiments, the immunoglobulin comprises a V_{H} with at least 85%, 90%, 95%, 98%, or at least 99% similarity to the V_{H} of a HER2-specific antibody known in the art. In certain embodiments, the immunoglobulin comprises the V_{H} of a HER2-specific antibody known in the art, comprising between 1 and 5 conservative amino acid substitutions. In certain embodiments, the immunoglobulin comprises a V_{L} with at least 85%, 90%, 95%, 98%, or at least 99% similarity to the V_{L} of a HER2-specific antibody known in the art. In certain embodiments, the immunoglobulin comprises the V_{L} of a HER2-specific antibody known in the art, comprising between 1 and 5 conservative amino acid substitutions. In certain embodiments, the immunoglobulin comprises a V_{H} of a heavy chain described in Table 2, below (e.g., the V_{H} of any one of SEQ ID NOs: 23, 27, 62, or 63). In certain embodiments, the immunoglobulin comprises a V_{L} of a light chain described in Table 3, below (e.g., the V_{L} of SEQ ID NO: 25).

The sequences of the variable regions of an anti-HER2 antibody may be modified by insertions, substitutions and deletions to the extent that the resulting antibody maintains the ability to bind to HER2, as determined by, for example, ELISA, flow cytometry, and BiaCore™. The ordinarily skilled artisan can ascertain the maintenance of this activity by performing the functional assays as described herein below, such as, for example, binding analyses and cytotoxicity analyses.

In certain embodiments of the bispecific binding molecules of the invention, the immunoglobulin that binds to HER2 is an IgG1 immunoglobulin.

Methods of producing human antibodies are known to one skilled in the art, such as, for example, phage display methods described above using antibody libraries derived from human immunoglobulin sequences. See also, U.S. Pat. Nos. 4,444,887 and 4,716,111; and PCT publications WO 98/46645, WO 98/60433, WO 98/24893, WO 98/16664, WO 96/34096, WO 96/33735, and WO 91/10741; each of which is incorporated herein by reference in its entirety. The techniques of Cole et al., and Boerder et al., are also available for the preparation of human monoclonal antibodies (Cole et al., Monoclonal Antibodies and Cancer Therapy, Alan R. Riss, (1985); and Boerner et al., J. Immunol., 147 (1): 86-95, (1991)).

In certain embodiments, human antibodies are produced using transgenic mice, which are incapable of expressing functional endogenous mouse immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, for example, all or a portion of a polypeptide provided herein. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, for example, PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,886,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Human monoclonal antibodies can also be made by immunizing mice transplanted with human peripheral blood leukocytes, splenocytes or bone marrows (e.g., Trioma techniques of XTL). Completely human antibodies which recognize a selected epitope can be generated using a technique referred to as “guided selection.” In this approach a selected non-human monoclonal antibody, for example, a mouse antibody, is used to guide the selection of a completely human antibody recognizing the same epitope. See, for example, Jespers et al., Bio/technology 12:899-903 (1988). Human antibodies may also be generated by in vitro activated B cells. See U.S. Pat. Nos. 5,567,610 and 5,229,275, which are incorporated in their entirety by reference.

Methods for making humanized antibodies are known to one skilled in the art. See, for example, Winter EP 0 239 400; Jones et al., Nature 321:522-525 (1986); Riechmann et al., Nature 332:323-327 (1988); Verhoeyen et al., Science 239:1534-1536 (1988); Queen et al., Proc. Nat. Acad. ScL USA 86:10029 (1989); U.S. Pat. No. 6,180,370; and Orlandi et al., Proc. Natl. Acad. Sd. USA 86:3833 (1989); the disclosures of all of which are incorporated by reference herein in their entireties. Generally, the transplantation of murine (or other non-human) CDRs onto a human antibody is achieved as follows. The cDNAs encoding heavy and light chain variable domains are isolated from a hybridoma. The DNA sequences of the variable domains, including the CDRs, are determined by sequencing. The DNAs, encoding the CDRs are inserted into the corresponding regions of a human antibody heavy or light chain variable domain coding sequences, attached to human constant region gene segments of a desired isotype (e.g., gamma-l for CH and K for C_{L}), are gene synthesized. The humanized heavy and light chain genes are co-expressed in mammalian host cells (e.g., CHO or NSO cells) to produce soluble humanized antibody. To facilitate large scale production of antibodies, it is often desirable select for high expressor using a DHFR gene or GS gene in the producer line. These producer cell lines are cultured in bioreactors, or hollow fiber culture system, or WAVE technology, to produce bulk cultures of soluble antibody, or to produce transgenic mammals (e.g., goats, cows, or sheep) that express the antibody in milk (see, e.g., U.S. Pat. No. 5,827,690).

Antibody fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH, domain and/or hinge region of the heavy chain. The various portions of antibodies can be joined together chemically by conventional techniques, or can be prepared as a contiguous protein using genetic engineering techniques. In certain embodiments, elements of a human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. See, for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g. Johnson and Chiswell, Current Opinion in Structural Biology 3:5564-571 (1993).

Antibody humanization can also be performed by, for example, synthesizing a combinatorial library comprising the six CDRs of a non-human target monoclonal antibody fused in frame to a pool of individual human frameworks. A human framework library that contains genes representative of all known heavy and light chain human germline genes can be utilized. The resulting combinatorial libraries can then be screened for binding to antigens of interest. This approach can allow for the selection of the most favorable combinations of fully human frameworks in terms of maintaining the binding activity to the parental antibody. Humanized antibodies can then be further optimized by a variety of techniques.

Antibody humanization can be used to evolve mouse or other non-human antibodies into “fully human” antibodies. The resulting antibody contains only human sequence and no mouse or non-human antibody sequence, while maintaining similar binding affinity and specificity as the starting antibody.

For full length antibody molecules, the immunoglobulin genes can be obtained from genomic DNA or mRNA of hybridoma cell lines. Antibody heavy and light chains are cloned in a mammalian vector system. Assembly is documented with double strand sequence analysis. The antibody construct can be expressed in other human or mammalian host cell lines. The construct can then be validated by transient transfection assays and Western blot analysis of the expressed antibody of interest. Stable cell lines with the highest productivity can be isolated and screened using rapid assay methods.

In one approach, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMAIWA, NEURO 2A), or the like, or heteromylomas, fusion products thereof, or any cell or fusion cell derived therefrom, or any other suitable cell line as known in the art. See, for example, the ATCC or LifeTech website, and the like, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, avian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, for example, Ausubel, supra, and Colligan, Immunology, supra, chapter 2, entirely incorporated herein by reference. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

In a preferred specific embodiment, the bispecific binding molecule comprises a variant Fc region, wherein said variant Fc region comprises at least one amino acid modification relative to a wild-type Fc region, such that said molecule does not bind or has reduced binding to an Fc receptor (FcR), in soluble form or cell-bound form (including on immune-effector cells, such as, for example, NK cells, monocytes, and neutrophils). These FcRs include, but are not limited to, FcR1 (CD64), FcRII (CD32), and FcRIII (CD16). The affinity to FcR (n), the neonatal Fc receptor, is not affected, and thus maintained in the bispecific binding molecule. For example, if the immunoglobulin is an IgG, preferably, the IgG has reduced or no affinity for an Fc gamma receptor. In certain embodiments, one or more positions within the Fc region that makes a direct contact with Fc gamma receptor, such as, for example, amino acids 234-239 (hinge region), amino acids 265-269 (B/C loop), amino acids 297-299 (C′/E loop), and amino acids 327-332 (F/G) loop, are mutated such that the bispecific binding molecule has a decreased or no affinity for an Fc gamma receptor. See, for example, Sondermann et al., 2000, Nature, 406:267-273, which is incorporated herein by reference in its entirety. Preferably, for an IgG, the mutation N297A is made to destroy Fc receptor binding. In certain embodiments, affinity of the bispecific binding molecule or fragment thereof for an Fc gamma receptor is determined by, for example, BiaCore™ assay, as described, for example, in Okazaki et al., 2004. J Mol Biol, 336 (5): 1239-49. See also, Section 6. In certain embodiments, the bispecific binding molecule comprising such a variant Fc region binds an Fc receptor on a FcR-bearing immune-effector cell with less than 25%, 20%, 15%, 10%, or 5% binding as compared to a reference Fc region. Without being bound by any particular theory, a bispecific binding molecule comprising such a variant Fc region will have a decreased ability to induce a cytokine storm. In preferred embodiments, the bispecific binding molecule comprising such a variant Fc region does not bind an Fc receptor in soluble form or as a cell-bound form.

In certain embodiments, the bispecific binding molecule comprises a variant Fc region, such as, for example, an Fc region with additions, deletions, and/or substitutions to one or more amino acids in the Fc region of an antibody provided herein in order to alter effector function, or enhance or diminish affinity of antibody to FcR. In a preferred embodiment, the affinity of the antibody to FcR is diminished. Reduction or elimination of effector function is desirable in certain cases, such as, for example, in the case of antibodies whose mechanism of action involves blocking or antagonism but not killing of the cells bearing a target antigen. In certain embodiments, the Fc variants provided herein may be combined with other Fc modifications, including but not limited to modifications that alter effector function. In certain embodiments, such modifications provide additive, synergistic, or novel properties in antibodies or Fc fusions. Preferably, the Fc variants provided herein enhance the phenotype of the modification with which they are combined.

In preferred embodiments, the bispecific binding molecule of the invention is aglycosylated. Preferably, this is achieved by mutating the anti-HER2 immunoglobulin portion of the bispecific binding molecule in its Fc receptor to destroy a glycosylation site, preferably an N-linked glycosylation site. In another specific embodiment, an immunoglobulin is mutated to destroy an N-linked glycosylation site. In certain preferred embodiments, the bispecific binding molecule has been mutated to destroy an N-linked glycosylation site. In certain embodiments, the heavy chain of the bispecific binding molecule has an amino acid substitution to replace an asparagine that is an N-linked glycosylation site, with an amino acid that does not function as a glycosylation site. In a preferred embodiment, the method encompasses deleting the glycosylation site of the Fc region of a bispecific binding molecule, by modifying position 297 from asparagine to alanine (N297A). For example, in certain embodiments, the bispecific binding molecule comprises a heavy chain with the sequence of SEQ ID NO: 20. As used herein, “glycosylation sites” include any specific amino acid sequence in an antibody to which an oligosaccharide (i.e., carbohydrates containing two or more simple sugars linked together) will specifically and covalently attach. Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. Methods for modifying the glycosylation content of antibodies are well known in the art, see, for example, U.S. Pat. No. 6,218,149; EP 0 359 096 B1; U.S. Publication No. US 2002/0028486; WO 03/035835; U.S. Publication No. 2003/0115614; U.S. Pat. Nos. 6,218,149; 6,472,511; all of which are incorporated herein by reference in their entirety. In another embodiment, aglycosylation of the bispecific binding molecules of the invention can be achieved by recombinantly producing the bispecific binding molecule in a cell or expression system incapable of glycosylation, such as, for example, bacteria. In another embodiment, aglycosylation of the bispecific binding molecules of the invention can be achieved by enzymatically removing the carbohydrate moieties of the glycosylation site.

In preferred embodiments, the bispecific binding molecule of the invention does not bind or has reduced binding affinity (relative to a reference or wild type immunoglobulin) to the complement component C1q. Preferably, this is achieved by mutating the anti-HER2 immunoglobulin portion of the bispecific binding molecule to destroy a C1q binding site. In certain preferred embodiments, the method encompasses deleting the C1q binding site of the Fc region of an antibody, by modifying position 322 from lysine to alanine (K322A). For example, in certain embodiments, the bispecific binding molecule comprises a heavy chain with the sequence of SEQ ID NO: 21. In certain embodiments, affinity of the bispecific binding molecule or fragment thereof for the complement component C1q is determined by, for example, BiaCore™ assay, as described, for example, in Okazaki et al., 2004. J Mol Biol, 336 (5): 1239-49. See also, Section 6. In certain embodiments, the bispecific binding comprising an anti-HER2-immunoglobulin comprising a destroyed C1q binding site binds the complement component C1q with less than 25%, 20%, 15%, 10%, or 5% binding compared to a reference or wild type immunoglobulin. In certain embodiments, the bispecific binding molecule does not activate complement.

In preferred embodiments, the bispecific binding molecule of the invention comprises an immunoglobulin, wherein the immunoglobulin (i) comprises at least one amino acid modification relative to a wild-type Fc region, such that said molecule does not bind or has reduced binding to an Fc receptor in soluble form or as cell-bound form; (ii) comprises one or more mutations in the Fc region to destroy an N-linked glycosylation site; and (iii) does not or has reduced binding to the complement component C1q. For example, in certain embodiments, the bispecific binding molecule comprises an IgG comprising a first mutation, N297A, in the Fc region to (i) abolish or reduce binding to an Fc receptor in soluble form or as cell-bound form; and (ii) destroy an N-linked glycosylation site in the Fc region; and a second mutation, K322A, in the Fc region to (iii) abolish or reduce binding to the complement component C1q. See, for example, SEQ ID NO: 27.

In a preferred embodiment, the immunoglobulin that binds to HER2 comprises the variable regions of trastuzumab (see, e.g., Tables 2 and 3), and preferably a human IgG1 constant region. In a preferred embodiment, the immunoglobulin that binds to HER2 comprises the variable regions of trastuzumab wherein the sequence of the heavy chain is SEQ ID NO: 27 and wherein the sequence of the light chain is SEQ ID NO: 25. In a preferred embodiment, the immunoglobulin that binds to HER2 is a variant of trastuzumab, wherein the heavy chain does not bind or has reduced binding to an Fc receptor in soluble form or as cell-bound form. In a preferred embodiment, the heavy chain that does not bind an Fc receptor in soluble form or as a cell-bound form comprises a mutation in the Fc region to destroy an N-linked glycosylation site. In a preferred embodiment, the heavy chain has an amino acid substitution to replace an asparagine that is an N-linked glycosylation site, with an amino acid that does not function as a glycosylation site. In a preferred embodiment, the mutation to destroy an N-linked glycosylation site is N297A in the Fc region (SEQ ID NO: 20). In a preferred embodiment, the immunoglobulin that binds to HER2 comprises the variable regions of trastuzumab, wherein the sequence of the heavy chain comprises a mutation in the Fc region to destroy a C1q binding site. In a preferred embodiment, the immunoglobulin does not activate complement. In a preferred embodiment, the mutation to destroy a C1q binding site is K322A in the Fc region (SEQ ID NO: 21). In an especially preferred embodiment, the immunoglobulin that binds to HER2 comprises the variable regions of trastuzumab, wherein the immunoglobulin heavy chain comprises a mutation in the Fc region to destroy an N-linked glycosylation site and a mutation in the Fc region to destroy a C1q binding site (see, for example, SEQ ID NO: 27). In an especially preferred embodiment, the immunoglobulin that binds to HER2 comprises the variable regions of trastuzumab wherein the sequence of the heavy chain of the immunoglobulin has been mutated in the Fc region and is SEQ ID NO: 27 and wherein the sequence of the light chain is SEQ ID NO: 25. In an especially preferred embodiment, the sequence of the light chain fusion polypeptide is SEQ ID NO: 29. In certain embodiments, the heavy chain comprises the constant region of trastuzumab. In certain embodiments, the heavy chain comprises the constant region of a heavy chain described in Table 2, below (e.g., the constant region of any one of SEQ ID NOs: 23, 27, 62, or 63). In certain embodiments, the sequence of the heavy chain is as described in Table 2, below (e.g., any one of SEQ ID NOs: 23, 27, 62, or 63). In certain embodiments, the light chain comprises the constant region of a light chain described in Table 3, below (e.g., the constant region of SEQ ID NO: 25). In certain embodiments, the sequence of the light chain is as described in Table 3, below (e.g., SEQ ID NO: 25).

In certain embodiments, the bispecific binding molecule has a trastuzumab-derived sequence that contains one or more of the modifications in the trastuzumab immunoglobulin, and has a huOKT3-derived sequence that contains one or more of the modifications in the huOKT3 V_{H} and V_{L} sequences, as described in Table 8, below. Bispecific binding molecules having other immunoglobulin or scFv sequences can contain analogous mutations at corresponding positions in these other immunoglobulin or scFv sequences. In certain embodiments, the bispecific binding molecule is (a) derived from trastuzumab and huOKT3; and (b) contains one or more of the modifications as described in Table 8, below. In certain embodiments, the sequence of the peptide linker conjugating the immunoglobulin light chain and the scFv is as described in Table 1, below (e.g., any one of SEQ ID NOs: 14 or 35-41). In certain embodiments, the sequence of the heavy chain is as described in Table 2, below (e.g., any one of SEQ ID NOs: 23, 27, 62, or 63). In certain embodiments, the sequence of the light chain is as described in Table 3, below (e.g., SEQ ID NO: 25). In certain embodiments, the sequence of the V_{H} of the scFv is as described in Table 4, below (e.g., any one of SEQ ID NOs: 15, 17, or 64). In certain embodiments, the sequence of the V_{L} of the scFv is as described in Table 5, below (e.g., any one of SEQ ID NOs: 16 or 65). In certain embodiments, the sequence of the scFv peptide linker is as described in Table 1, below (e.g., any one of SEQ ID NOs: 14 or 35-41). In certain embodiments, the sequence of the scFv is as described in Table 6, below (e.g., any one of SEQ ID NOs: 19 48-59, or 66). In certain embodiments, the sequence of the light chain fusion polypeptide is as described in Table 7, below (e.g., any one of SEQ ID NOs: 29, 34, 42-47, or 60)

In certain embodiments, the bispecific binding molecule comprises a glycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, wherein the first and second light chain fusion polypeptides are identical, wherein the sequence of each heavy chain is SEQ ID NO: 62, and wherein the sequence of each light chain fusion polypeptide is SEQ ID NO: 60.

In certain embodiments, the bispecific binding molecule comprises a glycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, wherein the first and second light chain fusion polypeptides are identical, wherein the sequence of each heavy chain is SEQ ID NO: 27, and wherein the sequence of each light chain fusion polypeptide is SEQ ID NO: 47.

In certain embodiments, the bispecific binding molecule comprises a glycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, wherein the first and second light chain fusion polypeptides are identical, wherein the sequence of each heavy chain is SEQ ID NO: 27, and wherein the sequence of each light chain fusion polypeptide is SEQ ID NO: 29.

In certain embodiments, the bispecific binding molecule has low immunogenicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (Elliott et al., Lancet 344:1125-1127 (1994), entirely incorporated herein by reference).

The bispecific binding molecules provided herein can bind HER2 and CD3 with a wide range of affinities. The affinity or avidity of an antibody for an antigen can be determined experimentally using any suitable method. See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH). Thus, measurements of affinity and other antigen-binding parameters are preferably made with standardized solutions of antibody and antigen, and a standardized buffer, such as the buffer described herein. The affinity, K_{D} is a ratio of kₒₙ/k_{off}. Generally, a Ko in the micromolar range is considered low affinity. Generally, a Ko in the picomolar range is considered high affinity. In another specific embodiment, the bispecific binding molecule has high affinity for HER2 and low affinity for CD3. In another specific embodiment, the bispecific binding molecule has high affinity for HER2 and average affinity for CD3. In a specific embodiment, the bispecific binding molecule has a K_{D} of between 70 nM and 1 μM for CD3. In a specific embodiment, the bispecific binding molecule has a K_{D} of between 70 nM and 500 nM for CD3. In a specific embodiment, the bispecific binding molecule has a K_{D} of between 500 nM and 1 μM for CD3.

In certain embodiments, the bispecific binding molecule binds to one or more HER2-positive carcinoma cell lines, as determined by assays known to one skilled in the art, such as, for example, ELISA, BiaCore™, and flow cytometry. In certain embodiments, the carcinoma cell line is a breast carcinoma cell line, such as, for example, MDA-MB-361, MDA-MB-468, AU565, SKBR3, HTB27, HTB26, HCC1954, and/or MCF7. In certain embodiments, the carcinoma cell line is an ovarian carcinoma cell line, such as, for example, OVCAR3 and/or SKOV3. In certain embodiments, the carcinoma cell line is a gastric carcinoma cell line, such as, for example, NCI-N87, KATO III, AGS, and/or SNU-16. In certain embodiments, the carcinoma cell line is a melanoma cell line, such as, for example, HT144, SKMEL28, M14, and/or HTB63. In certain embodiments, the carcinoma cell line is an osteosarcoma cell line, such as, for example, RG160, RG164, CRL1427, and/or U2OS. In certain embodiments, the carcinoma cell line is a Ewings sarcoma cell line, such as, for example, SKEAW and/or SKES-1. In certain embodiments, the carcinoma cell line is a rhabdomyosarcoma cell line, such as, for example, HTB82. In certain embodiments, the carcinoma cell line is a neuroblastoma cell line, such as, for example, NMB7, SKNBE (2) C, IMR32, SKNBE (2) S, SKNBE (1) N, and/or NB5. In certain embodiments, the carcinoma cell line is a squamous cell carcinoma head and neck (SCCHN) cell lines, such as, for example, 15B, 93-VU-147T, PCI-30, UD-SCC2, PCI-15B, SCC90, and/or UMSCC47. In certain embodiments, the carcinoma cell line is a cervical cancer cell line, such as, for example, HeLa. In certain embodiments, the carcinoma cell line is a small cell lung cancer cell line, such as, for example, NCI-H524, NCI-H69, and/or NCI-H345. In certain embodiments, the bispecific binding molecule binds to the HER2-positive carcinoma cell line with an EC50 in the picomolar range. See, for example, Section 6.1.3.4 and Section 6.1.3.6.

In certain embodiments, the bispecific binding molecule binds to CD3+ T cells, as determined by assays known to one skilled in the art, such as, for example, ELISA, BiaCore™ and flow cytometry. In certain preferred embodiments, the bispecific binding molecule binds to CD3+ T cells with greater than 15-fold less binding than huOKT3 binding to CD3+ T cells. See, for example, Section 6.1.3.1. In certain embodiments, the CD3+ T cells are human T cells.

In certain embodiments, the bispecific binding molecule the bispecific binding molecule mediates T cell cytotoxicity against HER2-positive cells, as determined by assays known to one skilled in the art, such as, for example, cytotoxicity assays. In preferred embodiments, the bispecific binding molecule mediates T cell cytotoxicity against HER2-positive cell lines with an EC50 in the picomolar range. In certain embodiments, the HER2-positive cells are breast carcinoma cell lines, such as, for example, MDA-MB-361, MDA-MB-468, AU565, SKBR3, HTB27, HTB26, and/or MCF7. In certain embodiments, the HER2-positive cells are of an ovarian carcinoma cell line, such as, for example, OVCAR3 and/or SKOV3. In certain embodiments, the HER-2 positive cells are of a gastric carcinoma cell line, such as, for example, NCI-N87, KATO III, AGS, and/or SNU-16. In certain embodiments, the HER2-positive cells are of a melanoma cell line, such as, for example, HT144, SKMEL28, M14, and/or HTB63. In certain embodiments, the HER2-positive cells are of an osteosarcoma cell line, such as, for example, RG160, RG164, CRL1427, and/or U2OS. In certain embodiments, the HER2-positive cells are of an Ewings sarcoma cell line, such as, for example, SKEAW and/or SKES-1. In certain embodiments, the HER2-positive cells are of a rhabdomyosarcoma cell line, such as, for example, HTB82. In certain embodiments, the HER2-positive cells are of a neuroblastoma cell line, such as, for example, NMB7, SKNBE (2) C, IMR32, SKNBE (2) S, SKNBE (1) N, and/or NB5. In certain embodiments, the HER2-positive cells are of a squamous cell carcinoma head and neck (SCCHN) cell line, such as, for example, 15B, 93-VU-147T, PCI-30, UD-SCC2, PCI-15B, SCC90, and/or UMSCC47. In certain embodiments, the HER2-positive cells are of a cervical cancer cell line, such as, for example, HeLa. In certain embodiments, the HER2-positive cells are of a small cell lung cancer cell line, such as, for example, NCI-H524, NCI-H69, and/or NCI-H345. See, for example, Section 6.1.3.4 and Section 6.1.3.6.

In certain embodiments, preincubation of HER2-positive cells with huOKT3 blocks the ability of the bispecific binding molecule to induce T cell cytotoxicity. In certain embodiments, preincubation of HER2-positive cells with trastuzumab blocks the ability of the bispecific binding molecule to induce T cell cytotoxicity. See, for example, Section 6.1.3.3.

In certain embodiments, the bispecific binding molecule mediates T cell cytotoxicity against HER2-positive cells, wherein the level of HER2-expression in said cells is below the threshold of detection by flow cytometry performed with the bispecific binding molecule. See, for example, Section 6.1.3.4.

In certain embodiments, the bispecific binding molecule mediates T cell cytotoxicity against HER2-positive cells resistant to other HER-targeted therapies, such as, for example, trastuzumab, cetuximab, lapatinib, erlotinib, neratinib, or any other small molecule or antibody that targets the HER family of receptors. In a specific embodiment, the tumor that is resistant to HER-targeted therapies, such as, for example, trastuzumab, cetuximab, lapatinib, erlotinib, neratinib, or any other small molecule or antibody that targets the HER family of receptors is responsive to treatment with a bispecific binding molecule to the invention. See, for example, Section 6.1.3.7, Section 6.1.3.8, Section 6.1.3.9, and Section 6.1.3.10.

In certain embodiments, the bispecific binding molecule reduces HER2-positive tumor progression, metastasis, and/or tumor size. See, for example, Section 6.1.3.11.

In certain embodiments, the bispecific binding molecule is bound to a T cell. In certain embodiments, the binding of the bispecific binding molecule to a T cell is noncovalently. In certain embodiments, the T cell is administered to a subject. In certain embodiments, the T cell is autologous to the subject to whom the T cell is to be administered. In certain embodiments, the T cell is allogeneic to the subject to whom the T cell is to be administered. In certain embodiments, the T cell is a human T cell.

In certain embodiments, the bispecific binding molecule is not bound to a T cell.

In certain embodiments, the bispecific binding molecule is conjugated to an organic moiety, a detectable marker, and/or isotope as described in Section 5.2.

In certain embodiments, the bispecific binding molecule or fragment thereof is produced as described in Section 5.3. In certain embodiments, the bispecific binding molecule or fragment thereof is encoded by a polynucleotide as described in Section 5.3.1. In certain embodiments, the bispecific binding molecule or fragment thereof is encoded by a vector (e.g., expression vector) as described in Section 5.3.2. In certain embodiments, the bispecific binding molecule or fragment thereof is produced from a cell as described in Section 5.3.2.

In certain embodiments, the bispecific binding molecule is a component of a composition (e.g., pharmaceutical composition) and/or as part of a kit as described in Section 5.5.

In certain embodiments, the bispecific binding molecule is used according to the methods provided in Section 5.6. In certain embodiments, the bispecific binding molecule is used as a diagnostic tool according to the methods provided in Section 5.6.2. In certain embodiments, the bispecific binding molecule is used as a therapeutic according to the methods provided in Section 5.6.1. In certain embodiments, the bispecific binding molecule is administered to a subject, such as a subject described in Section 5.7, for use according to the methods provided in Section 5.6. In certain embodiments, the bispecific binding molecule is administered to a subject as part of a combination therapy as described in Section 5.9, for use according to the methods provided in Section 5.6.

TABLE 1
Linker Sequence

| DESCRIPTION | SEQUENCE (SEQ ID NO:) |
|---|---|
| (G₄S)₃ | GGGGSGGGGSGGGGS |
| | (SEQ ID NO: 14) |
| TS(G₄S)₃ Linker | TSGGGGSGGGGSGGGGS |
| | (SEQ ID NO: 35) |
| G₄S Linker | GGGGS |
| | (SEQ ID NO: 36) |
| (G₄S)₂ Linker | GGGGSGGGGS |
| | (SEQ ID NO: 37) |
| (G₄S)₃ Linker | GGGGSGGGGSGGGGS |
| | (SEQ ID NO: 38) |
| (G₄S)₄ Linker | GGGGSGGGGSGGGGSGGGGS |
| | (SEQ ID NO: 39) |
| (G₄S)₅ Linker | GGGGSGGGGSGGGGSGGGGSGGGGS |
| | (SEQ ID NO: 40) |
| (G₄S)₆ Linker | GGGGSGGGGSGGGGSGGGGSGGGGSGGGGS |
| | (SEQ ID NO: 41) |

TABLE 2
Heavy Chain Sequence. The non-italicized, non-underlined sequence
represents the V_{H} domain. The italicized sequence represents the constant
region. The underlined, italicized, and bold sequences represent the mutations
described in the “DESCRIPTION” column.

| DESCRIPTION | SEQUENCE (SEQ ID NO:) |
|---|---|
| Trastuzumab V_{H} | EVQLVESGGGLVQPGGSLRLSCAASGFNIKDTYIHWVRQAPGKG |
| domain with human | LEWVARIYPTNGYTRYADSVKGRFTISADTSKNTAYLQMNSLRA |
| IgG1 constant region | EDTAVYYCSRWGGDGFYAMIDYWGQGTLVTVSSASTKGPSVFPL |
| | APSSKSTSGGTAALGCLVKDYFPEPVTVSWNSGALTSGVHTFPAVLQS |
| | SGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 23) |
| Trastuzumab V_{H} | EVQLVESGGGLVQPGGSLRLSCAASGFNIKDTYIHWVRQAPGKG |
| domain with human | LEWVARIYPTNGYTRYADSVKGRFTISADTSKNTAYLQMNSLRA |
| IgG1 constant | EDTAVYYCSRWGGDGFYAMDYWGQGTLVTVSSASTKGPSVFPL |
| region; N297A; | APSSKSTSGGTAALGCLVKDYFPEPVTVSWNSGALTSGVHTFPAVLQS |
| K322A | SGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEV |
| | KFNWYVDGVEVHNAKTKPREEQYASTYRVVSVLTVLHQDWLNGKEY |
| | KCAVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVMHEALHNHYTQKSLSLSPG (SEQ ID NO: 27) |
| Trastuzumab V_{H} | EVQLVESGGGLVQPGGSLRLSCAASGFNIKDTYIHWVRQAPGKG |
| domain with human | LEWVARIYPTNGYTRYADSVKGRFTISADTSKNTAYLQMNSLRA |
| IgG1 constant | EDTAVYYCSRWGGDGFYAMDYWGQGTLVTVSSASTKGPSVFPL |
| region; N297A | APSSKSTSGGTAALGCLVKDYFPEPVTVSWNSGALTSGVHTFPAVLQS |
| | SGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEV |
| | KFNWYVDGVEVHNAKTKPREEQYASTYRVVSVLTVLHQDWLNGKEY |
| | KCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 62) |
| Trastuzumab V_{H} | EVQLVESGGGLVQPGGSLRLSCAASGFNIKDTYIHWVRQAPGKG |
| domain with human | LEWVARIYPTNGYTRYADSVKGRFTISADTSKNTAYLQMNSLRA |
| IgG1 constant | EDTAVYYCSRWGGDGFYAMDYWGQGTLVTVSSASTKGPSVFPL |
| region; K322A | APSSKSTSGGTAALGCLVKDYFPEPVTVSWNSGALTSGVHTFPAVLQS |
| | SGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEY |
| | KCAVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 63) |

TABLE 3
Light Chain Sequence. The non-italicized sequence represents the V_{L}
domain. The italicized sequence represents the constant region.

| DESCRIPTION | SEQUENCE (SEQ ID NO:) |
|---|---|
| Trastuzumab light | DIQMTQSPSSLSASVGDRVTITCRASQDVNTAVAWYQQKPGKAP |
| chain | KLLIYSASFLYSGVPSRFSGSRSGTDFTLTISSLQPEDFATYYCQQH |
| | YTTPPTFGQGTKVEIKRTVAAPSVFIFPPSDEQLKSGTASVVCLLNNF |
| | YPREAKVQWKVDNALQSGNSQESVTEQDSKDSTYSLSSTLTLSKADYE |
| | KHKVYACEVTHQGLSSPVTKSFNRGECTS (SEQ ID NO: 25) |

TABLE 4
scFv V_{H} Sequence. The underlined, italicized, and bold sequences
represent the mutations described in the “DESCRIPTION” column.

| DESCRIPTION | SEQUENCE (SEQ ID NO:) |
|---|---|
| huOKT3 V_{H} | QVQLVQSGGGVVQPGRSLRLSCKASGYTFTRYTMHWVRQAPGK |
| | GLEWIGYINPSRGYTNYNQKFKDRFTISRDNSKNTAFLQMDSLRP |
| | EDTGVYFCARYYDDHYCLDYWGQGTPVTVSS (SEQ ID NO: 15) |
| huOKT3 V_{H}; C105S | QVQLVQSGGGVVQPGRSLRLSCKASGYTFTRYTMHWVRQAPGK |
| | GLEWIGYINPSRGYTNYNQKFKDRFTISRDNSKNTAFLQMDSLRP |
| | EDTGVYFCARYYDDHYSLDYWGQGTPVTVSS (SEQ ID NO: 17) |
| huOKT3 V_{H}; | QVQLVQSGGGVVQPGRSLRLSCKASGYTFTRYTMHWVRQAPGK |
| C105S + V_{H}-G44C | CLEWIGYINPSRGYTNYNQKFKDRFTISRDNSKNTAFLQMDSLRP |
| | EDTGVYFCARYYDDHYSLDYWGQGTPVTVSS (SEQ ID NO: 64) |

TABLE 5
scFv V_{L} Sequence. The underlined, italicized, and bold sequence
represent the mutations described in the “DESCRIPTION” column.

| DESCRIPTION | SEQUENCE (SEQ ID NO:) |
|---|---|
| huOKT3 V_{L} | DIQMTQSPSSLSASVGDRVTITCSASSSVSYMNWYQQTPGKAPKR |
| | WIYDTSKLASGVPSRFSGSGSGTDYTFTISSLQPEDIATYYCQQWS |
| | SNPFTFGQGTKLQITR (SEQ ID NO: 16) |
| huOKT3 V_{L}; V_{L}- | DIQMTQSPSSLSASVGDRVTITCSASSSVSYMNWYQQTPGKAPKR |
| Q100C | WIYDTSKLASGVPSRFSGSGSGTDYTFTISSLQPEDIATYYCQQWS |
| | SNPFTFGCGTKLQITR (SEQ ID NO: 65) |

TABLE 6
scFv Sequence. The uppercase, non-italicized, non-bold, non-underlined
sequence represents the V_{H} domain. The uppercase, italicized sequence
represents the V_{L} domain. The uppercase, underlined, italicized, and bold
sequences represent the mutations described in the “DESCRIPTION” column. The
lowercase bold sequences represent the intra-scFv linker.

| DESCRIPTION | SEQUENCE (SEQ ID NO:) |
|---|---|
| huOKT3 scFv | QVQLVQSGGGVVQPGRSLRLSCKASGYTFTRYTMHWVRQAPGK |
| C105S; 15 amino | GLEWIGYINPSRGYTNYNQKFKDRFTISRDNSKNTAFLQMDSLRP |
| acid intra-scFv | EDTGVYFCARYYDDHYSLDYWGQGTPVTVSSggggsggggsggggsDI |
| linker | QMTQSPSSLSASVGDRVTITCSASSSVSYMNWYQQTPGKAPKRWIYDT |
| | SKLASGVPSRFSGSGSGTDYTFTISSLQPEDIATYYCQQWSSNPFTFGQ |
| | GTKLQITR (SEQ ID NO: 19) |
| huOKT3 scFv | QVQLVQSGGGVVQPGRSLRLSCKASGYTFTRYTMHWVRQAPGK |
| C105S; 5 amino acid | GLEWIGYINPSRGYTNYNQKFKDRFTISRDNSKNTAFLQMDSLRP |
| intra-scFv linker | EDTGVYFCARYYDDHYSLDYWGQGTPVTVSSggggsDIQMTQSPSS |
| | LSASVGDRVTITCSASSSVSYMNWYQQTPGKAPKRWIYDTSKLASGVPS |
| | RFSGSGSGTDYTFTISSLQPEDIATYYCQQWSSNPFTFGQGTKLQITR |
| | (SEQ ID NO: 48) |
| huOKT3 scFv | QVQLVQ SGGGVVQPGRSLRLSCKASGYTFTRYTMHWVRQAPGK |
| C105S; 10 amino | GLEWIGYINPSRGYTNYNQKFKDRFTISRDNSKNTAFLQMDSLRP |
| acid intra-scFv | EDTGVYFCARYYDDHYSLDYWGQGTPVTVSSggggsggggsDIQMT |
| linker | QSPSSLSASVGDRVTITCSASSSVSYMNWYQQTPGKAPKRWIYDTSKLA |
| | SGVPSRFSGSGSGTDYTFTISSLQPEDIATYYCQQWSSNPFTFGQGTKL |
| | QITR (SEQ ID NO: 49) |
| huOKT3 scFv | QVQLVQSGGGVVQPGRSLRLSCKASGYTFTRYTIVIEIWVRQAPGK |
| C105S; 20 amino | GLEWIGYINPSRGYTNYNQKFKDRFTISRDNSKNTAFLQMDSLRP |
| acid intra-scFv | EDTGVYFCARYYDDHYSLDYWGQGTPVTVSSggggsggggsggggsgg |
| linker | ggsDIQMTQSPSSLSASVGDRVTITCSASSSVSYMNWYQQTPGKAPKRW |
| | IYDTSKLASGVPSRFSGSGSGTDYTFTISSLQPEDIATYYCQQWSSNPFT |
| | FGQGTKLQITR (SEQ ID NO: 50) |
| huOKT3 scFv | QVQLVQSGGGVVQPGRSLRLSCKASGYTFTRYTIVIEIWVRQAPGK |
| C105S; 25 amino | GLEWIGYINPSRGYTNYNQKFKDRFTISRDNSKNTAFLQMDSLRP |
| acid intra-scFv | EDTGVYFCARYYDDHYSLDYWGQGTPVTVSSggggsggggsggggsgg |
| linker | ggsggggsDIQMTQSPSSLSASVGDRVTITCSASSSVSYMNWYQQTPGKA |
| | PKRWIYDTSKLASGVPSRFSGSGSGTDYTFTISSLQPEDIATYYCQQWS |
| | SNPFTFGQGTKLQITR (SEQ ID NO: 51) |
| huOKT3 scFv | QVQLVQSGGGVVQPGRSLRLSCKASGYTFTRYTIVIEIWVRQAPGK |
| C105S; 30 amino | GLEWIGYINPSRGYTNYNQKFKDRFTISRDNSKNTAFLQMDSLRP |
| acid intra-scFv | EDTGVYFCARYYDDHYSLDYWGQGTPVTVSSggggsggggsggggsgg |
| linker | ggsggggsggggsDIQMTQSPSSLSASVGDRVTITCSASSSVSYMNWYQQTP |
| | GKAPKRWIYDTSKLASGVPSRFSGSGSGTDYTFTISSLQPEDIATYYCQ |
| | QWSSNPFTFGQGTKLQITR (SEQ ID NO: 52) |
| huOKT3 scFv | QVQLVQSGGGVVQPGRSLRLSCKASGYTFTRYTIVIEIWVRQAPGK |
| C105S; V_{L}-Q100C; | CLEWIGYINPSRGYTNYNQKFKDRFTISRDNSKNTAFLQMDSLRP |
| V_{H}-G44C; 5 amino | EDTGVYFCARYYDDHYSLDYWGQGTPVTVSSggggsDIQMTQSPSS |
| acid intra-scFv | LSASVGDRVTITCSASSSVSYMNWYQQTPGKAPKRWIYDTSKLASGVPS |
| linker | RFSGSGSGTDYTFTISSLQPEDIATYYCQQWSSNPFTFGCGTKLQITR |
| | (SEQ ID NO: 53) |
| huOKT3 scFv | QVQLVQSGGGVVQPGRSLRLSCKASGYTFTRYTIVIEIWVRQAPGK |
| C105S; V_{L}-Q100C; | CLEWIGYINPSRGYTNYNQKFKDRFTISRDNSKNTAFLQMDSLRP |
| V_{H}-G44C; 10 amino | EDTGVYFCARYYDDHYSLDYWGQGTPVTVSSggggsggggsDIQMT |
| acid intra-scFv | QSPSSLSASVGDRVTITCSASSSVSYMNWYQQTPGKAPKRWIYDTSKLA |
| linker | SGVPSRFSGSGSGTDYTFTISSLQPEDIATYYCQQWSSNPFTFGCGTKL |
| | QITR (SEQ ID NO: 54) |
| huOKT3 scFv | QVQLVQSGGGVVQPGRSLRLSCKASGYTFTRYTIVIEIWVRQAPGK |
| C105S; V_{L}-Q100C; | CLEWIGYINPSRGYTNYNQKFKDRFTISRDNSKNTAFLQMDSLRP |
| V_{H}-G44C; 15 amino | EDTGVYFCARYYDDHYSLDYWGQGTPVTVSSggggsggggsggggsDI |
| acid intra-scFv | QMTQSPSSLSASVGDRVTITCSASSSVSYMNWYQQTPGKAPKRWIYDT |
| linker | SKLASGVPSRFSGSGSGTDYTFTISSLQPEDIATYYCQQWSSNPFTFGC |
| | GTKLQITR (SEQ ID NO: 55) |
| huOKT3 scFv | QVQLVQSGGGVVQPGRSLRLSCKASGYTFTRYTIVIEIWVRQAPGK |
| C105S; V_{L}-Q100C; | CLEWIGYINPSRGYTNYNQKFKDRFTISRDNSKNTAFLQMDSLRP |
| V_{H}-G44C; 20 amino | EDTGVYFCARYYDDHYSLDYWGQGTPVTVSSggggsggggsggggsgg |
| acid intra-scFv | ggsDIQMTQSPSSLSASVGDRVTITCSASSSVSYMNWYQQTPGKAPKRW |
| linker | IYDTSKLASGVPSRFSGSGSGTDYTFTISSLQPEDIATYYCQQWSSNPFT |
| | FGCGTKLQITR (SEQ ID NO: 56) |
| huOKT3 scFv | QVQLVQSGGGVVQPGRSLRLSCKASGYTFTRYTIVIEIWVRQAPGK |
| C105S; V_{L}-Q100C; | CLEWIGYINPSRGYTNYNQKFKDRFTISRDNSKNTAFLQMDSLRP |
| V_{H}-G44C; 25 amino | EDTGVYFCARYYDDHYSLDYWGQGTPVTVSSggggsggggsggggsgg |
| acid intra-scFv | ggsggggsDIQMTQSPSSLSASVGDRVTITCSASSSVSYMNWYQQTPGKA |
| linker | PKRWIYDTSKLASGVPSRFSGSGSGTDYTFTISSLQPEDIATYYCQQWS |
| | SNPFTFGCGTKLQITR (SEQ ID NO: 57) |
| huOKT3 scFv | QVQLVQSGGGVVQPGRSLRLSCKASGYTFTRYTMHWVRQAPGK |
| C105S; V_{L}-Q100C; | CLEWIGYINPSRGYTNYNQKFKDRFTISRDNSKNTAFLQMDSLRP |
| V_{H}-G44C; 30 amino | EDTGVYFCARYYDDHYSLDYWGQGTPVTVSSggggsggggsggggsgg |
| acid intra-scFv | ggsggggsggggsDIQMTQSPSSLSASVGDRVTITCSASSSVSYMNWYQQTP |
| linker | GKAPKRWIYDTSKLASGVPSRFSGSGSGTDYTFTISSLQPEDIATYYCQ |
| | QWSSNPFTFGCGTKLQITR (SEQ ID NO: 58) |
| huOKT3; 15 amino | QVQLVQSGGGVVQPGRSLRLSCKASGYTFTRYTMHWVRQAPGK |
| acid intra-scFv | GLEWIGYINPSRGYTNYNQKFKDRFTISRDNSKNTAFLQMDSLRP |
| linker | EDTGVYFCARYYDDHYCLDYWGQGTPVTVSSggggsggggsggggsDI |
| | QMTQSPSSLSASVGDRVTITCSASSSVSYMNWYQQTPGKAPKRWIYDT |
| | SKLASGVPSRFSGSGSGTDYTFTISSLQPEDIATYYCQQWSSNPFTFGQ |
| | GTKLQITR (SEQ ID NO: 59) |

TABLE 7
Light Chain Fusion Polypeptide Sequence. The uppercase, non-italicized, non-bold,
non-underlined sequence represents the V_{L} domain of the trastuzumab light chain.
The uppercase, italicized sequence represents the constant region of the trastuzumab
light chain. The lowercase, non-italicized, non-bold, non-underlined sequence represents
the linker conjugating the light chain to the scFv. The uppercase, underlined sequence
represents the V_{H} domain of the scFv. The uppercase, bold sequence represents the V_{L}
domain of the scFv. The uppercase, underlined, italicized, and bold sequences represent
the mutations described in the “DESCRIPTION” column. The lowercase bold sequences
represent the intra-scFv linker.

| DESCRIPTION | SEQUENCE (SEQ ID NO:) |
|---|---|
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the scFv; 15 | KHKVYACEVTHQGLSSPVTKSFNRGECtsggggsggggsggggsQVQLVQS |
| amino acid intra- | GGGVVQPGRSLRLSCKASGYTFTRYTMEIWVRQAPGKGLEWIGY |
| scFv peptide linker | INPSRGYTNYNQKFKDRFTISRDNSKNTAFLQMDSLRPEDTGVYF |
| | CARYYDDHYSLDYWGQGTPVTVSSggggsggggsggggsDIQMTQSP |
| | SSLSASVGDRVTITCSASSSVSYMNWYQQTPGKAPKRWIYDTS |
| | KLASGVPSRFSGSGSGTDYTFTISSLQPEDIATYYCQQWSSNPF |
| | TFGQGTKLQITR (SEQ ID NO: 29) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the scFv; 5 | KHKVYACEVTHQGLSSPVTKSFNRGECtsggggsggggsggggsQVQLVQS |
| amino acid intra- | GGGVVQPGRSLRLSCKASGYTFTRYTMHWVRQAPGKGLEWIGY |
| scFv peptide linker | INPSRGYTNYNQKFKDRFTISRDNSKNTAFLQMDSLRPEDTGVYF |
| | CARYYDDHYSLDYWGQGTPVTVSSggggsDIQMTQSPSSLSASVG |
| | DRVTITCSASSSVSYMNWYQQTPGKAPKRWIYDTSKLASGVP |
| | SRFSGSGSGTDYTFTISSLQPEDIATYYCQQWSSNPFTFGQGTK |
| | LQITR (SEQ ID NO: 30) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the scFv; 10 | KHKVYACEVTHQGLSSPVTKSFNRGECtsggggsggggsggggsQVQLVQS |
| amino acid intra- | GGGVVQPGRSLRLSCKASGYTFTRYTMHWVRQAPGKGLEWIGY |
| scFv peptide linker | INPSRGYTNYNQKFKDRFTISRDNSKNTAFLQMDSLRPEDTGVYF |
| | CARYYDDHYSLDYWGQGTPVTVSSggggsggggsDIQMTQSPSSLS |
| | ASVGDRVTITCSASSSVSYMNWYQQTPGKAPKRWIYDTSKLA |
| | SGVPSRFSGSGSGTDYTFTISSLQPEDIATYYCQQWSSNPFTFG |
| | QGTKLQITR (SEQ ID NO: 31) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the scFv; 20 | KHKVYACEVTHQGLSSPVTKSFNRGECtsggggsggggsggggsQVQLVQS |
| amino acid intra- | GGGVVQPGRSLRLSCKASGYTFTRYTMHWVRQAPGKGLEWIGY |
| scFv peptide linker | INPSRGYTNYNQKFKDRFTISRDNSKNTAFLQMDSLRPEDTGVYF |
| | CARYYDDHYSLDYWGQGTPVTVSSggggsggggsggggsggggsDIQMT |
| | QSPSSLSASVGDRVTITCSASSSVSYMNWYQQTPGKAPKRWIY |
| | DTSKLASGVPSRFSGSGSGTDYTFTISSLQPEDIATYYCQQWSS |
| | NPFTFGQGTKLQITR (SEQ ID NO: 32) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the scFv; 25 | KHKVYACEVTHQGLSSPVTKSFNRGECtsggggsggggsggggsQVQLVQS |
| amino acid intra- | GGGVVQPGRSLRLSCKASGYTFTRYTMHWVRQAPGKGLEWIGY |
| scFv peptide linker | INPSRGYTNYNQKFKDRFTISRDNSKNTAFLQMDSLRPEDTGVYF |
| | CARYYDDHYSLDYWGQGTPVTVSSggggsggggsggggsggggsggggsDI |
| | QMTQSPSSLSASVGDRVTITCSASSSVSYMNWYQQTPGKAPK |
| | RWIYDTSKLASGVPSRFSGSGSGTDYTFTISSLQPEDIATYYCQ |
| | QWSSNPFTFGQGTKLQITR (SEQ ID NO: 33) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the scFv; 30 | KHKVYACEVTHQGLSSPVTKSFNRGECtsggggsggggsggggsQVQLVQS |
| amino acid intra- | GGGVVQPGRSLRLSCKASGYTFTRYTMHWVRQAPGKGLEWIGY |
| scFv peptide linker | INPSRGYTNYNQKFKDRFTISRDNSKNTAFLQMDSLRPEDTGVYF |
| | CARYYDDHYSLDYWGQGTPVTVSSggggsggggsggggsggggsggggsgg |
| | ggsDIQMTQSPSSLSASVGDRVTITCSASSSVSYMNWYQQTPGK |
| | APKRWIYDTSKLASGVPSRFSGSGSGTDYTFTISSLQPEDIATY |
| | YCQQWSSNPFTFGQGTKLQITR (SEQ ID NO: 34) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSLSSTLTLSKADYE |
| chain to the huOKT3 | KHKVYACEVTHQGLSSPVTKSFNRGECtsggggsggggsggggsQVQLVQS |
| scFv; 5 amino acid | GGGVVQPGRSLRLSCKASGYTFTRYTMHWVRQAPGKCLEWIGYI |
| intra-scFv peptide | NPSRGYTNYNQKFKDRFTISRDNSKNTAFLQMDSLRPEDTGVYF |
| linker; V_{L}-Q100C; | CARYYDDHYSLDYWGQGTPVTVSSggggsDIQMTQSPSSLSASVG |
| V_{H}-G44C | DRVTITCSASSSVSYMNWYQQTPGKAPKRWIYDTSKLASGVP |
| | SRFSGSGSGTDYTFTISSLQPEDIATYYCQQWSSNPFTFGCGTK |
| | LQITR (SEQ ID NO: 42) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the huOKT3 | KHKVYACEVTHQGLSSPVTKSFNRGECtsggggsggggsggggsQVQLVQS |
| scFv; 10 amino acid | GGGVVQPGRSLRLSCKASGYTFTRYTMHWVRQAPGKCLEWIGYI |
| intra-scFv peptide | NPSRGYTNYNQKFKDRFTISRDNSKNTAFLQMDSLRPEDTGVYF |
| linker; V_{L}-Q100C; | CARYYDDHYSLDYWGQGTPVTVSSggggsggggsDIQMTQSPSSLS |
| V_{H}-G44C | ASVGDRVTITCSASSSVSYMNWYQQTPGKAPKRWIYDTSKLA |
| | SGVPSRFSGSGSGTDYTFTISSLQPEDIATYYCQQWSSNPFTFG |
| | CGTKLQITR (SEQ ID NO: 43) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the huOKT3 | KHKVYACEVTHQGLSSPVTKSFNRGECtsggggsggggsggggsQVQLVQS |
| scFv; 15 amino acid | GGGVVQPGRSLRLSCKASGYTFTRYTMHWVRQAPGKCLEWIGYI |
| intra-scFv peptide | NPSRGYTNYNQKFKDRFTISRDNSKNTAFLQMDSLRPEDTGVYF |
| linker; V_{L}-Q100C; | CARYYDDHYSLDYWGQGTPVTVSSggggsggggsggggsDIQMTQSP |
| V_{H}-G44C | SSLSASVGDRVTITCSASSSVSYMNWYQQTPGKAPKRWIYDTS |
| | KLASGVPSRFSGSGSGTDYTFTISSLQPEDIATYYCQQWSSNPF |
| | TFGCGTKLQITR (SEQ ID NO: 44) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the huOKT3 | KHKVYACEVTHQGLSSPVTKSFNRGECtsggggsggggsggggsQVQLVQS |
| scFv; 20 amino acid | GGGVVQPGRSLRLSCKASGYTFTRYTMHWVRQAPGKCLEWIGYI |
| intra-scFv peptide | NPSRGYTNYNQKFKDRFTISRDNSKNTAFLQMDSLRPEDTGVYF |
| linker; V_{L}-Q100C; | CARYYDDHYSLDYWGQGTPVTVSSggggsggggsggggsggggsDIQMT |
| V_{H}-G44C | QSPSSLSASVGDRVTITCSASSSVSYMNWYQQTPGKAPKRWIY |
| | DTSKLASGVPSRFSGSGSGTDYTFTISSLQPEDIATYYCQQWSS |
| | NPFTFGCGTKLQITR (SEQ ID NO: 45) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the huOKT3 | KHKVYACEVTHQGLSSPVTKSFNRGECtsggggsggggsggggsQVQLVQS |
| scFv; 25 amino acid | GGGVVQPGRSLRLSCKASGYTFTRYTMEIWVRQAPGKCLEWIGYI |
| intra-scFv peptide | NPSRGYTNYNQKFKDRFTISRDNSKNTAFLQMDSLRPEDTGVYF |
| linker; V_{L}-Q100C; | CARYYDDHYSLDYWGQGTPVTVSSggggsggggsggggsggggsggggsDI |
| V_{H}-G44C | QMTQSPSSLSASVGDRVTITCSASSSVSYMNWYQQTPGKAPK |
| | RWIYDTSKLASGVPSRFSGSGSGTDYTFTISSLQPEDIATYYCQ |
| | QWSSNPFTFGQCGTKLQITR (SEQ ID NO: 46) |
| Trastuzumab light | DIQMTQSPSSLSASVGDRVTITCRASQDVNTAVAWYQQKPGKAP |
| chain; C105S; 17 | KLLIYSASFLYSGVPSRFSGSRSGTDFTLTISSLQPEDFATYYCQQH |
| amino acid linker | YTTPPTFGQGTKVEIKRTVAAPSVFIFPPSDEQLKSGTASVVCLLNNF |
| conjugating the light | YPREAKVQWKVDNALQSGNSQESVTEQDSKDSTYSTSSTLTLSKADYE |
| chain to the huOKT3 | KHKVYACEVTHQGLSSPVTKSFNRGECtsggggsggggsggggsQVQLVQS |
| scFv; 30 amino acid | GGGVVQPGRSLRLSCKASGYTFTRYTMEIWVRQAPGKCLEWIGYI |
| intra-scFv peptide | NPSRGYTNYNQKFKDRFTISRDNSKNTAFLQMDSLRPEDTGVYF |
| linker; V_{L}-Q100C; | CARYYDDHYSLDYWGQGTPVTVSSggggsggggsggggsggggsggggsgg |
| V_{H}-G44C | ggsDIQMTQSPSSLSASVGDRVTITCSASSSVSYMNWYQQTPGK |
| | APKRWIYDTSKLASGVPSRFSGSGSGTDYTFTISSLQPEDIATY |
| | YCQQWSSNPFTFGCGTKLQITR (SEQ ID NO: 47) |
| Trastuzumab light | DIQMTQSPSSLSASVGDRVTITCRASQDVNTAVAWYQQKPGKAP |
| chain; 17 amino acid | KLLIYSASFLYSGVPSRFSGSRSGTDFTLTISSLQPEDFATYYCQQH |
| linker conjugating | YTTPPTFGQGTKVEIKRTVAAPSVFIFPPSDEQLKSGTASVVCLLNNF |
| the light chain to the | YPREAKVQWKVDNALQSGNSQESVTEQDSKDSTYSTSSTLTLSKADYE |
| scFv; 15 amino acid | KHKVYACEVTHQGLSSPVTKSFNRGECtsggggsggggsggggsQVQLVQS |
| intra-scFv peptide | GGGVVQPGRSLRLSCKASGYTFTRYTMEIWVRQAPGKGLEWIGY |
| linker | INPSRGYTNYNQKFKDRFTISRDNSKNTAFLQMDSLRPEDTGVYF |
| | CARYYDDHYSLDYWGQGTPVTVSSggggsggggsggggsDIQMTQSP |
| | SSLSASVGDRVTITCSASSSVSYMNWYQQTPGKAPKRWIYDTS |
| | KLASGVPSRFSGSGSGTDYTFTISSLQPEDIATYYCQQWSSNPF |
| | TFGQGTKLQITR (SEQ ID NO: 60) |

TABLE 8
Modifications to bispecific binding molecules

| LOCATION OF | |
|---|---|
| MODIFICATION | DESCRIPTION |
| Heavy chain | Mutation to reduce binding to the Fc receptor |
| | (as an example, N297A mutation) |
| | Mutation to destroy a glycosylation site (as an |
| | example, N297A mutation) |
| | Mutation to reduce C1q binding (as an example, |
| | K322A mutation) |
| Linker conjugating | Increase or decrease the length of the linker |
| the light chain to | |
| the huOKT3 scFv | |
| huOKT3 scFv V_{H} | Mutation to increase stabilization and/or reduce |
| | aggregation (as an example, introduce disulfide |
| | binding between V_{H}40 and V_{L}100 (according |
| | to Kabat numbering), as an example, V_{H} G44C |
| | and V_{L} Q100C) |
| | Reduce aggregation (as an example, C105S |
| | mutation) |
| huOKT3 scFv V_{L} | Mutation to increase stabilization and/or reduce |
| | aggregation (as an example,, introduce disulfide |
| | binding between V_{H}40 and V_{L}100 (according |
| | to Kabat numbering), as an example, V_{H} G44C |
| | and V_{L} Q100C) |
| huOKT3 intra-scFv | Increase or decrease the length of the linker |
| linker | |

### 5.2 Bispecific Binding Molecule Conjugates

In preferred embodiments, a bispecific binding molecule provided herein is not conjugated to any other molecule, such as an organic moiety, a detectable label, or an isotope. In alternative embodiments, a bispecific binding molecule provided herein is conjugated to one or more organic moieties. In alternative embodiments, a bispecific binding molecule provided herein is conjugated to one or more detectable labels. In alternative embodiments, a bispecific binding molecule provided herein is conjugated to one or more isotopes.

### 5.2.1 Detectable Labels and Isotopes

In certain embodiments, a bispecific binding molecule provided herein is conjugated to one or more detectable labels or isotopes, e.g., for imaging purposes. In certain embodiments, a bispecific binding molecule is detectably labeled by covalent or non-covalent attachment of a chromogenic, enzymatic, radioisotopic, isotopic, fluorescent, toxic, chemiluminescent, nuclear magnetic resonance contrast agent or other label.

Non-limiting examples of suitable chromogenic labels include diaminobenzidine and 4-hydroxyazo-benzene-2-carboxylic acid.

Non-limiting examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Non-limiting examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ²²³Ra, ²²³Ra, ⁸⁹Zr, ¹⁷⁷Lu, and ¹⁰⁹Pd. In certain embodiments, 111 In is a preferred isotope for in vivo imaging as it avoids the problem of dehalogenation of ¹²⁵I or ¹³¹I-labeled bispecific binding molecules in the liver. In addition, ¹¹¹In has a more favorable gamma emission energy for imaging (Perkins et al., Eur. J. Nucl. Med. 70:296-301 (1985); Carasquillo et ah, J. Nucl. Med. 25:281-287 (1987)). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870 (1987)).

Non-limiting examples of suitable non-radioactive isotopic labels include ¹⁵⁷Gd, ⁵⁵Mn, ¹⁶²Dy, ⁵²Tr, and ⁵⁶Fe.

Non-limiting examples of suitable fluorescent labels include a 152Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label.

Non-limiting examples of chemiluminescent labels include a luminol label, an isoluminol label, an aromatic acridinium ester label, an imidazole label, an acridinium salt label, an oxalate ester label, a luciferin label, a luciferase label, and an aequorin label.

Non-limiting examples of nuclear magnetic resonance contrasting agents include heavy metal nuclei such as Gd, Mn, and iron.

Techniques known to one of ordinary skill in the art for binding the above-described labels to a bispecific binding molecule provided herein are described in, for example, Kennedy et at., Clin. CMm. Acta 70:1-31 (1976), and Schurs et al., Clin. CMm. Acta 81:1-40 (1977). Coupling techniques mentioned in the latter are the glutaraldehyde method, the periodate method, the dimaleimide method, the m-maleimidobenzyl-N-hydroxy-succinimide ester method, all of which methods are incorporated by reference herein.

In certain embodiments, the bispecific binding molecule is conjugated to a diagnostic agent. A diagnostic agent is an agent useful in diagnosing or detecting a disease by locating the cells containing the antigen. Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. Preferably, the diagnostic agents are selected from the group consisting of radioisotopes, enhancing agents for use in magnetic resonance imaging, and fluorescent compounds. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, for example, ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose. Chelates are coupled to the antibodies using standard chemistries. The chelate is normally linked to the antibody by a group which enables formation of a bond to the molecule with minimal loss of immunoreactivity and minimal aggregation and/or internal cross-linking other, more unusual, methods and reagents for conjugating chelates to antibodies are disclosed in U.S. Pat. No. 4,824,659 to Hawthorne, entitled “Antibody Conjugates,” issued Apr. 25, 1989, the disclosure of which is incorporated herein in its entirety by reference. Particularly useful metal-chelate combinations include 2-benzyl-DTPA and its monomethyl and cyclohexyl analogs, used with diagnostic isotopes for radio-imaging. The same chelates, when complexed with non-radioactive metals, such as manganese, iron and gadolinium are useful for MRI, when used along bispecific binding molecules provided herein. Macrocyclic chelates such as NOTA, DOTA, and TETA are of use with a variety of metals and radiometals, most particularly with radionuclides of gallium, yttrium and copper, respectively. Such metal-chelate complexes can be made very stable by tailoring the ring size to the metal of interest. Other ring-type chelates such as macrocyclic polyethers, which are of interest for stably binding nuclides, such as ²²³Ra for RAIT are encompassed herein.

### 5.2.2 Organic Conjugates

In certain embodiments, the bispecific binding molecules provided herein comprise one or more organic moieties that are covalently bonded, directly or indirectly, to the bispecific binding molecule. Such modification can produce an antibody or antigen-binding fragment with improved pharmacokinetic properties (e.g., increased in vivo serum half-life). The organic moiety can be a hydrophilic polymeric group, fatty acid group, or fatty acid ester group. As used herein, the term “fatty acid” encompasses mono-carboxylic acids and di-carboxylic acids. As used herein, a “hydrophilic polymeric group” refers to an organic polymer that is more soluble in water than in octane, e.g., polylysine. Hydrophilic polymers suitable for modifying a bispecific binding molecule provided herein can be linear or branched and include, for example, polyalkane glycols (e.g., polyethylene glycol, (PEG), monomethoxy-polyethylene glycol, and polypropylene glycol), carbohydrates (e.g., dextran, cellulose, oligosaccharides, and polysaccharides), polymers of hydrophilic amino acids (e.g., polylysine, polyarginine, and polyaspartate), polyalkane oxides (e.g., polyethylene oxide and polypropylene oxide) and polyvinyl pyrolidone. In certain embodiments, the hydrophilic polymer that modifies a bispecific binding molecule provided herein has a molecular weight of about 800 to about 150,000 Daltons as a separate molecular entity. For example PEG₅₀₀₀ and PEG_{20.000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying bispecific binding molecules provided herein can be saturated or can contain one or more units of unsaturation. Fatty acids that are suitable for modifying bispecific binding molecules provided herein include, for example, n-dodecanoate, n-tetradecanoate, n-octadecanoate, n-eicosanoate, n-docosanoate, n-triacontanoate, n-tetracontanoate, cis-delta-9-octadecanoate, all cis-delta-5,8, 11,14-eicosatetraenoate, octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The bispecific binding molecule conjugates provided herein can be prepared using suitable methods, such as by reaction with one or more modifying agents. As used herein, an “activating group” is a chemical moiety or functional group that can, under appropriate conditions, react with a second chemical group thereby forming a covalent bond between the modifying agent and the second chemical group. For example, amine-reactive activating groups include electrophilic groups such as, for example, tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see, for example, Hernanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group, wherein one or more carbon atoms can be replaced by a heteroatom such as oxygen, nitrogen or sulfur. Suitable linker moieties include, for example, tetraethylene glycol, (CH₂)₃, and NH. Modifying agents that comprise a linker moiety can be produced, for example, by reacting a mono-Boc-alkyldiamine (e.g., mono-Boc-ethylenediamine or mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

As used herein, a “modifying agent” refers to a suitable organic group (e.g., hydrophilic polymer, a fatty acid, and a fatty acid ester) that comprises an activating group. For example, the organic moieties can be bonded to the bispecific binding molecule in a non-site specific manner by employing an amine-reactive modifying agent, for example, an N-hydroxysuccinimide ester of PEG. Modified bispecific binding molecules can also be prepared by reducing disulfide bonds (e.g., intra-chain disulfide bonds) of bispecific binding molecule. The reduced bispecific binding molecule can then be reacted with a thiol-reactive modifying agent to produce the modified bispecific binding molecule provided herein. Modified bispecific binding molecules comprising an organic moiety that is bonded to specific sites of a bispecific binding molecule provided herein can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6 (10): 2233-2241 (1997); Itoh et al., Bioorg. Chem., 24 (1): 59-68 (1996); Capellas et al., Biotechnol. Bioeng., 56 (4): 456-463 (1997)), and the methods described in Hermanson, G. T., Bioconjugate Techniques, Academic Press: San Diego, Calif. (1996).

### 5.3 Bispecific Binding Molecule Production

Provided herein are methods for producing bispecific binding molecules as described in Section 5.1 and Section 5.2. In certain embodiments, provided herein are methods for producing a bispecific binding molecule comprising an aglycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFV), via a peptide linker, to create a first fusion polypeptide, and wherein the second light chain is fused to a second scFv, via a peptide linker, to create a second fusion polypeptide, wherein the first and second scFv (i) are identical, and (ii) bind to CD3, and wherein the first and second fusion polypeptides are identical.

Methods to produce bispecific binding molecules described herein are known to one of ordinary skill in the art, for example, by chemical synthesis, by purification from biological sources, or by recombinant expression techniques, including, for example, from mammalian cell or transgenic preparations. The methods described herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, for example, Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

A variety of methods exist in the art for the production of bispecific binding molecules. For example, the bispecific binding molecule may be made by recombinant DNA methods, such as those described in U.S. Pat. No. 4,816,567. The one or more DNAs encoding a bispecific binding molecule provided herein can be readily isolated and sequenced using conventional procedures (e.g., by using oligonucleotide probes that are capable of binding specifically to genes encoding the heavy and light chains of murine antibodies, or such chains from human, humanized, or other sources). Once isolated, the DNA may be placed into expression vectors, which are then transformed into host cells such as NSO cells, Simian COS cells, Chinese hamster ovary (CHO) cells, yeast cells, algae cells, eggs, or myeloma cells that do not otherwise produce immunoglobulin protein, to obtain the synthesis of the bispecific binding molecules in the recombinant host cells. The DNA also may be modified, for example, by substituting the coding sequence for human heavy and light chain constant domains of a desired species in place of the homologous human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of a bispecific binding molecule provided herein. In certain embodiments, the DNA is as described in Section 5.3.1.

Bispecific binding molecules provided herein can also be prepared using at least one bispecific binding molecule-encoding polynucleotide to provide transgenic animals or mammals, such as goats, cows, horses, sheep, and the like, that produce such antibodies in their milk. Such animals can be provided using known methods. See, for example, but not limited to, U.S. Pat. Nos. 5,827,690; 5,849,992; 4,873,316; 5,849,992; 5,994,616, 5,565,362; 5,304,489, and the like, each of which is entirely incorporated herein by reference.

In certain embodiments, bispecific binding molecules provided herein can additionally be prepared using at least one bispecific binding molecule-encoding polynucleotide provided herein to provide transgenic plants and cultured plant cells (for example, but not limited to tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, for example, using an inducible promoter. See, for example, Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, for example, Hood et al., Adv. Exp. Med. Biol. 464:127-147 (1999) and references cited therein. Antibodies have also been produced in large amounts from transgenic plant seeds including antibody fragments, such as scFvs, including tobacco seeds and potato tubers. See, for example, Conrad et al., Plant Mol. Biol. 38:101-109 (1998) and references cited therein. Thus, bispecific binding molecules can also be produced using transgenic plants, according to known methods. See also, for example, Fischer et al., Biotechnol. Appl. Biochem. 30:99-108 (October 1999), Ma et al., Trends Biotechnol. 13:522-7 (1995); Ma et al., Plant Physiol. 109:341-6 (1995); Whitelam et al., Biochem Soc. Trans. 22:940-944 (1994); and references cited therein. Each of the above references is entirely incorporated herein by reference.

In certain embodiments, bispecific binding molecules provided herein can be prepared using at least one bispecific binding molecule-encoding polynucleotide provided herein to provide bacteria that produce such bispecific binding molecules. As a non-limiting example, E. coli expressing recombinant proteins has been successfully used to provide large amounts of recombinant proteins. See, for example, Verma et al., 1998, 216 (1-2): 165-181 and references cited therein.

See, also, Section 6.1.2.1 for a detailed example for the design and production of a bispecific binding molecule described herein.

In certain embodiments, the bispecific binding molecules can be recovered and purified from recombinant cell cultures by well-known methods including, but not limited to, protein A purification, protein G purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, for example, Colligan, Current Protocols in Immunology, or Current Protocols in Protein Science, John Wiley & Sons, NY, N.Y., (1997-2001), e.g., chapters 1, 4, 6, 8, 9, and 10, each entirely incorporated herein by reference.

In certain embodiments, the bispecific binding molecules provided herein include naturally purified products, products of chemical synthetic procedures, and products produced by recombinant techniques from a eukaryotic host, including, for example, yeast, higher plant, insect and mammalian cells. In preferred embodiments, the bispecific binding molecule is generated in a host such that the bispecific binding molecule is aglycosylated. In another preferred embodiment, the bispecific binding molecule is generated in a bacterial cell such that the bispecific binding molecule is aglycosylated. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Purified antibodies can be characterized by, for example, ELISA, ELISPOT, flow cytometry, immunocytology, Biacore™ analysis, Sapidyne KinExA™ kinetic exclusion assay, SDS-PAGE and Western blot, or by HPLC analysis as well as by a number of other functional assays disclosed herein.

### 5.3.1 Polynucleotides

In certain embodiments, provided herein are polynucleotides comprising a nucleotide sequence encoding a bispecific binding molecule described herein or a fragment thereof (e.g., a heavy chain and/or a light chain fusion polypeptide) that immunospecifically binds to HER2 and CD3, as described in Section 5.1 and Section 5.2. Also provided herein are vectors comprising such polynucleotides. See, Section 5.3.2. Also provided herein are polynucleotides encoding antigens of the bispecific binding molecules provided herein. Also provided herein are polynucleotides that hybridize under stringent or lower stringency hybridization conditions to polynucleotides that encode a bispecific binding molecule or fragment thereof provided herein.

The language “purified” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific embodiment, a nucleic acid molecule(s) encoding a bispecific binding molecule described herein is isolated or purified.

Nucleic acid molecules provided herein can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

In certain embodiments, provided herein is a polynucleotide comprising nucleotide sequences encoding a bispecific binding molecule or fragment thereof as described in Section 5.1 and Section 5.2, wherein the bispecific binding molecule comprises an aglycosylated monoclonal antibody that is an immunoglobulin that binds to HER2, comprising two identical heavy chains and two identical light chains, said light chains being a first light chain and a second light chain, wherein the first light chain is fused to a first scFv, via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, and wherein the first and second light chain fusion polypeptides are identical.

For a detailed example for the generation of a bispecific binding molecule as described herein, see, Section 6.1.2.1 for a detailed example for the design and production of a bispecific binding molecule described herein.

In certain embodiments, provided herein is a polynucleotide comprising nucleotide sequences encoding a light chain fusion polypeptide comprising a light chain fused to a scFv, via a peptide linker, wherein the light chain binds to HER2 and wherein the scFv binds to CD3. In certain embodiments, the light chain is the light chain of a HER2-specific antibody known in the art, such as, for example, trastuzumab, M-111, pertuzumab, ertumaxomab, MDXH210, 2B1, and MM-302. In certain embodiments, the scFv comprises the V_{H} and V_{L} of an anti-CD3 antibody known in the art, such as, for example, huOKT3, YTH12.5, HUM291, teplizumab, huCLB-T¾, otelixizumab, blinatumomab, MT110, catumaxomab, 28F11, 27H5, 23F10, 15C3, visilizumab, and Hum291. In a preferred embodiment, the anti-CD3 antibody is huOKT3. In an especially preferred embodiment, the scFv comprises the V_{H} of huOKT3, further comprising the amino acid substitution at numbered position 105, wherein the cysteine is substituted with a serine. See, for example, Kipriyanov et al. 1997, Protein Eng. 445-453. In certain embodiments, the scFv is derived from the huOKT3 monoclonal antibody and comprises one or more mutations, relative to the native huOKT3 V_{H} and V_{L}, to stabilize disulfide binding. In certain embodiments, the stabilization of disulfide binding prevents aggregation of the bispecific binding molecule. In certain embodiments, the stabilization of disulfide binding reduces aggregation of the bispecific binding molecule as compared to aggregation of the bispecific binding molecule without the stabilization of disulfide binding. In certain embodiments of the bispecific binding molecule, the one or more mutations to stabilize disulfide binding comprise a V_{H} G44C mutation and a V_{L} Q100C mutation (e.g., as present in SEQ ID NOS: 54-59). In certain embodiments of the bispecific binding molecule, the one or more mutations to stabilize disulfide binding are the replacement of the amino acid residue at V_{H}44 (according to the Kabat numbering system) with a cysteine and the replacement of the amino acid residue at V_{L}100 (according to the Kabat numbering system) with a cysteine so as to introduce a disulfide bond between V_{H}44 and V_{L}100 (e.g., as present in SEQ ID NOS: 54-59). In certain embodiments, the peptide linker is between 5-30, 5-25, 5-15, 10-30, 10-20, 10-15, 15-30, or 15-25 amino acid residues in length. In certain embodiments, the sequence of the peptide linker is as described in Table 1, above (e.g., any one of SEQ ID NOs: 14 or 35-41). In a particularly preferred embodiment, the sequence of the peptide linker is SEQ ID NO: 14. In certain embodiments, the sequence to the scFv comprises one or more modifications as described in Table 8, above.

In particular aspects, provided herein are polynucleotides comprising nucleotide sequences encoding bispecific binding molecules or fragments thereof, which specifically bind to HER2 and CD3, and comprise an amino acid sequence as described herein, as well as antibodies which compete with such bispecific binding molecules for binding to HER2 and/or CD3, or which binds to the same epitope as that of such antibodies.

In a preferred embodiment, the sequence of the light chain is SEQ ID NO: 25. In a preferred embodiment, the nucleotide sequence encoding the light chain is SEQ ID NO: 24. In a preferred embodiment, the sequence of the scFv SEQ ID NO: 19. In a preferred embodiment, the nucleotide sequence encoding the scFv SEQ ID NO: 18. In a preferred embodiment, the sequence of the light chain is SEQ ID NO: 25 and the sequence of the scFv is SEQ ID NO: 19. In a preferred embodiment, the nucleotide sequence encoding the light chain is SEQ ID NO: 24 and the nucleotide sequence encoding the scFv is SEQ ID NO: 18. In a preferred embodiment, the sequence of the light chain fusion polypeptide is SEQ ID NO: 29. In a preferred embodiment, the nucleotide sequence encoding the light chain fusion polypeptide is SEQ ID NO: 28.

In certain embodiments, the bispecific binding molecule has a trastuzumab-derived sequence that contains one or more of the modifications in the trastuzumab immunoglobulin, and has a huOKT3-derived sequence that contains one or more of the modifications in the huOKT3 V_{H} and V_{L} sequences, as described in Table 8, below. Bispecific binding molecules having other immunoglobulin or scFv sequences can contain analogous mutations at corresponding positions in these other immunoglobulin or scFv sequences. In certain embodiments, the bispecific binding molecule is (a) derived from trastuzumab and huOKT3; and (b) contains one or more of the modifications as described in Table 8, above. In certain embodiments, the sequence of the peptide linker conjugating the immunoglobulin light chain and the scFv is as described in Table 1, above (e.g., any one of SEQ ID NOs: 14 or 35-41). In certain embodiments, the sequence of the heavy chain is as described in Table 2, above (e.g., any one of SEQ ID NOs: 23, 27, 62, or 63). In certain embodiments, the sequence of the light chain is as described in Table 3, above (e.g., SEQ ID NO: 25). In certain embodiments, the sequence of the V_{H} of the scFv is as described in Table 4, above (e.g., any one of SEQ ID NOs: 15, 17, or 64). In certain embodiments, the sequence of the V_{L} of the scFv is as described in Table 5, above (e.g., any one of SEQ ID NOs: 16 or 65). In certain embodiments, the sequence of the scFv peptide linker is as described in Table 1, above (e.g., any one of SEQ ID NOs: 14 or 35-41). In certain embodiments, the sequence of the scFv is as described in Table 6, above (e.g., any one of SEQ ID NOs: 19 or 48-59). In certain embodiments, the sequence of the light chain fusion polypeptide is as described in Table 7, above (e.g., any one of SEQ ID NOs: 29, 34, 42-47, or 60)

In certain embodiments, provided herein is a polynucleotide comprising nucleotide sequences encoding the heavy chain of a HER2-specific antibody described in Section 5.2. In certain embodiments, the heavy chain is the heavy chain a HER2-specific antibody known in the art, such as, for example, trastuzumab, M-111, pertuzumab, ertumaxomab, MDXH210, 2B1, and MM-302. In a preferred embodiment, the antibody comprises the V_{H} of trastuzumab, wherein the sequence of the heavy chain is SEQ ID NO: 27. In a preferred embodiment, the antibody comprises the V_{H} of trastuzumab, wherein the nucleotide sequence encoding the heavy chain is SEQ ID NO: 26. In a preferred embodiment, the sequence of the heavy chain is comprises the V_{H} of trastuzumab and comprises the amino acid substitution N297A in the Fc region (SEQ ID NO: 26). In a preferred embodiment, the nucleotide sequence encoding the heavy chain comprises the nucleotide sequence encoding the trastuzumab V_{H} and comprises the amino acid substitution N297A in the Fc region (SEQ ID NO: 26). In preferred embodiment, the sequence of the heavy chain comprises the sequence of the trastuzumab V_{H} and comprises the amino acid substitution K322A in the Fc region (SEQ ID NO: 27). In a preferred embodiment, the nucleotide sequence encoding the heavy chain comprises the nucleotide sequence encoding the trastuzumab V_{H} and comprises the amino acid substitution K322A in the Fc region (SEQ ID NO: 26). In an especially preferred embodiment, the sequence of the heavy chain comprises the sequence of the trastuzumab V_{H} and comprises the amino acid substitutions N297A and K322A in the Fc region (SEQ ID NO: 27). In an especially preferred embodiment, the nucleotide sequence encoding the heavy chain comprises the nucleotide sequence encoding the trastuzumab V_{H} and comprises the amino acid substitutions N297A and K322A in the Fc region (SEQ ID NO: 26).

The polynucleotides provided herein can be obtained by any method known in the art. For example, if the nucleotide sequence encoding a bispecific binding molecule or fragment thereof described herein is known, a polynucleotide encoding the bispecific binding molecule or fragment thereof can be may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding a bispecific binding molecule or fragment thereof may be generated from nucleic acid from a suitable source. If a clone containing a nucleic acid encoding a particular bispecific binding molecule or fragment thereof is not available, but the sequence of the bispecific binding molecule or fragment thereof is known, a nucleic acid encoding the bispecific binding molecule or fragment thereof may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody provided herein) by PCR amplification using synthetic primers that hybridize to the 3′ and 5′ ends of the sequence or by cloning using an oligonucleotide probe specific for the particular gene sequence to identify, for example, a cDNA clone from a cDNA library that encodes the antibody. Amplified nucleic acids generated by PCR may then be cloned into replicable cloning vectors using any method well known in the art. See, for example, Section 5.3.2.

In certain embodiments, the amino acid sequence of the antibody of the bispecific binding molecule is known in the art. In such embodiments, a polypeptide encoding such an antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, N.Y., which are both incorporated by reference herein in their entireties), to generate bispecific binding molecules having a different amino acid sequence, for example, to create amino acid substitutions, deletions, and/or insertions. For example, such manipulations can be performed to render the encoded amino acid aglycosylated, or to destroy the antibody's ability to bind to C1q, Fc receptor, or to activate the complement system.

Isolated nucleic acid molecules provided herein can include nucleic acid molecules comprising an open reading frame (ORF), optionally with one or more introns, for example, but not limited to, at least one specified portion of at least one complementarity determining region (CDR), as CDR1, CDR2 and/or CDR3 of at least one heavy chain or light chain; nucleic acid molecules comprising the coding sequence for an anti-HER2 antibody or variable region, an anti-CD3 scFv, or a single chain fusion polypeptide; and nucleic acid molecules which comprise a nucleotide sequence substantially different from those described above but which, due to the degeneracy of the genetic code, still encode at least one bispecific binding molecule as described herein and/or as known in the art.

Also provided herein are isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides provided herein can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

The nucleic acids can conveniently comprise sequences in addition to a polynucleotide provided herein. For example, a multi-cloning site comprising one or more endonuclease restriction sites can be inserted into the nucleic acid to aid in isolation of the polynucleotide. In addition, translatable sequences can be inserted to aid in the isolation of the translated polynucleotide provided herein. For example, a hexa-histidine marker sequence provides a convenient means to purify the polypeptides provided herein. The nucleic acid provided herein-excluding the coding sequence—is optionally a vector, adapter, or linker for cloning and/or expression of a polynucleotide provided herein.

Additional sequences can also be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

In a specific embodiment, using routine recombinant DNA techniques, one or more of the CDRs of an antibody described herein may be inserted within framework regions. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds HER2. One or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are provided herein and within the skill of the art.

In certain embodiments, the isolated or purified nucleic acid molecule, or fragment thereof, upon linkage with another nucleic acid molecule, can encode a fusion protein. The generation of fusion proteins is within the ordinary skill in the art and can involve the use of restriction enzyme or recombinational cloning techniques (see, for example, Gateway. TM . . . (Invitrogen)). See, also, U.S. Pat. No. 5,314,995.

In certain embodiments, a polynucleotide provided herein is in the form of a vector (e.g., expression vector) as described in Section 5.3.2.

### 5.3.2 Cells and Vectors

In certain embodiments, provided herein are cells (e.g., ex vivo cells) expressing (e.g., recombinantly) bispecific binding molecules as described herein. Also provided herein are vectors (e.g., expression vectors) comprising nucleotide sequences (see, for example, Section 5.3.1) encoding a bispecific binding molecule or fragment thereof described herein for recombinant expression in host cells, preferably in mammalian cells. Also provided herein are cells (e.g., ex vivo cells) comprising such vectors or nucleotide sequences for recombinantly expressing a bispecific binding molecule described here. Also provided herein are methods for producing a bispecific binding molecule described herein, comprising expressing such bispecific binding molecule from a cell (e.g., ex vivo cell). In a preferred embodiment, the cell is an ex vivo cell.

A vector (e.g., expression vector) is a DNA molecule comprising a gene that is expressed in a cell (e.g., ex vivo cell). Typically, gene expression is placed under the control of certain regulatory elements, including constitutive or inducible promoters, tissue-specific regulatory elements and enhancers. Such a gene is said to be “operably linked to” the regulatory elements, e.g., a promoter. A recombinant host may be any prokaryotic or eukaryotic cell that contains either a cloning vector or expression vector. This term also includes those prokaryotic or eukaryotic cells, as well as a transgenic animal, that have been genetically engineered to contain the cloned gene(s) in the chromosome or genome of the host cell or cells of the host cells (e.g., ex vivo cells).

In a preferred embodiment, the promoter is the CMV promoter.

In certain embodiments, provided herein is a vector comprising one or more polynucleotide as described in Section 5.3.1.

In certain embodiments, a polynucleotide as described in Section 5.3.1 can be cloned into a suitable vector and can be used to transform or transfect any suitable host. Vectors and methods to construct such vectors are known to one of ordinary skill in the art and are described in general technical references (see, in general, “Recombinant DNA Part D,” Methods in Enzymology, Vol. 153, Wu and Grossman, eds., Academic Press (1987)). In certain embodiments, the vector comprises regulatory sequences, such as transcription and translation initiation and termination codons, which are specific to the type of host (e.g., bacterium, fungus, plant, insect, or mammal) into which the vector is to be introduced, as appropriate and taking into consideration whether the vector is DNA or RNA. In certain embodiments, the vector comprises regulatory sequences that are specific to the genus of the host. In certain embodiments, the vector comprises regulatory sequences that are specific to the species of the host.

In certain embodiments, the vector comprises one or more marker genes, which allow for selection of transformed or transfected hosts. Non-limiting examples of marker genes include biocide resistance, e.g., resistance to antibiotics, heavy metals, etc., complementation in an auxotrophic host to provide prototrophy, and the like. In a preferred embodiment, the vector comprises ampicillin and hygromycin selectable markers.

In certain embodiments, an expression vector can comprise a native or normative promoter operably linked to a polynucleotide as described in Section 5.3.1. The selection of promoters, for example, strong, weak, inducible, tissue-specific and developmental-specific, is within the skill in the art. Similarly, the combining of a nucleic acid molecule, or fragment thereof, as described above with a promoter is also within the skill in the art.

Non-limiting examples of suitable vectors include those designed for propagation and expansion or for expression or both. For example, a cloning vector can be selected from the group consisting of the pUC series, the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as lamda-GT10, lamda-GT11, lamda-ZapII (Stratagene), lamda-EMBL4, and lamda-NM1149, can also be used. Non-limiting examples of plant expression vectors include pBI110, pBI101.2, pBI101.3, pBI121 and pBIN19 (Clontech). Non-limiting examples of animal expression vectors include pEUK-C₁, pMAM and pMAMneo (Clontech). The TOPO cloning system (Invitrogen, Carlsbad, Calif.) can also be used in accordance with the manufacturer's recommendations.

In certain embodiments, the vector is a mammalian vector. In certain embodiments, the mammalian vector contains at least one promoter element, which mediates the initiation of transcription of mRNA, the bispecific binding molecule coding sequence, and signals required for the termination of transcription and polyadenylation of the transcript. In certain embodiments, the mammalian vector contains additional elements, such as, for example, enhancers, Kozak sequences and intervening sequences flanked by donor and acceptor sites for RNA splicing. In certain embodiments, highly efficient transcription can be achieved with, for example, the early and late promoters from SV40, the long terminal repeats (LTRS) from retroviruses, for example, RSV, HTLVI, HIVH and the early promoter of the cytomegalovirus (CMV). However, cellular elements can also be used (e.g., the human actin promoter). Non-limiting examples of mammalian expression vectors include, vectors such as pIRESIneo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Non-limiting example of mammalian host cells that can be used in combination with such mammalian vectors include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

In certain embodiments, the vector is a viral vector, for example, retroviral vectors, parvovirus-based vectors, e.g., adeno-associated virus (AAV)-based vectors, AAV-adenoviral chimeric vectors, and adenovirus-based vectors, and lentiviral vectors, such as Herpes simplex (HSV)-based vectors. In certain embodiments, the viral vector is manipulated to render the virus replication deficient. In certain embodiments, the viral vector is manipulated to eliminate toxicity to the host. These viral vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994).

In certain embodiments, a vector or polynucleotide described herein can be transferred to a cell (e.g., an ex vivo cell) by conventional techniques and the resulting cell can be cultured by conventional techniques to produce a bispecific binding molecule described herein. Accordingly, provided herein are cells comprising a polynucleotide encoding a bispecific binding molecule or fragment thereof, a heavy or light chain thereof, or a light chain fusion polypeptide thereof, operably linked to a promoter for expression of such sequences in the host cell. In certain embodiments, a vector encoding the heavy chain operably linked to a promoter and a vector encoding the light chain fusion polypeptide operably linked to a promoter can be co-expressed in the cell for expression of the entire bispecific binding molecule, as described below. In certain embodiments, a cell comprises a vector comprising a polynucleotide encoding both the heavy chain and the light chain fusion polypeptide of a bispecific binding molecule described herein operably linked to a promoter. In certain embodiments, a cell comprises two different vectors, a first vector comprising a polynucleotide encoding a heavy chain operably linked to a promoter, and a second vector comprising a polynucleotide encoding a light chain fusion polypeptide operably linked to a promoter. In certain embodiments, a first cell comprises a first vector comprising a polynucleotide encoding a heavy chain of a bispecific binding molecule described herein, and a second cell comprises a second vector comprising a polynucleotide encoding a light chain fusion polypeptide of a bispecific binding molecule described herein. In certain embodiments, provided herein is a mixture of cells comprising such first cell and such second cell. In a preferred embodiment, the cell expresses the vector or vectors such that the oligonucleotide is both transcribed and translated efficiently by the cell.

In embodiment, the cell expresses the vector, such that the oligonucleotide, or fragment thereof, is both transcribed and translated efficiently by the cell.

In certain embodiments, the cell is present in a host, which can be an animal, such as a mammal. Examples of cells include, but are not limited to, a human cell, a human cell line, E. coli (e.g., E. coli TB-1, TG-2, DH5a, XL-Blue MRF′ (Stratagene), SA2821 and Y1090), B. subtilis, P. aerugenosa, S. cerevisiae, N. crassa, insect cells (e.g., Sf9, Ea4) and others set forth herein below. In a preferred embodiment, the cell is a CHO cell. In an especially preferred embodiment, the cell is a CHO-S cell.

In certain embodiments, a polynucleotide described herein can be expressed in a stable cell line that comprises the polynucleotide integrated into a chromosome by introducing the polynucleotide into the cell. In certain embodiments, the polynucleotide is introduced into the cell by, for example, electroporation. In certain embodiments, the polynucleotide is introduced into the cell by, for example, transfection of a vector comprising the polynucleotide into the cell. In certain embodiments, the vector is co-transfected with a selectable marker such as DHFR, GPT, neomycin, or hygromycin to allow for the identification and isolation of the transfected cells. In certain embodiments, the transfected polynucleotide can also be amplified to express large amounts of the encoded bispecific binding molecule. For example, the DHFR (dihydrofolate reductase) marker can be utilized to develop cell lines that carry several hundred or even several thousand copies of the polynucleotide of interest. Another example of a selection marker is the enzyme glutamine synthase (GS) (Murphy, et al., Biochem. J. 227:277-279 (1991); Bebbington, et al., Bio/Technology 10:169-175 (1992)). Using these markers, the cells are grown in selective medium and the cells with the highest resistance are selected. These cell lines contain the amplified gene(s) integrated into a chromosome. Chinese hamster ovary (CHO) and NSO cells are often used for the production of bispecific binding molecules.

In a preferred embodiment, the vector comprises (i) a first polynucleotide sequence encoding a light chain fusion polypeptide comprising an immunoglobulin light chain fused to a scFv, via a peptide linker, wherein the light chain binds to HER2 and wherein the scFv binds to CD3, operably linked to a first promoter and (ii) a second polynucleotide encoding an immunoglobulin heavy chain that binds to HER2 operably linked to a second promoter. In certain embodiments, the vector is a viral vector.

### 5.4 T Cells Bound to Bispecific Binding Molecules

Without being bound by any theory, it is believed that when the bispecific binding molecules provided herein are bound to T cells, by, for example, procedures such as those described herein, an anti-CD3 scFv of the bispecific binding molecule binds to CD3 on the surface of the T cell. Without being bound by any theory, it is believed that binding of the bispecific binding molecule to the T cell (i.e., binding of an anti-CD3 scFv to CD3 expressed on the T cell) activates the T cell, and consequently, allows for the T cell receptor-based cytotoxicity to be redirected to desired tumor targets, bypassing MHC restrictions.

Thus, the invention also provides T cells which are bound to a bispecific binding molecule of the invention (e.g., as described in Section 5.1 and Section 5.2). In specific embodiments, the T cells are bound to the bispecific binding molecule noncovalently. In specific embodiments, the T cells are autologous to a subject to whom the T cells are to be administered. In specific embodiments, the T cells are allogeneic to a subject to whom the T cells are to be administered. In specific embodiments, the T cells are human T cells.

In specific embodiments, the T cells which are bound to bispecific binding molecules of the invention are used in accordance with the methods described in Section 5.6. In specific embodiments, the T cells which are bound to bispecific binding molecules of the invention are used as part of a combination therapy as described in Section 5.9.

### 5.5 Pharmaceutical Compositions and Kits

In certain embodiments, provided herein are compositions (e.g., pharmaceutical compositions) and kits comprising a pharmaceutically effective amount of one or more bispecific binding molecule as described in Section 5.1 or Section 5.2. Compositions may be used in the preparation of individual, single unit dosage forms. Compositions provided herein can be formulated for parenteral, subcutaneous, intramuscular, intravenous, intrarticular, intrabronchial, intraabdominal, intracapsular, intracartilaginous, intracavitary, intracelial, intracerebellar, intracerebroventricular, intra-Ommaya, intraocular, intravitreous, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, intrathecal, intraventricular in the brain, intraparenchymal in the brain, or transdermal administration. In a preferred embodiment, the composition is formulated for parenteral administration. In an especially preferred embodiment, the composition is formulated for intravenous administration. In a preferred embodiment, the composition is formulated for intraperitoneal administration. In a specific embodiment, the composition is formulated for intraperitoneal administration to treat peritoneal metastases. In a preferred embodiment, the composition is formulated for intrathecal administration. In a specific embodiment, the composition is formulated for intrathecal administration to treat brain metastases. See, for example, Kramer et al., 2010, 97:409-418. In a preferred embodiment, the composition is formulated for intraventricular administration in the brain. In a specific embodiment, the composition is formulated for intraventricular administration to treat brain metastases. See, for example, Kramer et al., 2010, 97:409-418. In a preferred embodiment, the composition is formulated for intraparenchymal administration in the brain. In a specific embodiment, the composition is formulated for intraparenchymal administration to treat a brain tumor or brain tumor metastases. See, for example, Luther et al., 2014, Neuro Oncol, 16:800-806, and Clinical Trial ID NO NCT01502917.

In a specific embodiment, the composition is formulated for intraperitoneal administration for peritoneal metastases.

In certain embodiments, provided herein is a composition comprising one or more polynucleotide comprising nucleotide sequences encoding a bispecific binding molecule as described herein. In certain embodiments, provided herein is a composition comprising a cell, wherein the cell comprises one or more polynucleotide comprising nucleotide sequences encoding a bispecific binding molecule as described herein. In certain embodiments, provided herein is a composition comprising a vector, wherein the vector comprises one or more polynucleotide comprising nucleotide sequences encoding a bispecific binding molecule as described herein. In certain embodiments, provided herein is a composition comprising a cell, wherein the cell comprises a vector, wherein the vector comprises one or more polynucleotide comprising nucleotide sequences encoding a bispecific binding molecule as described herein.

In certain embodiments, a composition described herein is a stable or preserved formulation. In certain embodiments, the stable formulation comprises a phosphate buffer with saline or a chosen salt. In certain embodiments, a composition described is a multi-use preserved formulation, suitable for pharmaceutical or veterinary use. In certain embodiments, a composition described herein comprises a preservative. Preservatives are known to one of ordinary skill in the art. Non-limiting examples of preservatives include phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben(methyl, ethyl, propyl, butyl and the like), benzalkonium chloride, benzethonium chloride, and sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

It can be sometimes desirable to deliver the compositions provided herein to a subject over prolonged periods of time, for example, for periods of one week to one year or more from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g., a zinc tannate salt. Additionally, a composition provided herein, preferably, a relatively insoluble salt such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer, for example, as described in U.S. Pat. No. 3,773,919. The compounds or, preferably, relatively insoluble salts such as those described above can also be formulated in cholesterol matrix silastic pellets, particularly for use in animals. Additional slow release, depot or implant compostions, e.g., gas or liquid liposomes are known in the literature (U.S. Pat. No. 5,770,222 and “Sustained and Controlled Release Drug Delivery Systems”, J. R. Robinson ed., Marcel Dekker, Inc., N. Y., 1978).

The range of at least one bispecific binding molecule composition provided herein includes amounts yielding upon reconstitution, if in a wet/dry system, concentrations from about 1.0 microgram/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

In certain embodiments, compositions provided herein comprise at least one of any suitable auxiliary, such as, but not limited to, diluent, binder, stabilizer, buffers, salts, lipophilic solvents, preservative, adjuvant or the like. In certain embodiments, pharmaceutically acceptable auxiliaries are preferred. Non-limiting examples of, and methods of preparing such sterile solutions are well known in the art, such as, but not limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the bispecific binding molecule as described herein.

In certain embodiments, compositions provided herein contain one or more pharmaceutical excipient and/or additive. Non-limiting examples of pharmaceutical excipients and additives are proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin such as human serum albumin (HSA), recombinant human albumin (rHA), gelatin, casein, and the like. Non-limiting examples of amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. In certain embodiments, the amino acid is glycine. Non-limiting examples of carbohydrate excipients include monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. In certain embodiments, the carbohydrate excipient is mannitol, trehalose, or raffinose.

In certain embodiments, a composition provided herein includes one or more buffer or a pH adjusting agent; typically, the buffer is a salt prepared from an organic acid or base. Non-limiting examples of buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In certain embodiments, the buffer is an organic acid salts such as citrate. Other excipients, e.g., isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The compositions can cover a wide range of pHs, such as from about pH 4 to about pH 10, and preferred ranges from about pH 5 to about pH 9, and a most preferred range of about 6.0 to about 8.0. Preferably, the compositions provided herein have pH between about 6.8 and about 7.8. Preferred buffers include phosphate buffers, most preferably sodium phosphate, particularly phosphate buffered saline (PBS).

In certain embodiments, a composition provided herein includes one or more polymeric excipient/additive such as, for example, polyvinylpyrrolidones, ficolls (a polymeric sugar), dextrates (e.g., cyclodextrins, such as 2-hydroxypropyl-. beta.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and/or chelating agents (e.g., EDTA).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic.RTM. polyls, other block co-polymers, and chelators such as EDTA and EGTA can optionally be added to the compositions to reduce aggregation. These additives are particularly useful if a pump or plastic container is used to administer the composition. The presence of pharmaceutically acceptable surfactant mitigates the propensity for the protein to aggregate.

Additional pharmaceutical excipients and/or additives suitable for use in a composition provided herein are known to one of skill in the art and are referenced in, for example, “Remington: The Science & Practice of Pharmacy”, 19.sup.th ed., Williams & Williams, (1995), and in the “Physician's Desk Reference”, 52^{nd} ed., Medical Economics, Montvale, N.J. (1998), which are entirely incorporated herein by reference. In certain preferred embodiments, the carrier or excipient materials are carbohydrates (e.g., saccharides and alditols) and buffers (e.g., citrate) or polymeric agents.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben(methyl, ethyl, propyl, butyl and the like), benzalkonium chloride, benzethonium chloride, sodium dehydroacetate and thimerosal, or mixtures thereof. The concentration of preservative used in the composition is a concentration sufficient to yield an anti-microbial effect. Such concentrations are dependent on the preservative selected and are readily determined by the skilled artisan.

The compositions provided herein can be prepared by a process which comprises mixing at least one bispecific binding molecule as described herein and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one bispecific binding molecule and preservative in an aqueous diluent is carried out using conventional dissolution and mixing procedures. To prepare a suitable composition, for example, a measured amount of at least one bispecific binding molecule in buffered solution is combined with the desired preservative in a buffered solution in quantities sufficient to provide the bispecific binding molecule and preservative at the desired concentrations. The compositions provided herein can be prepared by a process that comprises mixing at least one bispecific binding molecule as described herein and a selected buffer, preferably a phosphate buffer containing saline or a chosen salt. Mixing the at least one bispecific binding molecule and buffer in an aqueous diluent is carried out using conventional dissolution and mixing procedures. To prepare a suitable composition, for example, a measured amount of at least one bispecific binding molecule in water or buffer is combined with the desired buffering agent in water in quantities sufficient to provide the protein and buffer at the desired concentrations. Variations of these processes would be recognized by one of ordinary skill in the art. For example, the order the components are added, whether additional additives are used, the temperature and pH at which the composition is prepared, are all factors that can be optimized for the concentration and means of administration used.

In specific embodiments involving combination therapy with infusion of T cells, provided herein is a pharmaceutical composition comprising (a) a bispecific binding molecule described herein (see, e.g., Section 5.1 or 5.2); (b) T cells; and/or (c) a pharmaceutically effective carrier. In specific embodiments, the T cells are autologous to the subject to whom the T cells are administered. In certain embodiments, the T cells are allogeneic to the subject to whom the T cells are administered. In specific embodiments, the T cells are bound to the bispecific binding molecule. In specific embodiments, the binding of the T cells to the bispecific binding molecule is noncovalently. In specific embodiments, the T cells are human T cells. Methods that can be used to bind bispecific binding molecules to T cells are known in the art. See, e.g., Lum et al., 2013, Biol Blood Marrow Transplant, 19:925-33, Janeway et al., Immunobiology: The Immune System in Health and Disease, 5^{th} edition, New York: Garland Science; Vaishampayan et al., 2015, Prostate Cancer, 2015:285193, and Stromnes et al., 2014, Immunol Rev. 257 (1): 145-164. See, also, Vaishampayan et al., 2015, Prostate Cancer, 2015:285193, which describes the following exemplary, non-limiting method for binding bispecific binding molecules to T cells:
Peripheral blood mononuclear cells (PBMCs) are collected to obtain lymphocytes for activated T cell expansion from 1 or 2 leukopheresis. PBMCs are activated with, for example, 20 ng/ml of OKT3 and expanded in 100 IU/mL of IL-2 to generate 40-320 billion activated T cells during a maximum of 14 days of culture under cGMP conditions as described in Ueda et al., 1993, Transplantation, 56 (2): 351-356 and Uberti et al., 1994, Clinical Immunology and Immunopathology, 70 (3): 234-240. Cells are grown in breathable flasks (FEP Bag Type 750-Cl, American Fluoroseal Corporation, Gaithersburg, MD) in RPMI 1640 medium (Lonza) supplemented with 2% pooled heat inactivated human serum. Activated T cells are split approximately every 2-3 days based on cell counts. After 14 days, activated T cells are cultured with 50 ng of a bispecific binding molecule described herein per 10⁶ activated T cells. The mixture is then washed and cryopreserved.

In certain embodiments, a pharmaceutical composition described herein is to be used in accordance with the methods provided herein (see, e.g., Section 5.6).

### 5.5.1 Parenteral Formulations

In certain embodiments, a composition provided herein is formulated for parenteral injectable administration. As used herein, the term “parenteral” includes intravenous, intravascular, intramuscular, intradermal, subcutaneous, and intraocular. For parenteral administration, the composition can be formulated as a solution, suspension, emulsion or lyophilized powder in association, or separately provided, with a pharmaceutically acceptable parenteral vehicle. Non-limiting examples of such vehicles are water, saline, Ringer's solution, dextrose solution, glycerol, ethanol, and 1-10% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aqueous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### 5.5.2 Pulmonary Formulations

In certain embodiments, a composition comprising a bispecific binding molecule described herein is formulated for pulmonary administration. For pulmonary administration, the composition is delivered in a particle size effective for reaching the lower airways of the lung or sinuses. Compositions for pulmonary administration can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of bispecific binding molecules described herein are also known in the art. All such devices use formulations suitable for the administration for the dispensing of a bispecific binding molecule described herein in an aerosol. Such aerosols can be comprised of either solutions (both aqueous and non aqueous) or solid particles. Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler®. (Glaxo), Diskus® (Glaxo), devices marketed by Inhale Therapeutics, to name a few, use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. Such examples of commercially available inhalation devices are non-limiting examples are not intended to be limiting in scope.

In certain embodiments, a spray comprising a bispecific binding molecule as described herein can be produced by forcing a suspension or solution of at least one bispecific binding molecule as described herein through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of a composition comprising at least one bispecific binding molecule described herein delivered by a sprayer have a particle size less than about 10 μm, preferably in the range of about 1 μm to about 5 μm, and most preferably about 2 μm to about 3 um.

Formulations of a composition comprising at least one bispecific binding molecule described herein suitable for use with a sprayer typically include the at least one bispecific binding molecule in an aqueous solution at a concentration of about 0.1 mg to about 100 mg per ml of solution or mg/gm, or any range or value therein, e.g., but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/ml or mg/gm. The formulation can include agents such as an excipient, a buffer, an isotonicity agent, a preservative, a surfactant, and, preferably, zinc. The formulation can also include an excipient or agent for stabilization of the bispecific binding molecule composition, such as a buffer, a reducing agent, a bulk protein, or a carbohydrate. Bulk proteins useful in formulating such a composition include albumin, protamine, or the like. Typical carbohydrates useful in formulating antibody composition proteins include sucrose, mannitol, lactose, trehalose, glucose, or the like. The composition can also include a surfactant, which can reduce or prevent surface-induced aggregation of the composition caused by atomization of the solution in forming an aerosol. Various conventional surfactants can be employed, such as polyoxyethylene fatty acid esters and alcohols, and polyoxy ethylene sorbitol fatty acid esters. Amounts will generally range between 0.001 and 14% by weight of the formulation. Preferred surfactants are polyoxyethylene sorbitan monooleate, polysorbate 80, polysorbate 20, or the like.

In certain embodiments, the composition is administered via a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of antibody composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of antibody composition protein either directly or through a coupling fluid, creating an aerosol including the antibody composition protein. Advantageously, particles of antibody composition protein delivered by a nebulizer have a particle size less than about 10 μm, preferably in the range of about 1 μm to about 5 μm, and most preferably about 2 μm to about 3 um.

In certain embodiments, the composition is administered via a metered dose inhaler (MDI), wherein a propellant, at least one bispecific binding molecule described herein, and any excipients or other additives are contained in a canister as a mixture including a liquefied compressed gas. Actuation of the metering valve releases die mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of antibody composition protein produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of a bispecific binding molecule described herein for use with a metered-dose inhaler device will generally include a finely divided powder containing at least one Anti-IL-6 antibody as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1, 1,2-tetrafluoroethane, HFA-134a (hydrofluoroalkane-134a), HFA-227 (hydrofluoroalkane-227), or the like. Preferably the propellant is a hydrofluorocarbon. The surfactant can be chosen to stabilize the at least one bispecific binding molecule as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. Additional agents known in the art for formulation of a protein can also be included in the formulation.

### 5.5.3 Oral Formulations

In certain embodiments, a composition provided herein is formulated for oral administration. In certain embodiments, for oral administration, compositions and methods of administering at least one bispecific binding molecule described herein rely on the co-administration of adjuvants such as, for example, resorcinols and nonionic surfactants such as polyoxyethylene oleyl ether and n-hexadecylpolyethylene ether, to artificially increase the permeability of the intestinal walls, as well as the co-administration of enzymatic inhibitors such as, for example, pancreatic trypsin inhibitors, diisopropylfluorophosphate (DFF) and trasylol, to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, such as, for example, inactive diluting agent, lubricant such as magnesium stearate, paraben, preserving agent such as sorbic acid, ascorbic acid, alpha.-tocopherol, antioxidant such as cysteine, disintegrator, binder, thickener, buffering agent, sweetening agent, flavoring agent, perfuming agent, etc.

In certain embodiments, tablets and pills for oral administration can be further processed into enteric-coated preparations. In certain embodiments, liquid preparations for oral administration include, for example, emulsion, syrup, elixir, suspension and solution preparations allowable for medical use. These preparations can contain inactive diluting agents ordinarily used in said field, for example, water. Liposome preparations can be utilized for oral administration preparations, for example, as described for insulin and heparin (U.S. Pat. No. 4,239,754). Additionally, microspheres of artificial polymers of mixed amino acids (proteinoids) can be utilized to in oral administration of pharmaceuticals, for example, as described in U.S. Pat. No. 4,925,673. Furthermore, carrier compounds, such as those described in U.S. Pat. Nos. 5,879,681 and 5,871,753, are used in oral administration of biologically active agents.

### 5.5.4 Mucosal Formulations

In certain embodiments, a composition provided herein is formulated for absorption through mucosal surfaces. In certain embodiments, for absorption through mucosal surfaces, compositions and methods of administering at least one bispecific binding molecule described herein include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions provided herein can include, for example, corneal, conjunctival, buccal, sublingual, nasal, vaginal, pulmonary, stomachic, intestinal, and rectal routes of administration. Formulations for vaginal or rectal administration, for example, suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include, for example, sugars, calcium stearate, magnesium stearate, pregelinatined starch, and the like (U.S. Pat. No. 5,849,695).

### 5.5.5 Transdermal Formulations

In certain embodiments, a composition provided herein is formulated for transdermal administration. In certain embodiments, for transdermal administration, the composition comprises at least one bispecific binding molecule described herein encapsulated in a delivery device such as, for example, a liposome or polymeric nanoparticles, microparticle, microcapsule, or microspheres (referred to collectively as microparticles unless otherwise stated). A number of suitable devices are known for transdermal administration, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. No. 5,814,599).

### 5.5.6 Kits

Provided herein are kits comprising one or more bispecific binding molecule as described herein, or one or more composition as described herein. In certain embodiments, the kit comprises packaging material and at least one vial comprising a composition comprising a bispecific binding molecule or composition described herein. In certain embodiments, the vial comprises a solution of at least one bispecific binding molecule or composition as described herein with the prescribed buffers and/or preservatives, optionally in an aqueous diluents. In certain embodiments, the packaging material comprises a label that indicates that such solution can be held over a period of 1, 2, 3, 4, 5, 6, 9, 12, 18, 20, 24, 30, 36, 40, 48, 54, 60, 66, 72 hours or greater. In certain embodiments, the kit comprises two vials. In certain embodiments, the first vial comprises at least one lyophilized bispecific binding molecule or composition as described herein and the second vial comprises aqueous diluents of prescribed buffer or preservative. In certain embodiments, the packaging material comprises a label that instructs a subject to reconstitute the at least one lyophilized bispecific binding molecule in the aqueous diluents to form a solution that can be held over a period of twenty-four hours or greater. In certain embodiments, the packaging material comprises a label that indicates that such solution can be held over a period of 1, 2, 3, 4, 5, 6, 9, 12, 18, 20, 24, 30, 36, 40, 48, 54, 60, 66, 72 hours or greater.

In certain embodiments, the compositions provided herein can be provided to a subject as solutions or as dual vials comprising a vial of lyophilized at least one bispecific binding molecule or composition that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of subject treatment and thus can provide a more convenient treatment regimen than currently available.

In certain embodiments, a kit comprising a bispecific binding molecule or composition described herein is useful for administration over a period of immediately to twenty-four hours or greater. Accordingly, the kit offers significant advantages to the patient. In certain embodiments, a kit comprising a bispecific binding molecule or composition described herein can optionally be safely stored at temperatures of from about 2° C. to about 40° C. and retain the biologically activity of the protein for extended periods of time, thus, allowing a package label indicating that the solution can be held and/or used over a period of 6, 12, 18, 24, 36, 48, 72, or 96 hours or greater. In certain embodiments, the kit comprises a

If preserved diluent is used, such label can include use up to 1-12 months, one-half, one and a half, and/or two years.

The kits can be provided indirectly to a subject, such as a subject as described in Section 5.7, by providing to pharmacies, clinics, or other such institutions and facilities, solutions or dual vials comprising a vial of lyophilized at least one bispecific binding molecule or composition that is reconstituted with a second vial containing the aqueous diluent. The solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising these single vial systems include those pen-injector devices for delivery of a solution such as BD Pens, BD Autojector®, Humaject®, e.g., as made or developed by Becton Dickensen (Franklin Lakes, N.J.,), Disetronic (Burgdorf, Switzerland; Bioject, Portland, Oreg.; National Medical Products, Weston Medical (Peterborough, UK), Medi-Ject Corp (Minneapolis, Minn.). Recognized devices comprising a dual vial system include those pen-injector systems for reconstituting a lyophilized drug in a cartridge for delivery of the reconstituted solution such as the HumatroPen®.

In certain embodiments, the kits comprise packaging material. In certain embodiments, the packaging material provides, in addition to the information required by a regulatory agencies, the conditions under which the product can be used. In certain embodiments, the packaging material provides instructions to the subject to reconstitute the at least one bispecific binding molecule in the aqueous diluent to form a solution and to use the solution over a period of 2-24 hours or greater for the two vial, wet/dry, product. For the single vial, solution product, the label indicates that such solution can be used over a period of 2-24 hours or greater. In a preferred embodiment, the kit is useful for human pharmaceutical product use. In certain embodiments, the kit is useful for veterinarian pharmaceutical use. In a preferred embodiment, the kit is useful for canine pharmaceutical product use. In a preferred embodiment, the kit is useful for intravenous administration. In another preferred embodiment, the kit is useful for intraperitoneal, intrathecal, intraventricular in the brain, or intraparenchymal in the brain administration.

### 5.6 Uses and Methods

### 5.6.1 Therapeutic Uses

In certain embodiments, provided herein are methods for treating a HER2-positive cancer in a subject comprising administering to the subject in need thereof a therapeutically effective amount of a bispecific binding molecule as described in Section 5.1 or in Section 5.2, a therapeutically effective amount of a cell, polynucleotide, or vector encoding such a bispecific binding molecule as described in Section 5.3, or a therapeutically effective amount of a pharmaceutical composition as described in Section 5.5, or a therapeutically effective amount of T cells bound to a bispecific binding molecule as described in Section 5.4. In a specific embodiment, the subject is a subject as described in Section 5.7. In a specific embodiment, the bispecific binding molecule is administered at a dose as described in Section 5.8. In a specific embodiment, the bispecific binding molecule is administered according to the methods as described in Section 5.5. In a preferred embodiment, the bispecific binding molecule is administered intravenously. In another preferred embodiment, the bispecific binding molecule is administered intrathecally, intraventricularly in the brain, intraparenchymally in the brain, or intraperitoneally. In a specific embodiment, the bispecific binding molecule is administered in combination with one or more additional pharmaceutically active agents as described in Section 5.9.

In certain embodiments, provided herein are methods for treating a HER2-positive cancer in a subject comprising administering to the subject in need thereof a pharmaceutical composition as described in Section 5.1 or in Section 5.2. In a specific embodiment, the pharmaceutical composition is a composition as described in Section 5.5. In a specific embodiment, the subject is a subject as described in Section 5.7. In a specific embodiment, the pharmaceutical composition is administered at a dose as described in Section 5.8. In a specific embodiment, the pharmaceutical composition is administered according to the methods as described in Section 5.5. In a preferred embodiment, the pharmaceutical composition is administered intravenously. In another preferred embodiment, the bispecific binding molecule is administered intrathecally, intraventricularly in the brain, intraparenchymally in the brain, or intraperitoneally. In a specific embodiment, the pharmaceutical composition is administered in combination with one or more additional pharmaceutically active agents as described in Section 5.9.

For use of a bispecific binding molecule in a subject of a particular species, a bispecific binding molecule is used that binds to the HER2 and the CD3 of that particular species. For example, to treat a human, the bispecific binding molecule comprises an aglycosylated monoclonal antibody that is an immunoglobulin that binds to human HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to human CD3, and wherein the first and second light chain fusion polypeptides are identical. In another example, to treat a canine, the bispecific binding molecule comprises an aglycosylated monoclonal antibody that is an immunoglobulin that binds to canine HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to canine CD3, and wherein the first and second light chain fusion polypeptides are identical. Bispecific binding molecules that are cross-reactive with HER2 and/or CD3 of various species can be used to treat subjects in those species. For example, trastuzumab is expected to bind both human and canine HER2 due to the relative conservation of the epitope in HER2 recognized by trastuzumab. See, also, for example, Singer et al., 2012, Mol Immunol, 50:200-209.

In addition, for use of a bispecific binding molecule in a subject of a particular species, the bispecific binding molecule, preferably, the constant region of the immunoglobulin portion, is derived from that particular species. For example, to treat a human, the bispecific binding molecule can comprise an aglycosylated monoclonal antibody that is an immunoglobulin, wherein the immunoglobulin comprises a human constant region. In another example, to treat a canine, the bispecific binding molecule can comprise an aglycosylated monoclonal antibody that is an immunoglobulin, wherein the immunoglobulin comprises a canine constant region. In a specific embodiment, when treating a human, the immunoglobulin is humanized. In a specific embodiment, the subject is a human. In a specific embodiment, the subject is a canine.

In a specific embodiment, the HER2-positive cancer is breast cancer, gastric cancer, an osteosarcoma, desmoplastic small round cell cancer, squamous cell carcinoma of head and neck cancer, ovarian cancer, prostate cancer, pancreatic cancer, glioblastoma multiforme, gastric junction adenocarcinoma, gastroesophageal junction adenocarcinoma, cervical cancer, salivary gland cancer, soft tissue sarcoma, leukemia, melanoma, Ewing's sarcoma, rhabdomyosarcoma, neuroblastoma, or any other neoplastic tissue that expresses the HER2 receptor.

In a specific embodiment, the HER2-positive cancer is resistant to treatment with trastuzumab, cetuximab, lapatinib, erlotinib, or any other small molecule or antibody that targets the HER family of receptors. In a specific embodiment, the tumor that is resistant to treatment with trastuzumab, cetuximab, lapatinib, erlotinib, or any other small molecule or antibody that targets the HER family of receptors is responsive to treatment with a bispecific binding molecule of the invention. In a specific embodiment, the HER2-positive cancer is resistant to treatment with necitumumab, pantitumumab, pertuzumab, or ado-trastuzumab emtansine. In a specific embodiment, the HER2-positive cancer that is resistant to treatment with necitumumab, pantitumumab, pertuzumab, or ado-trastuzumab emtansine is responsive to treatment with a bispecific binding molecule of the invention.

In a specific embodiment, the HER2-positive cancer is a cancer that expresses programmed death-ligand 1 (“PDL1” or “PDL-1”) (i.e., a HER2-positive, PDL1-positive cancer). Thus, in a specific embodiment, provided herein is a method of treating a HER2-positive, PDL 1-positive cancer in a subject in need thereof, comprising administering to the subject a therapeutically effective amount of a bispecific binding molecule as described in Section 5.1 or in Section 5.2, a therapeutically effective amount of a cell, polynucleotide, or vector encoding such a bispecific binding molecule as described in Section 5.3, or a therapeutically effective amount of a pharmaceutical composition as described in Section 5.5, or a therapeutically effective amount of T cells bound to a bispecific binding molecule as described in Section 5.4. In a specific embodiment, the HER2-positive, PDL1-positive cancer overexpresses PDL1. In a specific embodiment, the HER2-positive, PDL1-positive cancer overexpresses PDL1 in cancerous cells relative to expression of PDL1 in analogous noncancerous cells of the same tissue type as the HER2-positive, PDL1-positive cancer. The noncancerous cells are analogous to the cancerous cells by virtue of the fact that they, for example, are from the same tissue or organ type or are otherwise suitable for comparison of PDL1 expression. The level of PDL1 expression in analogous noncancerous cells can be a known, standard level for a population or for particular individual(s) or for the subject having cancer, or can be newly measured. The overexpression can be shown, for example, by detecting increased PDL1 expression in a test specimen comprising cancerous cells relative to expression in a control specimen comprising analogous noncancerous cells. In contrast to the test specimen, the control specimen does not contain a significant amount of cancerous cells. In a specific embodiment, a HER2-positive, PDL1-positive cancer is deemed to overexpress PDL1 when the test specimen expresses a detectable level of PDL1 above background (i.e., experimental noise), preferably as measured by immunohistochemistry (“IHC”) since most normal tissue should be PDL1-negative. In a specific embodiment, the detectable level of PDL 1 above background is 1% to 5%, or is at least 1%, at least 2%, at least 3%, at least 4%, or at least 5% above background. In a specific embodiment in which the HER2-positive, PDL1-positive cancer is a melanoma, in a particular embodiment, the melanoma is deemed to overexpress PDL1 when the test specimen expresses a detectable level of PDL1 that is at least 5% above background. In a specific embodiment in which the HER2-positive, PDL1-positive cancer is a non-small cell lung carcinoma, in a particular embodiment, the non-small cell lung carcinoma is deemed to overexpress PDL1 if the test specimen expresses a detectable level of PDL 1 that is at least 5% above background. In a specific embodiment where binding to an anti-PDL1 antibody is used to measure the level of PDL1 expression in the test specimen and the control specimen, in a particular embodiment, the background level is measured by measuring nonspecific signal, for example, arising from binding to an antibody that recognizes an antigen known not to be expressed by the test or control specimen, e.g., an anti-IgG antibody. In a specific embodiment, PDL1 expression is measured by measuring PDL1 protein levels. In a specific embodiment, PDL1 expression is measured by measuring PDL1 nucleic acid levels (e.g., cDNA or RNA encoding PDL1). In a specific embodiment, PDL1 protein level is measured according to any assay known in the art, such as, e.g., IHC, western blot, enzyme-linked immunosorbent assay, or fluorescence-activated cell sorting. In a specific embodiment, PDL1 nucleic acid level is measured according to any assay known in the art, such as, e.g., in situ hybridization (“ISH”), southern blot, northern blot, quantitative reverse transcriptase polymerase chain reaction, or deep sequencing. The test specimen comprises cancer cells from the subject having cancer, and may be in the form of various biological specimens known in the art, e.g., from a biopsy or surgical resection. In a specific embodiment, a test specimen comprises cancerous cells from a primary tumor from the subject having cancer. In a specific embodiment, the test specimen comprises cancerous cells from a metastatic tumor from the subject having cancer. In a specific embodiment, the control specimen comprising analogous noncancerous cells (analogous to the cancerous cells in the test specimen) is a specimen obtained or derived from the subject who has cancer. Alternatively, a control specimen may be a specimen obtained or derived from a healthy subject or a subject who does not have cancer. In a specific embodiment, the control specimen does not comprise cancerous cells. In a specific embodiment, the test specimen and control specimen are from the same subject. In a specific embodiment, the test specimen and the control specimen are from different subjects. In a specific embodiment, the test specimen contains cancerous cells from breast tissue and the control specimen contains noncancerous cells from breast tissue.

Nonlimiting examples of HER2-positive cancers that express PDL1 and thus can be treated according to the methods described herein include breast cancer, gastric cancer, an osteosarcoma, desmoplastic small round cell cancer, ovarian cancer, prostate cancer, pancreatic cancer, glioblastoma multiforme, gastric junction adenocarcinoma, gastroesophageal junction adenocarcinoma, cervical cancer, salivary gland cancer, soft tissue sarcoma, leukemia, melanoma, Ewing's sarcoma, rhabdomyosarcoma, or neuroblastoma. In a specific embodiment, the HER2-positive, PDL1-positive cancer is not a head and neck cancer.

In a specific embodiment, a HER2-positive, PDL1-positive cancer treated according to the methods described herein is resistant to PDL1 blockade with an anti-PDL1 therapy. In a specific embodiment, the HER2-positive, PDL1-positive cancer is resistant to programmed cell death 1 (“PD1” or “PD-1”) blockade with an anti-PD1 therapy. In a specific embodiment, the HER2-positive, PDL1-positive cancer is resistant to (i) PDL1 blockade with an anti-PDL1 therapy, and (ii) PD1 blockade with an anti-PD1 therapy.

In a specific embodiment, PDL 1 blockade refers to (i) inhibition of PDL1-dependent PD1 activation, or (ii) blocking of PDL1 binding to PD1. In a specific embodiment, the inhibition or blocking is partial. In another specific embodiment, the inhibition or blocking is complete. In a specific embodiment, PDL 1 blockade refers to least 5%, 10%, 15%, 20%, 25%, 30%, 35%, 40%, 45%, 50%, 55%, 60%, 65%, 70%, 75%, 80%, 85%, 90%, 95%, 98%, or 99% inhibition of PDL1-dependent PD1 activation as assessed by any method known to one of skill in the art, such as, e.g., a phosphorylation assay, as compared to PDL1-dependent PD1 activation in the presence of a negative control therapy (e.g., an anti-IgG antibody). For example, in a specific embodiment, PDL1 blockade that is inhibition of activation is assessed by (a) contacting a PDL 1-expressing cell and a PD1-expressing activated T cell with an anti-PDL1 therapy (e.g., an anti-PDL1 antibody) or a negative control therapy (e.g., an anti-IgG antibody), and (b) measuring the phosphorylation of PD1 or dephosphorylation of a downstream signaling molecule, such as, e.g., Lck or Zap-70, as assessed by, for e.g., ELISA or western blot, in the presence of the anti-PDL1 therapy as compared to the phosphorylation of PD1 or dephosphorylation of a downstream signaling molecule, such as, e.g., Lck or Zap-70, as assessed by, for e.g., ELISA or western blot, in the presence of the negative control therapy. In a specific embodiment, PDL1 blockade that is blocking of PDL 1 binding to PD1 is assessed by (a) contacting a PDL 1-expressing cell and a PD1-expressing activated T cell with an anti-PDL1 therapy (e.g., an anti-PDL1 antibody) or a negative control therapy (e.g., an anti-IgG antibody), and (b) measuring the interaction between PDL1 and PD1 by, for example, co-localization (as assessed by, e.g., immunohistochemistry) or co-immunoprecipitation (as assessed by, e.g., western blot) of PDL1 and PD1, in the presence of the anti-PDL1 therapy as compared to the interaction between PDL1 and PD1 by, for example, co-localization (as assessed by, e.g., immunohistochemistry) or co-immunoprecipitation (as assessed by, e.g., western blot), in the presence of the negative control therapy.

In a specific embodiment, PD1 blockade refers to (i) inhibition of ligand-dependent PD1 activation, or (ii) blocking of ligand binding to PD1. In a specific embodiment, the inhibition or blocking is partial. In another specific embodiment, the inhibition or blocking is complete. In a specific embodiment, PD1 blockade refers to least 5%, 10%, 15%, 20%, 25%, 30%, 35%, 40%, 45%, 50%, 55%, 60%, 65%, 70%, 75%, 80%, 85%, 90%, 95%, 98%, or 99% inhibition of ligand-dependent PD1 activation as assessed by any method known to one of skill in the art, such as, e.g., a phosphorylation assay, as compared to ligand-dependent PD1 activation in the presence of a negative control therapy (e.g., an anti-lgG antibody). For example, in a specific embodiment, PD1 blockade that is inhibition of activation is assessed by (a) contacting a PD1 ligand-expressing cell and a PD1-expressing activated T cell with an anti-PD1 therapy (e.g., an anti-PD1 antibody) or a negative control therapy (e.g., an anti-IgG antibody), and (b) measuring the phosphorylation of PD1 or dephosphorylation of a downstream signaling molecule, such as, e.g., Lck or Zap-70, as assessed by, for e.g., ELISA or western blot, in the presence of the anti-PD1 therapy as compared to the phosphorylation of PD1 or dephosphorylation of a downstream signaling molecule, such as, e.g., Lck or Zap-70, as assessed by, for e.g., ELISA or western blot, in the presence of the negative control therapy. In a specific embodiment, PD1 blockade that is blocking of ligand binding to PD1 is assessed by (a) contacting a ligand-expressing cell and a PD1-expressing activated T cell with an anti-PD1 therapy (e.g., an anti-PD1 antibody) or a negative control therapy (e.g., an anti-IgG antibody), and (b) measuring the interaction between ligand and PD1 by, for example, co-localization (as assessed by, e.g., immunohistochemistry) or co-immunoprecipitation (as assessed by, e.g., western blot) of ligand and PD1, in the presence of the anti-PD1 therapy as compared to the interaction between ligand and PD1 by, for example, co-localization (as assessed by, e.g., immunohistochemistry) or co-immunoprecipitation (as assessed by, e.g., western blot), in the presence of the negative control therapy.

In a specific embodiment, an anti-PDL1 therapy is a PDL1-targeted therapy that is effective in the treatment of one or more cancers expressing PDL1. In a specific embodiment, the anti-PDL1 therapy comprises an antibody or antigen-binding fragment thereof (e.g., a Fab fragment, a F(ab′)2 fragment, or a disulfide-linked Fv) or antigen-binding derivative thereof (e.g., a bispecific antibody, an scFv, an intrabody, or a camelized antibody), a polypeptide, a RNAi-inducing nucleic acid (e.g., an antisense oligonucleotide, a small interfering RNA, a microRNA, or a short hairpin RNA), or a small molecule that targets PDL1. Nonlimiting examples of an anti-PDL1 therapy include mpdl3280A (see, e.g., Herbst et al., J Clin Oncol. 2013; 31 (suppl): abstr 3000), durvalumab (e.g., for bladder cancer) (also referred to as “medi-4736”; see, e.g., Lutzky et al., J Clin Oncol. 2014; 32 (suppl 5S): abstr 3001), avelumab (e.g., for Merkel cell carcinoma) (also referred to as “MSB0010718C”; see, e.g., Heery et al. J Clin Oncol. 2014; 32 (suppl 5S): abstr 3064), and bms-936559 (see, e.g., Brahmer et al. N. Engl. J. Med. 2012; 366, 2455-2465), and atezolizumab (see, e.g., McDermott et al., J Clin Oncol. 2016; 34 (8): 833-842). In a preferred embodiment, the anti-PDL1 therapy is an anti-PDL1 antibody. In a preferred embodiment, the anti-PDL1 antibody is atezolizumab. In a specific embodiment, the anti-PDL 1 therapy is a therapy approved by the U.S. Food and Drug Administration (“FDA”) for treatment of one or more cancers. A nonlimiting example of an anti-PDL1 therapy approved by the U.S. Food and Drug Administration for treatment of cancer is atezolizumab. In a specific embodiment, the anti-PDL1 therapy is a PDL1-targeted therapy approved by the European Medicines Agency (“EMA”) for treatment of one or more cancers. A nonlimiting example of an anti-PDL 1 therapy approved by the EMA for treatment of a PDL1-expressing cancer is atezolizumab.

In a specific embodiment, an anti-PD1 therapy is a PD1-targeted therapy that is effective in the treatment of one or more cancers expressing PDL1. In a specific embodiment, the anti-PD1 therapy comprises an antibody or antigen-binding fragment thereof (e.g., a Fab fragment, a F(ab′)2 fragment, or a disulfide-linked Fv) or antigen-binding derivative thereof (e.g., a bispecific antibody, an scFv, an intrabody, or a camelized antibody), a polypeptide, a RNAi-inducing nucleic acid (e.g., an antisense oligonucleotide, a small interfering RNA, a microRNA, or a short hairpin RNA), or a small molecule that targets PD1. Nonlimiting examples of an anti-PD1 therapy include nivolumab (see, e.g., Topalian et al., N Engl J Med. 2012; 366:2443-54), pidilizumab (see, e.g., Atkins et al., J Clin Oncol. 2014; 32 (suppl 5S): abstr 9001), AMP-224 (see, e.g., Infante et al., J Clin Oncol. 2013; 31 (suppl): abstr 3044), MEDI0680 (also referred to as “AMP-514”; see, e.g., Hamid et al., Ann Oncol. 2016; 27 (suppl_6): 1050PD), and pembrolizumab (see, e.g., Hamid et al., N Engl J Med. 2013; 369:134-44). In a preferred embodiment, the anti-PD1 therapy is an anti-PD1 antibody. In a preferred embodiment, the anti-PD1 antibody is pembrolizumab. In a specific embodiment, the anti-PD1 therapy is a therapy approved by the U.S. FDA for treatment of one or more cancers. Nonlimiting examples of an anti-PD1 therapy approved by the U.S. FDA for treatment of cancer include pembrolizumab and nivolumab. In a specific embodiment, the anti-PD1 therapy is a therapy approved by the EMA for treatment of one or more cancers. Nonlimiting examples of an anti-PD1 therapy approved by the EMA for treatment of cancer include pembrolizumab and nivolumab.

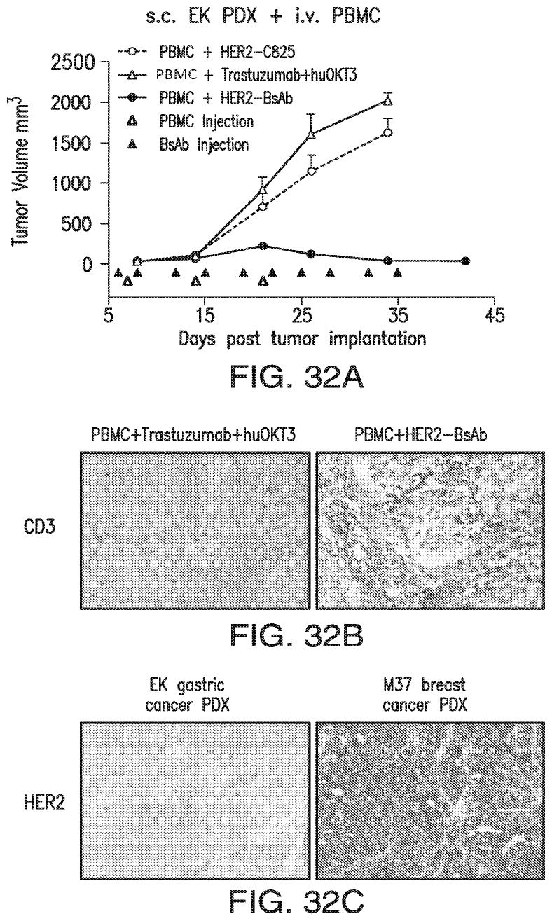

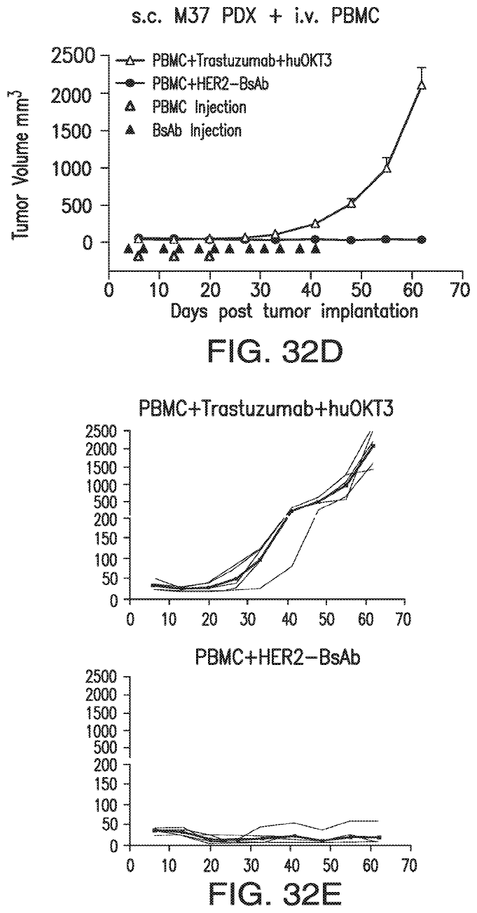

In contrast to trastuzumab (which is indicated for treatment of HER2-overexpressing breast cancer, metastatic gastric cancer, and gastroesophageal junction adenocarcinoma (see Trastuzumab [Highlights of Prescribing Information], South San Francisco, CA: Genentech, Inc.; 2014)), the bispecific binding molecules described herein are therapeutically effective against HER2-positive cancers that express low levels of HER2. See, e.g., the working example of Section 6.3, in particular, FIG. 32, which demonstrates that tumor growth was completely suppressed in a gastric cancer patient-derived xenograft model with low HER2 expression when treated with a bispecific binding molecule described herein; in contrast, treatment of the gastric cancer patient-derived xenograft model with trastuzumab did not suppress tumor growth. Thus, also provided herein is a method of treating a HER2-positive cancer in a subject in need thereof, comprising administering to the subject a therapeutically effective amount of a bispecific binding molecule as described in Section 5.1 or in Section 5.2, a therapeutically effective amount of a cell, polynucleotide, or vector encoding such a bispecific binding molecule as described in Section 5.3, or a therapeutically effective amount of a pharmaceutical composition as described in Section 5.5, or a therapeutically effective amount of T cells bound to a bispecific binding molecule as described in Section 5.4, wherein the cancer is not indicated for treatment with trastuzumab, and wherein the cancer is not a head and neck cancer. In a specific embodiment, the cancer is breast cancer. In a specific embodiment, the cancer is gastric cancer. In a specific embodiment, the cancer is gastroesophageal junction adenocarcinoma.

In a specific embodiment, the HER2-positive cancer is determined not to be indicated for treatment with trastuzumab according to applicable American Society of Clinical Oncology/College of American Pathologists (“ASCO/CAP”) guideline recommendations for HER2 testing in cancer (“ASCO HER2 Testing Guidelines”) (see, e.g., Wolff et al., Journal of Clinical Oncology, 2013, 31 (31): 3997-4013 and Bartley et al., Journal of Clinical Oncology, 2016, 146 (6): 647-669). The applicable ASCO HER2 Testing Guideline will be known to one of skill in the art. In a specific embodiment, the applicable ASCO HER2 Testing Guideline is the current (i.e., most recently published and updated) ASCO HER2 Testing Guideline as of the date of using the ASCO HER2 Testing Guideline to determine that the cancer is not indicated for treatment with trastuzumab. In an alternative, preferred embodiment, the applicable ASCO HER2 Testing Guideline is the current (e.g., most recently published and updated) ASCO/CAP guideline recommendations for HER2 testing in breast cancer (“ASCO HER2 Breast Cancer Testing Guideline”) (see, e.g., Wolff et al., Journal of Clinical Oncology, 2013, 31 (31): 3997-4013) as of the date of determining that the cancer is not indicated for treatment with trastuzumab, regardless of the type of cancer that is determined not to be indicated for treatment with trastuzumab (e.g., the type of cancer may be breast cancer or any other HER2-positive cancer). In another embodiment, the applicable ASCO HER2 Testing Guideline is the current (e.g., most recently published and updated) ASCO HER2 Testing Guideline as of the date of determining that the cancer is not indicated for treatment with trastuzumab and the applicable ASCO HER2 Testing Guideline is for the same type of cancer (e.g., same tissue type, for example, both being breast cancers, or both being gastric cancers) as the cancer that is determined not to be indicated for treatment with trastuzumab.

In a specific embodiment, the HER2-positive cancer is determined not to be indicated for treatment with trastuzumab based on the following characterization of the cancer (see, e.g., the 2013 ASCO HER2 Breast Cancer Testing Guideline (e.g., as set forth in Wolff et al., Journal of Clinical Oncology, 2013, 31 (31): 3997-4013)): (a) a first determination of a level of HER2 in a test specimen comprising cells of the cancer is reported as negative, or (b) a first determination of a level of HER2 in a test specimen comprising cells of the cancer is reported as equivocal, and a second determination of a level of HER2 in a test specimen comprising cells of the cancer is reported as equivocal or negative. In a specific embodiment, the HER2-positive cancer is determined not to be indicated for treatment with trastuzumab when a first determination of a level of HER2 in a test specimen comprising cells of the cancer is reported as negative according to the applicable ASCO HER2 Testing Guideline (e.g., the 2013 ASCO HER2 Breast Cancer Testing Guideline (e.g., as set forth in Wolff et al., Journal of Clinical Oncology, 2013, 31 (31): 3997-4013)). In a specific embodiment, the HER2-positive cancer is determined not to be indicated for treatment with trastuzumab when a first determination of a level of HER2 in a test specimen comprising cells of the cancer is reported as equivocal according to the applicable ASCO HER2 Testing Guideline (e.g., the 2013 ASCO HER2 Breast Cancer Testing Guideline (e.g., as set forth in Wolff et al., Journal of Clinical Oncology, 2013, 31 (31): 3997-4013)) and a second determination of a level of HER2 in a test specimen comprising cells of the cancer is reported as equivocal or negative according to the applicable ASCO HER2 Testing Guideline (e.g., the 2013 ASCO HER2 Breast Cancer Testing Guideline (e.g., as set forth in Wolff et al., Journal of Clinical Oncology, 2013, 31 (31): 3997-4013)). The test specimen can be from a primary tumor or a metastatic tumor.

In a specific embodiment, the determination of the level of HER2 in the test specimen is reported as negative when the level of HER2 in the test specimen is characterized as (see, e.g., the 2013 ASCO HER2 Breast Cancer Testing Guideline (e.g., as set forth in Wolff et al., Journal of Clinical Oncology, 2013, 31 (31): 3997-4013)): (i) (1) IHC 1+, wherein the level of HER2 in the test specimen is characterized as IHC 1+ when the test specimen exhibits an incomplete HER2 membrane staining that is faint/barely perceptible and within greater than 10% of the invasive tumor cells, wherein the staining is readily appreciated using a low-power objective; (2) IHC 0, wherein the level of HER2 in the test specimen is characterized as IHC 0 when the test specimen exhibits no staining observed, wherein the lack of staining is readily appreciated using a low-power objective, or a HER2 membrane staining that is incomplete and is faint/barely perceptible and within less than or equal to 10% of the invasive tumor cells, wherein the staining is readily appreciated using a low-power objective; or (ii) ISH negative, wherein the level of HER2 in the test specimen is characterized as ISH negative when the test specimen exhibits (1) a single-probe average HER2 copy number of less than 4.0 signals per cell; or (2) a dual-probe HER2/CEP17 ratio of less than 2.0 with an average HER2 copy number of less than 4.0 signals per cell.

In a specific embodiment, the determination of the level of HER2 in the test specimen is reported as equivocal when the level of HER2 in the test specimen is characterized as (see, e.g., the 2013 ASCO HER2 Breast Cancer Testing Guideline (e.g., as set forth in Wolff et al., Journal of Clinical Oncology, 2013, 31 (31): 3997-4013)): (i) IHC 2+, wherein the level of HER2 in the test specimen is characterized as IHC 2+ when the test specimen exhibits (1) a circumferential HER2 membrane staining that is incomplete and/or weak/moderate and within greater than 10% of invasive tumor cells, wherein the staining is observed in a homogenous and contiguous population, and wherein the staining is readily appreciated using a low-power objective; or (2) a complete and circumferential HER2 membrane staining that is intense and within less than or equal to 10% of invasive tumor cells, wherein the staining is readily appreciated using a low-power objective; or (ii) ISH equivocal, wherein the level of HER2 in the test specimen is characterized as ISH equivocal when the test specimen exhibits which comprises: (1) a single-probe ISH average HER2 copy number of greater than or equal to 4.0 and less than 6.0 signals/cell, wherein the copy number is determined by counting at least 20 cells within the area and is observed in a homogenous and contiguous population; or (2) a dual-probe HER2/CEP17 ratio of less than 2.0 with an average HER2 copy number of greater than or equal to 4.0 and less than 6.0 signals per cell, wherein the copy number is determined by counting at least 20 cells within the area and is observed in a homogenous and contiguous population. In a specific embodiment, when two determinations of the level of HER2 in a subject are made to determine that a cancer is not indicated for treatment with trastuzumab (e.g., a first determination is reported as equivalent and a second determination is reported as equivalent or negative), the two determinations are either: (1) based on the same test specimen using different assays; or (2) based on different test specimens using the same assay. For example, if the first determination is based on a first test specimen using ISH, the second determination is based on the first test specimen using IHC. In an alternative example, if the first determination is made based on a first test specimen using ISH, the second determination is based on a second test specimen using ISH.

In a specific embodiment, the level of HER2 in the test specimen is determined according to one or more assays approved by the U.S. Food and Drug Administration (“FDA”) for determining the level of HER2. Nonlimiting examples of U.S. Food and Drug Administration-approved assays for determining a level of HER2 include HercepTest™ (manufactured by DAKO), PATHWAY® (manufactured by Ventana Medical Systems Inc.), InSite® (manufactured by Biogenex Laboratories Inc.), Bond Oracle™ (manufactured by Leica Biosystems), Path Vysion® (manufactured by Abbott Molecular Inc.), PharmDx™ Kit (manufactured by DAKO), SPOT-Light® (manufactured by Life Technologies Inc.), INFORM HER2 dual IDS DNA probe cocktail (manufactured by Ventana Medical Systems Inc.), and PharmDx™ (manufactured by DAKO). In another specific embodiment, the level of HER2 in the test specimen is determined according to a laboratory-developed test performed in a Clinical Laboratory Improvement Amendments-certified laboratory.

Also provided herein is a method of treating a HER2-positive cancer in a subject in need thereof, comprising administering to the subject a therapeutically effective amount of a bispecific binding molecule as described in Section 5.1 or in Section 5.2, a therapeutically effective amount of a cell, polynucleotide, or vector encoding such a bispecific binding molecule as described in Section 5.3, or a therapeutically effective amount of a pharmaceutical composition as described in Section 5.5, or a therapeutically effective amount of T cells bound to a bispecific binding molecule as described in Section 5.4, wherein the cancer expresses a low level of HER2, and wherein the cancer is not a head and neck cancer. In a specific embodiment, the cancer is breast cancer. In another specific embodiment, the cancer is gastric cancer. In a specific embodiment, the cancer is gastroesophageal junction adenocarcinoma.

In a preferred embodiment, the HER2-positive cancer is deemed to express a low level of HER2 when a level of HER2 in a test specimen comprising cells of the cancer is characterized as IHC 2+ or less (e.g., IHC 1+ or IHC 0) according to the applicable ASCO HER2 Testing Guideline. In a specific embodiment, the HER2-positive cancer is deemed to express a low level of HER2 when a level of HER2 in a test specimen comprising cells of the cancer is characterized as IHC 2+ according to the applicable ASCO HER2 Testing Guideline. In a specific embodiment, the HER2-positive cancer is deemed to express a low level of HER2 when a level of HER2 in a test specimen comprising cells of the cancer is characterized as IHC 1+ according to the applicable ASCO HER2 Testing Guideline. In a specific embodiment, the HER2-positive cancer is deemed to express a low level of HER2 when a level of HER2 in a test specimen comprising cells of the cancer is characterized as IHC 0 according to the applicable ASCO HER2 Testing Guideline. The applicable ASCO HER2 Testing Guideline will be known to one of skill in the art. In a specific embodiment, the applicable ASCO HER2 Testing Guideline is the current (e.g., most recently published and updated) ASCO HER2 Testing Guideline as of the date of characterizing the level of HER2 in the test specimen comprising cells of the cancer as IHC 2+ or less (e.g., IHC 1+ or IHC 0). In a preferred embodiment, the applicable ASCO HER2 Testing Guideline is the current (e.g., most recently published and updated) ASCO HER2 Breast Cancer Testing Guideline (see, e.g., Wolff et al., Journal of Clinical Oncology, 2013, 31 (31): 3997-4013) as of the date of characterizing the level of HER2 in the test specimen, regardless of the type of cancer of the test specimen (e.g., the test specimen may be of breast cancer or any other HER2-positive cancer). In another embodiment, the applicable ASCO HER2 Testing Guideline is for the same type of cancer (e.g., same tissue type, for example, both being breast cancers, or both being gastric cancers) as the cancer of the test specimen. In another embodiment, the applicable ASCO HER2 Testing Guideline is the current (e.g., most recently published and updated) ASCO HER2 Testing Guideline as of the date of characterizing the level of HER2 in the test specimen and the applicable ASCO HER2 Testing Guideline is for the same type of cancer (e.g., same tissue type, for example, both being breast cancers, or both being gastric cancers) as the test specimen. In a specific embodiment, the level of HER2 in the test specimen comprising cells of the cancer is characterized as IHC 2+ when the test specimen exhibits (see, e.g., Wolff et al., Journal of Clinical Oncology, 2013, 31 (31): 3997-4013) (1) a circumferential HER2 membrane staining that is incomplete and/or weak/moderate and within greater than 10% of invasive tumor cells, wherein the staining is observed in a homogenous and contiguous population, and wherein the staining is readily appreciated using a low-power objective; or (2) a complete and circumferential HER2 membrane staining that is intense and within less than or equal to 10% of invasive tumor cells, wherein the staining is readily appreciated using a low-power objective. In a specific embodiment, the level of HER2 in a test specimen comprising cells of the cancer is characterized as IHC 1+ when the test specimen exhibits (see, e.g., Wolff et al., Journal of Clinical Oncology, 2013, 31 (31): 3997-4013) an incomplete HER2 membrane staining that is faint/barely perceptible and within greater than 10% of the invasive tumor cells, wherein the staining is readily appreciated using a low-power objective. In a specific embodiment, the level of HER2 in the test specimen comprising cells of the cancer is characterized as IHC when the test specimen exhibits (see, e.g., Wolff et al., Journal of Clinical Oncology, 2013, 31 (31): 3997-4013) no HER2 staining observed, wherein the lack of staining is readily appreciated using a low-power objective, or a HER2 membrane staining that is incomplete and is faint/barely perceptible and within less than or equal to 10% of the invasive tumor cells, wherein the staining is readily appreciated using a low-power objective.

In another embodiment, the HER2-positive cancer is deemed to express a low level of HER2 when the cancer expresses a lower level of HER2 than the level of HER2 expressed by cancers that are indicated for treatment with trastuzumab and are of the same type (e.g., same tissue type, for example, both being breast cancers, or both being gastric cancers) as the HER2-positive cancer. In a specific embodiment, the HER2 positive cancer is deemed to express a low level of HER2 when the HER2-positive cancer expresses a level of HER2 that is at least 10%, 20%, 30%, 40%, 50%, 60%, 70%, 80%, 90%, or 100% lower than the level of HER2 expressed by cancers that are indicated for treatment with trastuzumab and are of the same type (e.g., same tissue type, for example, both being breast cancers, or both being gastric cancers) as the HER2-positive cancer. In a specific embodiment, HER2 expression is measured by measuring HER2 protein levels. In a specific embodiment, HER2 expression is measured by measuring HER2 nucleic acid levels (e.g, genomic DNA, cDNA, or RNA encoding HER2). In a specific embodiment, HER2 protein level is measured according to any assay known in the art, such as, e.g., IHC, western blot, enzyme-linked immunosorbent assay, or fluorescence-activated cell sorting. In a preferred embodiment, HER2 protein level is measured according to IHC. In a specific embodiment, HER2 nucleic acid level is measured according to any assay known in the art, such as, e.g., ISH, southern blot, northern blot, quantitative reverse transcriptase polymerase chain reaction, or deep sequencing. In a preferred embodiment, HER2 nucleic acid level is measured according to ISH.

In a specific embodiment, the level of HER2 in the test specimen is determined according to one or more assays approved by the U.S. Food and Drug Administration (“FDA”) for determining the level of HER2. Nonlimiting examples of U.S. Food and Drug Administration-approved assays for determining a level of HER2 include HercepTest™ (manufactured by DAKO), PATHWAY® (manufactured by Ventana Medical Systems Inc.), InSite® (manufactured by Biogenex Laboratories Inc.), Bond Oracle™ (manufactured by Leica Biosystems), Path Vysion® (manufactured by Abbott Molecular Inc.), PharmDx™ Kit (manufactured by DAKO), SPOT-Light® (manufactured by Life Technologies Inc.), INFORM HER2 dual IDS DNA probe cocktail (manufactured by Ventana Medical Systems Inc.), and PharmDx™ (manufactured by DAKO). In another specific embodiment, the level of HER2 in the test specimen is determined according to a laboratory-developed test performed in a Clinical Laboratory Improvement Amendments-certified laboratory.

In a specific embodiment, the HER2-positive cancer that expresses a low level of HER2 is a breast cancer, a gastric cancer, an osteosarcoma, desmoplastic small round cell cancer, an ovarian cancer, a prostate cancer, a pancreatic cancer, glioblastoma multiforme, gastric junction adenocarcinoma, gastroesophageal junction adenocarcinoma, a cervical cancer, a salivary gland cancer, a soft tissue sarcoma, a leukemia, a melanoma, a Ewing's sarcoma, a rhabdomyosarcoma, a brain tumor, or neuroblastoma. In a preferred embodiment, the HER2-positive cancer that expresses a low level of HER2 is breast cancer. In a specific embodiment, the HER2-positive cancer that expresses a low level of HER2 is gastric cancer. In a specific embodiment, the HER2-positive cancer that expresses a low level of HER2 is ovarian cancer, pancreatic cancer, a desmoplastic small round cell tumor, an osteosarcoma, a melanoma, a brain tumor, a cervical cancer, a prostate cancer, or a salivary gland cancer. In a specific embodiment, the HER2-positive cancer that expresses a low level of HER2 is not a head and neck cancer.

In a specific embodiment, the HER2-positive cancer that expresses a low level of HER2 is resistant to treatment with trastuzumab, cetuximab, lapatinib, erlotinib, or any other small molecule or antibody that targets the HER family of receptors, and is responsive to treatment with a bispecific binding molecule of the invention. In a specific embodiment, the HER2-positive cancer that expresses a low level of HER2 is resistant to treatment with necitumumab, pantitumumab, pertuzumab, or ado-trastuzumab emtansine, and is responsive to treatment with a bispecific binding molecule of the invention.

In a specific embodiment, a cancer is considered resistant to a therapy (e.g., an anti-PDL1 therapy, an anti-PD1 therapy, trastuzumab, cetuximab, necitumumab, panitumumab, pertuzumab, ado-trastuzumab emtansine, lapatinib, erlotinib, or any small molecule that targets the HER family of receptors) if it has no response, or has an incomplete response (a response that is less than a complete remission), or progresses, or relapses after the therapy.

In specific embodiments, treatment can be to achieve beneficial or desired clinical results including, but not limited to, alleviation of a symptom, diminishment of extent of a disease, stabilizing (i.e., not worsening) of state of a disease, delay or slowing of disease progression, amelioration or palliation of the disease state, and remission (whether partial or total), whether detectable or undetectable. In a specific embodiment, “treatment” can also be to prolong survival as compared to expected survival if not receiving treatment.

### 5.6.2 Diagnostic Uses

In certain embodiments, bispecific binding molecules described herein can be used for diagnostic purposes to detect, diagnose, or monitor a condition described herein (e.g., a condition involving HER2-positive cancer cells). In certain embodiments, bispecific binding molecules for use in diagnostic purposes are labeled as described in Section 5.2.

In certain embodiments, provided herein are methods for the detection of a condition described herein comprising (a) assaying the expression of HER2 in cells or a tissue sample of a subject using one or more bispecific binding molecules described herein; and (b) comparing the level of HER2 expression with a control level, for example, levels in normal tissue samples (e.g., from a subject not having a condition described herein, or from the same patient before onset of the condition), whereby an increase or decrease in the assayed level of HER2 expression compared to the control level of HER2 expression is indicative of a condition described herein.

Antibodies described herein can be used to assay HER2 levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

In certain embodiments, monitoring of a condition described herein (e.g., a HER2-positive cancer), is carried out by repeating the method for diagnosing for a period of time after initial diagnosis.

Presence of the labeled molecule can be detected in the subject using methods known in the art for in vivo scanning. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

### 5.7 Patient Population

A subject treated in accordance with the methods provided herein can be any mammal, such as a rodent, a cat, a canine, a horse, a cow, a pig, a monkey, a primate, or a human, etc. In a preferred embodiment, the subject is a human. In another preferred embodiment, the subject is a canine.

In certain embodiments, a subject treated in accordance with the methods provided herein has been diagnosed with a HER2-positive cancer, including but not limited to, breast cancer, gastric cancer, an osteosarcoma, desmoplastic small round cell cancer, squamous cell carcinoma of head and neck cancer, ovarian cancer, prostate cancer, pancreatic cancer, glioblastoma multiforme, gastric junction adenocarcinoma, gastroesophageal junction adenocarcinoma, cervical cancer, salivary gland cancer, soft tissue sarcoma, leukemia, melanoma, Ewing's sarcoma, rhabdomyosarcoma, neuroblastoma, or any other neoplastic tissue that expresses the HER2 receptor.

In a specific embodiment, a subject treated in accordance with the methods provided herein has not been diagnosed with HER2-positive squamous cell carcinoma of head and neck cancer.

In certain embodiments, the subject is resistant to treatment with trastuzumab, cetuximab, lapatinib, erlotinib, or any other small molecule or antibody that targets the HER family of receptors. In a specific embodiment, the tumor that is resistant to treatment with trastuzumab, cetuximab, lapatinib, erlotinib, or any other small molecule or antibody that targets the HER family of receptors is responsive to treatment with a bispecific binding molecule to the invention.

In certain embodiments, a subject treated in accordance with the methods provided herein has a HER2-positive cancer that is resistant to treatment with trastuzumab, cetuximab, lapatinib, erlotinib, or any other small molecule or antibody that targets the HER family of receptors. In certain embodiments, a subject treated in accordance with the methods provided herein has a HER2-positive cancer that is responsive to treatment with a bispecific binding molecule to the invention.

In certain embodiments, the subject treated in accordance with the methods provided herein has previously received one or more chemotherapy regimens for metastatic disease, e.g., brain or peritoneal metastases. In certain embodiments, the subject has not previously received treatment for metastatic disease.

### 5.8 Doses and Regimens

In certain embodiments, the dose of a bispecific binding molecule as described in Section 5.1 administered to a subject according to the methods provided herein is a dose determined by the needs of the subject. In certain embodiments, the dose is determined by a physician according to the needs of the subject.

In a specific embodiment, the dose of a bispecific binding molecule provided herein is less than the dose of trastuzumab. See, for example, Trastuzumab [Highlights of Prescribing Information]. South San Francisco, CA: Genentech, Inc.; 2014. In a specific embodiment, the dose of a bispecific binding molecule provided herein is approximately between 20 and 40 fold less than an FDA-approved dose of trastuzumab.

In certain embodiments, the dose of a bispecific binding molecule as described in Section 5.1 administered to a subject according to the methods provided herein is between 0.01 mg/kg and 0, 025 mg/kg, is between 0.025 mg/kg and 0.05 mg/kg, is between 0.05 mg/kg and 0.1 mg/kg, is between 0.1 mg/kg and 0.5 mg/kg, between 0.1 mg/kg and 0.6 mg/kg, between 0.2 mg/kg and 0.7 mg/kg, between 0.3 mg/kg and 0.8 mg/kg, between 0.4 mg/kg and 0.8 mg/kg, between 0.5 mg/kg and 0.9 mg/kg, or between 0.6 mg/kg and 1.

In certain embodiments, the dose of a bispecific binding molecule as described in Section 5.1 administered to a subject according to the methods provided herein is an initial dose followed by an adjusted dose that is the maintenance dose. In certain embodiments, the initial dose is administered once. In certain embodiments, the initial dose is between 0.01 mg/kg and 0.025 mg/kg, is between 0.025 mg/kg and 0.05 mg/kg, is between 0.05 mg/kg and 0.1 mg/kg, is between 0.1 mg/kg and 0.5 mg/kg, between 0.1 mg/kg and 0.6 mg/kg, between 0.2 mg/kg and 0.7 mg/kg, between 0.3 mg/kg and 0.8 mg/kg, between 0.4 mg/kg and 0.8 mg/kg, between 0.5 mg/kg and 0.9 mg/kg, or between 0.6 mg/kg and 1. In certain embodiments, the initial dose is administered via intravenous infusion over 90 minutes. In certain embodiments, the adjusted dose is administered once every about 4 weeks. In certain embodiments, the adjusted dose is administered for at least 13, at least 26, or at most 52 weeks. In certain embodiments, the adjusted dose is administered for 52 weeks. In certain embodiments, the adjusted dose is between 0.01 mg/kg and 0.025 mg/kg, is between 0.025 mg/kg and 0.05 mg/kg, is between 0.05 mg/kg and 0.1 mg/kg, is between 0.1 mg/kg and 0.5 mg/kg, between 0.1 mg/kg and 0.6 mg/kg, between 0.2 mg/kg and 0.7 mg/kg, between 0.3 mg/kg and 0.8 mg/kg, between 0.4 mg/kg and 0.8 mg/kg, between 0.5 mg/kg and 0.9 mg/kg, or between 0.6 mg/kg and 1. In certain embodiments, the adjusted dose is administered via intravenous infusion over 30 minutes. In certain embodiments, the adjusted dose is administered via intravenous infusion over 30 to 90 minutes.

In another specific embodiment, a bispecific binding molecule as described in Section 5.1 for use with the methods provided herein is administered 1, 2, or 3 times a week, every 1, 2, 3, or 4 weeks. In certain embodiments, the bispecific binding molecule is administered according to the following regimen: (i) 1, 2, or 3 administrations in a first week; (ii) 1, 2, 3, or 4 administrations a week after the first week; followed by (iii) 1, 2, or 3 administrations in one week each month for a total of 1, 2, 3, 4, 5, 6, 7, 8, 9, 10, 11, or 12 months. In certain embodiments, the bispecific binding molecule is administered according to the following regimen: (i)₃ administrations in a first week; (ii)₃ administrations a week after the first week; followed by (iii)₃ administrations in one week each month for a total of 1, 2, 3, 4, 5, 6, 7, 8, 9, 10, 11, or 12 months. In certain embodiments, the bispecific binding molecule is administered according to the following regimen: (i)₃ administrations in a first week; (ii)2 administrations a week after the first week; followed by (iii)2 administrations in one week each month for a total of 1, 2, 3, 4, 5, 6, 7, 8, 9, 10, 11, or 12 months. In certain embodiments, the bispecific binding molecule is administered according to the following regimen: (i)₃ administrations in a first week; (ii) 1 administrations a week after the first week; followed by (iii) 1 administrations in one week each month for a total of 1, 2, 3, 4, 5, 6, 7, 8, 9, 10, 11, or 12 months. In certain embodiments, the bispecific binding molecule is administered according to the following regimen: (i)2 administrations in a first week; (ii)2 administrations a week after the first week; followed by (iii)2 administrations in one week each month for a total of 1, 2, 3, 4, 5, 6, 7, 8, 9, 10, 11, or 12 months. In certain embodiments, the bispecific binding molecule is administered according to the following regimen: (i)2 administrations in a first week; (ii) 1 administrations a week after the first week; followed by (iii) 1 administrations in one week each month for a total of 1, 2, 3, 4, 5, 6, 7, 8, 9, 10, 11, or 12 months. In certain embodiments, the bispecific binding molecule is administered according to the following regimen: (i) 1 administrations in a first week; (ii) 1 administrations a week after the first week; followed by (iii) 1 administrations in one week each month for a total of 1, 2, 3, 4, 5, 6, 7, 8, 9, 10, 11, or 12 months.

In certain embodiments, a bispecific binding molecule as described in Section 5.1 is administered to a subject according to the methods provided herein in combination with a second pharmaceutically active agent as described in Section 5.9.

In another preferred embodiment, the bispecific binding molecule is administered intrathecally, intraventricularly in the brain, intraparenchymally in the brain, or intraperitoneally.

### 5.9 Combination Therapy

In certain embodiments, a bispecific binding molecule provided herein, or polynucleotide, vector, or cell encoding the bispecific binding molecule, may be administered in combination with one or more additional pharmaceutically active agents, e.g., a cancer chemotherapeutic agent. In certain embodiments, such combination therapy may be achieved by way of simultaneous, sequential, or separate dosing of the individual components of the treatment. In certain embodiments, the bispecific binding molecule or polynucleotide, vector, or cell encoding the bispecific binding molecule, and one or more additional pharmaceutically active agents may be synergistic, such that the dose of either or of both of the components may be reduced as compared to the dose of either component that would be given as a monotherapy. Alternatively, In certain embodiments, the bispecific binding molecule or polynucleotide, vector, or cell encoding the bispecific binding molecule and the one or more additional pharmaceutically active agents may be additive, such that the dose of the bispecific binding molecule and of the one or more additional pharmaceutically active agents is similar or the same as the dose of either component that would be given as a monotherapy.

In certain embodiments, a bispecific binding molecule provided herein, or polynucleotide, vector, or cell encoding the bispecific binding molecule is administered on the same day as one or more additional pharmaceutically active agents. In certain embodiments, the bispecific binding molecule or polynucleotide, vector, or cell encoding the bispecific binding molecule is administered 1, 2, 3, 4, 5, 6, 7, 8, 9, 10, 11, or 12 hours before the one or more additional pharmaceutically active agents. In certain embodiments, the bispecific binding molecule or polynucleotide, vector, or cell encoding the bispecific binding molecule is administered 1, 2, 3, 4, 5, 6, 7, 8, 9, 10, 11, or 12 hours after the one or more additional pharmaceutically active agents. In certain embodiments, the bispecific binding molecule or polynucleotide, vector, or cell encoding the bispecific binding molecule is administered 1, 2, 3, or more days before the one or more additional pharmaceutically active agents. In certain embodiments, the bispecific binding molecule or polynucleotide, vector, or cell encoding the bispecific binding molecule is administered 1, 2, 3 or more days after the one or more additional pharmaceutically active agents. In certain embodiments, the bispecific binding molecule or polynucleotide, vector, or cell encoding the bispecific binding molecule is administered 1, 2, 3, 4, 5, or 6 weeks before the one or more additional pharmaceutically active agents. In certain embodiments, the bispecific binding molecule or polynucleotide, vector, or cell encoding the bispecific binding molecule is administered 1, 2, 3, 4, 5, or 6 weeks after the one or more additional pharmaceutically active agents.

In certain embodiments, the additional pharmaceutically active agent is doxorubicin. In certain embodiments, the additional pharmaceutically active agent is cyclophosphamide. In certain embodiments, the additional pharmaceutically active agent is paclitaxel. In certain embodiments, the additional pharmaceutically active agent is docetaxel. In certain embodiments, the one or more additional pharmaceutically active agents is carboplatin.

In certain embodiments, the additional pharmaceutically active agent is a cytokine, such as, for example, IL15, IL15R/IL15 complex, IL2, or GMCSF.

In certain embodiments, the additional pharmaceutically active agent is an agent that increases cellular HER2 expression, such as, for example, external beam or radioimmunotherapy. See, for example, Wattenberg et al., 2014, British Journal of Cancer, 110:1472.

In certain embodiments, the additional pharmaceutically active agent is a radiotherapeutic agent.

In certain embodiments, the additional pharmaceutically active agent is an agent that directly controls the HER2 signaling pathway, e.g., lapatinib. See, for example, Scaltiri et al., 2012, 28 (6): 803-814.

In certain embodiments, the additional pharmaceutically active agent is an agent that increases cell death, apoptosis, autophagy, or necrosis of tumor cells.

In certain embodiments, a bispecific binding molecule provided herein, or polynucleotide, vector, or cell encoding the bispecific binding molecule is administered in combination with two additional pharmaceutically active agents, e.g., those used in combination with trastuzumab (see, Trastuzumab [Highlights of Prescribing Information]. South San Francisco, CA: Genentech, Inc.; 2014). In certain embodiments, the two additional pharmaceutically active agents are doxorubicin and paclitaxel. In certain embodiments, the two additional pharmaceutically active agents are doxorubicin and docetaxel. In certain embodiments, the two additional pharmaceutically active agents are cyclophosphamid and paclitaxel. In certain embodiments, the two additional pharmaceutically active agents are cyclophosphamide and docetaxel. In certain embodiments, the two additional pharmaceutically active agents are docetaxel and carboplatin. In certain embodiments, the two additional pharmaceutically active agents are cisplatin and capecitabine. In certain embodiments, the two additional pharmaceutically active agents are cisplatin and 5-fluorouracil.

In certain embodiments, a bispecific binding molecule provided herein, or polynucleotide, vector, or cell encoding the bispecific binding molecule is administered as a single agent following multi-modality anthracycline based therapy.

In certain embodiments, a bispecific binding molecule provided herein, or polynucleotide, vector, or cell encoding the bispecific binding molecule is administered after one or more chemotherapy regimens for metastatic disease, e.g., brain or peritoneal metastases. In specific embodiments, a bispecific binding molecule provided herein, or polynucleotide, vector, or cell encoding the bispecific binding molecule is administered in combination with cytoreductive chemotherapy. In a specific embodiment, the administering is performed after treating the subject with cytoreductive chemotherapy.

In specific embodiments, a bispecific binding molecule provided herein, polynucleotide, vector, or cell encoding the bispecific binding molecule, or a pharmaceutical composition comprising the bispecific binding molecule, is administered in combination with T cell infusion. In specific embodiments, the bispecific binding molecule is not bound to a T cell. In specific embodiments, the bispecific binding molecule is bound to a T cell. In specific embodiments, the binding of the bispecific binding molecule to the T cell is noncovalently. In a specific embodiment, the administering of a bispecific binding molecule provided herein, polynucleotide, vector, or cell encoding the bispecific binding molecule, or a pharmaceutical composition comprising the bispecific binding molecule is performed after treating the patient with T cell infusion. In specific embodiments the T cell infusion is performed with T cells that are autologous to the subject to whom the T cells are administered. In specific embodiments, the T cell infusion is performed with T cells that are allogeneic to the subject to whom the T cells are administered. In specific embodiments, the T cells can be bound to molecules identical to a bispecific binding molecule as described herein. In specific embodiments, the binding of the T cells to molecules identical to the bispecific binding molecule is noncovalently. In specific embodiments, the T cells are human T cells. Methods that can be used to bind bispecific binding molecules to T cells are known in the art. See, e.g., Lum et al., 2013, Biol Blood Marrow Transplant, 19:925-33, Janeway et al., Immunobiology: The Immune System in Health and Disease, 5^{th} edition, New York: Garland Science; Vaishampayan et al., 2015, Prostate Cancer, 2015:285193, and Stromnes et al., 2014, Immunol Rev. 257 (1): 145-164. See, also, Vaishampayan et al., 2015, Prostate Cancer, 2015:285193, which describes the following exemplary, non-limiting method for binding bispecific binding molecules to T cells:
Peripheral blood mononuclear cells (PBMCs) can be collected to obtain lymphocytes for activated T cell expansion from 1 or 2 leukopheresis. PBMCs can be activated with, for example, 20 ng/ml of OKT3 and expanded in 100 IU/mL of IL-2 to generate 40-320 billion activated T cells during a maximum of 14 days of culture under cGMP conditions as described in Ueda et al., 1993, Transplantation, 56 (2): 351-356 and Uberti et al., 1994, Clinical Immunology and Immunopathology, 70 (3): 234-240. Cells are grown in breathable flasks (FEP Bag Type 750-Cl, American Fluoroseal Corporation, Gaithersburg, MD) in RPMI 1640 medium (Lonza) supplemented with 2% pooled heat inactivated human serum. Activated T cells are split approximately every 2-3 days based on cell counts. After 14 days, activated T cells are cultured with 50 ng of a bispecific binding molecule described herein per 106 activated T cells. The mixture is then washed and cryopreserved.

### 6. EXAMPLES

### 6.1 Example 1

### 6.1.1 Introduction

This example describes a HER2/CD3 bi-specific binding molecule (herein referred to as “HER2-BsAb”) based on an IgG1 platform. This platform was utilized to allow for: (1) an optimal size to maximize tumor uptake, (2) bivalency towards the tumor target to maintain avidity, (3) a scaffold that is naturally assembled like any IgG (heavy chain and light chain) in CHO cells, purifiable by standard protein A affinity chromatography, (4) structural arrangement to render the anti-CD3 component functionally monovalent, hence reducing spontaneous activation of T cells, and (5) a platform with proven tumor targeting efficiency in animal models. This bispecific binding molecule has the same specificity as trastuzumab; but also recruits and activates CD3 (+) T cells redirecting them against HER2 expressing tumor cells, generating robust anti-tumor responses. Without being bound by any theory, the effectiveness of this BsAb centers on the exploitation of the cytotoxic potential of polyclonal T cells, and its unique capacity to target tumor cells that express even low levels of HER2, independent of the activation status of the HER2 pathway.

### 6.1.2 Materials and Methods

### 6.1.2.1 HER2-BsAb Design, Production, and Purification Analyses

The HER2-BsAb format was designed as a huOKT3 scFv fusion to the C-terminus of the light chain of a human IgG1. The V_{H} was identical to that of Trastuzumab IgG1, except N297A mutation in a standard human IgG1 Fc region for aglycosylated form (SEQ ID NO: 62), while the light chain is constructed as V_{L}-CK-(G4S)₃-scFv (SEQ ID NO: 60). Nucleotide sequences encoding V_{H} and V_{L} domains from Trastuzumab, and the huOKT3 scFv were synthesized by GenScript with appropriate flanking restriction enzyme sites, and were subcloned into a standard mammalian expression vector. HER2-C825 control BsAb (C825 is a murine scFv antibody with high affinity for 1,4,7,10-tetraazacyclododecane-1,4,7,10-tetraacetic acid (DOTA)-metal complexes with lanthanides including lutetium and yttrium) was constructed in a similar way.

Linearized plasmid DNA was used to transfect CHO-S cells (Invitrogen) for stable production of BsAb. 2×10⁶ cells were transfected with 5 μg of plasmid DNA by Nucleofection (Lonza) and then recovered in CD OptiCHO medium supplemented with 8 mM L-glutamine (Invitrogen) for 2 d at 37° C. in 6-well culture plates. Stable pools were selected with 500 μg/mL hygromycin for approximately two weeks and single clones were then selected out with limited dilution. HER2-BsAb titer was determined by HER2 (+) AU565 cell and CD3 (+) Jurket cell ELISA, respectively, and stable clones with highest expression were selected.

The BsAb producer line was cultured in OptiCHO medium and the mature supernatant harvested. A protein A affinity column (GE Healthcare) was pre-equilibrated with 25 mM sodium citrate buffer with 0.15 M NaCl, pH 8.2. Bound BsAb was eluted with 0.1 M citric acid/sodium citrate buffer, pH 3.9 and neutralized with 25 mM sodium citrate, pH 8.5 (1:10 v/v ratio). For storage, BsAb was dialyzed into 25 mM sodium citrate, 0.15 M NaCl, pH 8.2 and frozen in aliquots at −80° C. Two micrograms of the protein was analyzed by SDS-PAGE under reducing conditions using 4-15% Tris-Glycine Ready Gel System (Bio-Rad). Invitrogen SeeBlue Plus2 Pre-Stained Standard was used as the protein MW marker. After electrophoresis, the gel was stained using Coomassie G-250 (GelCode Blue Stain Reagent; Pierce). The purity of HER2-BsAb was also evaluated by size-exclusion high-performance liquid chromatography (SE-HPLC). Approximately 20 μg of protein was injected into a TSK-GEL G3000SWXL 7.8 mm×30 cm, 5 μm column (TOSOH Bioscience) with 0.4 M NaClO₄, 0.05 M NaH₂PO₄, pH 6.0 buffer at flow rate of 0.5 mL/min, and UV detection at 280 nm. Ten microliters of gel-filtration standard (Bio-Rad) was analyzed in parallel for MW markers.

### 6.1.2.2 FACS Analyses

Cells were incubated with 5 μg/mL of primary antibody (trastuzumab, HER2-BsAb, or cetuximab) for thirty minutes at 4° C. in PBS, and a secondary phycoerythrin-labeled antibody specific for human Fc was used after wash of excess primary antibody. Cells were fixed with 1% paraformaldehyde (PFA) prior to analysis on FACSCalibur cytometer (BD biosciences). Controls were cells with secondary antibody only, for which the mean fluorescent intensity (MFI) was set to 5. FACS data display the MFI in the upper right panel of each plot.

### 6.1.2.3 51Cr Release Assay

The ⁵¹Cr release assay was performed with effector T cells cultured in vitro in the presence of anti-CD3 and anti-CD28 for about 14 days. All target tumor cells were harvested with 2 mM EDTA in PBS, labeled with ⁵¹Cr (Amersham, Arlington Height, IL) at 100 μCi/106 cells at 37° C. for 1 h. 5000 target cells/well were mixed with 50,000 effector cells (E:T=10:1) and BsAb antibodies in 96-well polystyrene round-bottom plates (BD Biosciences) to a final volume of 250 μl/well. The plates were incubated at 37° C. for 4 h. The released ⁵¹Cr in supernatant was counted in a y-counter (Packed Instrument, Downers Grove, IL). Percentage of specific release was calculated using the formula: (experimental cpm-background cpm)/(total cpm-background cpm)×100%, where cpm represented counts per minute of ⁵¹Cr released. Total release was assessed by lysis with 10% SDS (Sigma, St Louis, Mo), and background release was measured in the absence of effector cells. EC50 was calculated using SigmaPlot software.

### 6.1.2.4 Competition Assay

To assess the ability of trastuzumab and/or huOKT3 to interfere with HER2-BsAb binding, the HER2-positive SKOV3 cell line was incubated for thirty minutes a 4° C. with PBS or with 10 μg/mL of trastuzumab or huOKT3. Cells were subsequently stained with 10 μg/mL of Alexa-Fluor 488-conjugated HER2-BsAb and analyzed by flow cytometry. Alexa-Fluor 488-conjugated HER2-BsAb was generated with the Zenon® Alexa Fluor® 488 Human IgG Labeling Kit (Life Technologies) according to the manufacturer's instructions.

### 6.1.2.5 Binding Assay

Binding assays were performed by Surface Plasmon Resonance using Biacore T100 similar as described in Okazaki et al., 2004, J Mol Biol; 336 (5): 1239-1249.

### 6.1.2.6 Avidity Assay

To compare the avidity of HER2-BsAb and trastuzumab, HER2-positive SKOV3 cells were incubated with 10 fold dilutions (from 10 to 1×10⁻⁵ μg/mL) of trastuzumab or HER2-BsAb and analyzed by flow cytometry with FITC-labeled human Fc-specific antibody as the secondary antibody. MFI was plotted against the antibody concentration and the curves were compared.

### 6.1.2.7 Proliferation Assay

To determine anti-proliferative effects, cells were treated with isotype control monoclonal antibody, 10 nM lapatinib (as a positive control), 10 μg/mL HER2-BsAb, 10 μg/mL Trastuzumab, 10 nM lapatinib, 10 nM erlotinib, 10 nM neratinib, or 10 μg/mL cetuximab for 72 hours and cell proliferation assayed. Cell proliferation was determined using an ELISA plate reader and the WST-8 kit (Dojindo technologies) following the manufacturer's instructions and using the formula: % survival rate=(Sample-Background)/(Negative control-Background). Lapatinib (MSKCC pharmacy) was ground using a mortar and pestle and suspended in DMSO as previously described. To determine statistical significance, the results were analyzed using one-way ANOVA using Prism 6.0.

6.1.2.8 qRT-PCR

RNA was extracted when cells were at 70% confluence and cDNA was analyzed in a prism 7700 sequence detection system using the HER2 specific, commercially available kit Hs01001580_ml from Applied Biosciences.

### 6.1.2.9 Animals and In Vivo Assays

For in vivo studies, BALB-Rag2-KO-IL-2R-yc-KO (DKO) mice (derived from colony of Dr. Mamoru Ito, CIEA, Kawasaki, Japan). See, for example, Koo et al., 2009, Expert Rev Vaccines, 8:113-120 and Andrade et al., 2011, Arthritis Rheum, 2011, 63:2764-2773. MCF7 cells or HCC1954 were mixed at a 1:1 ratio with PMBCs (unactivated, from buffy coat) and implanted in DKO mice subcutaneously. Four days post implantation, mice were treated with PBS, 10 μg of trastuzumab, or 10 μg of HER2-BsAb twice a week for two weeks. Tumor size was measured at the indicated days post implantation. Tumor size was determined by either calipers with the formula V=0.5 (length×width×width), or by using the Peira TM900 optical system.

For the metastatic model, MCF7 cells expressing luciferase were administered to DKO mice intravenously. Four days post administration, mice were treated with 100 μg of HER2-BsAb, 20 μg or HER2-BsAb, or 20 μg of a HER2-BsAb lacking CD3 targeting (HER2-C825) twice a week for three weeks, with or without intravenous administration of 5×10⁶ PBMC. Tumor size was quantified at the indicated timepoints using IVIS 200 (Xenogen) to quantify luciferin bioluminescence.

### 6.1.3 Results

### 6.1.3.1 HER2-BsAb Binds to Both Tumor Cells and T Cells.

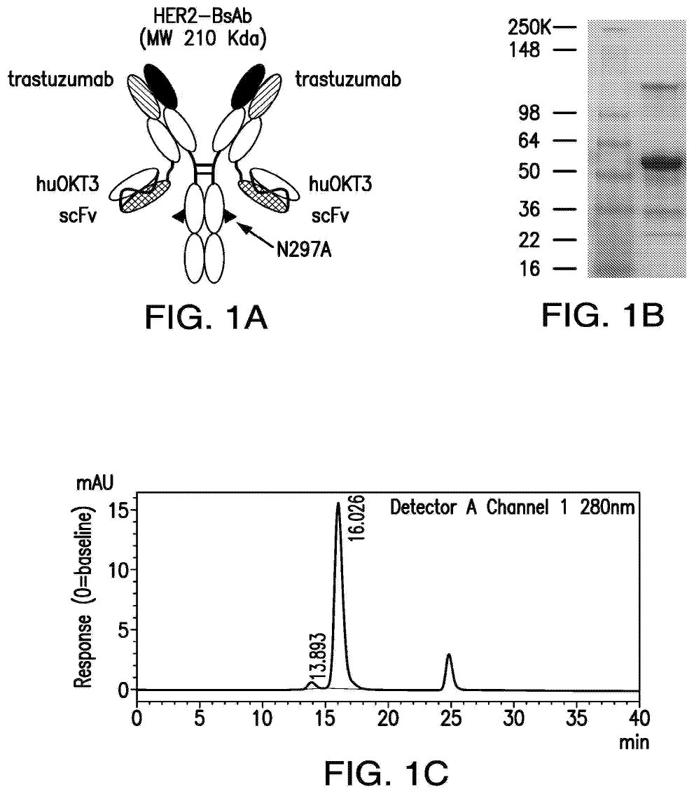

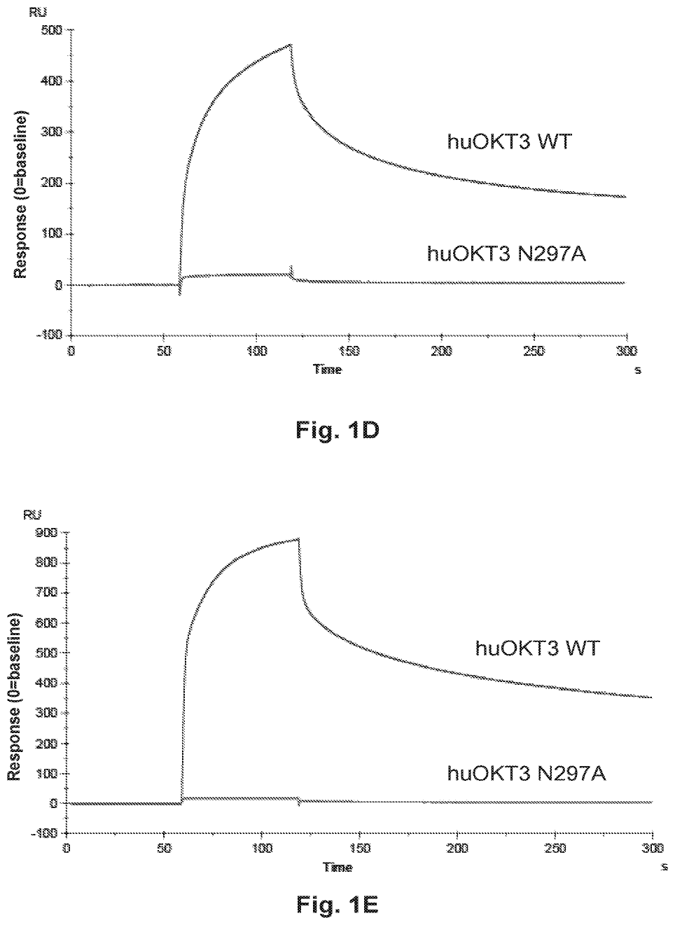

The HER2-BsAb was generated utilizing a trastuzumab variant comprising a N297A mutation in the human IgG1 Fc region to remove glycosylation (SEQ ID NO: 62). The BsAb light chain fusion polypeptide was generated by attaching the anti-CD3 humanized OKT3 (huOKT3) single chain Fv fragment (ScFv) to the carboxyl end of the trastuzumab IgG1 light chain via a C-terminal (G₄S)₃ linker (FIG. 1A and SEQ ID NO: 60). To avoid aggregation, a cysteine at position 105 of the variable heavy chain of huOKT3 was substituted with serine. A N297A mutation was also introduced into the HER2-BsAb Fc region to eliminate binding of HER2-BsAb to Fc receptors. This mutation has previously been shown to eliminate the capacity of human IgG1-Fc binding to CD16A (FIG. 1D) and CD32A Fc receptors (FIG. 1E).

To produce the HER2-BsAb, a mammalian expression vector encoding both the heavy chain and the light chain fusion polypeptide was transfected into CHO-S cells, stable clones were selected, supernatants collected, and the HER2-BsAb was purified by protein A affinity chromatography. Biochemical purity analysis of the BsAb is depicted in FIG. 1B and FIG. 1C. Under reducing SDS-PAGE conditions, HER2-BsAb gave rise to two bands at around 50 KDa, since the huOKT3 scFv fusion to trastuzumab light chain increased the MW to ˜50 KDa. SEC-HPLC showed a major peak (97% by UV analysis) with an approximate MW of 210 KDa, as well as a minor peak of multimers removable by gel filtration. The HER2-BsAb was stable by SDS-PAGE and SEC-HPLC after multiple freeze and thaw cycles.

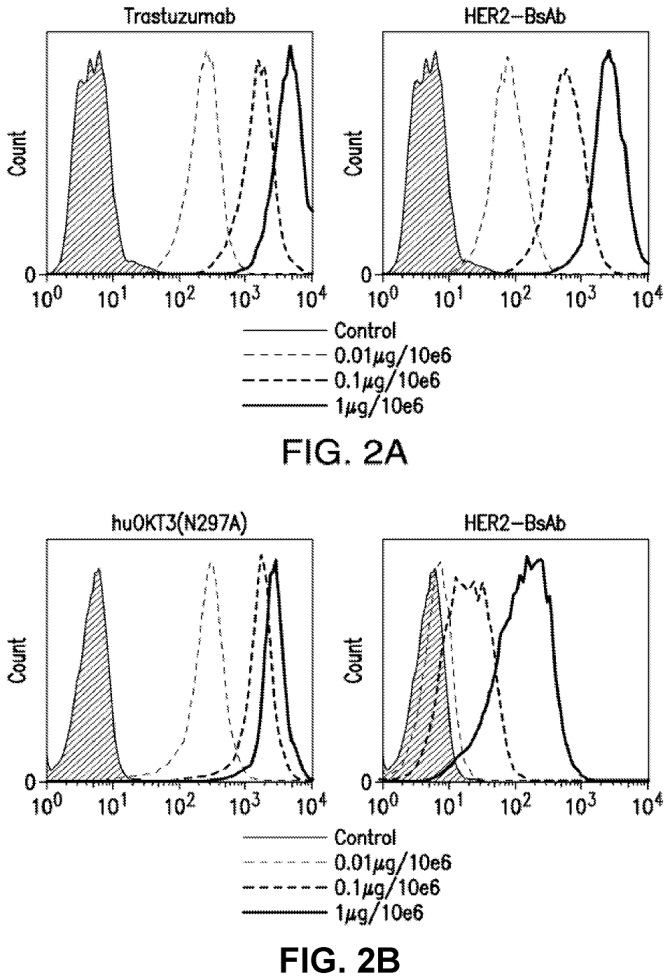

FACS and immunostaining were performed to assess the binding of HER2-BsAb to both target cells and effector cells. Trastuzumab and HER2-BsAb displayed comparable binding to the HER2-positive breast carcinoma cell line, AU565 (FIG. 2A). In contrast, HER2-BsAb demonstrated more than 20-fold less binding to CD3+ T cells than huOKT3 (FIG. 2B). This is consistent with the observation that light chain-anchored scFv had lower avidity for T cells than regular huOKT3 IgG1, purposely designed to minimize cytokine release in the absence of target tumor cells.

The lower avidity of HER2-BsAb for T cells was further confirmed by the binding affinity analysis by Biacore as described in Cheung et al. 2012, OncoImmunology, 1:477-486. For antigen CD3, HER2-BsAb had a kₒₙ at 4.53×10⁵ M⁻¹s⁻¹. a k_{off} at 8.68×10⁻² s⁻¹, and overall K_{D}) at 192 nM; comparable to parental huOKT3 IgG1-aGlyco at k_{off} (1.09×10⁻¹ s⁻¹), but less at kₒₙ (1.68×10⁶ M⁻¹s⁻¹) and overall K_{D} (64.6 nM). In summary, HER2-BsAb had much lower kₒₙ than its parental huOKT3-aGlyco, suggesting less chance of BsAb binding to and activating T cells under the same condition, hence less cytokine release.

### 6.1.3.2 HER2-BsAb Redirected T Cell Killing of Human Tumor Cell Lines.

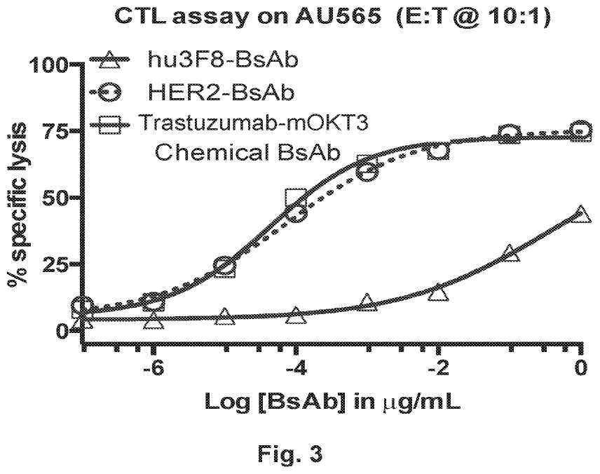

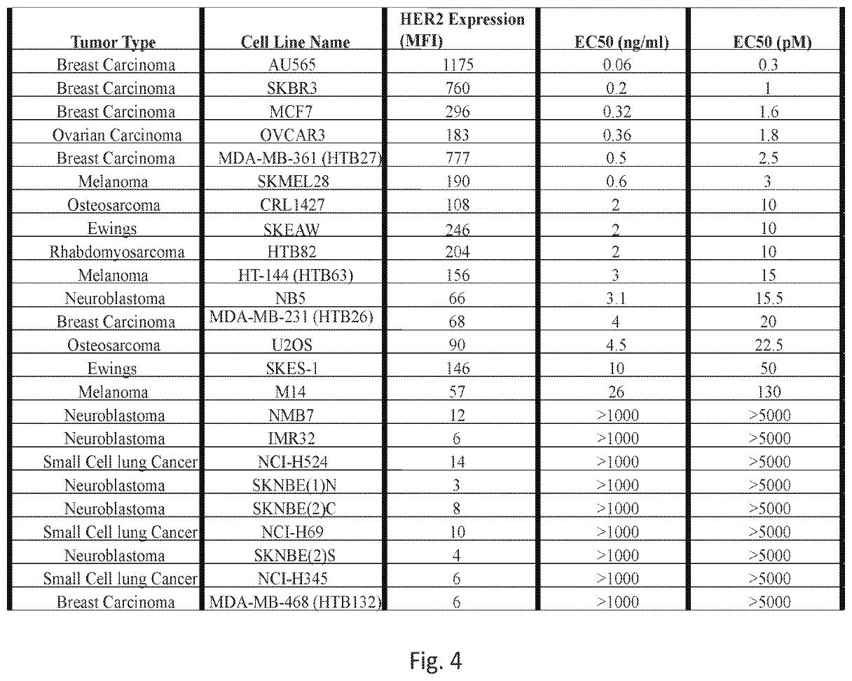

To evaluate whether HER2-BsAb could redirect T cells to kill tumor cells, T cell cytotoxicity on HER2 (+) breast cancer AU565 cells was tested in a standard 4-hour ⁵¹Cr release assay. Substantial killing of tumor cells was observed n the presence of HER2-BsAb, with an EC50 at 300 fM (FIG. 3). Moreover, the killing was effective for an extensive panel of human tumor cell lines including breast carcinoma, ovarian carcinoma, melanoma, osteosarcoma, Ewing's sarcoma, rhabdomyosarcoma, and neuroblastoma, wherein the killing potency correlated with the HER2 expression level in the cells by FACS (FIG. 4).

### 6.1.3.3 HER2-BsAb Mediates Tumor Antigen Specific T Cell Cytotoxicity.

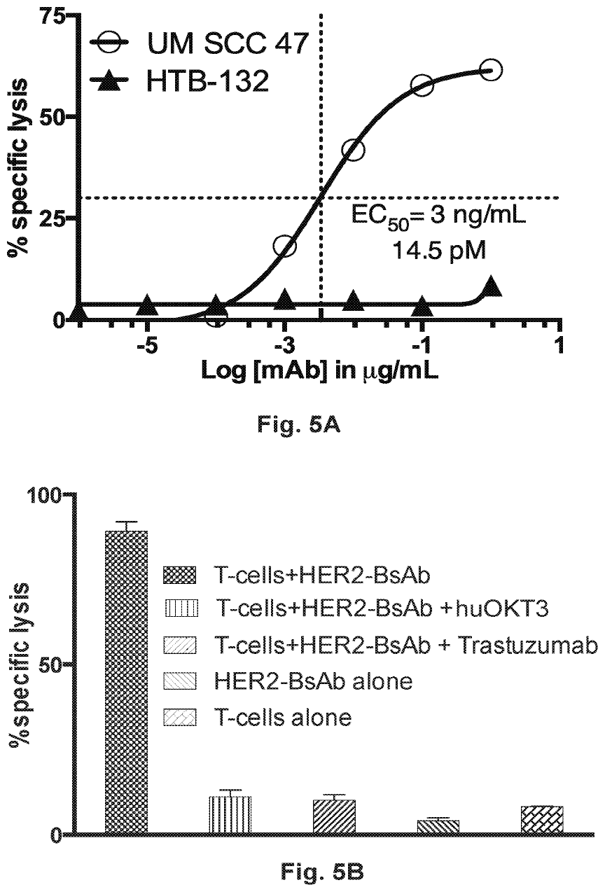

To investigate the tumor antigen specificity of HER2-BsAb in T cell cytotoxicity, a cytotoxicity assay was performed in the HER2-positive UM SCC 47 cells (a model for head and neck cancer) and in the HER2-negative HTB-132 cells (a model for breast cancer). HER2-BsAb mediated T cell cytotoxicity against the HER2-positive UM-SCC47 cells (EC50 of 14.5 pM), but not against the HER2-negative HTB-132 cells (FIG. 5A).

To investigate the specificity of HER2-BsAb in the T cell cytotoxicity, HER2-positive cells were first blocked with huOKT3 or with trastuzumab. In the absence of HER2-BsAb, the T cells displayed minimal cytotoxicity, reassuring that T cells on their own have minimum non-specific cytotoxicity. Both huOKT3 and trastuzumab blocked the ability of HER2-BsAb to induce T cell cytotoxicity.

### 6.1.3.4 HER2-BsAb Mediates T Cell Cytotoxicity Against HER2-Positive Cells Below the HER2 Threshold of Detection by Flow Cytometry.

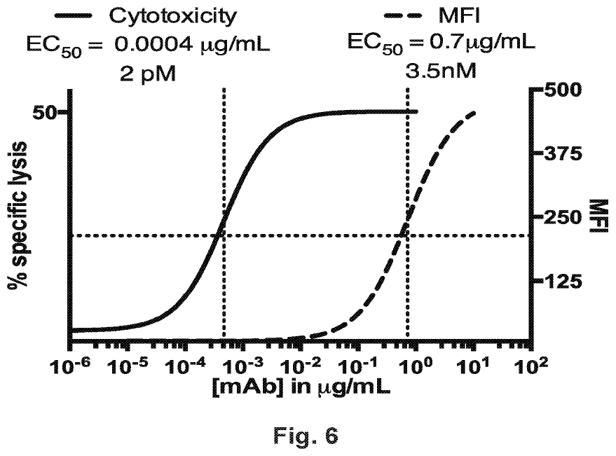

The HER2+ ovarian carcinoma cell line SKOV3 was used in a ⁵¹Cr cytotoxicity assay with 10 fold dilutions of HER2-BsAb in the presence of T cells. These same cells were stained using HER2-BsAb at the same concentrations and analyzed by flow cytometry, MFI was plotted over the same x-axis as cytotoxicity, and EC50 was calculated for both curves. HER2-BsAb mediated T cell cytotoxicity against HER2-positive cells even when HER2-BsAb binding was not detected by flow cytometry (FIG. 6). Comparing the EC50 for the cytotoxicity assay (2 pM) vs EC50 for flow cytometry curve (3.5 nM) suggests that T cells in the presence of HER2-BsAb were 2500× more effective in detecting HER2-positive cells than flow cytometry.

### 6.1.3.5 HER2-BsAb has the Same Specificity, Affinity and Antiproliferative Effects as Trastuzumab.

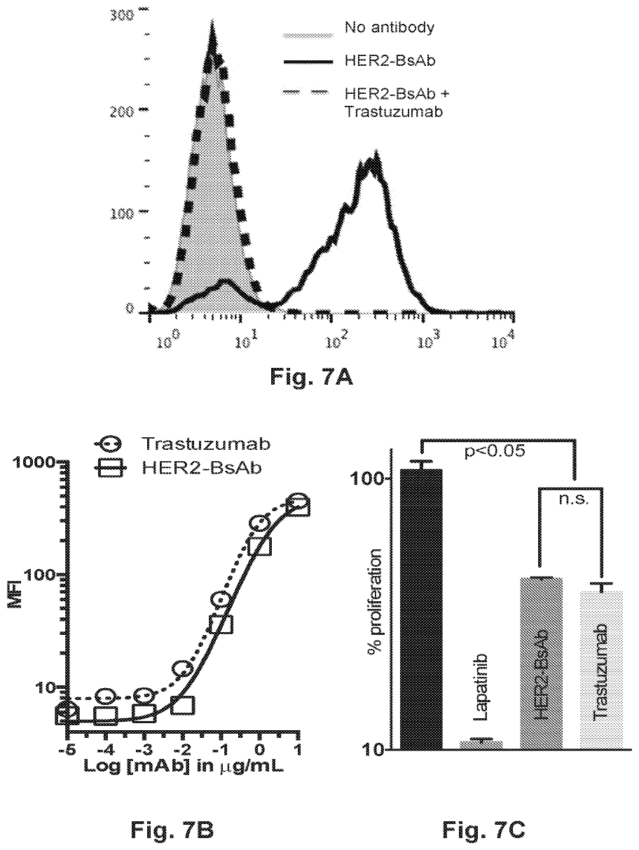

Prior to treatment with HER2-BsAb, HER2-positive cells were pre-incubated with trastuzumab to determine if HER2-BsAb shares the same antigen specificity as trastuzumab. Pre-incubation with trastuzumab blocked HER2-BsAb binding to the cells, demonstrating a shared specificity (FIG. 7A). To compare the affinity of HER2-BsAb to trastuzumab, HER2-positive cells were incubated with dilutions of trastuzumab or HER2-BsAb and analyzed by flow cytometry for cellular binding. Plotting of MFI against the antibody concentration revealed similar curves for trastuzumab and HER2-BsAb, demonstrating a similar binding affinity (FIG. 7B). Further, trastuzumab and HER2-BsAb demonstrated similar anti-proliferative effects against HER2-positive cells (FIG. 7C).

6.1.3.6 HER2-BsAb Mediated T Cell Cytotoxicity Against SCCHN with an EC50 in the Picomolar Range.

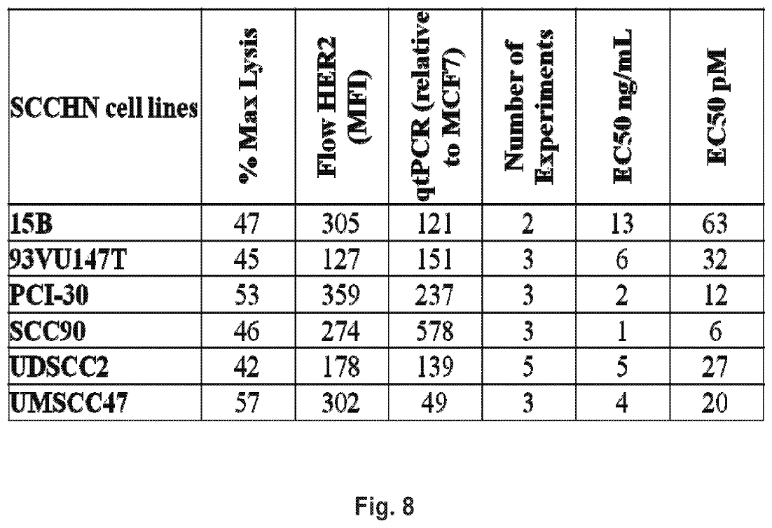

The level and frequency of HER2 in the previously characterized head and neck cancer cell lines 93-VU-147T, PCI-30, UD-SCC2, SCC90, UMSCC47 and PCI-15B were assessed via flow cytometry with trastuzumab. The cells were also tested for HER2 expression by qRT-PCR (FIG. 8). HER2 was comparably expressed in the panel of head and neck cancer cell lines. Finally, the level of cytotoxicity in the presence of T cells and HER2-BsAb was correlated with the level of HER2 in the cells, revealing HER2-BsAb displays an EC50 in the picomolar range for these head and neck cell lines (FIG. 8).

### 6.1.3.7 HER2-BsAb Mediates T Cell Cytotoxicity Against SCCHN Resistant to Other HER Targeted Therapies.

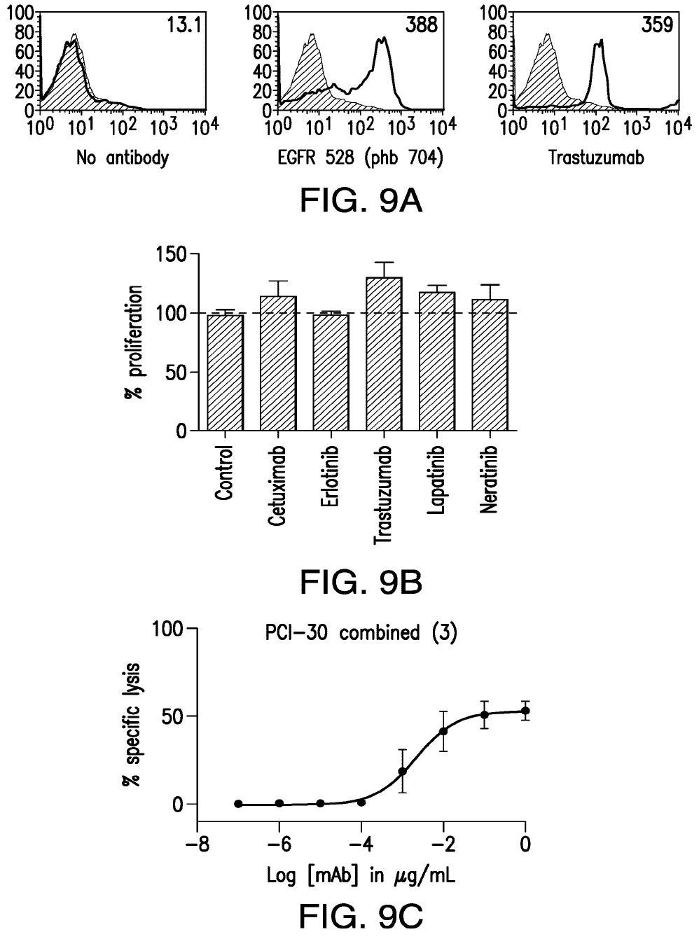

To determine the EGFR and HER2 status of the SCCHN cell line PCI-30, cells were stained with trastuzumab or cetuximab and analyzed by flow cytometry as previously described (FIG. 9A). A proliferation assay demonstrated that these cells are resistant to the HER-specific targeted therapies, trastuzumab, cetuximab, lapatinib, erlotinib and pan-HER inhibitor neratinib (FIG. 9B). However, PCI-30 cells were sensitive to treatment with HER2-BsAb utilizing three different cytotoxicity assays (FIG. 9C). HER2-BsAb generated potent cytotoxic responses against PCI-30 independent of their sensitivity to other HER targeted therapies, even when these drugs target more than one of these receptors. These assays suggest that HER2-BsAb was able to generate powerful cytotoxic responses, regardless of target cell sensitivity to EGFR or HER2 targeted therapies.

6.1.3.8 HER2-BsAb Mediated T Cell Cytotoxicity Against Osteosarcoma Cell Lines with an EC50 in the Picomolar Range.

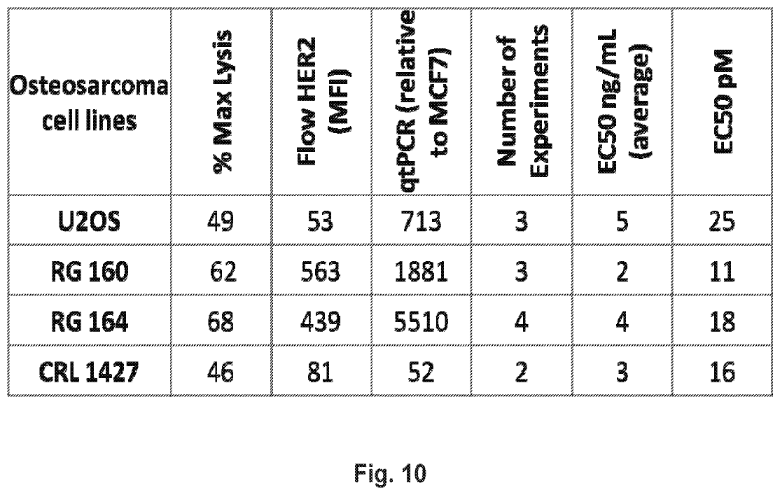

The previously characterized osteosarcoma cell lines, RG-160, CRL 1427 and U2OS, were assessed for their HER2 expression by flow cytometry with trastuzumab (FIG. 10) and by qRT-PCR, and the levels of HER2 were correlated with cytotoxicity in the presence of T cells and HER2-BsAb (FIG. 10). All tested cell lines were positive for HER2, although the expression level varied. Further, all HER2-positive cells were sensitive to T cell cytotoxicity mediated by HER2 BsAb, with an EC50 ranging from 11-25 pM.

### 6.1.3.9 HER2-BsAb Mediates T Cell Cytotoxicity Against HER-Therapy Resistant Osteosarcoma Cell Lines.

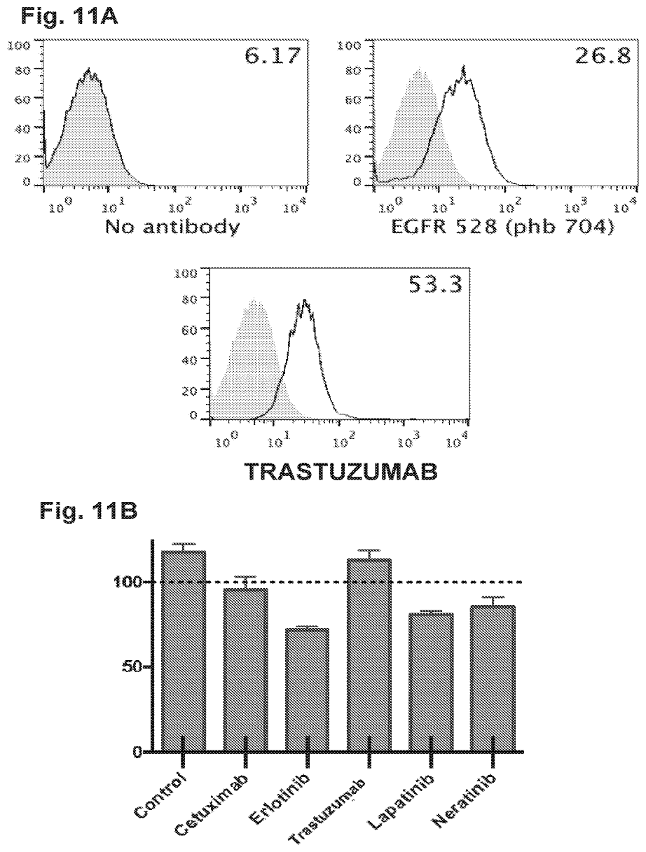

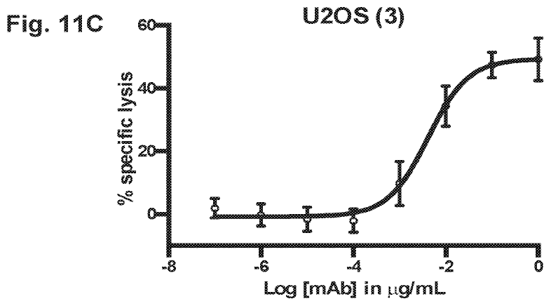

U2OS cells are a HER2-positive, EGFR-positive osteosarcoma cell line (FIG. 11A). U2OS cells were analyzed for their sensitivity to trastuzumab, cetuximab, lapatinib and the pan-HER inhibitor Neratinib by proliferation assay in the presence of each of the inhibitors. These cells were resistant to cetuximab and trastuzumab with minimal sensitivity to Lapatinib, erlotinib and neratinib (FIG. 11B). These same cells were tested for sensitivity for T cell cytotoxic responses mediated by HER2-BsAb. HER2-BsAb generated potent cytotoxic responses against U2OS cells using three different cytotoxicity assays, independent of its sensitivity to other HER targeted therapies (FIG. 11C).

### 6.1.3.10 HER2-BsAb Mediates T Cell Cytotoxicity Against HER-Therapy Resistant Cervical Cancer HeLa Cells.

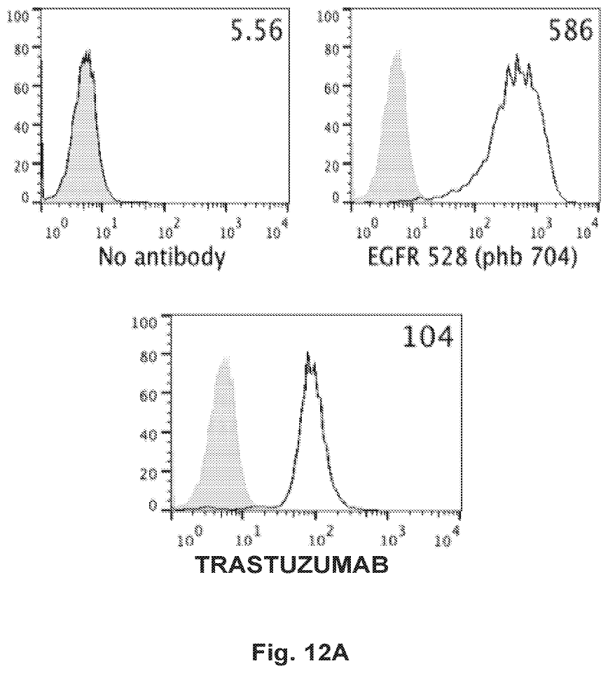

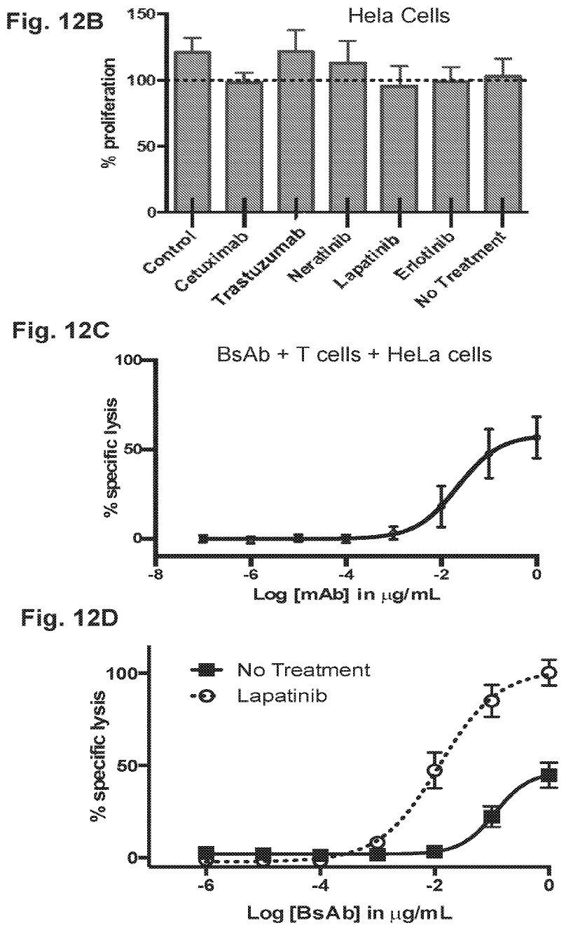

HeLa cells are a HER2-positive, EGFR-positive cervical carcinoma cell line (FIG. 12A). HeLa cells were analyzed for their sensitivity to HER family tyrosine kinase inhibitors, Erlotinib, Lapatinib or Neratinib, or to the HER specific antibodies, Cetuximab or trastuzumab. These results demonstrated that HeLa cells are pan-resistant to these therapies (FIG. 12B). However, these same cells were tested for sensitivity for T cell cytotoxic responses mediated by HER2-BsAb. HER2-BsAb generated potent cytotoxic responses against HeLa cells using three different cytotoxicity assays, independent of its sensitivity to other HER targeted therapies (FIG. 12C). Interestingly, pretreatment with lapatinib increased sensitivity to HER2-BsAb mediated cytotoxicity, even when lapatinib alone had no effect on cell proliferation.

### 6.1.3.11 HER2-BsAb is Effective Against Human Breast Cancer in Humanized Mice.

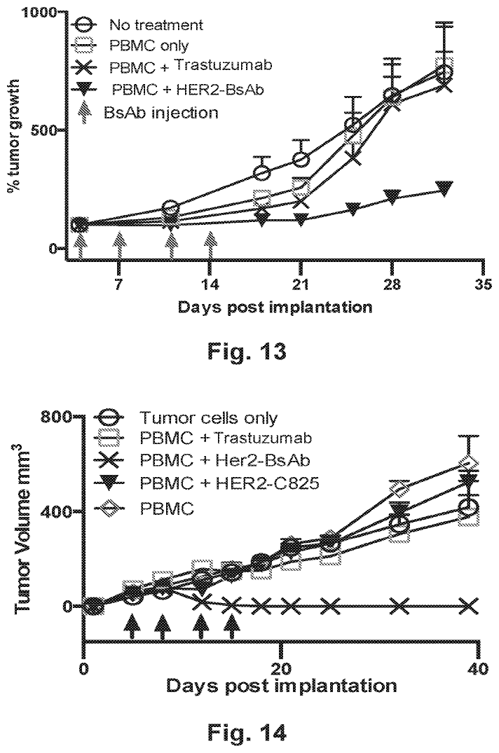

For in vivo therapy studies, BALB-Rag2-KO-IL-2R-yc-KO (DKO) mice (derived from colony of Dr. Mamoru Ito, CIEA, Kawasaki, Japan) were used. See, for example, Koo et al. 2009, Expert Rev Vaccines 8:113-120 and Andrade et al. 2011, Arthritis Rhem 63:2764-2773. MCF7-Luciferase breast cancer cells were mixed with peripheral blood mononuclear cells (PBMC) and planted subcutaneously. Four days post cell implantation, the mice were treated with HER2-BsAb or with trastuzumab and the tumor size was analyzed over time (FIG. 13). HER2-BsAb demonstrated a significant suppression of tumor progression. HER2-BsAb was also effective against tumor progression when the trastuzumab resistant HCC1954 breast cancer cells (See, for example, Huang et al., 2011, Breast Cancer Research, 13: R84) were planted subcutaneously with PBMCs (FIG. 14).

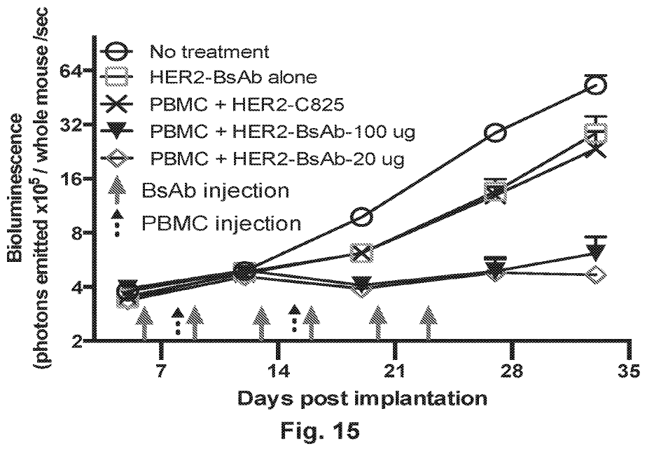

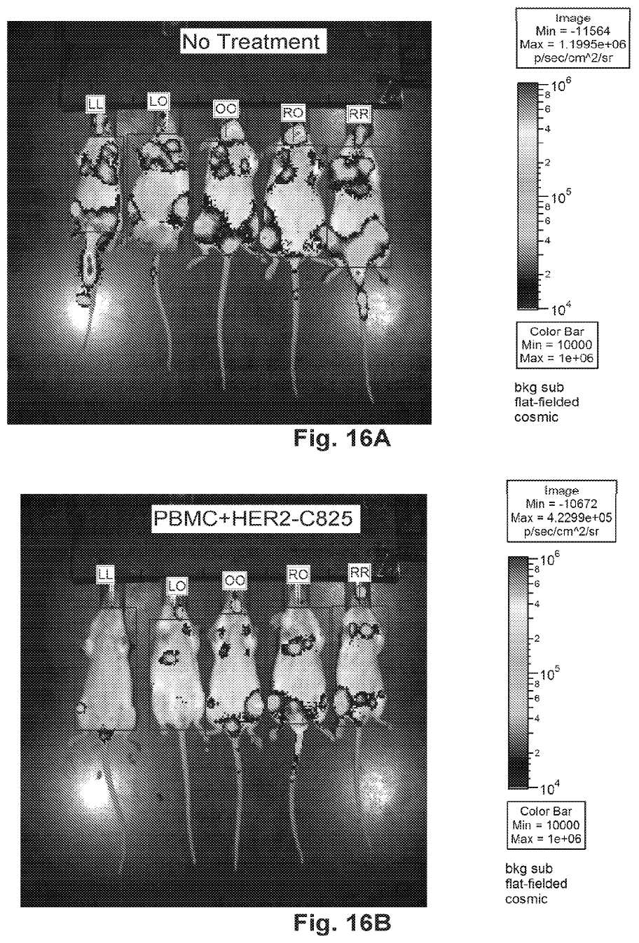

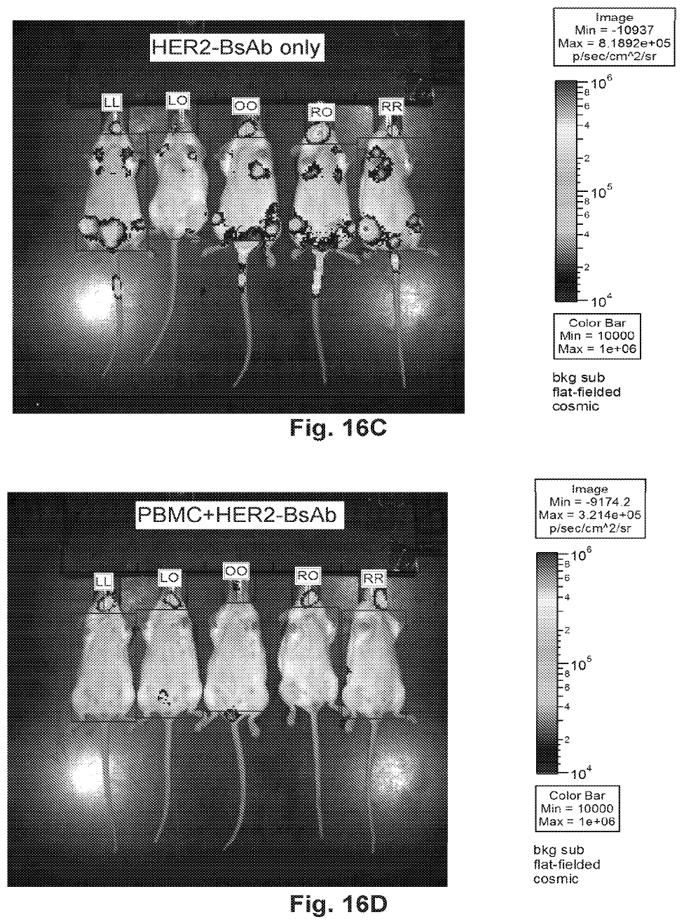

To assess a metastatic tumor model, MCF7-Luciferace cells were inoculated intravenously. HER2-BsAb was administered and subsequently in combination with PBMC. Tumor luciferin bioluminescence signal demonstrated HER2-BsAb plus PBMC showed complete suppression of tumor progression (FIG. 15, FIG. 16A, FIG. 16B, FIG. 16C, and FIG. 16D).

### 6.1.4 Conclusions

The aglycosylated HER2-BsAb allowed for minimized Fc functions and avoidance of a cytokine storm and elimination of all complement activation, complement mediated and complement receptor mediated immune adherence. In addition, despite bivalency of huOKT3 in the IgG-scFv platform, binding to CD3 was functionally monovalent; hence there was no spontaneous activation of T cells in the absence of tumor target. HER2-BsAb displayed potent cytotoxicity against HER2-positive tumor cells in vitro, even against cells with low antigen expression, or cells that are resistant to trastuzumab, cetuximab, lapatinib, erlotinib or the pan-HER inhibitor neratinib. HER2-BsAb also displayed potent cytotoxicity against breast cancer, ovarian cancer, SCCHN, osteosarcomas, and sarcomas. Finally, HER2-BsAb displayed strong in vivo efficacy against tumor xenografts, substantially better than the trastuzumab hIgG1 counterpart.

### 6.2 Example 2

This example provides (a) a more detailed description of certain of the experiments described in Example 1 (Section 6.1); and (b) additional experiments as compared to Example 1 (Section 6.1).

### 6.2.1 Introduction

Trastuzumab has significantly improved patient outcomes in breast cancer and has also been key in the design and implementation of other targeted therapies (Singh et al., 2014, Br J Cancer 111:1888-98). However, HER2 expression does not guarantee a clinical response to trastuzumab or other HER2 targeted therapies (Gajria et al., 2011, Expert Review of Anticancer Therapy, 11 (2): 263-75; Lipton et al., 2013, Breast Cancer Research and Treatment, 141 (1): 43-53). Less than 35% of patients with HER2 positive breast cancer initially respond to trastuzumab and 70% of the initial responders will ultimately progress with metastatic disease within a year (Vu and Claret., 2011, Frontiers in Oncology 2:62). In osteosarcoma and Ewing's sarcoma, where high levels of HER2 expression are associated with decreased survival (Gorlick et al., 1999, Journal of Clinical Oncology: Official Journal of The American Society of Clinical Oncology 17:2781-2788), trastuzumab has not shown any benefit even when used in conjunction with cytotoxic chemotherapy (Ebb et al., 2012, Journal of Clinical Oncology: Official Journal of the American Society of Clinical Oncology 30:2545-2551). Furthermore, trastuzumab, like other HER targeted therapies, has shown modest or no benefit against HER2-positive head and neck cancer (Pollock et al., 2014, Clinical Cancer Research, 21 (3): 526-33).

The reasons for these failures are complex and only partially understood. The genomic diversity and constant evolution of malignancies make them less prone to oncogene addiction, a requirement for the success of targeted therapy. Furthermore, even when oncogene addiction is present, resistance can emerge from selection pressure induced by the use of targeted therapies (Lipton et al., 2013, Breast Cancer Research and Treatment, 141 (1): 43-53). In fact, despite the initial enthusiasm received, the majority of targeted therapies have not produced a significant benefit in the overall cure of patients receiving it (Nathanson et al., 2014, Science, 343:72-76). A different approach, one that selectively targets malignant cells that overexpress HER family receptors, and that can generate cytotoxic anti-tumor responses independently of the receptor activation status can be beneficial.

Blinatumomab-a CD19/CD3 BsAb was approved in 2014 for treating Acute Lymphoplastic Leukemia (Sanford, 2015, Drugs 75:321-7). However, despite its promising results, the unfavorable PK of these small size molecules necessitates prolonged infusions, complicating their administration (Shalaby et al., 1995, Clin Immunol Immunopathol 74:185-92, 1995; Portell et al., 2013, Clin Pharmacol 5:5-11). Furthermore, the resulting cytokine release syndrome (CRS) still poses costly and often life-threatening complications. Importantly, despite the ability of bispecific antibodies to activate T cells, the same inhibitory pathways that regulate classic T cell function might still limit their effectiveness. For example, the heterodimeric design of a monovalent binding HER2/CD3 bispecific antibody was inhibited by the PD-1/PD-L1 inhibitory axis (Junttila et al., 2014, Cancer Res 74:5561-71).

The present example provides a bispecific binding molecule (herein referred to as “HER2-BsAb”) that offers two distinct advantages over the existing technologies: (1) it is based on the fully humanized HER2 specific IgG1 mAb Trastuzumab, preserving its pharmacologic advantages (Wittrup et al., 2012, Methods Enzymol 503:255-68) and bivalent binding to HER2; maximizing tumor avidity; and (2) its binding to CD3 is functionally monovalent through the scFv derived from the humanized huOKT3 mAb sequence. Thus, HER2-BsAb is built on two mAbs with extensive records of clinical safety. Furthermore, this is a platform with its Fc function deleted to eliminate all antibody-dependent cell-mediated cytotoxicity (ADCC) and CMC activities in order to reduce the cytokine release syndrome.

The data presented in this example demonstrate the ability of HER2-BsAb to produce potent anti-tumor responses, both in vitro and in vivo, against tumor cells that are resistant to HER2 targeted therapy or trastuzumab.

### 6.2.2 Materials and Methods

### 6.2.2.1 Cell Lines

All cell lines were purchased from ATCC (Manassas Va) except: UM-SCC47, obtained from Dr. Carey at the University of Michigan; SCC-90, PCI-30 and PCI-15B, obtained from Dr. Robert Ferris at the University of Pittsburgh; HCC1954, obtained from Dr. Sarat Chandarlapaty at Memorial Sloan Kettering Cancer Center; 93-VU-147T and HeLa, obtained from Dr. Luc Morris; and UD-SCC2, obtained from Henning Bier at Hals-Nasen-Ohrenklinik und Poliklinik. All cells were authenticated by short tandem repeat profiling using PowerPlex 1.2 System (Promega), and periodically tested for mycoplasma using a commercial kit (Lonza). The luciferase-labeled tumor cell lines MCF7-Luc were generated by retroviral infection with a SFG-GFLuc vector.

### 6.2.2.2 HER2-BsAb Design and Expression in CHO-S Cells

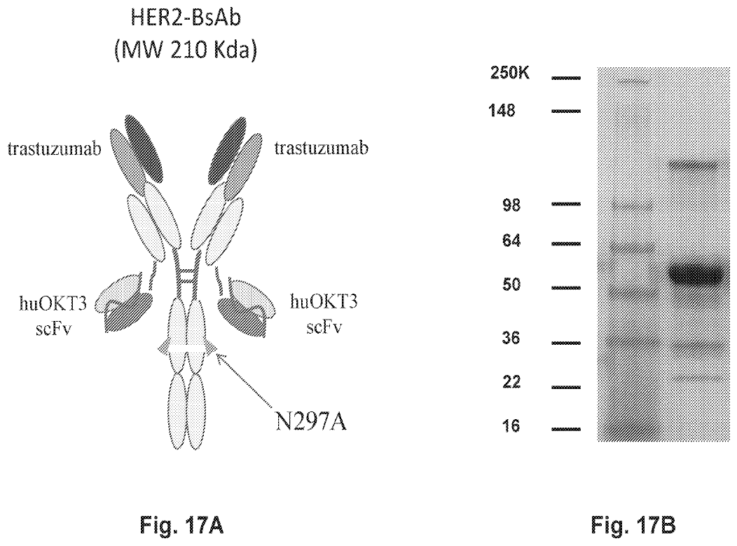

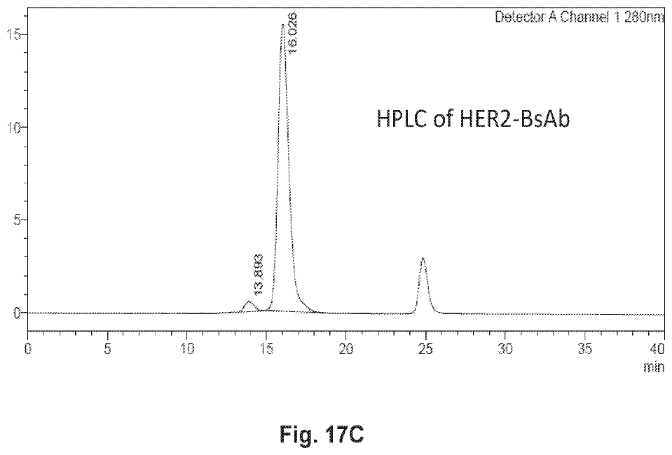

In the HER2-BsAb IgG-scFv format (FIG. 17A, “HER2-BsAb”), the V_{H} was identical to that of the trastuzumab IgG1 V_{H}, except that an N297A mutation in the Fc region was introduced into the HER2-BsAb to remove glycosylation, thereby depleting Fc function (SEQ ID NO: 62). The light chain fusion polypeptide was constructed by extending the trastuzumab IgG1 light chain with a C-terminal (G4S)₃ linker followed by huOKT3 scFv (SEQ ID NO: 60). The DNA encoding both the heavy chain and the light chain was inserted into a mammalian expression vector, transfected into CHO-S cells, and stable clones of the highest expression were selected. Supernatants were collected from shaker flasks and the HER2-BsAb was purified by protein A affinity chromatography. The control BsAb, HER2-C825 (composed of SEQ ID NOS: 71 and 72), was generated as previously described (Xu et al., 2015, Cancer Immunol Res 3:266-77; Cheal et al., 2014, Mol Cancer Ther 13:1803-12).

### 6.2.2.3 Other Antibodies and Small Molecules

Fluorophore-labeled HER2-BsAb was generated with the Zenon® Alexa Fluor® 488 Human IgG Labeling Kit from Life Technologies following the manufacturer's instructions. Pembrolizumab, cetuximab, trastuzumab, Erlotinib, Lapatinib and Neratinib were purchased from the Memorial Sloan Kettering Cancer Center pharmacy. Small molecules were re-suspended in DMSO. The CD4, CD8, CD16 and CD56 antibodies were purchased from BD Biosciences (San Jose CA). The commercially available PE labeled PD-L1 specific mAb 10F.9G2 was purchased from BioLegend.

### 6.2.2.4 Cell Proliferation Assays

For cell proliferation assays, 5,000 tumor cells were plated using RPMI-1640 supplemented with 10% FBS in a 96 well plate for 36 hours before being treated with lapatinib or the antibodies at the specified concentrations. Cell proliferation was determined using an ELISA plate reader and the WST-8 kit (Dojindo technologies) following the manufacturer's instructions and using the formula: % survival rate=(Sample-Background)/(Negative control-Background). Lapatinib (Memorial Sloan Kettering Cancer Center pharmacy) was ground using a mortar and pestle and suspended in DMSO as previously described (Chen et al., 2012, Molecular cancer therapeutics 11:660-669). To determine statistical significance, the results were analyzed using one-way ANOVA using Prism 6.0.

### 6.2.2.5 Cytotoxicity Assays (51Chromium Release Assay)

Cell cytotoxicity was assayed by ⁵¹Cr release as previously described (Xu et al., 2015, Cancer Immunol Res 3:266-77), and EC50 was calculated using SigmaPlot software. Effector T cells were purified from human PBMC using Pan T cell isolation kit (Miltenyi Biotec), and then activated and expanded with CD3/CD28 Dynabeads (Invitrogen) according to the manufacturer's protocol.

### 6.2.2.6 PD-1/PD-L1 Expression

To overexpress PD-L1 in HEK293 cells, cells were cultured in DMEM (Cellgro) supplemented with 10% heat-inactivated FBS and Penicillin (100 IU/ml) and streptomycin (100 μg/ml). On Day (−1), HEK293 cells were trypsinized, counted and plated into 6 well plates at 0.5 M cells/well and kept in 2 mL of fresh media. On the day of transfection, Day (0), the media was exchanged with 2 mL of fresh media. Transfection reagents were prepared as follows for both hPD-L1 and control plasmids: 2.5 ug of DNA was diluted into 250 μl of unsupplemented DMEM (no serum). 5 μl of Lipofectamine 2000 (Invitrogen) was diluted into a separate 250 μl of DMEM (no serum), and incubated for 5 minutes at room temperature. After 5 minutes, the diluted DNA was combined with the diluted Lipofectamine 2000 (Invitrogen) and incubated for another 30 minutes at room temperature. After 30 minutes, the entire 500 μl reaction was added, dropwise, onto a single well of HEK293 cells. The plate was rocked back and forth briefly to help mix the reagents. For the untransfected control, 500 μl of unsupplemented DMEM without DNA or Lipofectamine 2000 was added to one well. Cells were incubated at 37° C. for 24-48 hours before harvesting. On Day (l) or Day (2), cells were lifted from the plate using 2 mM EDTA in PBS, and counted. 100,000-200,000 cells were used for FACS analysis and the rest were used for the killing assays.

To induce PD-1 expression of activated T cells (ATCs), effector cells were incubated in a 3:1 ratio for 24 hours with the HER2-high Breast Carcinoma Cell line HCC1954 after these target cells were incubated with HER2-BsAb at a concentration of 10 μg/mL for 30 minutes and antibody excess was removed. Cells were harvested and used in cytotoxicity assays as previously described against the HEK293 cells transfected with PD-L1.

### 6.2.2.7 In Vivo Experiments

For in vivo therapy studies, BALB-Rag2−/−IL-2R-yc-KO (“DKO”) mice (derived from colony of Dr. Mamoru Ito, CIEA, Kawasaki, Japan; see, e.g., Koo et al., 2009, Expert Rev Vaccines 8:113-20 and Andrade et al., 2011, Arthritis Rheum 63:2764-73) were used. Three humanized mouse xenograft models were used: (1) intravenous tumor plus intravenous effector cells; (2) subcutaneous tumor plus subcutaneous effector cells; and (3) subcutaneous tumor plus intravenous effector cells. Subcutaneous xenografts were created with 5×10⁶ cells suspended in Matrigel (Corning Corp, Tewksbury MA) and implanted in the flank of DKO mice. Effector peripheral blood mononuclear cell (PBMC) cells were purified from buffy coats purchased from the New York Blood Center. Prior to every experimental procedure, PBMCs were analyzed for their percentage of CD3, CD4, CD8 and CD56 cells to ensure consistency. HER2-BsAb was injected intravenously twice a week at 100 μg/injection, beginning two days before effectors cells for three weeks, given as 5-10×10⁶ PBMC per injection, once a week for 2 weeks. Tumor size was measured using (1) hand-held TM900 scanner (Pieira, Brussels, BE); (2) Calipers; or (3) bioluminescence. Bioluminescence imaging was conducted using the Xenogen In Vivo Imaging System (IVIS) 200 (Caliper LifeSciences). Briefly, mice were injected intravenously with 0.1 mL solution of D-luciferin (Gold Biotechnology; 30 mg/mL stock in PBS). Images were collected 1 to 2 minutes after injection using the following parameters: a 10- to 60-second exposure time, medium binning, and an 8 f/stop. Bioluminescence image analysis was performed using Living Image 2.6 (Caliper LifeSciences).

### 6.2.3 Results

### 6.2.3.1 Her2-Bsab

HER2-BsAb was designed using an IgG-scFv format (FIG. 17A). The V_{H} was identical to that of trastuzumab IgG1, except for the N297A mutation in the Fc region of HER2-BsAb to remove glycosylation (SEQ ID NO: 62). The light chain fusion polypeptide was constructed by extending the trastuzumab IgG1 light chain with a C-terminal (G4S)₃ linker followed by huOKT3 scFv (Xu et al., 2015, Cancer Immunol Res 3:266-77) (SEQ ID NO: 60). The DNAs encoding both heavy chain and light chain were inserted into a mammalian expression vector, transfected into CHO-S cells, and stable clones of highest expression were selected. Supernatants were collected from shaker flasks and purified on protein A affinity chromatography.

SEC-HPLC and SDS-PAGE of the HER2-BsAb is shown in FIG. 17B and FIG. 17C, respectively. Under reducing SDS-PAGE conditions, HER2-BsAb gave rise to two bands at around 50 kDa, since the huOKT3 scFv fusion to trastuzumab light chain increased the molecular weight to approximately 50 kDa. SEC-HPLC showed a major peak (97% by UV analysis) with an approximate molecular weight of 200 KDa, as well as a minor peak of multimers removable by gel filtration. The BsAb remained stable by SDS-PAGE and SEC-HPLC after multiple freeze and thaw cycles.

### 6.2.3.2 HER2-BsAb Retained Specificity, Affinity and Anti-Proliferative Effects of Trastuzumab

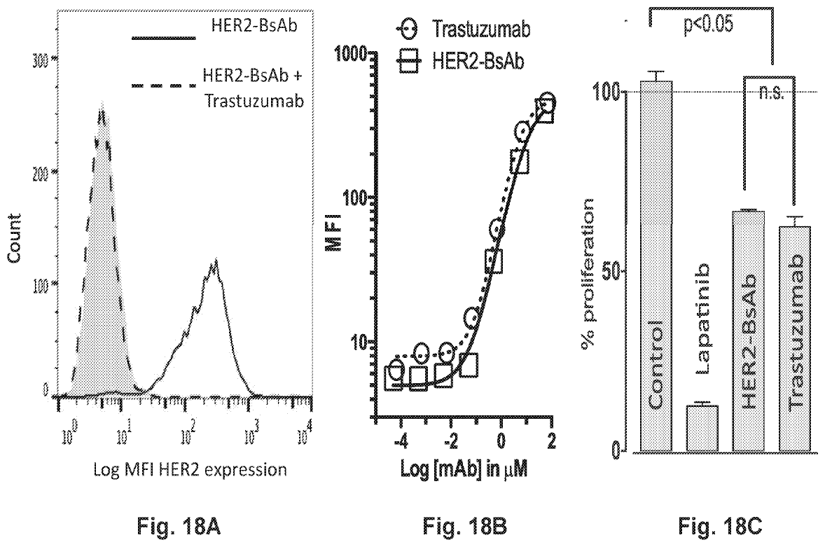

To determine if HER2-BsAb retained the specificity and anti-proliferative effects of trastuzumab, the HER2-positive-high SKOV3 ovarian carcinoma cell line was pre-incubated with 10 μg/mL of trastuzumab for 30 minutes and then immunostained using HER2-BsAb labeled with Alexa 488 (FIG. 18A). Incubation with trastuzumab prevented HER2-BsAb binding to SKOV3 cells, demonstrating that these antibodies shared the same specificity. To compare the avidity of HER2-BsAb to trastuzumab, the same cell line was incubated with 10-fold downward dilutions (from 10 μg/ml to 1×10⁻⁵ μg/mL) of trastuzumab or HER2-BsAb and analyzed by flow cytometry. The mean fluorescence intensity (MFI) was plotted against the antibody concentration in μM. The similarity in the binding curves confirmed that trastuzumab and HER2-BsAb had similar binding avidities for their common HER2 target (FIG. 18B).

Finally, the trastuzumab-sensitive breast carcinoma cell line SKBR3 was treated with isotype control mAb, 10 mM Lapatinib (as a positive control), 10 μg/mL HER2-BsAb, or 10 μg/mL trastuzumab for 72 hours and cell proliferation was assayed. As shown in FIG. 18C, trastuzumab and HER2-BsAb had similar anti-proliferative effects that were significant as compared to the negative control. As expected, lapatinib showed the strongest inhibition of cell proliferation.

### 6.2.3.3 HER2-BsAb Redirected T Cell Cytotoxicity was HER2-Specific and Dependent on CD3

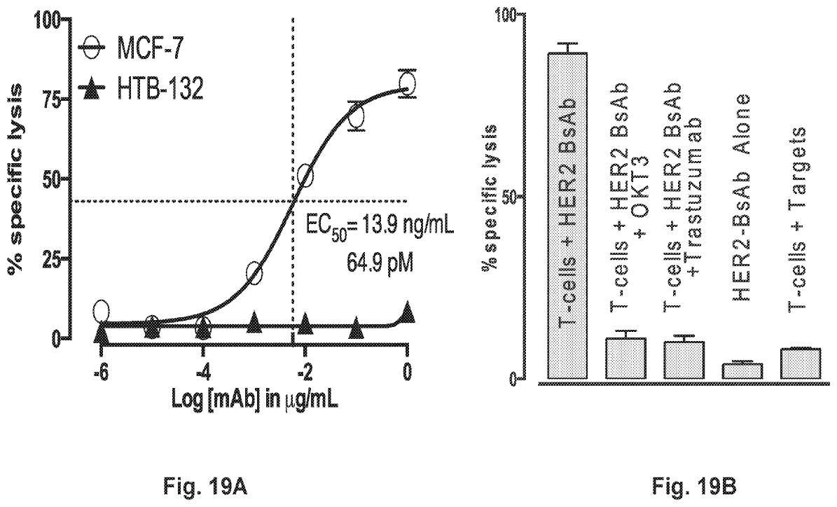

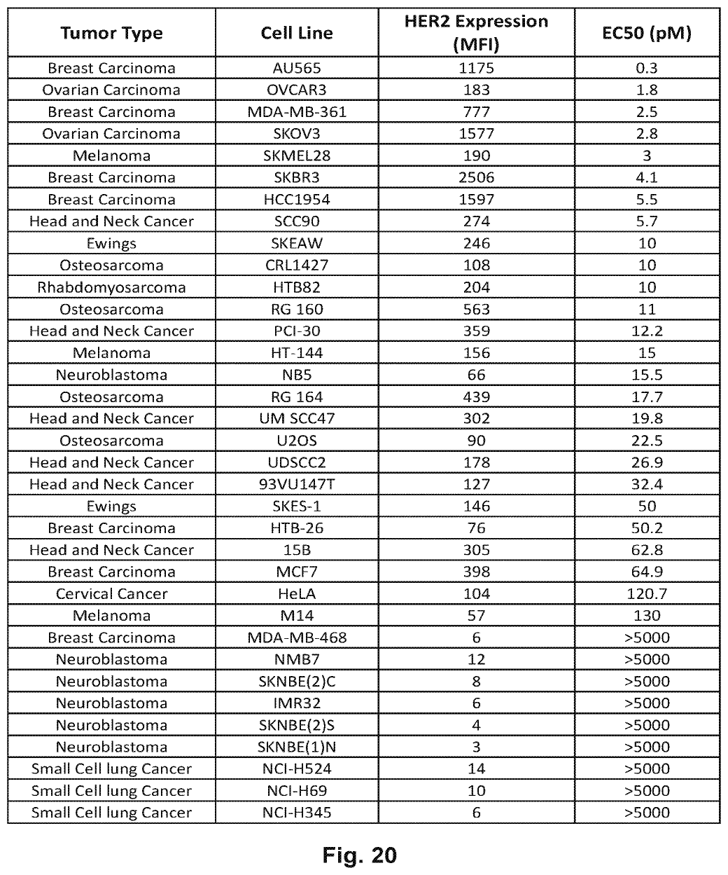

To establish the specificity of cytotoxic responses by T cells in the presence of HER2-BsAb; HER2-negative and HER2-positive cell lines were assayed in a cytotoxicity assays using ATCs (effector: T cell (“E: T”) ratio of 10:1) and HER2-BsAb at decreasing concentrations (FIG. 19A and FIG. 20). Cytotoxicity was absent for HER2-negative cell lines. To demonstrate the dependency of cytotoxicity on CD3, HER2-BsAb cytotoxicity was tested in the presence of the CD3 specific blocking mAb OKT3 (FIG. 19B). Pre-incubation with either trastuzumab or OKT3 prevented HER2-BsAb T cell mediated cytotoxicity.

6.2.3.4 HER2-BsAb Mediated Cytotoxicity Against HER2-Positive Cell Lines that were Resistant to Other HER2 Targeted Therapies.

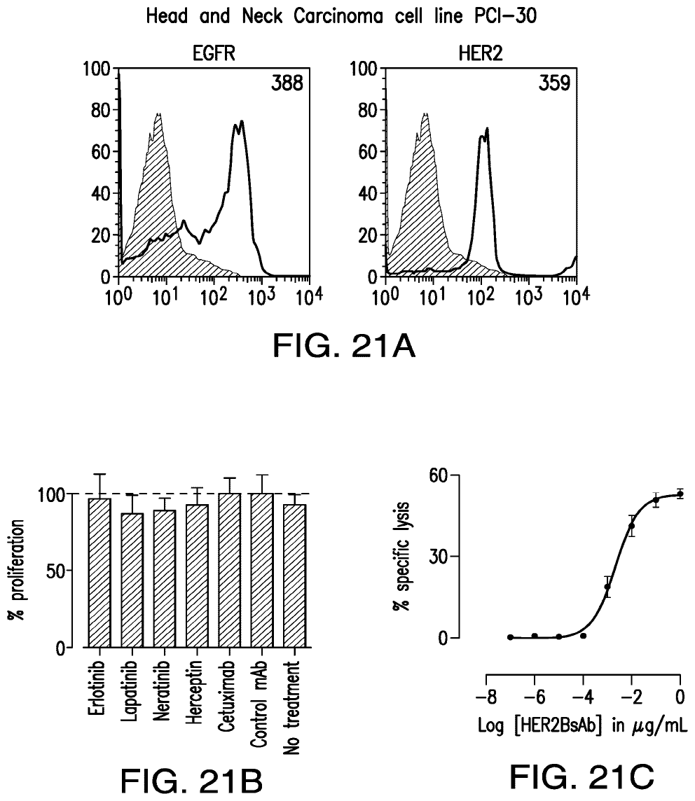

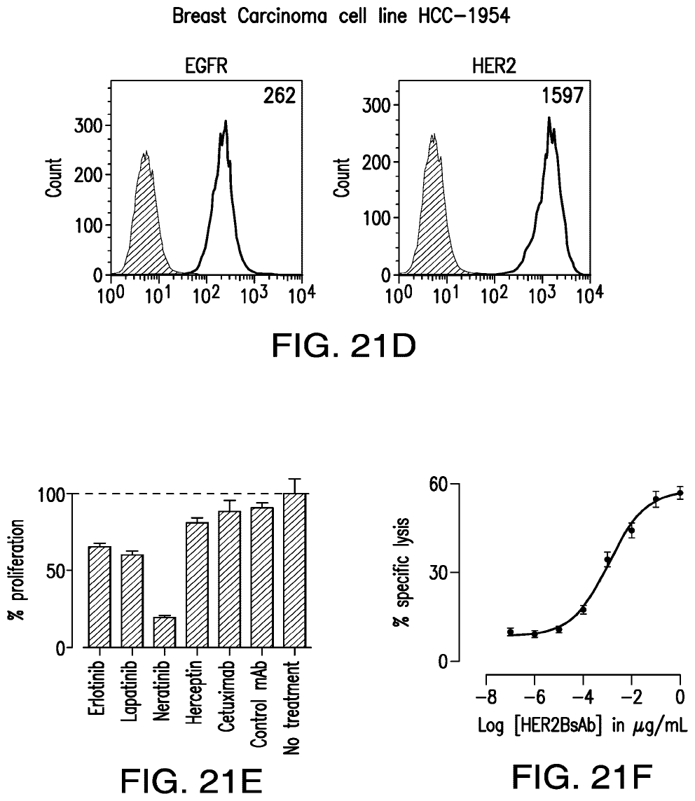

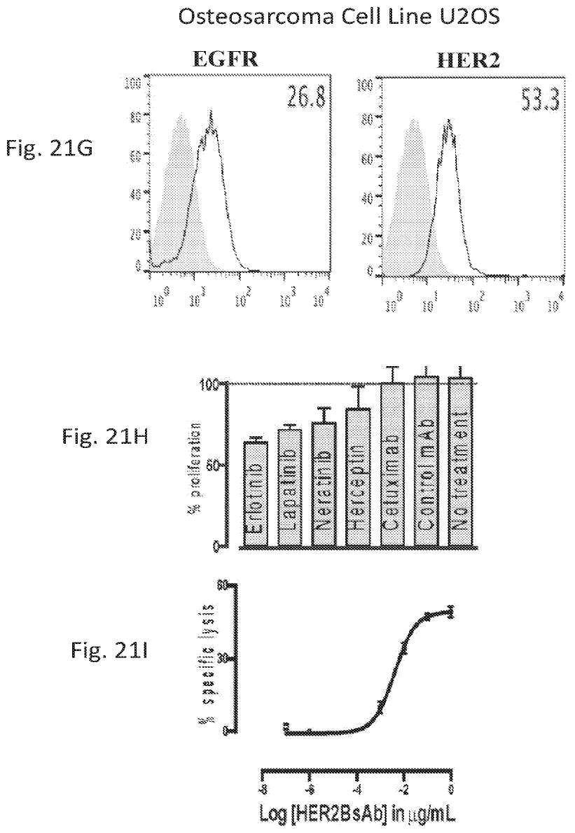

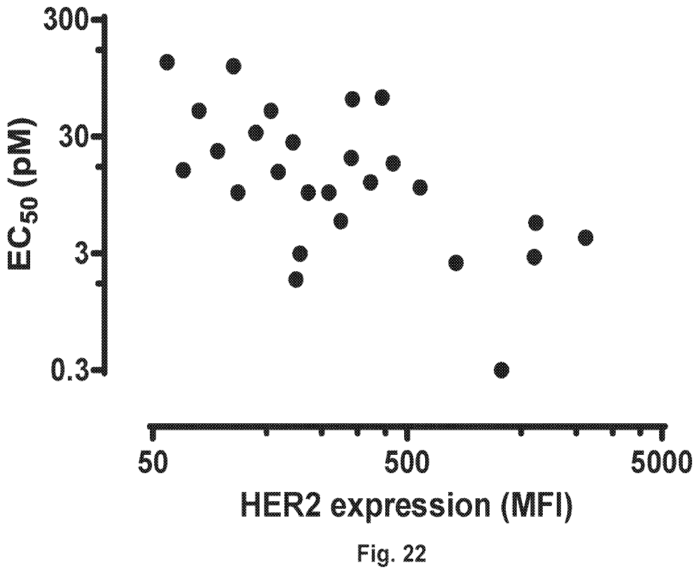

Several cell lines from different tumor systems (e.g., head and neck, breast, and sarcoma) were characterized for their HER2 level of expression by flow cytometry (FIG. 20). In this panel, 75% of these cells tested positive for HER2 expression by flow cytometry. Representative cell lines were assayed for their sensitivity to tyrosine kinase inhibitors (e.g., erlotinib, lapatinib, and neratinib), or HER antibodies (e.g., trastuzumab and cetuximab), as well as HER2-BsAb mediated T cell cytotoxicity. FIG. 21 shows representative examples of these experiments from three different lines from three different tumor systems. As shown, HER2 expression-even in low quantities—was sufficient to mediate T cell cytotoxicity in the presence of ATC and HER2-BsAb in cell lines otherwise resistant in vitro to HER-targeted therapies. When these cell lines were tested for cytotoxicity in the presence of ATC and HER2-BsAb, sensitivity to HER2-BsAb, expressed as EC50, strongly correlated with surface HER2 expression (FIG. 22)

### 6.2.3.5 HER2-BsAb Mediated T Cell Cytotoxicity was Relatively Insensitive to PD-L1 Expression on the Tumor Target or PD-1 Expression on T Cells.

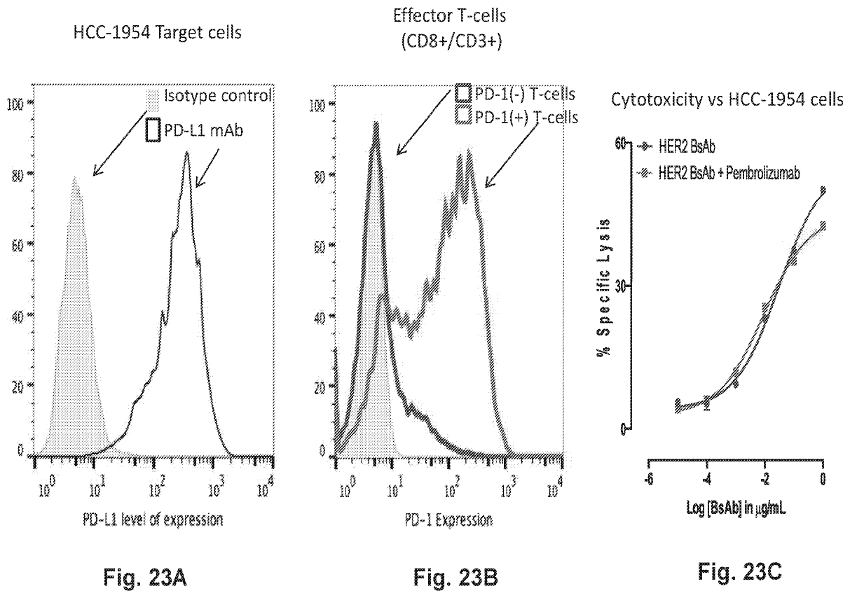

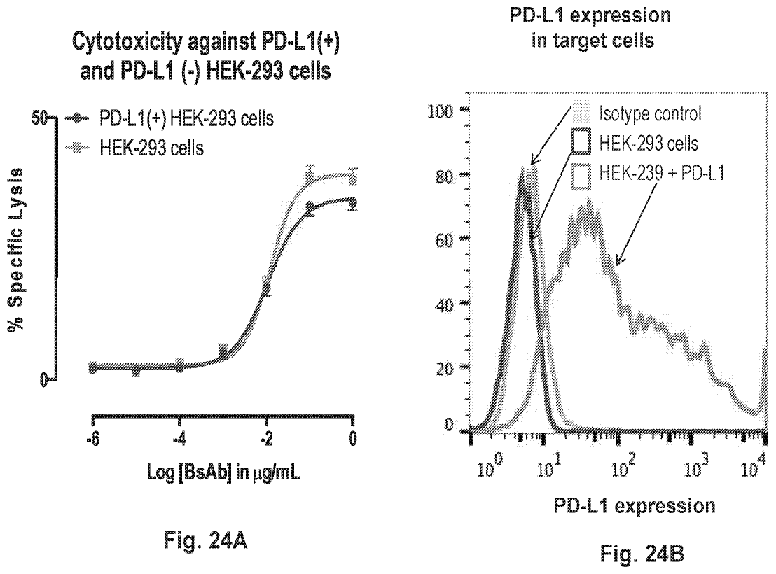

Activation of tumor-specific CTL in the tumor microenvironment is known to promote expression of PD-1/PD-L1, leading to T cell exhaustion or suppression, a phenomenon termed “adaptive immune resistance” (Tumeh et al., 2014, Nature 515:568-71). The presence of the PD-1/PD-L1 pathway has also been reported to limit the anti-tumor effects of T cell engaging bispecific antibodies (Junttila et al., 2014, Cancer Res 74:5561-71). To determine if HER2-BsAb had the same limitations, PD-1-positive ATCs were used against the HER2-positive, PD-L1-positive breast carcinoma cell line HCC1954, with or without the PD-1-specific mAb pembrolizumab. As shown in FIG. 23A, FIG. 23B, and FIG. 23C, PD-1-positive T cells generated similar cytotoxic responses in the presence of HER2-BsAb, independently of the presence of pembrolizumab. When HER2-positive human embryonic kidney cells (HEK-293) were transfected with the full sequence of PD-L1 and used as targets, cytotoxicity against cells expressing PD-L1 was not significantly different to the cytotoxicity observed in non-transfected HEK-293 cells (although maximal cytotoxicity was slightly less with PD-L1-positive HEK-293 versus PD-L1-negative HEK-293) (FIG. 24A and FIG. 24B shows the average of six experiments, and error bars represent standard error).

### 6.2.3.6 HER2-BsAb was Effective Against HER2-Positive Xenografts

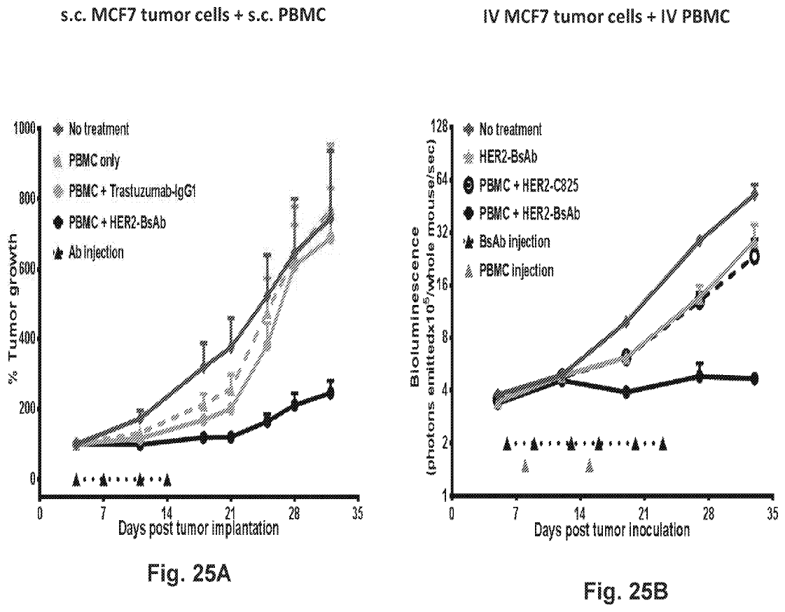

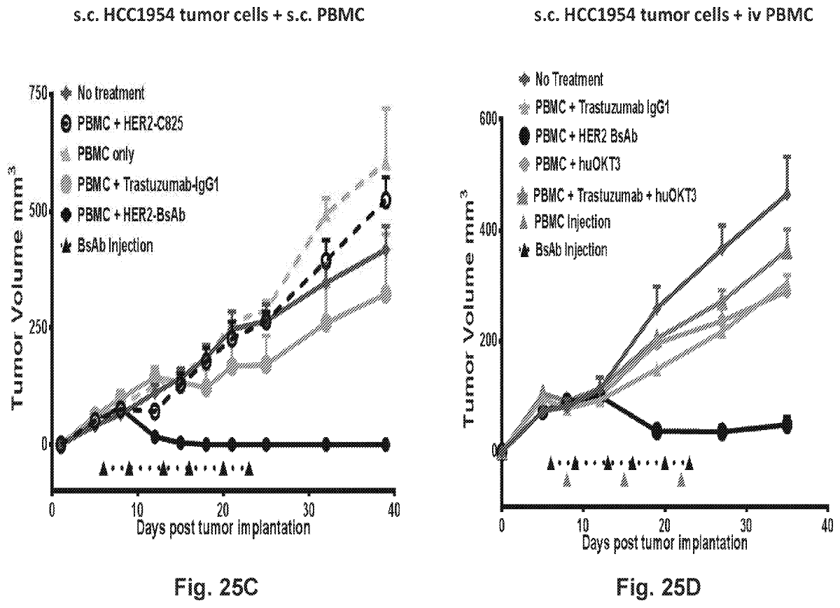

To determine the in vivo efficacy of HER2-BsAb, the breast carcinoma cell lines HCC1954 (HER2-high) and MCF-7 (HER2-low) were used in xenograft models in DKO mice. Three tumor models differing in tumor locations and effector routes were used: (1) intravenous tumor cells and intravenous effector PBMCs; (2) subcutaneous tumor cells and SC PBMCs; and (3) subcutaneous tumor cells and intravenous PBMCs. FIG. 25 summarizes the results of these experiments. The HER2-low MCF-7-luc (carrying luciferase reporter) cells were inoculated via tail vein injection into DKO. When tumor presence was confirmed by bioluminescence, mice were treated with six doses of intravenous HER2-BsAb or control BsAb twice a week for 3 weeks. Intravenous effector PBMCs were administered 48 hours after the first dose of HER2-BsAb, and again (one week later). Mice were evaluated for tumor burden using luciferin bioluminescence every week. In this hematogenous disease model, MCF-7 cells were completely eradicated without disease progression (FIG. 25B). This same cell line was implanted subcutaneously mixed with effector PBMCs subcutaneously and treated with four injections of HER2-BsAb twice a week for 2 weeks (totaling 4 injections in the first experiment) or twice a week for 3 weeks (totaling 6 injections in 2^{nd} experiment). In both experiments, HER2-BsAb caused a significant delay in tumor progression while PBMC+ trastuzumab or PBMC alone were ineffective (FIG. 25A). In two other separate experiments, subcutaneous HER2-positive breast carcinoma cell line HCC1954 was mixed with subcutaneous PBMCs. Again, both 4 or 6 injections of HER2-BsAb resulted in a complete suppression of tumor growth, while trastuzumab or control BsAb HER2-C825 had no effect (FIG. 25C). In the third model, where subcutaneous HCC1954 xenografts were treated with intravenous PBMC (once a week for 3 weeks), and intravenous HER2-BsAb twice a week for 3 weeks, tumor growth was substantially delayed (in 2 separate experiments), in contrast to only modest effects for trastuzumab+ huOKT3+ PBMC, control antibody (HER2-C825)+ PBMC, huOKT3+ PBMC, or HER2-BsAb alone without PBMC (FIG. 25D). The following observation were made: when effector PBMCs were mixed with tumor cells subcutaneously, complete tumor regression without recurrence was seen for mice over 90 days post-tumor implantation. When effector PBMCs were administered intravenously, there was significant reduction in the size of the tumors, but complete regression was only observed in a subset of animals.

### 6.2.4 Conclusions

This example describes a HER2-specific BsAb that has been shown to have potent T cell-mediated anti-tumor activity in vitro and in vivo, ablating tumors or delaying tumor growth in 3 separate tumor models in the presence of human PBMCs. Unlike monovalent bispecific antibodies, this HER2-BsAb had identical anti-proliferative capacity as trastuzumab. In addition, the serum half-life and area under the curve of HER2-BsAb were similar to IgG. Unlike other bispecific antibodies, which tended to aggregate, HER2-BsAb was stable at −20° C. and at 37° C., despite long term storage. Most importantly, the T cell-mediated cytotoxicity it induced was relatively insensitive to inhibition by the PD-1/PD-L1 pathway.

When compared to the existing platforms that target HER2, HER2-BsAb offers advantages. The F(ab)×F(ab) format, though effective in vitro, was similar in size to Blinatumomab (Sanford, 2015, Drugs, 75:321-7) and was expected to share similar pharmacokinetic and toxicity profiles (Shalaby et al., 1995, Clin Immunol Immunopathol 74:185-92, 1995), having a short half-life, thus requiring daily infusions, potential leakage into the central nervous system (CNS), potential CNS toxicity, and potential significant cytokine release syndrome. In addition, the anti-proliferative capacity of this F(ab)×F(ab) univalent system was 10-fold lower than trastuzumab. The IgG×IgG chemical conjugate between trastuzumab and OKT3 was useful for arming T cells ex vivo, but was not useful as an injectable, likely due to impurities associated with chemical conjugates (Lum and Thakur, 2011, BioDrugs 25:365-79; Lum et al., Clin Cancer Res 21:2305, 2015); in contrast, the HER2-BsAb provided herein is tolerated as an injectable. A heterodimer format was recently described using a monovalent system (Junttila et al., 2014, Cancer Res 74:5561-71) that does not preserve trastuzumab's anti-proliferative effects retained in HER2-BsAb.

There are other design features that distinguish HER2-BsAb from other known candidates of this class. Unlike most bispecific antibodies, HER2-BsAb's bivalent binding to the HER2 target was preserved, providing anti-proliferative activity similar to that of trastuzumab IgG1. Unlike F(ab)×F(ab) (Shalaby et al., 1995, Clin Immunol Immunopathol 74:185-92) or tandem scFv constructs (Sanford, 2015, Drugs, 75:321-7), HER2-BsAb had a molecular weight high enough to behave in pharmacokinetic analyses like a wild-type IgG. Unlike other bivalent bispecifics (Reusch et al., MAbs, 7:584, 2015), HER2-BsAb's reaction with CD3 was functionally monovalent. HER2-BsAb also differed from man heterodimeric bispecifics in its modified Fc, where aglycosylation removed both ADCC and CMC functions, thereby reducing cytokine release syndrome without affecting serum pharmacokinetics or compromising T cell activation. The other advantage is manufacturability; HER2-BsAb was produced in CHO cells and purified using procedures standard for IgG, without significant aggregation despite prolonged incubation at 37° C. HER2-BsAb is an important salvage option for patients who progress on standard HER2-based therapies, or a replacement for trastuzumab given its dual anti-proliferative and T cell retargeting properties.

### 6.3 Example 3

This example provides (a) a more detailed description of certain of the experiments described in Example 1 (Section 6.1); and (b) additional experiments as compared to Example 1 (Section 6.1).

T-cell based therapies have emerged as one of the most clinically effective ways to target solid and non-solid tumors. HER2 is responsible for the oncogenesis and treatment resistance of several human solid tumors. As a member of the HER family of tyrosine kinase receptors, its over-activity confers unfavorable clinical outcome. Targeted therapies directed at this receptor have achieved responses, although development of resistance is common. This example explores a novel HER2/CD3 bispecific antibody (HER2-BsAb) platform that, while preserving the anti-proliferative effects of trastuzumab, recruits and activates non-specific circulating T-cells, promoting T cell tumor infiltration and ablating HER2-positive (“HER2 (+)”) tumors, even when these are resistant to standard HER2 targeted therapies. Its in vitro tumor cytotoxicity, when expressed as EC50, correlated with the surface HER2 expression in a large panel of human tumor cell lines, irrespective of lineage or tumor type. HER2-BsAb-mediated cytotoxicity was relatively insensitive to PD-1/PD-L1 immune checkpoint inhibition. In four separate humanized mouse models of human breast cancer and ovarian cancer cell line xenografts, as well as in human breast cancer and gastric cancer patient-derived xenografts (“PDXs”), HER2-BsAb was highly effective in promoting T cell infiltration and suppressing tumor growth when used in the presence of human peripheral blood mononuclear cells (“PBMC”) or activated T cells (“ATC”). The in vivo and in vitro antitumor properties of this BsAb support its further clinical development as a cancer immunotherapeutic.

### 6.3.1 Introduction

Trastuzumab has significantly improved patient outcomes in breast cancer, and has also been key in the design and implementation of other targeted therapies (Singh et al., Br J Cancer 2014; 111:1888-98). However, HER2 expression does not guarantee a clinical response to trastuzumab or other HER2 targeted therapies (Devika & Sarat, Expert Review Of Anticancer Therapy 2011; 11 (2): 263-75; Lipton et al., Breast Cancer Research and Treatment 2013; 141 (1): 43-53). HER2-positive breast cancer patients with metastatic disease initially respond to trastuzumab and/or other HER2 targeted therapies, but almost all eventually will develop resistance and relapse (Montemurro & Scaltriti, J Pathol 2014; 232:219-29). In osteosarcoma and Ewing's sarcoma, where high levels of HER2 expression was associated with decreased survival (Gorlick et al., J Clin Oncol 1999; 17:2781-8), trastuzumab has not shown any benefit even when used in conjunction with cytotoxic chemotherapy (Ebb et al., J Clin Oncol 2012; 30:2545-51). Furthermore, trastuzumab, like other HER targeted therapies, has shown modest or no benefit against HER2 (+) positive head and neck cancer (Pollock & Grandis, Clinical Cancer Research 2014; 21 (3): 526-33).

The reasons for these failures are complex and only partially understood. The genomic diversity and constant evolution of malignancies make them less prone to oncogene addiction, a requirement for the success of targeted therapy. Furthermore, even when oncogene addiction is present, resistance can emerge from selection pressure induced by the use of targeted therapies (Lipton et al., Breast Cancer Research and Treatment 2013; 141 (1): 43-53). In fact, despite the initial enthusiasm received, the majority of targeted therapies have not produced a significant benefit in the overall cure of patients receiving them (Nathanson et al., Science 2014; 343:72-6). A different approach, one that selectively targets malignant cells that overexpress HER family receptors, and that can generate cytotoxic anti-tumor responses independently of the receptor activation status could be beneficial.

Redirecting the immune system against tumor cells has gained acceptance as an effective strategy to overcome resistance to cytotoxic chemotherapy and targeted therapy. In the forefront of these treatments, T-cell based therapies constitute the most promising approach. Both T-cell engaging bispecific antibodies and immune checkpoint antibody blockade have received accelerated approval from the FDA based on their outstanding clinical performance (Asher, Nature Reviews Drug Discovery 2015). The clinical success of chimeric antigen receptor (CAR) gene modified T-cells against non-solid tumors has further added to the enthusiasm among scientists, clinicians and the pharmaceutical industry.

The outstanding clinical responses seen with these therapies have consolidated T-cells as the most powerful effector cells within the immune system to eradicate tumor cells (Kershew et al., Clinical & Translational Immunology 2014; 3 (5): e16). Thus, a number of approaches that redirect them against tumor cells have been proposed and tested by many investigators. In this regard, Bispecific antibodies (“BsAb”) with specificity for T-cells and for tumor antigens have attracted the attention of researchers and big pharma. BsAb, in opposition to other antibody based therapies, only requires expression of its target of interest to be effective. By recruiting polyclonal T-cells through the CD3 surface receptor, BsAb activate T-cells irrespective of their lineage, antigen specificity, maturation, HLA restriction or co-stimulatory receptors. The direct activation of T-cells, bypassing the classic T cell receptor (“TCR”), removes the limitations imposed by HLA restriction and its level of expression (Brischwein et al., Journal of Immunotherapy 2006; 30:798-807), a well-established immune resistance mechanism (Sabbatino et al., Clinical transplants 2013:453-63).

Blinatumomab-a CD19/CD3 BsAb was approved in 2014 for treating Acute Lymphoplastic Leukemia (Sanford, Drugs 2015; 75:321-7). However, despite its promising results, the unfavorable pharmacokinetics of these small size molecules necessitate prolonged infusions, complicating their administration (Shalaby et al., J Exp Med 1992; 175:217-25; Portell et al., Clinical Pharmacology: Advances And Applications 2013; 5:5-11). Furthermore, the resulting cytokine release syndrome (“CRS”) still poses costly and often life-threatening complications. Importantly, despite the ability of bispecific antibodies to activate T-cells, the same inhibitory pathways that regulate classic T-cell function might still limit their effectiveness. For example, the heterodimeric design of a monovalent binding HER2/CD3 bispecific antibody was inhibited by the PD-1/PD-L1 inhibitory axis (Junttila et al., Cancer Research 2014; 74:5561-71).

This example reports a BsAb against the HER2 tumor antigen that offers two distinct advantages over the existing technologies: (1) it is based on the fully humanized HER2-specific IgG1 trastuzumab, preserving its pharmacologic advantages (Wittrup et al., Methods Enzymol 2012; 503:255-68) and bivalent binding to HER2, maximizing tumor avidity; (2) its binding to CD3 is functionally monovalent through the scFv derived from the humanized huOKT3 IgG1 sequence. Thus, HER2-BsAb is built on two mAbs with an extensive record of clinical safety. Previous studies have also shown that scFv linked to the carboxyl end of the light chain did not affect the targeting ability of these IgG forms (Cheal et al., Mol Cancer Ther 2014; 13:1803-12; Orcutt et al., Protein Eng Des Sel 2010; 23:221-8). Furthermore, this is a platform with its Fc function silenced to reduce the cytokine release syndrome. This example presents data to show that this HER2-BsAb has potent anti-tumor properties both in vitro and in vivo, against tumor cells that are resistant to HER2 targeted therapy or to trastuzumab.

### 6.3.2 Materials and Methods

### 6.3.2.1 Cell Lines

All cell lines were purchased from ATCC (Manassas Va) except: UM-SCC47 obtained from Dr. Carey at the University of Michigan, SCC-90, PCI-30 and PCI-15B from Dr. Robert Ferris at the University of Pittsburgh, SKOV3-luc from Dr. Dmitry Pankov at MSK, 93-VU-147T and HeLa from Dr. Luc Morris and UD-SCC2 from Henning Bier at Hals-Nasen-Ohrenklinik und Poliklinik. All cells were authenticated by short tandem repeat profiling using PowerPlex 1.2 System (Promega), and periodically tested for mycoplasma using a commercial kit (Lonza). The luciferase-labeled tumor cell lines MCF7-Luc were generated by retroviral infection with a SFG-GFLuc vector.

### 6.3.2.2 HER2-BsAb Design and Expression in CHO-S Cells

In the HER2-BsAb IgG-scFv format, V_{H} was identical to that of trastuzumab IgG1, except N297A mutation in the Fc region was introduced to remove glycosylation, thereby depleting Fc function. The sequence of the heavy chain is set forth in SEQ ID NO: 62. The light chain fusion polypeptide (SEQ ID NO: 60) was constructed by extending the trastuzumab IgG1 light chain with a C-terminal (G4S)₃ linker followed by huOKT3 scFv. The DNA encoding both heavy chain and light chain was inserted into a mammalian expression vector, transfected into CHO-S cells, and stable clones of highest expression were selected. Supernatants were collected from shaker flasks and the HER2-BsAb was purified by protein A affinity chromatography. The other control BsAb, HER2-C825, was generated as previously described (Cheal et al., Mol Cancer Ther 2014; 13:1803-12). HuOKT3 IgG1 was made using the same variable sequences as in huOKT3 scFv, and huOKT3 Fab was prepared from huOKT3 IgG1 using the Pierce Fab Preparation Kit (Thermo Scientific).

### 6.3.2.3 Other Antibodies And Small Molecules

Fluorophore-labeled HER2-BsAb was generated with the Zenon® Alexa Fluor® 488 Human IgG Labeling Kit from Life Technologies following the manufacturer's instructions. Pembrolizumab, cetuximab, trastuzumab, Erlotinib, Lapatinib and Neratinib were purchased from the Memorial Sloan Kettering Cancer Center pharmacy. Small molecules were re-suspended in dimethylsulfoxide (“DMSO”). The CD3, CD4, CD8 and CD56 antibodies were purchased from BD Biosciences (San Jose CA). The commercially available PE labeled PD-L1 specific mAb 10F.9G2 was purchased from BioLegend.

### 6.3.2.4 Cell Proliferation Assays

For tumor cell proliferation, 5,000 tumor cells were plated using RPMI-1640 supplemented with 10% fetal bovine serum (“FBS”) in a 96 well plate for 36 hours before being treated with—kinase inhibitors or the antibodies at the specified concentration. Cell proliferation was determined using the cell counting WST-8 kit (Dojindo technologies) following the manufacturer's instructions and using the formula: % survival rate=(Sample-Background)/(Negative control-Background). Lapatinib was ground using a mortar and pestle and suspended in DMSO as previously described (Chen et al., Molecular cancer therapeutics 2012; 11:660-9). To determine statistical significance, the results were analyzed using one-way ANOVA using Prism 6.0.

For T cell proliferation, naïve T cells were purified from human PBMC using Pan T cell isolation kit (Miltenyi Biotec). 2×10⁵ purified T cells were mixed with different antibodies in 96-well cell culture plate to a final volume of 250 μl/well. T cells were cultured and maintained in RPMI-1640 supplemented with 10% FBS in 37° C. for 6 days. T cell proliferation was quantitated using the WST-8 kit as described above.

6.3.2.5 Cytotoxicity Assays (51chromium Release Assay)

Cell cytotoxicity was assayed by ⁵¹Cr release as previously described (Xu et al., Cancer immunology research 2015; 3:266-77), and EC50 was calculated using SigmaPlot software. Effector PBMC cells were purified from buffy coats purchased from the New York Blood Center. ATCs were first purified from human PBMC using Pan T cell isolation kit, and then activated and expanded for approximately 14 days with CD3/CD28 Dynabeads (Invitrogen) according to manufacturer's protocol. For pre-incubation experiment, HER2-BsAb was pre-incubated with either ATCs (T cells pre-armed) or chromium-labeled tumor target cells (AU565 pre-targeted) for 30 minutes at room temperature, and unbound BsAb was washed off for two times before adding the other cells.

### 6.3.2.6 Cytokine Release Assay

Cytokine release was assayed as previously described (Ahmed et al., OncoImmunology 2015; 4: e989776), using naïve T cells prepared as described above. T cells (200,000/well) were cultured with or without NCI-N87 tumor cells (10,000/well) for 24 hours before supernatants being harvested for ELISA-based cytokine assay.

### 6.3.2.7 PD-1/PD-L1 Expression

To overexpress PD-L1 in HEK293 cells, cells were cultured in DMEM (Cellgro) supplemented with 10% heat-inactivated FBS and Penicillin (100 IU/ml) and streptomycin (100 μg/ml). HEK293 cells were plated into 6 well plates at 0.5 million cells/well with 2 ml fresh media the day before transfection. Transfection was done with 2.5 ug hPD-L1 plasmid DNA using Lipofectamine 2000 (Invitrogen) according to manufacturer's protocol. Cells were incubated at 37° C. for 48 hours before harvesting with 2 mM EDTA in PBS. 100,000-200,000 cells were used for FACS analysis and the rest were used for the killing assays.

To induce PD-1 expression in ATCs, effector cells were incubated in a 3:1 ratio for 24 hours with the HER2 (+) breast carcinoma cell line HCC1954, after these target cells were incubated with HER2-BsAb at a concentration of 10 μg/mL for 30 minutes and excessive antibody was removed. Cells were harvested and used in cytotoxicity assays as previously described against the HEK293 cells or HCC1954 cells. For PD-1 blockade, PD-1-induced ATCs were pre-incubated with 10 μg/mL pembrolizumab for 30 minutes before adding to the well.

### 6.3.2.8 In Vivo Experiments

All animal procedures were performed in compliance with Institutional Animal Care and Use Committee (IACUC) guidelines. For in vivo therapy studies, BALB-Rag2−/−IL-2R-yc-KO (DKO) mice (derived from colony of Dr. Mamoru Ito, CIEA, Kawasaki, Japan) (Koo et al., Expert Rev Vaccines 2009; 8:113-20; Andrade et al., Arthritis Rheum 2011; 63:2764-73) were used. Four humanized mouse xenograft models were used: (1) intravenous (“i.v.”) tumor plus i.v. effector cells, (2) subcutaneous (“sc”) tumor plus sc effector cells, (3) sc tumor plus i.v. effector cells, and (4) intraperitoneal (“i.p.”) tumor plus i.p./i.v. effector cells. Patient derived xenografts (“PDXs”) were established from fresh surgical specimens with Memorial Sloan Kettering Cancer Center Institutional Review Board approval. Effector PBMC cells and ATCs were prepared as described above. Prior to every experimental procedure, PBMCs and ATCs were analyzed by FACS for their percentage of CD3, CD4, CD8 and CD56 cells to ensure consistency. Antibodies were injected i.v. or i.p. twice a week started two days before effectors cells for 3-6 weeks, given as i.v. 5-10×10⁶ PBMC/ATC per injection, once a week for 2-3 weeks. s.c. xenografts were created with tumor cells suspended in Matrigel (Corning Corp, Tewksbury MA) and implanted in the flank of DKO mice. Tumor size was measured using 1) hand-held TM900 scanner (Pieira, Brussels, BE), 2) Calipers, or 3) Bioluminescence. Bioluminescence imaging was conducted using the Xenogen In Vivo Imaging System (IVIS) 200 (Caliper LifeSciences). Briefly, mice were injected i.v. with 0.1 mL solution of D-luciferin (Gold Biotechnology; 30 mg/mL stock in PBS). Images were collected 1 to 2 minutes after injection using the following parameters: a 10- to 60-second exposure time, medium binning, and an 8 f/stop. Bioluminescence image analysis was performed using Living Image 2.6 (Caliper LifeSciences).

### 6.3.2.9 Immunohistochemistry Staining

The immunohistochemical detection was performed at Molecular Cytology Core Facility of Memorial Sloan Kettering using Discovery XT processor (Ventana Medical Systems). Paraffin-embedded tumor sections were deparaffinized with EZPrep buffer (Ventana Medical Systems), antigen retrieval was performed with CC1 buffer (Ventana Medical Systems) and sections were blocked for 30 minutes with Background Buster solution (Innovex). Anti-CD3 (DAKO, cat #A0452, 1.2 μg/ml), anti-HER2 (Enzo, cat #ALX-810-227, 5 μg/ml), and anti-PD-1 (Ventana, cat #760-4895, 3. 1 μg/ml) antibodies were applied and sections were incubated for 5 hours, followed by 60 minutes incubation with biotinylated goat anti-rabbit IgG (Vector labs, cat #PK6101) for CD3 and HER2 antibodies, or biotinylated horse anti-mouse IgG (Vector Labs, cat #MKB-22258) for PD-1 antibodies at 1:200 dilution. The detection was performed with DAB detection kit (Ventana Medical Systems) according to manufacturer's instruction. Slides were counterstained with hematoxylin and coverslipped with Permount (Fisher Scientific). For PD-L1 staining, the sections were pre-treated with Leica Bond ER2 Buffer (Leica Biosystems) for 20 minutes at 100° C. The staining was done on Leica Bond RX (Leica Biosystems) with PD-L1 mouse monoclonal antibody (Cell Signaling, cat #29122, 2.5 μg/ml) for 1 hour on Leica Protocol F. All images were captured from tumor sections using Nikon ECLIPSE Ni-U microscope and NIS-Elements 4.0 imaging software.

### 6.3.2.10 Statistics

Differences between samples indicated in the figures were tested for significance by one-way ANOVA using Prism 6.0, and p<0.05 was considered statistically significant.

### 6.3.3 Results

### 6.3.3.1 HER2-BsAb

HER2-BsAb heavy chain was constructed using the standard human IgG1, except for the N297A mutation in the Fc region to remove glycosylation. The light chain was constructed by extending the trastuzumab IgG1 light chain with a C-terminal (G4S)₃ linker followed by huOKT3 scFv (Xu et al., Cancer Immunology Research 2015; 3:266-77). The DNAs encoding both heavy chain and light chain were inserted into a mammalian expression vector, transfected into CHO-S cells, and stable clones of highest expression were selected. Supernatants were collected from shaker flasks and purified on protein A affinity chromatography (Xu et al., Cancer Immunology Research 2015; 3:266-77).

The SEC-HPLC and SDS-PAGE of the HER2-BsAb was analyzed. Under reducing SDS-PAGE conditions, HER2-BsAb gave rise to two bands at around 50 KDa, since the huOKT3 scFv fusion to trastuzumab light chain increased the MW to ˜50 KDa (data not shown). SEC-HPLC showed a major peak (97% by UV analysis) with an approximate MW of 210 KDa, as well as a minor peak of multimers (data not shown). The BsAb remained stable by SDS-PAGE and SEC-HPLC after multiple freeze and thaw cycles (data not shown).

### 6.3.3.2 HER2-BsAb Retained Specificity, Affinity and Anti-Proliferative Effects of Trastuzumab

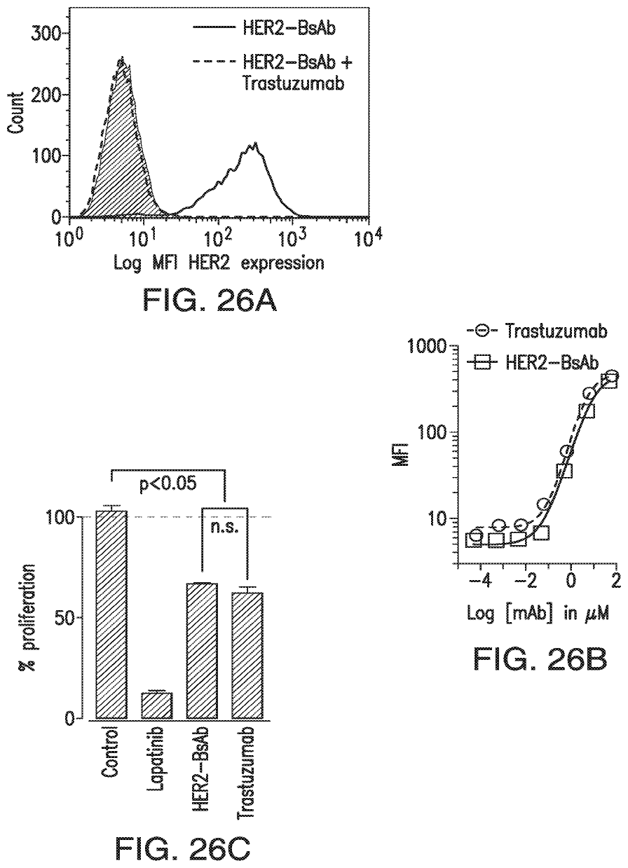

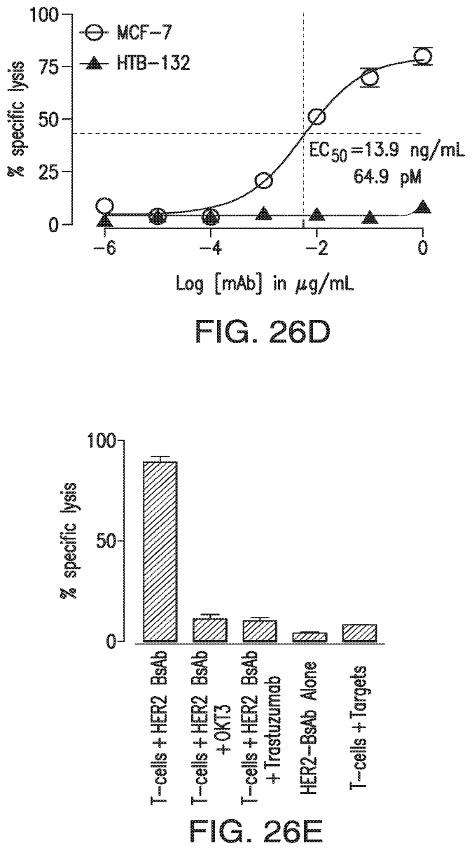

To determine if HER2-BsAb retained the specificity of trastuzumab, the HER2 (+) high SKOV3 ovarian carcinoma cell line was pre-incubated with 10 μg/mL of trastuzumab for 30 minutes and then immunostained using 1 μg/mL HER2-BsAb labeled with Alexa 488 (FIG. 26A). Pre-incubation with trastuzumab prevented HER2-BsAb from binding to SKOV3 cells, demonstrating that these antibodies shared the same specificity. To compare the avidity of HER2-BsAb to trastuzumab, the same cell line was incubated with 10 fold downward dilutions (from 10 μg/ml to 1×10⁻⁵ μg/mL) of trastuzumab or HER2-BsAb and analyzed by flow cytometry. The mean fluorescence intensity (“MFI”) was plotted against the antibody concentration in μM. Again the similarity in the binding curves confirmed that trastuzumab and HER2-BsAb had similar binding avidities for their common HER2 target (FIG. 26B).

Finally, the trastuzumab-sensitive breast carcinoma cell line SKBR3 was treated with Isotype control mAb, 10 nM lapatinib (as a positive control), 10 μg/mL HER2-BsAb or 10 μg/mL trastuzumab for 72 hours and cell proliferation was assayed. As shown in FIG. 26C, trastuzumab and HER2-BsAb had similar anti-proliferative effects that were significant compared to the negative control. As expected, lapatinib showed the strongest inhibition of cell proliferation.

### 6.3.3.3 HER2-BsAb Redirected T Cell Cytotoxicity was HER2 Specific and Dependent on CD3

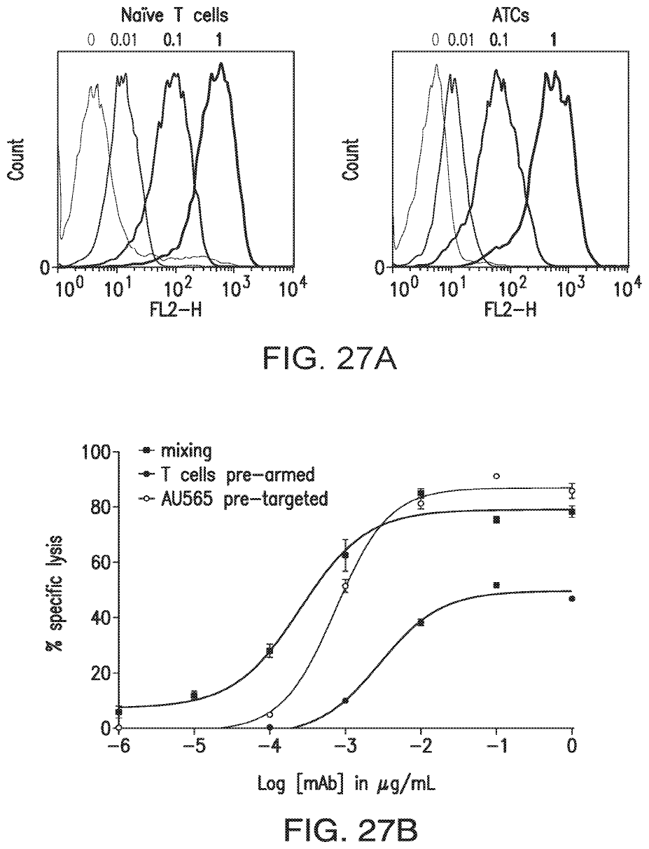

Prior to the cytotoxicity assay, HER2-BsAb was shown capable of binding different T cells at the similar level (MFI around 450 with the BsAb concentration of 1 μg/10⁶ cells), no matter whether they were naïve T cells purified from fresh PBMC or activated T cells (ATCs) (FIG. 27A). To establish the specificity of cytotoxic responses by T cells in the presence of HER2-BsAb, HER2-negative (“HER2 (−)”) breast carcinoma HTB-132 cells and HER2 (+) MCF-7 cells were tested in a cytotoxicity assays using ATCs (E: T ratio of 10:1) and HER2-BsAb at decreasing concentrations (FIG. 26D). Cytotoxicity was robust for HER2 (+) cells but absent for HER2 (−) cells. In fact, HER2-BsAb was able to redirect efficient T cell killing no matter whether BsAb was present throughout the 4 hour assay (mixing), or used to pre-arm T cells and then washed off, or to pre-target AU565 tumor cells and then washed off. Although pre-targeted AU565 cells were killed as well as mixing all three together, pre-armed T cells were less potent due to the low avidity of BsAb binding to CD3 on T cells (FIG. 27B). To demonstrate the dependency of cytotoxicity on both HER2 and CD3, HER2-BsAb cytotoxicity against HER2 (+) SCCHN cell line PCI-13 was tested in the presence of trastuzumab, or the CD3 specific blocking huOKT3 IgG1 (FIG. 26E). Pre-incubation with either trastuzumab or huOKT3 prevented HER2-BsAb mediated T-cell cytotoxicity.

6.3.3.4 HER2-BsAb Mediated Cytotoxicity Against HER2 (+) Cell Lines that were Resistant To Other HER2 Targeted Therapies.

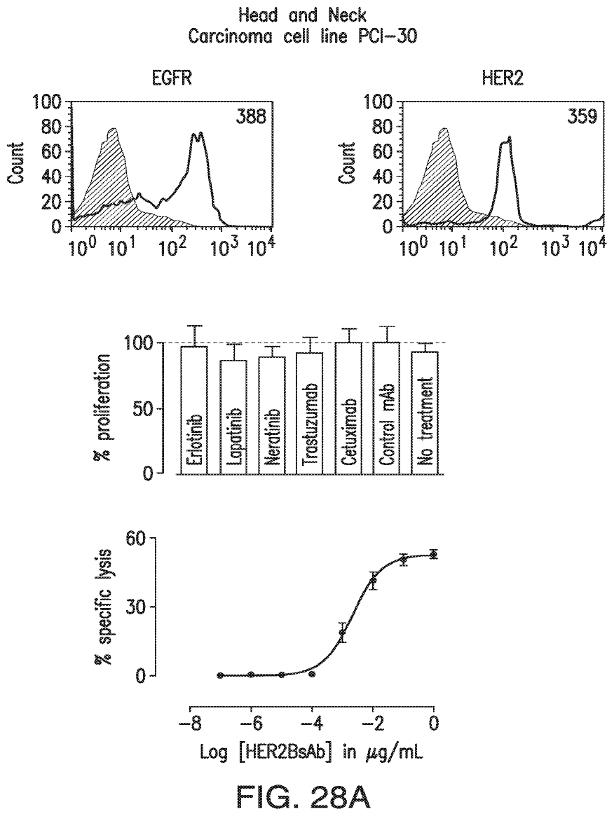

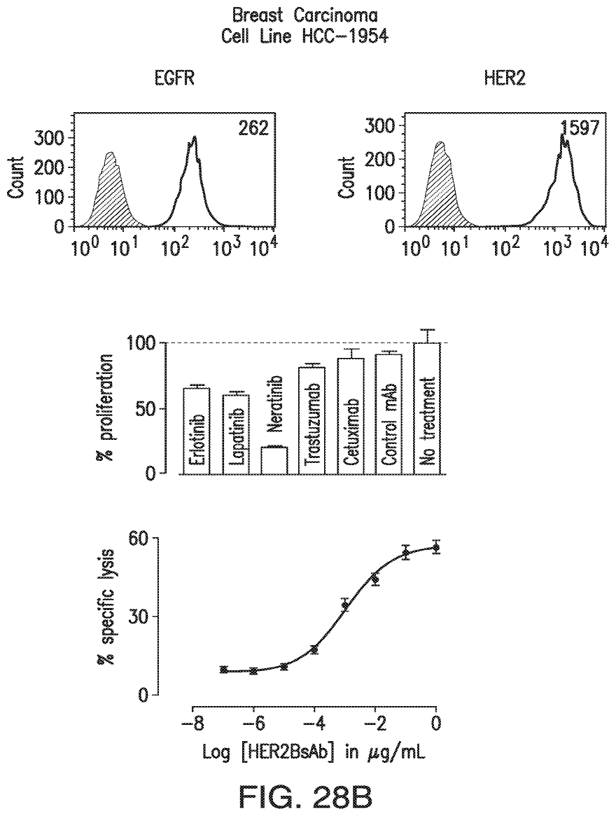

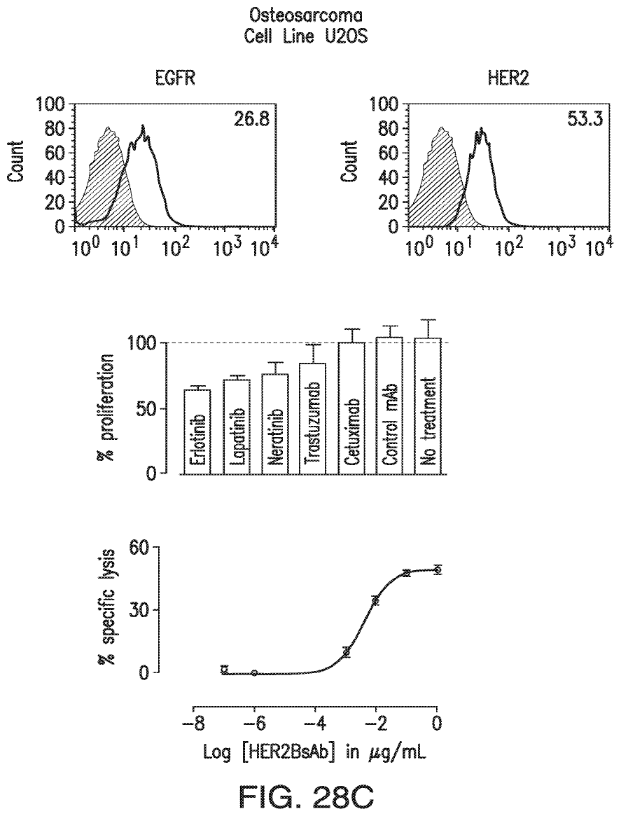

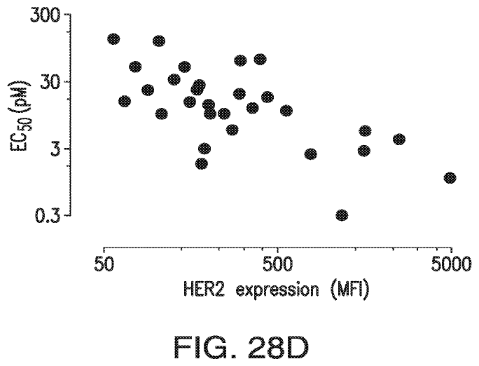

A panel of a total of 39 cell lines from different tumor systems (breast, ovarian, gastric, head and neck, sarcoma, etc.) was characterized for their HER2 expression levels by flow cytometry and CTL activity (Table 9). In this panel, 75% of these cells were tested positive for HER2 expression. Representative cell lines were assayed for their sensitivity to tyrosine kinase inhibitors (10 nM each of Erlotinib, Lapatinib, Neratinib), or HER antibodies (10 μg/mL each of trastuzumab and cetuximab), as well as HER2-BsAb mediated T-cell cytotoxicity. FIG. 28A, FIG. 28B and FIG. 28C showed three representative lines from three different tumor systems. As shown, HER2 expression, even in low quantities, was sufficient to mediate T-cell cytotoxicity in the presence of ATC and HER2-BsAb in cell lines otherwise resistant in vitro to HER-targeted therapies. When these cell lines were tested for cytotoxicity in the presence of ATC and HER2-BsAb, sensitivity to HER2-BsAb expressed as EC50 inversely correlated with surface HER2 expression in general (FIG. 28D, Table 9).

TABLE 9

| | | HER2 | |
|---|---|---|---|
| | | Expression | EC50 |
| Tumor Type | Cell Line | (MFI)* | (pM)** |
| Breast carcinoma | AU565 | 1175 | 0.3 |
| Gastric Carcinoma | NCI-N87 | 4900 | 1.1 |
| Ovarian Carcinoma | OVCAR3 | 183 | 1.8 |
| Breast Carcinoma | MDA-MB-361 | 777 | 2.5 |
| Ovarian Carcinoma | SKOV3 | 1577 | 2.8 |
| Melanoma | SKMEL28 | 190 | 3 |
| Breast Carcinoma | SKBR3 | 2506 | 4.1 |
| Breast Carcinoma | HCC1954 | 1597 | 5.5 |
| Head and Neck Cancer | SCC90 | 274 | 5.7 |
| Ewings | SKEAW | 246 | 10 |
| Osteosarcoma | CRL1427 | 108 | 10 |
| Rhabdomyosarcoma | HTB82 | 204 | 10 |
| Osteosarcoma | RG 160 | 563 | 11 |
| Head and Neck Cancer | PCI-30 | 359 | 12.2 |
| Gastric Carcinoma | KATO III | 201 | 13.5 |
| Melanoma | HT-144 | 156 | 15 |
| Neuroblastoma | NB5 | 66 | 15.5 |
| Osteosarcoma | RG 164 | 439 | 17.7 |
| Head and Neck Cancer | UM SCC47 | 302 | 19.8 |
| Osteosarcoma | U2OS | 90 | 22.5 |
| Gastric Adenocarcinoma | AGS | 172 | 23 |
| Head and Neck Cancer | UDSCC2 | 178 | 26.9 |
| Gastric Carcinoma | SNU-16 | 29 | 30.5 |
| Head and Neck Cancer | 93VU147T | 127 | 32.4 |
| Ewings | SKES-1 | 146 | 50 |
| Breast Carcinoma | MDA-MB-231 | 76 | 50.2 |
| Head and Neck Cancer | 15B | 305 | 62.8 |
| Breast Carcinoma | MCF7 | 398 | 64.9 |
| Cervical Cancer | HeLa | 104 | 120.7 |
| Melanoma | M14 | 57 | 130 |
| Breast Carcinoma | MDA-MB-468 | 6 | >5000 |
| Neuroblastoma | NMB7 | 12 | >5000 |
| Neuroblastoma | SKNBE(2)C | 8 | >5000 |
| Neuroblastoma | IMR32 | 6 | >5000 |
| Neuroblastoma | SKNBE(2)S | 4 | >5000 |
| Neuroblastoma | SKNBE(1)N | 3 | >5000 |
| Small Cell lung Cancer | NCI-H524 | 14 | >5000 |
| Small Cell lung Cancer | NCI-H69 | 10 | >5000 |
| Small Cell lung Cancer | NCI-H345 | 6 | >5000 |

### 6.3.3.5 HER2-BsAb Mediated In Vitro T-Cell Cytotoxicity was Relatively Insensitive to PD-L1 Expression on the Tumor Targets or PD-1 Expression on T Cells.

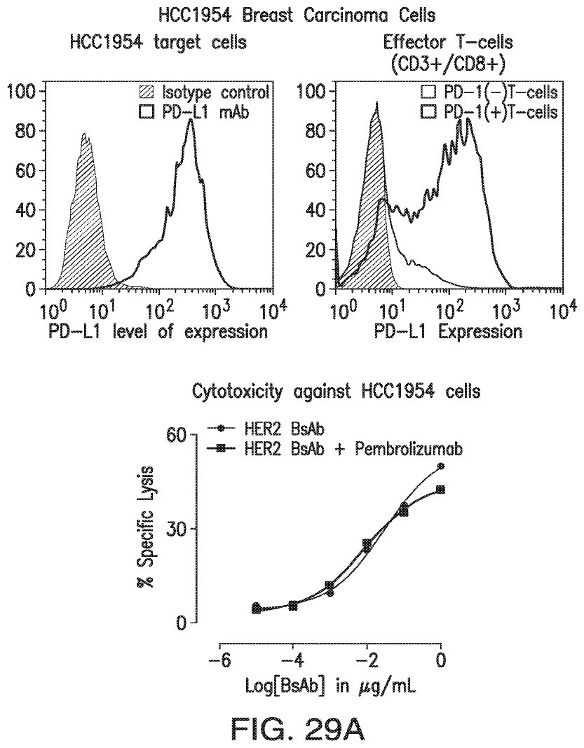

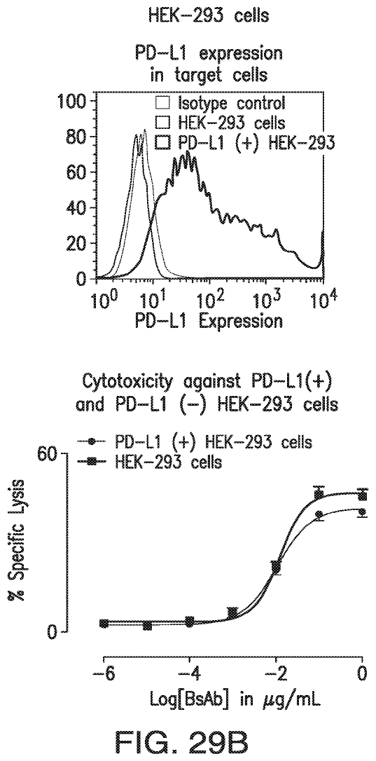

Activation of tumor-specific CTL in the tumor microenvironment is known to promote expression of PD-1/PD-L1, leading to T-cell exhaustion or suppression, a phenomenon termed “adaptive immune resistance” (Tumeh et al., Nature 2014; 515:568-71). The presence of PD-1/PD-L1 pathway has also been reported to limit the anti-tumor effects of T-cell engaging bispecific antibodies (Junttila et al., Cancer Research 2014; 74:5561-71). To determine if HER2-BsAb had this same limitation, PD-1-positive (“PD-1 (+)”) ATCs were used against the HER2 (+) PD-L1-positive (“PD-L1 (+)”) breast carcinoma cell line HCC1954 with or without the PD-1-specific antibody pembrolizumab. As shown in FIG. 29A, PD-1 (+) T cells generated similar cytotoxic responses in the presence of HER2-BsAb no matter whether pembrolizumab was present or not. When HER2 (+) human embryonic kidney cells (HEK-293) were transfected with the full sequence of PD-L1 and used as targets, cytotoxicity against cells expressing PD-L1 was not significantly different to the cytotoxicity observed in non-transfected HEK-293 cells, although maximal cytotoxicity was slightly less with PD-L1 (+) HEK-293 versus PD-L1-negative (“PD-L1 (−)”) parental HEK-293 (FIG. 29B).

### 6.3.3.6 HER2-BsAb was Effective Against HER2 (+) Xenografts

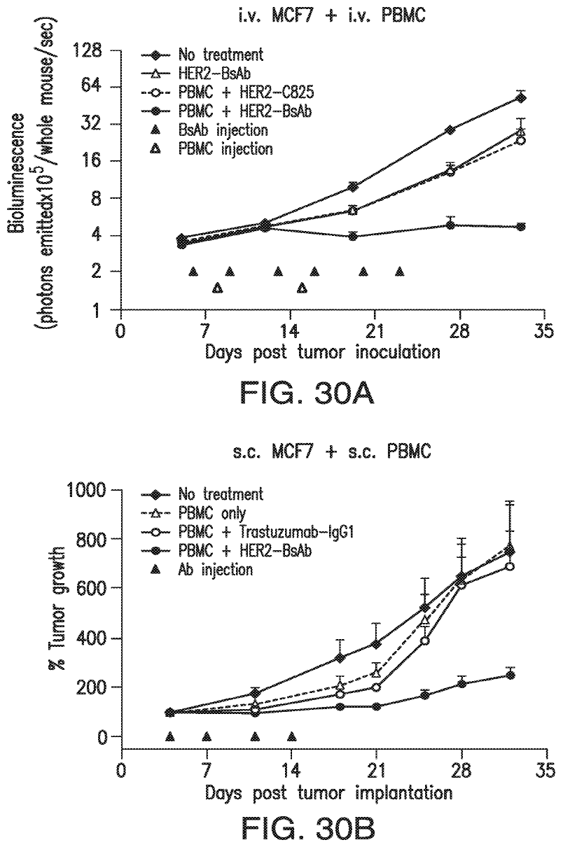

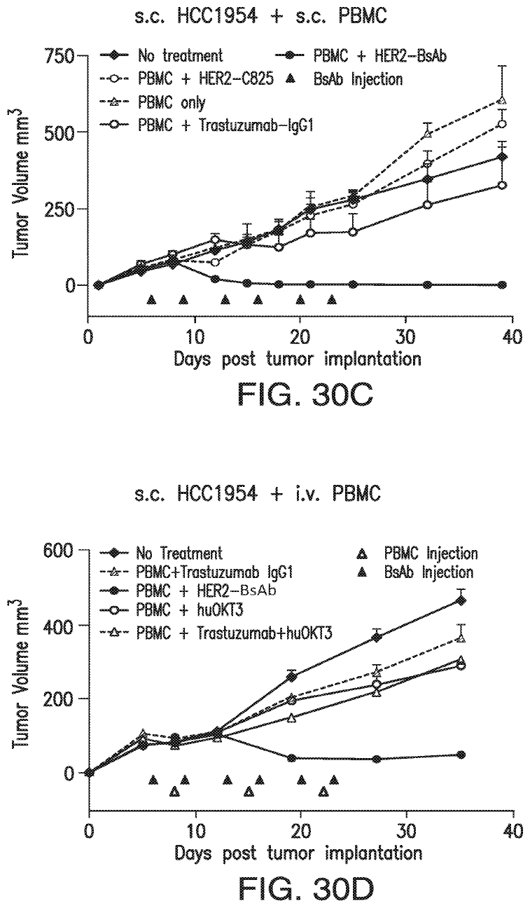

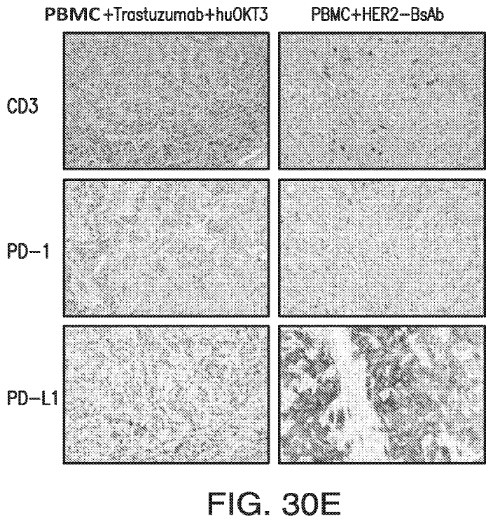

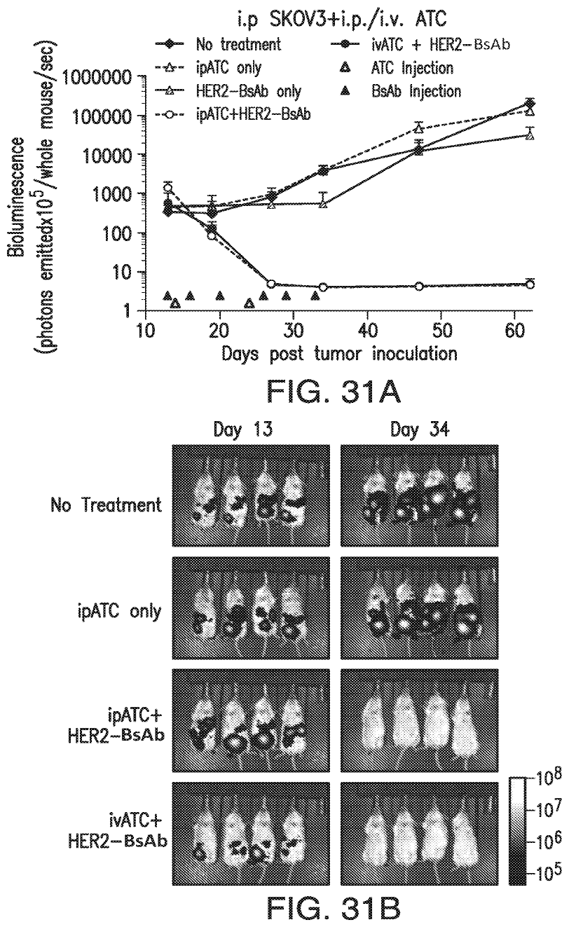

To determine the in vivo efficacy of HER2-BsAb, the breast carcinoma cell lines HCC1954 (HER2high) and MCF-7 (HER2low), ovarian carcinoma cell line SKOV3, and HER2 (+) patient-derived breast cancer and gastric cancer xenografts (“PDXs”) were used in DKO mice xenograft models. Four tumor models differing in tumor locations and effector routes were used, with the first three described before (Xu et al., Cancer Immunology Research 2015; 3:266-77) to simulate different clinical situations: (1) intravenous (“i.v.”) tumor cells/i.v. effector PBMC; (2) subcutaneous (“s.c.” tumor cells/s.c. PBMC; (3) s.c. tumor cells/i.v. PBMC; and (4) intraperitoneal (“i.p.” tumor cells plus i.p. or i.v. effector T cells to simulate ovarian cancer metastasizing to the peritoneal cavity. FIG. 30 and FIG. 31 summarize the results of these experiments using cell lines, and FIG. 32 summarizes the results of these experiments using PDXs (M37 breast cancer and EK gastric cancer).

3×10⁶ HER2low MCF-7-luc (carrying luciferase reporter) cells were inoculated via tail vein i.v. injection into DKO mice. When tumor presence was confirmed by bioluminescence, mice were treated with HER2-BsAb or control BsAb (20 μg i.v., 2 times per week for 3 weeks), in combination with PBMC (5×10⁶ i.v., once per week for 2 weeks). Mice were evaluated for tumor burden using luciferin bioluminescence every week. In this hematogenous disease model, MCF-7 cells were completely eradicated without disease progression (FIG. 30A). This same cell line was implanted s.c. mixed with effector PBMC (1:1, 7×10⁶ each), and treated with HER2-BsAb (10 μg i.v., 2 times per week for 2 weeks in the 1st experiment; or 20 μg i.v. 2 times per week for 3 weeks in the 2^{nd} experiment). In both experiments, HER2-BsAb caused a significant delay in tumor progression, while PBMC+ trastuzumab or PBMC alone was ineffective (FIG. 30B). In two other separate experiments, 5×106 HER2 (+) breast carcinoma HCC1954 cells were implanted s.c. mixed with 2.5×10⁶ PBMC (2:1). Again, after either 4 or 6 injections of HER2-BsAb (20 μg i.v. per dose), there was complete suppression of tumor growth, while trastuzumab or control BsAb HER2-C825 almost had no effect (FIG. 30C). In the third model, where s.c. 5×10⁶ HCC1954 xenografts were treated with i.v. PBMC (5×10⁶, once per week for 3 weeks) and i.v. HER2-BsAb (100 μg, twice per week for 3 weeks), tumor growth was substantially delayed (2 separate experiments), in contrast to only modest effects for PBMC+ trastuzumab+ huOKT3, PBMC+ trastuzumab, or PBMC+ huOKT3 (FIG. 30D). For HCC1954 xenografts, the following observations were made: (1) when effector PBMCs were mixed with tumor cells s.c., complete tumor regression without recurrence was seen past 90 days from tumor implantation; and (2) when effector PBMCs were administered i.v., there was significant reduction in the size of the tumors, but complete regression was only observed in a subset of animals (data not shown).

Since T cell homing into tumor is critical for anti-tumor response in cancer immunotherapy (Tang et al., Cancer Cell 2016; 29:285-96), T-cell tumor infiltration was studied using the s.c. tumor model described in FIG. 30D. Tumors were collected 5 days after i.v. PBMC and immunohistochemistry (“IHC”) was performed (FIG. 30E). T-cell tumor infiltration by CD3 (+) staining was detected only in PBMC+HER2-BsAb-treated group, but not in control group (PBMC+ Trastuzumab+ huOKT3). These infiltrated T cells also had PD-1 expression, although it was very weak. Interestingly, PD-L1 expression in the tumor cells was strongly upregulated in the HER2-BsAb-treated mice, presumably induced by the cytokines released by the infiltrated T cells in the vicinity. But still, HER2-BsAb treatment eradicated these tumors. This was consistent with the in vitro data (FIG. 29) showing HER2-BsAb-mediated T-cell cytotoxicity was relatively insensitive to or sufficient to overcome the PD-1/PD-L1 immune checkpoint inhibition.

To simulate ovarian cancer that metastasized into the peritoneal cavity, 1×10⁵ ovarian cancer SKOV3-luc cells were injected peritoneally in DKO mice, and treatments were started after confirming tumor growth by bioluminescence. Besides i.v.ATC, i.p.ATC was also tested as a source of effectors. As shown in FIG. 31A and FIG. 31B, after treatment with ATC (7.5×10⁶ i.v. or i.p., once a week for 2 weeks), and i.p. HER2-BsAb (100 μg, twice per week for 3 weeks), tumors were completely eradicated without evidence of recurrence at followup. Both i.v.ATC and i.p.ATC were equally effective in this fourth model.

HER2-BsAb was next tested using PDXs, since they could approximate the tumor heterogeneity and microenvironment typically found in fresh human tumor specimens. To determine whether HER2-BsAb is effective against PDXs, two HER2 (+) PDXs (gastric cancer PDX (EK) and breast cancer PDX (M37)) were tested using the s.c. tumor cells/i.v. PBMC model similar to the one described in FIG. 30D. The PDXs were recently characterized by IHC using the PATHWAY anti-HER2/neu (4B5) Rabbit Monoclonal Primiary Antibody (VENTANA) and scored (according to the 2013 ASCO HER2 Breast Cancer Testing Guidelines (see, e.g., Wolff et al., Journal of Clinical Oncology, 2013, 31 (31): 3997-4013)) as IHC 3+for breast cancer PDX M37 and IHC 2+for gastric cancer PDX EK. When the gastric PDX (EK) was passaged s.c. in DKO mice and treated with i.v. PBMC (1×10⁷, once a week for 3 weeks) and i.v. HER2-BsAb (100 μg, twice per week for 5 weeks), tumors were completely eradicated without disease progression, (FIG. 32A), accompanied by substantial amount of T cell tumor infiltration (FIG. 32B), even though the HER2 expression level was relatively low compared to the M37 breast cancer PDX (FIG. 32C). In this next experiment, M37 PDX was passaged s.c. in DKO mice, and treated with i.v. PBMC (7.5×10⁶, once a week for 3 weeks) and i.v. HER2-BsAb (100 μg, twice per week for 6 weeks). Tumor growth was completely suppressed in the group treated with HER2-BsAb and PBMC (FIG. 32D). Interestingly, despite characterization of the M37 PDX as IHC 3+, which is a HER2 level indicative of suitability for treatment with trastuzumab (see, e.g., Wolff et al., Journal of Clinical Oncology, 2013, 31 (31): 3997-4013), tumor growth was not suppressed in the M37 PDX group treated with PBMC+ trastuzumab+ huOKT3 (FIG. 32E). Taken together, these experiments showed that HER2-BsAb was effective against early passaged HER2 (+) human tumor specimens.

### 6.3.4 Conclusions

This example described a HER2-specific BsAb with potent T cell mediated anti-tumor activity in vitro and in vivo, ablating tumors or delaying tumor growth in four separate tumor-human PBMC compartment models. Unlike monovalent bispecifics, this HER2-BsAb had identical anti-proliferative capacity to its parental trastuzumab. Its serum half-life and area under the curve were similar to IgG (data not shown). Most importantly, the T cell-mediated cytotoxicity it induced was relatively insensitive to inhibition by the PD-1/PD-L1 pathway, not previously described for this IgG-scFv platform (Xu et al., Cancer Immunology Research 2015; 3:266-77). To date, other than the anti-GD2 hu3F8-BsAb (Xu et al., Cancer Immunology Research 2015; 3:266-77), no published T-cell redirecting bispecific antibodies have used this format. The ability of this IgG-scFv antibody platform to recruit circulating lymphocytes into the tumor stroma is critical, given the importance of tumor-infiltrating lymphocyte (TIL) cells for a successful anti-tumor effect in most checkpoint blockade studies to date (Tumeh et al., Nature 2014; 515:568-71), distinguishing responders from nonresponders (Gajewski et al., Semin Oncol 2015; 42:663-71).

Schreiber has proposed that tumor cells evolve to evade the immune system through a process termed “immuno-editing”. Broadly speaking, this process occurs at two levels: by changes within the (1) tumor cells or (2) the tumor microenvironment. Tumor cells can evade T-cell responses by down-regulating MHC/peptide complexes or by decreasing tumor-antigen expression or through the loss of antigen presenting machinery components. On the other hand, suppression of the immune response in the tumor microenvironment is the result of T-regulatory cells, Myeloid-derived suppressor cells, M2 macrophages (Diaz-Montero et al., Semin Oncol 2014; 41:174-84; Laoui et al., Frontiers in immunology 2014; 5:489; Nishikawa & Sakaguchi, Curr Opin Immunol 2014; 27:1-7), immuno-suppressive cytokines (including IDO) (Munn & Mellor, Trends Immunol 2013; 34:137-43), immune checkpoint molecules (Callahan et al., Frontiers in Oncology 2014; 4:385; Postow et al., J Clin Oncol 2015; 33 (17): 1974-82) and the consumption of IL-2 (Schreiber et al., Science 2011; 331:1565-70).

Immune checkpoint antibodies that target the CTLA-4 and PD-1/PD-L1 inhibitory pathways are capable of reversing the inhibitory tumor-microenvironment and producing significant and long-lasting clinical responses (Farolfi et al., Melanoma research 2012; 22:263-70). However, these strategies are not effective against all tumor types and their success is limited to a subset of patients. Durable clinical responses to the CTLA-4 blockade were recently correlated with tumor mutational load and the expression of antigenic tetra-peptides that resembled those found in viral and bacterial pathogens (Snyder et al., N Engl J Med 2014; 371:2189-99). Clonal neoantigens were shown to elicit T cell immunoreactivity and sensitivity to blockade of the PD-1/PD-L1 axis (McGranahan et al., Science 2016; 351 (6280): 1463-9). Based on these data, the pre-existence of CD8 (+) T-cells in the tumor (TILs) would be critical. More importantly, IHC evidence of negative regulation of tumor infiltrating lymphocytes (TIL) by the PD-1/PD-L1 axis, was correlated with clinical response to checkpoint blockade (Tumeh et al., Nature 2014; 515:568-71).

As data continues to accumulate, a consensus is emerging that these immune modulations would likely be ineffective against tumors with low immunogenicity because the presence of tumor specific lymphocytes is required for their clinical activity. Indeed, HER2 has been linked to immune resistance (Seliger & Kiessling, Trends in Molecular Medicine 2013; 19:677-84). This subset of patients, with “T-cell resistant” HER2 (+) tumor cells and/or insufficient clonal frequency of tumor-specific T-cells, would likely not benefit from immune checkpoint blockade alone. The unique property of the HER2-BsAb described in this example to recruit T-cells of any specificity and direct them against established tumors with relative insensitivity to the PD-1 immune checkpoint pathway is of interest, as it directly addresses the known limitations of immune checkpoint blockade. In fact, preliminary in vivo data showed no additional benefit of PD-1 blockade to the HER2-BsAb therapeutic efficacy (data not shown), even though tumor PD-L1 expression was up-regulated substantially following T cell infiltration (FIG. 30E).

When compared to the existing platforms that target HER2, HER2-BsAb offers advantages. Shalaby and colleagues described the development of a bispecific (Fab′)2 antibody (anti-HER2 Fab′×anti-CD3 Fab′) through expressing each Fab′ separately and ligating the two together by chemical conjugation (Shalaby et al., J Exp Med 1992; 175:217-25). More recently, Junttila and colleagues developed a heterodimeric bispecific IgG (anti-HER2× anti-CD3) using “knob-and-hole” format (Junttila et al., Cancer Research 2014; 74:5561-71). Both formats have monovalent binding to either HER2 or CD3, and are substantially different, both structurally and functionally, when compared to the HER2-BsAb described herein for the following reasons. First, the bivalent binding to HER2 is critical for the anti-proliferation capability, which is preserved in the HER2-BsAb construct described herein (FIG. 26C) but not in those two monovalent systems, as demonstrated by Juntilla et al. Juntilla et al. showed that the anti-proliferation capability of monovalent binding to HER2 (either heterodimeric bispecific IgG or trastuzumab-Fab) was 10-fold lower than bivalent trastuzumab (Junttila et al., Cancer Research 2014; 74:5561-71). Without being bound by any particular theory, it is hypothesized that the dual mechanism (anti-proliferation plus T cell cytotoxicity) may create synergism and partly explains the potent efficacy of the HER2-BsAb in vivo. This may provide a salvage option for patients who progress on standard HER2-based therapies, or a replacement for trastuzumab given its dual anti-proliferative and T cell retargeting properties. Second, the bivalent binding to HER2 in our BsAb maintains high avidity (FIG. 26B) so as to maximize tumor binding, while the monovalent binding to HER2 (either heterodimeric bispecific IgG or trastuzumab-Fab) is 10-fold lower than trastuzumab (Junttila et al., Cancer Research 2014; 74:5561-71). Higher avidity results in higher T cell dependent cell cytotoxicity, a phenomenon that has been demonstrated in T cell engaging bispecific antibodies (Ahmed et al., OncoImmunology 2015; 4: e989776). Additionally, without being bound by any particular theory, it is hypothesized that the high avidity of the HER2-BsAb contributes to overcoming PD-1/PD-L1 checkpoints (FIG. 29), whereas the monovalent system by Juntilla was shown to be inhibited by the PD-1/PD-L1 axis. Third, the BsAb described in this example has the trastuzumab IgG backbone, preserving its pharmacologic advantages, while Shalaby's construct doesn't have FcR (n) affinity, should have much shorter serum half-life, and probably needs to be administered as a continuous infusion (as for Blinatumomab) to be effective in vivo. Fourth, the other advantage is manufacturability: once a CHO stable line established, the HER2-BsAb can be produced in large scale and purified like normal IgG without significant aggregation despite prolonged incubation at 37° C., while chemical conjugates require more complicated syntheses and downstream processing—each Fab′ expressed and purified separately, chemically modified, and then the two chemically conjugated and repurified. To ensure a final product that is pure and chemically stable for direct clinical infusion is technically challenging and costly. Such chemically crosslinked reagents have only been feasible for ex vivo arming of T cells, but not for direct parenteral injections in the clinic (Lum & Thakur A, BioDrugs 2011; 25:365-79).

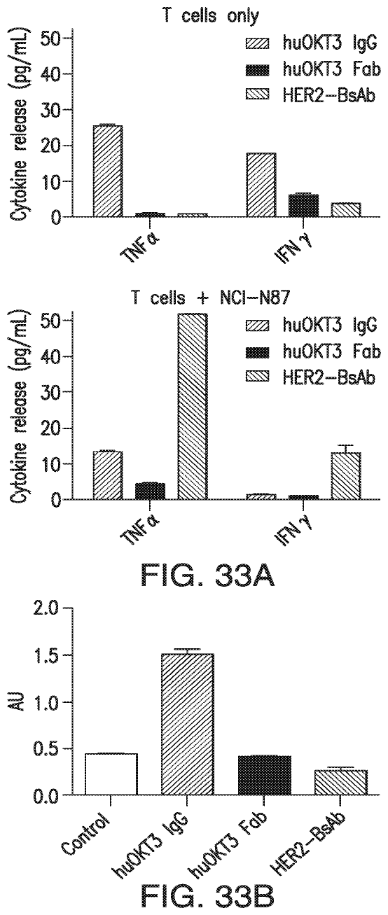

A primary goal was to build a BsAb that has the bivalent binding to tumor targets (to preserve high avidity and/or anti-proliferation capability) and the monovalent binding to CD3 on effector T cells (to minimize spontaneous T cell activation in the absence of tumor targets). A number of uniquely different bivalent formats were surveyed, including chemical conjugation (Yankelevich et al., Pediatr Blood Cancer 2012; 59:1198-205), dual-variable-domain (DVD), or attaching huOKT3 scFv to different positions in the IgG backbone (C-terminal of heavy chain or C-terminal of light chain) (Kontermann, MAbs 2012; 4), and it was found that the last option gave the best functionality. Although the HER2-BsAb has anti-CD3 scFv attached to both light chains, its reaction with CD3 on T cells was considered as functionally monovalent primarily for the following reasons. First, although the HER2-BsAb format contains two anti-CD3 scFvs positioned at the end of the light chains, these scFvs are oriented in geometrically opposed directions which restrict their ability to cooperatively bind to neighboring CD3 on T cells. This restricts the BsAb from binding bivalently and hence results in functional monovalency to CD3. It has previously been shown in a different BsAb format that geometrical restriction of two anti-CD3 scFv can result in functionally monovalent binding to T cell and lower cytokine release (Ahmed et al., OncoImmunology 2015; 4: e989776). Second, the functional consequence of bivalent binding to CD3 on T cells is the triggering of spontaneous T cell activation, hence strong cytokine release in the absence of tumor targets. As shown in FIG. 33A, HER2-BsAb only stimulated background cytokine release similar to that of the monovalent huOKT3 Fab, while bivalent huOKT3 IgG induced substantially more cytokines in the absence of tumor targets (left panel). However, in the presence of HER2 (+) NCI-N87 tumor target, anti-tumor TH1 cytokines (TNFα and IFNγ) were released but only in the presence of BsAb (right panel), a format previously shown to induce immunologic synapse formation between the T cells and tumor targets (Xu et al., Cancer Immunology Research 2015; 3:266-77). Furthermore, only bivalent huOKT3 IgG induced robust T cell proliferation, while HER2-BsAb and monovalent huOKT3 Fab had negligible effects comparable to the T cells only Control (FIG. 33B). In addition, the aglycosylation of the Fc removed both ADCC and most CMC functions, thereby further reducing cytokine release without affecting serum pharmacokinetics or compromising T cell activation.

Chimeric antigen receptor technology has rapidly accelerated investigations into HER2-directed gene modified T cells in several clinical trials: NCT00902044 (for sarcoma), NCT00889954 (all HER2 (+) cancers), NCT01109095 (GBM), NCT00924287 (metastatic cancer), and NCT01935843 (HER2 (+) solid tumors). Toxicities from off target effects were initially concerning (Morgan et al., Mol Ther 2010; 18:843-51), although subsequent patients have been safely managed pharmacologically. There, the ability of T cells to overcome low levels of antigen expression was again observed. Osteosarcoma was a good example where the expression level has been controversial (Thomas et al., Clin Cancer Res 2002; 8:788-93), and where CAR-modified T cells were highly efficient against locoregional and metastatic xenografts (Ahmed et al., Mol Ther 2009; 17:1779-87), and against osteosarcoma tumor initiating cells (Rainusso et al., Cancer Gene Ther 2012; 19:212-7). Although the successful clinical application of CAR T cells has reassured many skeptics, there remain obstacles, including the necessity of cytoreductive chemotherapy prior to T cell infusion for meaningful clinical responses, logistics of cell harvest, processing, storage, transport and product release, T cell exhaustion (Long et al., Nat Med 2015; 21:581-90) and inadequate T cell persistence after infusion.

In summary, this example demonstrates a successful IgG-scFv platform to engage T cells for HER2-directed immunotherapy. This BsAb for retargeting T cells was built with structural considerations for bivalency towards the target, and functionally monovalency towards CD3 on effector T cells, plus Fc aglycosylation for minimal spontaneous cytokine release. Its relative insensitivity to the PD-1/PD-L1 axis was novel. It has excellent anti-tumor activity both in vitro and in vivo, which is superior to trastuzumab.

### 6.4 Example 4

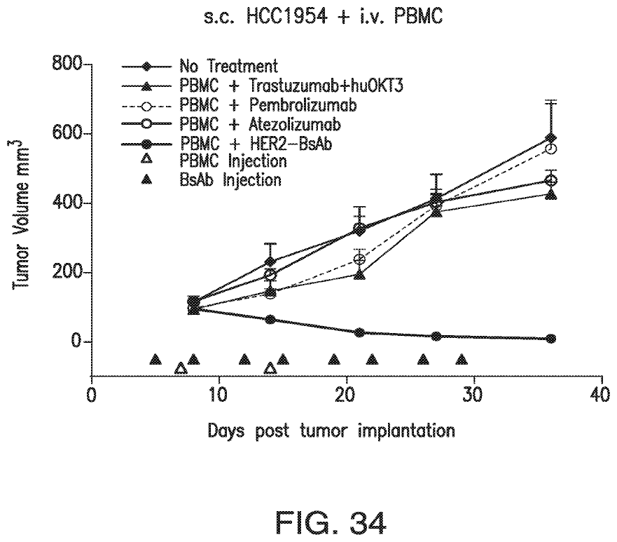

This example demonstrates that HER2-BsAb described in section 6.3.3.1 above (comprising a heavy chain consisting of the amino acid sequence set forth in SEQ ID NO: 62 and a light chain fusion polypeptide consisting of the amino acid sequence set forth in SEQ ID NO: 60) is effective against HER2 (+) breast cancer cell line xenografts that express PDL1 but are resistant to PD1 or PDL1 treatment (FIG. 34). See Section 6.3.2.8 for materials and methods. In particular, 5×10⁶ HCC1954 xenografts were implanted subcutaneously in mice and mice were treated intravenously with 7.5×10⁶ PBMC once per week for two weeks and intravenously with HER2-BsAb, anti-PD1 antibody Pembrolizumab, or anti-PDL1 antibody Atezolizumab. HER2-BsAb, anti-PD1 antibody, and anti-PDL1 antibody treatments were performed with 100 μg each, twice per week for 4 weeks. HER2-BsAb-treated tumors were completely eradicated. In contrast, there was no effect on tumors treated with PD1/PDL1 blockade (i.e., treatment with anti-PD1 antibody Pembrolizumab or anti-PDL1 antibody Atezolizumab).

### 7. Equivalents

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1-78. (canceled)

79. A method of treating a cancer that expresses a low level of HER2 in a subject in need thereof comprising administering a therapeutically effective amount of a pharmaceutical composition to the subject,
wherein the level of HER2 in the cancer is characterized as IHC 0,
wherein the pharmaceutical composition comprises T cells bound to a bispecific binding molecule comprising
a heavy chain-aglycosylated monoclonal antibody that is an immunoglobulin that binds to HER2, comprising two identical heavy chains and two identical light chains, said light chains being a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, and wherein the first and second light chain fusion polypeptides are identical,
wherein each heavy chain comprises a heavy chain variable domain (VH) present in any one of SEQ ID NOs: 23, 27, 62 or 63, and wherein each light chain comprises a light chain variable domain (V_{L}) present in SEQ ID NO: 25; and
wherein each scFv comprises a VH sequence of any one of SEQ ID NOs: 15, 17 or 64, and a VL sequence of any one of SEQ ID NOs: 16 or 65.
